(19) United States
(12) Patent Application Publication
(10) Pub. No.: US 2023/0242647 A1
(43) Pub. Date: Aug. 3, 2023

(54) ENGINEERED IMMUNOGLOBULINS

(71) Applicant: Novartis AG, Basel (CH)

(72) Inventors: John BLANKENSHIP, Acton, MA (US); Justine Celine Patricia GUYOT, Zimmersheim (FR); Brian HOLMBERG, Somerville, MA (US); Sebastien IRIGARAY, Zimmersheim (FR); Darko SKEGRO, Riehen (CH)

(21) Appl. No.: 17/997,482

(22) PCT Filed: Apr. 29, 2021
(86) PCT No.: PCT/IB2021/053582

§ 371 Date: Oct. 28, 2022

Related U.S. Application Data
(60) Provisional application No. 63/018,694, filed on May 1, 2020.

(51) Int. Cl. C07K 16/28; A61K 39/395; A61P 35/00

(52) U.S. Cl. CPC C07K 16/283; A61K 39/3955; A61P 35/00; A61K 2039/505

(57) ABSTRACT

The present invention provides engineered immunoglobulins or fragments thereof and methods for their preparation and uses. The engineered immunoglobulins have been derived from human IgG1 and engineered to confer Fc alpha receptor binding ability. In addition, the engineered IgG1 immunoglobulins or fragments thereof can retain binding to Fc gamma receptors and/or FcRn.

### SEQUENCE LISTING

The application contains a Sequence Listing which has been submitted electronically in ASCII format and is hereby incorporated by reference in its entirety. Said ASCII copy, created on Apr. 9, 2021 is named PAT058736-WO-PCT_SL.txt and is 975 Kbytes in size.

### FIELD OF THE INVENTION

The invention relates to engineered immunoglobulins and fragments thereof comprising modifications that promote recruitment of neutrophils and methods for their preparation. The engineered immunoglobulins and fragments thereof can be useful in the treatment of tumors, particularly solid tumors.

### BACKGROUND OF THE INVENTION

At present, all clinically approved antibodies comprise the immunoglobulin IgG isotype. Antibody-dependent cell-mediated cytotoxicity (ADCC) is a key mechanism for tumor cell killing mediated by IgG antibodies that recognize and bind to Fc gamma receptors (FcγR). However, in patients with a high tumor burden, relapses can occur and IgG antibody therapeutics may lose efficacy. This is primarily due to tumor and host related factors but may involve altered interaction with the target, cross talk between cell survival pathways, and involvement of anti-apoptotic proteins (Reslan et al., (2009) MABS., 1(3): 222-9). IgA represents an alternative isotype for antibody therapy by engaging Fc alpha receptors (FcαR) expressed by myeloid effector cells, such as neutrophils and tumor-resident myeloid-derived suppressor cells (MDSC). IgA is the second most abundant immunoglobulin in human serum after IgG; both monomeric IgA allotypes (IgA1 and IgA2) comprise up to 25% of human serum immunoglobulins. In the past, neutrophils were generally not considered as potential effector cells. However, neutrophils are the most abundant population of circulating white blood cells and have also been shown to infiltrate solid tumors (Gregory & Houghton (2011) Cancer Res., 71: 2411-16; Vogt Sionov et al., (2015) Cancer Microenviron., 8(3): 125-58; Uribe-Querol & Rosales (2015) J. Immunol. Res., Article ID: 983698; Rosales (2018) Front Physiol., 9: 113). MDSCs are also derived from myeloid lineages and are one of the most immunosuppressive cell types. IgA antibodies have been shown to effectively kill tumor cells by recruitment of neutrophils and MDSCs, thereby enhancing ADCC. Unfortunately, the use of IgA antibodies as therapeutics is hampered by several liabilities and limitations such as low expression yields and expensive purification schemes. In addition, the production suffers from heterogeneous glycosylation. IgA has multiple glycosylation sites that can be susceptible to glycan heterogeneity. Transient expression levels for monomeric IgA have been reported for human IgA1 at 30-70 μg/L (Lombana et al., (2019) MABS, 11: 1122-38; Meyer et al., (2016) MABS, 8: 87-98).

Therefore, there remains a need for an effective method of tumor cell killing by recruiting neutrophils to enhance ADCC. We have engineered immunoglobulin IgG Fc regions to develop an IgG that is capable of binding to FcγRs as well as to FcαRI (CD89) to effect tumor cell killing by the recruitment of neutrophils, MDSCs and enhanced ADCC.

### SUMMARY OF THE INVENTION

The present invention relates to engineered IgG1 immunoglobulins that comprise modified Fc regions such that the Fc region can bind to FcαRI (CD89). To generate IgG1 immunoglobulins with this property, a protein engineering strategy was designed to identify the specific amino acid residues and stretches of amino acid residues in an IgA immunoglobulin that are critical for binding to FcαRI. The extensive protein engineering work we performed is set out in the Examples. Initially a stepwise transfer of IgG1 constant domain to an IgA antibody was performed followed by IgG1/IgA hinge replacement. This was followed by crystal structure analysis of IgG1 and IgA1 Fc regions, which led to the identification of IgG1 residues structurally equivalent to IgA residues that were involved in IgA Fc/FcαRI interactions. The length of the IgG1 CH2/CH3 elbow was also shortened to correspond to that of IgA. The subsequent use of rational design led to modification of various amino acid residues in the CH2 and/or CH3 domains of IgG1 to substitute IgG1 residues with the corresponding residues from IgA. Semi-rational design and domain cutting were used to further refine the engineering of the IgG1 CH2 and CH3 domains.

As such, in a first aspect, the present invention provides engineered IgG1 immunoglobulins capable of recruiting FcαRI function. In the present disclosure the term “engineered IgG1 immunoglobulin” refers to a non-naturally occurring immunoglobulin of the IgG1 isotype in which at least one amino acid residue has been modified compared to the wild-type IgG1 immunoglobulin. In one embodiment, the present disclosure provides an engineered IgG1 immunoglobulin or fragment thereof comprising an Fc region comprising a first and second Fc domain wherein the first Fc domain comprises at least one amino acid modification, and wherein the first Fc domain has an amino acid sequence identity to an Fc domain from wild-type IgG1 (amino acids CH2-1.6 to CH3-125 (IMGT numbering for C-domain), equivalent to amino acids 231 to 445 (EU numbering) of SEQ ID NO: 1) of at least about 65%, and wherein the engineered IgG1 immunoglobulin or fragment thereof binds to and activates human FcαRI. For example, the first Fc domain can have an amino acid sequence identity to an Fc domain from wild-type IgG1 of at least about 65%, 70%, 75%, 80%. 90% or 95%. In one embodiment, the first Fc domain has an amino acid sequence identity to an Fc domain from wild-type IgG1 of at least about 70%. In a preferred embodiment, the first Fc domain has an amino acid sequence identity to an Fc domain from wild-type IgG1 of at least about 75%.

Binding of the engineered IgG1 immunoglobulin to FcαRI can be defined in terms of binding affinity as measured by KD, or selectivity of the engineered IgG1 immunoglobulin for FcαRI over other Fc receptors, or by competition binding to FcαRI when compared to wild-type IgG1 immunoglobulin.

Activation of an Fc receptor, e.g., a FcαRI or FcγRs, by an engineered IgG1 immunoglobulin of the present disclosure can be evaluated by measuring the level of ADCC in, e.g., a cell killing assay using effector cells such as polymorphonuclear cells (PMN) or peripheral blood mononuclear cells (PBMC). PMN can be used to characterize alpha effector function of engineered IgG1 immunoglobulins, whereas PBMC can be used to characterize gamma effector function. Cell killing assays can be run using a different cell lines including SK-BR-3, Calu-3, MDA-MB-453 or MDA-MB-175 cells.

As defined herein an Fc domain comprises a CH2 and CH3 domain. The modified first and/or modified second Fc domain can comprise modifications in the CH2 domain or in the CH3 domain or in both the CH2 and CH3 domains. The modifications can comprise an addition or insertion, a deletion or a substitution. Preferably, the modification is a substitution wherein the amino acid modification in the first Fc domain is a substitution that corresponds to an amino acid in an Fc domain of IgA, for example an Fc domain of wild-type IgA1 (SEQ ID NO: 254), an Fc domain of wild-type IgA2 (amino acids CH2-1.2 to CH3-125 of SEQ ID NO: 2 (IMGT numbering for C-domain)), an Fc domain of the m2 allotype of IgA2 (amino acids CH2-1.2 to CH3-125 of SEQ ID NO: 3 (IMGT numbering for C-domain); Lombana et al., (2019) MABS, 11: 1122-38; referred to herein as ‘parental IgA2’) or an affinity matured variant Fc domain of IgA1 or IgA2. Corresponding amino acids between two or more sequences can be determined by aligning sequences according to methods known in the art and described in detail below.

The amino acid modification(s) optionally provide one or more optimized properties relative to unmodified or wild-type Fc domains, although in some cases, the variants exhibit substantially identical biological properties to unmodified or wild-type Fc domains. Properties that may be optimized include, but are not limited to, binding to Fc receptors, e.g. FcαRI. Binding to FcαRI can be enhanced or reduced as demonstrated by an enhanced or reduced affinity for FcαRI. In one embodiment, the engineered IgG1 immunoglobulins of the present invention are optimized to possess enhanced affinity for a human FcαRI. Activation of FcαRI stimulates phagocytic or cytotoxic cells to destroy microbes, or infected cells by the mechanism of ADCC and therefore an engineered IgG1 immunoglobulin can have improved ADCC compared to a wild-type IgG1 immunoglobulin. Such optimized properties are anticipated to provide engineered IgG1 immunoglobulins or fragments thereof with enhanced therapeutic properties in humans, for example enhanced effector function and greater anti-cancer potency.

An “affinity matured variant Fc domain of IgA1 or IgA2” is defined herein as an Fc domain that includes amino acid modifications in the CH2 domain and/or CH3 domain. The amino acid modification(s) optionally provide one or more optimized properties relative to non-affinity matured variant Fc domains, although in some cases, the variants exhibit substantially identical biological properties to non-affinity matured variant Fc domains. Properties that may be optimized include, but are not limited to, binding to FcαRI. Binding to FcαRI can be enhanced or reduced as demonstrated by an enhanced or reduced affinity for FcαRI. In one embodiment, the affinity matured variant Fc domains of the present invention are optimized to possess enhanced affinity for a human FcαRI. In one embodiment, the Fc domain of the Fc variant has been affinity matured, whereby amino acid modifications have been made in the CH2 and/or CH3 domains to enhance binding of the Fc region to its target FcαRI. Such types of modifications may improve the association and/or the dissociation kinetics for binding to the target antigen. This optimized property is anticipated to provide engineered IgG1 immunoglobulins or fragments thereof with enhanced therapeutic properties in humans, for example enhanced effector function and greater anti-cancer potency.

In one embodiment, the engineered IgG1 immunoglobulin or fragment thereof comprising an Fc region that comprises modified first and second Fc domains is expressed as a homodimer, wherein the first and second Fc domains are the same. Binding affinity of the Fc region homodimer to FcαRI was measured using surface plasmon resonance and was found to be similar to the binding affinity (KD) of wild-type IgA for FcαRI. As tested herein, the binding affinity (KD) of parental IgA2 for FcαRI was found to be between about 2E-07M and about 6E-07M and the binding affinity of the engineered IgG1 immunoglobulins of the present disclosure was found to be between about 2E-10M and about 3.5E-06M (see Tables 23, 24 and 47). In an embodiment, an engineered IgG1 immunoglobulin of the present disclosure binds FcαRI with an affinity comparable to parental IgA2, or with an affinity improved by at least about 2-fold, about 3-fold, about 5-fold, about 10-fold, about 100-fold or about 1000-fold over parental IgA2.

In an embodiment of the present disclosure the engineered IgG1 immunoglobulin or fragment thereof can comprise an Fc domain selected from and comprised within the following sequences: SEQ ID Nos: 99 to 123, 146 to 149, 165 to 181. In one embodiment, the engineered IgG1 immunoglobulin or fragment thereof comprises an Fc domain comprised within SEQ ID NO: 122. In one embodiment, the engineered IgG1 immunoglobulin or fragment thereof comprises an Fc domain comprised within SEQ ID NO: 148.

Engineering of the IgG1 immunoglobulins of the present disclosure to confer FcαRI binding properties did however result in loss of binding to FcRn. Therefore studies were performed to determine the amino acid residues in the wild-type IgG1 Fc domain that were important for FcRn binding. Transfer of these residues to an IgA2 antibody was found to restore FcRn binding. In one embodiment, the present disclosure provides an engineered IgG1 immunoglobulin that binds to and activates human FcαRI and that also binds to human FcRn. In a preferred embodiment, the engineered IgG1 immunoglobulin binds to human FcRn with an affinity comparable to wild-type IgG1.

Engineering of the IgG1 immunoglobulins of the present disclosure to confer FcαRI binding properties also had an effect on the ability of the engineered IgG1 immunoglobulins to bind to FcγRs and recruit gamma effector function. Therefore studies were performed to substitute IgA residues with corresponding residues from IgG1 as well as making the additional amino acid modifications S239D and 1332E/S_CH2.3_D and I_CH2.117_E (EU/IMGT numbering for C-domain; ‘SDIE’ mutations) to the CH2 domain. In one embodiment, the present disclosure provides an engineered IgG1 immunoglobulin that binds to FcαRI, FcγRIa and FcγRIIIa. In one embodiment, the engineered IgG1 immunoglobulin or fragment thereof comprises an Fc domain comprised within SEQ ID NO: 148. In one embodiment, the engineered IgG1 immunoglobulin or fragment thereof comprises an Fc domain comprised within SEQ ID NO: 152.

The engineered IgG1 immunoglobulins of the aforementioned embodiments were constructed as homodimers. As such, it was challenging to maintain both Fcα and Fcγ effector functions to a level comparable to parental IgA or wild-type IgG1. To address this, engineered IgG1 immunoglobulins were constructed as heterodimers utilising Fc domains with different binding properties. As such, in a second aspect, the present invention provides an engineered IgG1 immunoglobulin that binds to FcαRI, FcRn and Fcγ receptors with an affinity comparable to or improved over parental IgA and wild-type IgG1. In one embodiment, the present disclosure provides an engineered IgG1 immunoglobulin comprising a first Fc domain engineered to bind to FcαRI and a second Fc domain comprising the amino acid sequence of wild-type IgG1 to bind to Fcγ receptors and FcRn. Alternatively, the second Fc domain comprises residues spatially located on top of CH2 derived from IgA, for example, packed top loops and disulfide bonds; however with this embodiment, FcγR are no longer recruited and additional mutations are desired to restore FcRn binding, such as ‘LS’ or ‘YTE’ mutations (described in more detail below).

To ensure correct heterodimer pairing upon expression of the engineered immunoglobulins, mutations were introduced to the first and second Fc domains to create a protuberance and a corresponding cavity. Such ‘knob into hole’ mutations are described in the art (Merchant et al., (1998) Nat. Biotechnol., 16: 677-681). In one embodiment, the present disclosure provides an engineered IgG1 immunoglobulin wherein the first Fc domain comprises the amino acid mutation T336W and S354C/T_CH3.22_W and S_CH3.10_C to introduce a ‘knob’, and the second Fc domain comprises the amino acid mutations Y349C, T366S, L368A and Y407V/Y_CH3.5_C, T_CH3.22_S, L_CH3.24_A and Y_CH3.86_V to introduce a ‘hole’ (EU/IMGT numbering for C-domain). In an embodiment, the first Fc domain can comprise an Fc domain comprised within SEQ ID NO: 132, 134, 136, 138, 140, 142, 144, 154, 159, 160, 161, 162, 163 or 164. In an embodiment, the second Fc domain can comprise an Fc domain comprised within SEQ ID NO: 133, 135, 137, 139, 141, 143, 145, 155, 156, 157 or 158. To further improve binding of the engineered IgG1 immunoglobulin to FcRn, additional mutations can be made to the Fc domain, for example, the ‘LS’ mutations M428L and N434S/M_CH3.107_L and N_CH3.114_S (EU/IMGT numbering for C-domain) and/or the ‘YTE’ mutations M252Y, S254T and T256E/M_CH2.15.1_Y, S_CH3.16_T and T_CH2.18_E (EU/IMGT numbering for C-domain). In an embodiment, the first Fc domain comprises the ‘LS’ mutations and can comprise an Fc domain comprised within SEQ ID NO: 154 or 162. In another embodiment, the second Fc domain comprises the ‘YTE’ mutations and can comprise an Fc domain comprised within SEQ ID NO: 163.

For the engineered IgG1 immunoglobulin of the present disclosure to function optimally, for example, to bind to FcαRI, FcRn and Fcγ receptors with an affinity comparable to or improved over parental IgA and wild-type IgG1, the amino acid modifications for introducing a ‘hole’ are made in the Fc domain comprising the amino acid modifications for binding to and activating FcαRI. In one embodiment, the present disclosure provides an engineered IgG1 immunoglobulin comprising a first Fc domain that binds to human Fcγ receptors and FcRn and comprises amino acid mutations to create a ‘knob’, and a second Fc domain that binds to human FcαRI and comprises amino acid mutations to create a ‘hole’. In another embodiment, the first Fc domain can additionally comprise the ‘LS’ mutations or ‘SDIE’ mutations or both to restore full FcγR effector function, for example, the first Fc domain can comprise an Fc domain comprised within SEQ ID NO: 161, 162 or 164.

In an embodiment of the present disclosure, the engineered IgG1 immunoglobulin comprises a first Fc domain selected from an Fc domain comprised within SEQ ID NO: 154, 159, 160, 161 or 162, and a second Fc domain selected from an Fc domain comprised within SEQ ID NO: 137 or 157. In an embodiment, the engineered IgG1 immunoglobulin comprises a first Fc domain comprised within SEQ ID NO: 137 and a second Fc domain comprised within SEQ ID NO: 154. In one embodiment, the engineered IgG1 immunoglobulin comprises a first Fc domain comprised within SEQ ID NO: 157 and a second Fc domain comprised within SEQ ID NO: 159. In an embodiment, the engineered IgG1 immunoglobulin comprises a first Fc domain comprised within SEQ ID NO: 157 and a second Fc domain comprised within SEQ ID NO: 160. In one embodiment, the engineered IgG1 immunoglobulin comprises a first Fc domain comprised within SEQ ID NO: 157 and a second Fc domain comprised within SEQ ID NO: 161. In an embodiment, the engineered IgG1 immunoglobulin comprises a first Fc domain comprised within SEQ ID NO: 157 and a second Fc domain comprised within SEQ ID NO: 162.

Binding affinity of the engineered IgG1 immunoglobulins of the present invention was determined using surface plasmon resonance (SPR). The resulting engineered immunoglobulins were generated to have the following binding properties (see Table 37):
a. FcαRI binding in a range from parental IgA2-like binding to 10-fold lower than parental IgA2;
b. FcαRI binding in a range from parental IgA2-like binding to 10-fold lower, and wild-type IgG1-like binding to Fcγ receptors;
c. FcαRI binding in a range from parental IgA2-like binding to 10-fold lower, and wild-type IgG1-like binding to FcRn;
d. FcαRI binding in a range from parental IgA2-like binding to 10-fold lower, and wild-type IgG1-like binding to Fcγ receptors and FcRn.

Binding affinity of the engineered IgG1 immunoglobulins of the present invention to FcαRI could be further enhanced by including amino acids determined by affinity maturation to contribute to improved binding. IgA2 Fc libraries were generated and screened to identify amino acid mutations that conferred enhanced FcαRI binding affinity to IgA2 variants compared to parental IgA2. These mutations improved the binding to FcαRI of more than about 225-fold when incorporated into an IgA2. These mutations were then incorporated into the engineered IgG1 immunoglobulins generated by rational design. These mutations improved the binding of these engineered IgG1 immunoglobulins to FcαRI by more than about 1200-fold. This was a significant improvement in binding to FcαRI for the IgA2 variants and it was very surprising to observe that when the mutations were incorporated into an engineered IgG1 immunoglobulin the binding to FcαRI improved again, by about 5-fold, over the IgA2 variants.

In an embodiment of the present disclosure, the engineered IgG1 immunoglobulin comprises a first Fc domain comprised within SEQ ID NO: 252, and a second Fc domain selected from an Fc domain comprised within SEQ ID NO: 159 or 161. The engineered IgG1 immunoglobulin with a first and second Fc domain from amino acid sequences SEQ ID NO: 252 and 159, respectively was preferred if gamma effector function (i.e. binding to FcγRs) was not desired. The engineered IgG1 immunoglobulin with a first and second Fc domain from amino acid sequences SEQ ID NO: 252 and 161, respectively was preferred if gamma effector function (i.e. binding to FcγRs) was desired.

In one embodiment, an engineered IgG1 immunoglobulin of the present disclosure comprises an amino acid modification at a position selected from the group consisting of: CH2.10, CH2.89, CH2.91, CH2.94, CH2.97, CH2.99, CH3.45, CH3.105, CH3.109, CH3.118 and CH3.124, wherein numbering is according to IMGT numbering for C-domain. In one embodiment, the engineered IgG1 immunoglobulin comprises an amino acid modification at positions CH2.94, CH2.97 and CH3.45. In a preferred embodiment, an engineered IgG1 immunoglobulin of the present disclosure comprises the amino acid modifications Q_CH2.94_E, L_CH2.97_Y and S_CH3.45_D. When characterised in an in vitro cell killing assay, these engineered IgG1 immunoglobulin were shown to have improved killing properties in a PMN assay compared to the parental engineered IgG1 immunoglobulin and parental IgA2.

### BRIEF DESCRIPTION OF THE FIGURES

FIG. 1: This schematic figure shows the overall strategy of the rational design used in the present application for the transfer of amino acid residues from IgA to IgG. The resulting IgG structures contain residues from IgA in both or one Fc domains of the Fc region.

FIG. 2a-g: These schematic figures depict a full length IgA with the tail piece (FIG. 2a) and without the tailpiece (FIG. 2b), the transfer of constant domains CH1 (FIG. 2c) and hinge (FIG. 2d) from IgG1 to an IgA, the position of the “CH2/CH3 elbow” region (FIG. 2e), IgG/IgA CH2/CH2 modifications (FIG. 2f) and IgG/IgA CH3/CH3 modifications (FIG. 2g).

FIG. 3a-g: These schematic figures summarize the semi-rational design used to identify amino acid residue positions involved in the IgA2/FcαRI interaction for subsequent transfer of these residues into an IgG1 Fc. Modifications were made following beta-strand scanning (FIG. 3a, FIG. 3b) and domain cutting (top/down, front/side) (FIG. 3c-3g). FIG. 3h and FIG. 3i depict modifications made to the CH2 and CH3 domains, respectively, to determine residues involved in indirect FcαRI binding.

FIG. 4: These schematic figures illustrate the positions on the engineered IgG1 immunoglobulins where modifications were made to the CH2, CH3 and CH2/CH3 elbow regions to incorporate IgA residues. FIG. 4a also comprises a hinge region from IgA, whereas FIG. 4b comprises a hinge region from IgG1. These engineered IgG1 immunoglobulins retained the FcαRI binding capacity of an IgA immunoglobulin.

FIG. 5a-b: These schematic figures illustrate the approximate position of modifications made to restore FcRn binding (FIG. 5a) and FcγR binding (FIG. 5b). In FIG. 5a, the position of the amino acids in an IgA immunoglobulin CH3 domain that were replaced with the corresponding residues from an IgG1 immunoglobulin to restore FcRn binding are shown with a star. In FIG. 5b, the position of the SDIE mutations made to the IgG1 immunoglobulin CH2 domain to restore FcγR binding are shown with a star.

FIG. 6a-e: These schematic figures summarize the engineered IgG1 heterodimeric immunoglobulins generated to have hFcαRI, FcRn and FcγR binding. All constructs have “knob into hole” modifications with the addition of LS mutations (FIGS. 6a, 6c and 6e) and/or with the addition of SDIE mutations (FIGS. 6d and 6e).

FIG. 7a-d: These schematic figures indicate the approximate positions of the mutation sets generated by affinity maturation of IgA and their application to the engineered IgG1 heterodimeric immunoglobulins. FIG. 7a shows the position of the mutations in a full-length IgA2 immunoglobulin and FIGS. 7b and 7c show incorporation of the same mutation set into an IgG1 immunoglobulin capable of binding to FcαRI. FIG. 7d schematically shows an engineered IgG1 heterodimeric immunoglobulin to which the affinity matured mutation set from IgA2 was applied.

FIG. 8: This schematic figure shows the position of the CH2/CH3 elbow region at the interface of the CH2 and CH3 domains of IgA and IgG. Dark grey=IgG (PDB 1FC1)/IgA FC (PDB 1OWO) overlapped, Light grey=IgG1 (PDB 1FC1)/IgA FC (PDB 1OWO) CH2-CH3 hinge overlapped. Arrows indicate elbows regions.

FIG. 9: This schematic figure of IgA2 Fc shows the location of the disulfide bonds and packed loops spatially located on top of IgA2 Fc.

FIG. 10: This figure shows the effect on PMN cytotoxicity in a cell killing assay of homodimer lead candidates on SK-BR-3 cells compared to parental IgA2 (SEQ ID NO: 3 (●)). The tested candidates had the following sequences: SEQ ID NO: 119 (▪), SEQ ID NO: 120 (▴), SEQ ID NO: 122 (▾) and SEQ ID NO: 123 (♦).

FIG. 11: This figure shows the effect on PMN cytotoxicity in a cell killing assay of the homodimer lead candidate with sequence SEQ ID NO: 122 (▪) on Calu-3 cells, compared to parental IgA2 (SEQ ID NO: 3 (●)).

FIG. 12: This figure shows the phagocytotic activity of the homodimer lead candidate with sequence SEQ ID NO: 122 (▴) on SK-BR-3 cells, compared to parental IgA2 (SEQ ID NO: 3 (●)) and wild-type IgG1 (SEQ ID NO: 1 (▪)).

FIG. 13a-d: This figure shows the effect on PMN cytotoxicity of homodimer lead candidates having the sequences SEQ ID NO: 148 or 152 on SK-BR-3 cells. In FIG. 13a the effect of the candidate with sequence SEQ ID NO: 148 (▾) is shown in a PMN killing assay compared to parental IgA2 (SEQ ID NO: 3 (●)). FIG. 13b shows the percentage specific killing with increasing concentration of the candidate with sequence SEQ ID NO: 148 (▪) and parental IgA2. In FIG. 13c the effect of the candidate with sequence SEQ ID NO: 152 (♦) is shown in a PMN killing assay compared to parental IgA2 (SEQ ID NO: 3 (●)). FIG. 13d shows the percentage specific killing with increasing concentration of the candidate with sequence SEQ ID NO: 152 (▪) and parental IgA2.

FIG. 14a-b: This figure shows the PMN cytotoxicity of the homodimer lead candidate having the sequence SEQ ID NO: 148 (▴) on Calu-3 cells, compared to parental IgA2 (SEQ ID NO: 3 (●)). In FIG. 14a the effect in a PMN killing assay and FIG. 14b shows the percentage specific killing with increasing concentration of the candidate or parental IgA2.

FIG. 15a-d: This figure shows the PBMC cytotoxicity of homodimer lead candidates having the sequences SEQ ID NO: 148 or 152 on SK-BR-3 cells. In FIG. 15a the effect of the candidate with sequence SEQ ID NO: 148 (▾) is shown in a PMN killing assay compared to parental IgA2 (SEQ ID NO: 3 (●)). FIG. 15b shows the percentage specific killing with increasing concentration of the candidate with sequence SEQ ID NO: 148 (▪) and parental IgA2. In FIG. 15c the effect of the candidate with sequence SEQ ID NO: 152 (♦) is shown in a PMN killing assay compared to parental IgA2 (SEQ ID NO: 3 (●)). FIG. 15d shows the percentage specific killing with increasing concentration of the candidate with sequence SEQ ID NO: 152 (▪) and parental IgA2.

FIG. 16: This figure shows the phagocytosis activity of the homodimer lead candidates with sequence SEQ ID NO: 122 (▴), SEQ ID NO: 148 (▾) and SEQ ID NO: 152 (♦) on SK-BR-3 cells, compared to parental IgA2 (SEQ ID NO: 3 (●)) and wild-type IgG1 (SEQ ID NO: 1 (▪)).

FIG. 17a-j: This figure shows the PMN cytotoxicity of five heterodimer lead candidates having the sequences SEQ ID NO: 137-154, 157-159, 157-160, 157-161, 157-162 on SK-BR-3 cells. In FIG. 17a the effect of the heterodimer candidate with sequence SEQ ID NO: 137-154 (♦) is shown in a PMN killing assay compared to parental IgA2 (SEQ ID NO: 3 (●)). FIG. 17b shows the percentage specific killing with increasing concentration of the same heterodimeric candidate (▪) and parental IgA2 (●). In FIG. 17c the effect of the heterodimeric candidate with sequence SEQ ID NO: 157-159 (▾) is shown in a PMN killing assay compared to parental IgA2 (SEQ ID NO: 3 (●)). FIG. 17d shows the percentage specific killing with increasing concentration of the same heterodimeric candidate (▪) and parental IgA2 (●). In FIG. 17e the effect of the heterodimeric candidate with sequence SEQ ID NO: 157-160 (┌) is shown in a PMN killing assay compared to parental IgA2 (SEQ ID NO: 3 (●)). FIG. 17f shows the percentage specific killing with increasing concentration of the same heterodimeric candidate (▪) and parental IgA2 (●). In FIG. 17g the effect of the heterodimeric candidate with sequence SEQ ID NO: 157-161 (▾) is shown in a PMN killing assay compared to parental IgA2 (SEQ ID NO: 3 (●)). FIG. 17h shows the percentage specific killing with increasing concentration of the same heterodimeric candidate (▪) and parental IgA2 (●). In FIG. 17i the effect of the heterodimeric candidate with sequence SEQ ID NO: 157-162 (V) is shown in a PMN killing assay compared to parental IgA2 (SEQ ID NO: 3 (●)). FIG. 17j shows the percentage specific killing with increasing concentration of the same heterodimeric candidate (▪) and parental IgA2 (●).

FIG. 18a-j: This figure shows the PBMC cytotoxicity of five heterodimer lead candidates having the sequences SEQ ID NO: 137-154, 157-159, 157-160, 157-161, 157-162 on SK-BR-3 cells. In FIG. 18a the effect of the heterodimer candidate with sequence SEQ ID NO: 137-154 (♦) is shown in a PMN killing assay compared to parental IgA2 (SEQ ID NO: 3 (●)). FIG. 18b shows the percentage specific killing with increasing concentration of the same heterodimeric candidate (▪) and parental IgA2 (●). In FIG. 18c the effect of the heterodimeric candidate with sequence SEQ ID NO: 157-159 (▾) is shown in a PMN killing assay compared to parental IgA2 (SEQ ID NO: 3 (●)). FIG. 18d shows the percentage specific killing with increasing concentration of the same heterodimeric candidate (▪) and parental IgA2 (●). In FIG. 18e the effect of the heterodimeric candidate with sequence SEQ ID NO: 157-160 (▾) is shown in a PMN killing assay compared to parental IgA2 (SEQ ID NO: 3 (●)). FIG. 18f shows the percentage specific killing with increasing concentration of the same heterodimeric candidate (▪) and parental IgA2 (●). In FIG. 18g the effect of the heterodimeric candidate with sequence SEQ ID NO: 157-161 (▾) is shown in a PMN killing assay compared to parental IgA2 (SEQ ID NO: 3 (●)). FIG. 18h shows the percentage specific killing with increasing concentration of the same heterodimeric candidate (▪) and parental IgA2 (●). In FIG. 18i the effect of the heterodimeric candidate with sequence SEQ ID NO: 157-162 (▾) is shown in a PMN killing assay compared to parental IgA2 (SEQ ID NO: 3 (●)). FIG. 18j shows the percentage specific killing with increasing concentration of the same heterodimeric candidate (▪) and parental IgA2 (●).

FIG. 19: This figure shows the phagocytosis activity of five heterodimer lead candidates having the sequences SEQ ID NO: 137-154 (●), 157-159 (▪), 157-160 (▴), 157-161 (▾) and 157-162 (♦) on SK-BR-3 cells, compared to parental IgA2 (SEQ ID NO: 3 (●)) and wild-type IgG1 (SEQ ID NO: 1 (▪)).

FIG. 20: This figure shows the PMN cytotoxicity of increasing concentrations of the homodimeric candidates with sequence: SEQ ID NOs: 122 (▪), 148 (▴), 204 (▾), 209 (♦) and 214 ( [symbol] ), and parental IgA2 (SEQ ID NO: 3 (●)) on SK-BR-3 cells, in an ADCC assay as described in Example 6. The efficacy (Emax %) for each homodimeric candidates was as follows: SEQ ID NO: 3: 25%, SEQ ID NO: 122: 32%, SEQ ID NO: 148: 27%, SEQ ID NO: 204: 28%, SEQ ID NO: 209: 35% and SEQ ID NO: 214: 34%.

FIG. 21: This figure shows the PMN cytotoxicity of increasing concentrations of the homodimeric candidates with sequence: SEQ ID NOs: 122 (▪), 148 (▴), 204 (▾), 209 (▴) and 214 ( [symbol] ), and parental IgA2 (SEQ ID NO: 3 (●)) on Calu-3 cells, in an ADCC assay as described in Example 6. The efficacy (Emax %) for each homodimeric candidates was as follows: SEQ ID NO: 3: 42%, SEQ ID NO: 122: 44%, SEQ ID NO: 148: 41%, SEQ ID NO: 204: 71%, SEQ ID NO: 209: 76% and SEQ ID NO: 214: 81%.

FIG. 22: This figure shows the PMN cytotoxicity of increasing concentrations of the homodimeric candidate with sequence SEQ ID NO: 214 ( [symbol] ) and parental IgA2 (SEQ ID NO: 3 (●)) on MDA-MB-453 cells, in an ADCC assay as described in Example 6. The EC50 value for the homodimeric candidate with sequence SEQ ID NO: 3 was 2.45 nM and the EC50 value for the homodimeric candidate with sequence SEQ ID NO: 214 was 0.36 nM.

FIG. 23 This figure shows the PMN cytotoxicity of increasing concentrations of the homodimeric candidate with sequence SEQ ID NO: 214 ( [symbol] ) and parental IgA2 (SEQ ID NO: 3 (●)) on MDA-MB-175 cells, in an ADCC assay as described in Example 6.

FIG. 24a-d: This figure shows the PMN and PBMC cytotoxicity of increasing concentrations of the heterodimeric candidates on SK-BR-3 cells, in an ADCC assay as described in Example 6. FIGS. 24a and 24b show the heterodimeric candidates with sequences SEQ ID NOs: 157-159 (▪) and 252-159 (▴), compared to parental IgA2 (SEQ ID NO: 3 (●); FIG. 24a) and wild-type IgG1 (SEQ ID NO: 1 (●); FIG. 24b), respectively. FIGS. 24c and 24d show the heterodimeric candidates with sequences SEQ ID NOs: 157-161 (▾) and 252-161 (♦) compared to parental IgA2 (SEQ ID NO: 3 (●); FIG. 24c) and wild-type IgG1 (SEQ ID NO: 1 (●); FIG. 24d), respectively.

FIG. 25: This figure shows overall improvement of thermal stability of engineered immunoglobulins compared to both IgA2 and IgG1. Individual thermograms of engineered immunoglobulins of sequence SEQ ID NO: 119, 120, 122, 123 and their parental IgG1 and IgA2 (SEQ ID NO: 1 and 2, respectively) were obtained by DSC measurement and overlapped. Arrows represent increase of CH2 melting temperature (TM) compared to CH2 TM of IgG1 or IgA2, measured as molar heat capacity (kJ/mol K).

FIG. 26: This figure shows thermal stability improvement of both CH2 and CH3 domains of engineered immunoglobulins compared to IgA2. Individual thermograms of engineered immunoglobulins of sequence SEQ ID NO: 126, 127, 128, 129 and their parental IgA2 Fc (SEQ ID NO: 125) were obtained by DSC measurement and overlapped, measured as molar heat capacity (kJ/mol K). Melting temperatures (TM) of individual CH2 and CH3 domains were extracted and presented in Table 48.

FIG. 27: This figure shows serum-time concentration profiles of IgG, IgA and engineered immunoglobulins in mice. Concentration in serum of (♦) immunoglobulin with sequence SEQ ID NO: 1 from HEK293T, (▴) immunoglobulin with sequence SEQ ID NO: 3 from HEK293T, (●) engineered immunoglobulin with sequence SEQ ID NOs:157-159 from HEK293T, (x) engineered immunoglobulin with sequence SEQ ID NOs: 252-159 from HEK293T.

FIG. 28: Serum-time concentration profiles of IgG, IgA and glyco-engineered immunoglobulins in mice. Concentration in serum of (♦) immunoglobulin with sequence SEQ ID NO: 1 from CHO-S, (▴) immunoglobulin with SEQ ID NO: 3 from CHO-S, (x) engineered immunoglobulin with sequence SEQ ID NOs: 252-159 from CHO-S, (▪) engineered immunoglobulin with sequence SEQ ID NOs: 212 from CHO-S, (∘) engineered immunoglobulin with sequence SEQ ID NO: 256 from CHO-S, (□) engineered immunoglobulin with sequence SEQ ID NO: 257 from CHO-S, (●) engineered immunoglobulin with sequence SEQ ID NO: 258 from CHO-S.

### DETAILED DESCRIPTION

Disclosed herein are engineered immunoglobulins, e.g., IgG1 or fragments thereof comprising mutated Fc regions such that the modified IgG1 binds to Fcα receptors thereby recruiting alpha effector function. The engineered immunoglobulins can also recruit IgG effector function through binding to Fcγ receptors. Furthermore, the engineered immunoglobulins can also bind to FcRn and therefore have an extended half-life.

### Definitions

In order that the present disclosure may be more readily understood, certain terms are specifically defined throughout the detailed description. Unless defined otherwise, all technical and scientific terms used herein have the same meaning as commonly understood by those of ordinary skill in the art to which this disclosure pertains.

In all cases where the term “comprise”, “comprises”, “comprising” or the like are used in reference to a sequence (e.g., an amino acid sequence), it shall be understood that said sequence may also be limited by the term “consist”, “consists”, “consisting” or the like. As used herein, the phrase “consisting essentially of” refers to the genera or species of active pharmaceutical agents included in a method or composition, as well as any excipients inactive for the intended purpose of the methods or compositions. In some aspects, the phrase “consisting essentially of” expressly excludes the inclusion of one or more additional active agents other than a multi-specific binding molecule of the present disclosure. In some aspects, the phrase “consisting essentially of” expressly excludes the inclusion of one or more additional active agents other than a multi-specific binding molecule of the present disclosure and a second co-administered agent.

As used herein, the term “antibody” refers to a polypeptide of the immunoglobulin family that is capable of binding a corresponding antigen non-covalently, reversibly, and in a specific manner. The basic functional unit of each antibody is an immunoglobulin monomer containing only one Ig unit, defined herein as an “Ig monomer”. Secreted antibodies can also be dimeric with two Ig units (e.g. IgA), tetrameric with four Ig units or pentameric with five Ig units (e.g. mammalian IgM). The term “antibody” includes, for example, a monoclonal antibody (including a full length antibody which has an immunoglobulin Fc region). The Ig monomer is a Y-shaped molecule that consists of four polypeptide chains; two identical heavy chains and two identical light chains connected by disulfide bonds (Woof & Burton (2004) Nature Reviews Immunology, 4(2): 89-99). Each chain comprises a number of structural domains containing about 70-110 amino acids that are classified into two categories: variable or constant, according to their size and function. The heavy chain comprises one variable domain (variable heavy chain domain; abbreviated as VH) and three constant domains (abbreviated as CH1, CH2 and CH3). Each light chain comprises one variable domain (abbreviated as VL) and one constant domain (abbreviated as CL). Immunoglobulin domains have a characteristic immunoglobulin fold in which two beta sheets create a ‘sandwich’ shape, held together by interactions between conserved cysteine residues and other charged amino acids. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, and FR4. The variable regions of the heavy and light chains contain an antigen binding domain or antigen binding site that interacts with an antigen.

The term “antibody” includes, but is not limited to, monoclonal antibodies, human antibodies, humanized antibodies, camelid antibodies, chimeric antibodies, and anti-idiotypic (anti-Id) antibodies (including, e.g., anti-Id antibodies to antibodies of the present disclosure). The antibodies can be of any isotype/class (e.g., IgG, IgE, IgM, IgD, IgA and IgY), or subclass (e.g., IgG1, IgG2, IgG3, IgG4, IgA1 and IgA2).

The term “monospecific molecule,” as used herein, refers to a molecule that binds to one epitope on a target antigen. In some embodiments, a mono-specific molecule of the present disclosure is a monospecific antibody-like molecule. In some embodiments, a mono-specific molecule of the present disclosure is a monospecific antibody. The term “bispecific molecule” refers to a multi-specific binding molecule that binds to two different antigens. In some embodiments, a bispecific molecule of the present disclosure is a bispecific antibody-like molecule. The term “multi-specific binding molecule” as used herein refers to a molecule that binds to two or more different antigens. Recognition of each antigen is generally accomplished via an “antigen-binding domain” In some embodiments, a multi-specific binding molecule of the present disclosure is a multi-specific antibody-like molecule, such as a bispecific antibody-like molecule.

The term “antigen-binding site” refers to the part of an antibody that comprises determinants that form an interface that binds to the antigen, or an epitope thereof. The term “antigen binding site” may be used interchangeably with the term “antigen binding domain”. With respect to proteins (or protein mimetics), the antigen-binding site typically includes one or more loops (of at least four amino acids or amino acid mimics) that form an interface that binds to the antigen polypeptide. Typically, the antigen-binding site of an antibody molecule includes at least one or two CDRs and/or hypervariable loops, or more typically at least three, four, five or six CDRs and/or hypervariable loops.

“Complementarity-determining regions” (“CDRs”) as used herein, refer to the hypervariable regions of VL and VH. The CDRs are the target protein-binding site of the antibody chains that harbors specificity for such target protein. There are three CDRs (CDR1-3, numbered sequentially from the N-terminus) in each human VL or VH, constituting in total about 15-20% of the variable domains. CDRs can be referred to by their region and order. For example, “VHCDR1” or “HCDR1” both refer to the first CDR of the heavy chain variable region. The CDRs are structurally complementary to the epitope of the target protein and are thus directly responsible for the binding specificity. The remaining stretches of the VL or VH, the so-called framework regions, exhibit less variation in amino acid sequence (Kuby (2000) Immunology, 4th ed., Chapter 4. W.H. Freeman & Co., New York). The positions of the CDRs and framework regions can be determined using various known definitions in the art, e.g., Kabat, Chothia, IMGT, AbM, and combined definitions (see, e.g., Johnson et al., (2001) Nucleic Acids Res., 29:205-206; Chothia & Lesk, (1987) J. Mol. Biol., 196:901-917; Chothia et al., (1989) Nature, 342:877-883; Chothia et al., J. Mol. Biol., (1992) 227:799-817; Lefranc, M. P., (2001) Nucleic Acids Res., 29:207-209; Al-Lazikani et al., (1987) J. Mol. Biol., 273:927-748 and Kabat et al., (1991) Sequences of proteins of immunological interest. 5th Edition—US DHHS, NIH publication n° 91-3242, pp 662, 680, 689). Definitions of antigen combining sites are also described in the following: Ruiz et al., (2000) Nucleic Acids Res., 28:219-221; MacCallum et al., (1996) J. Mol. Biol., 262:732-745; and Martin et al., (1989) PNAS. USA, 86:9268-9272; Martin et al., (1991) Methods Enzymol., 203:121-153; and Rees et al., (1996) In Sternberg M. J. E. (ed.), Protein Structure Prediction, Oxford University Press, Oxford, 141-172. In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and amino acid residues 24-34 (LCDR1), 50-56 (LCDR2), and 89-97 (LCDR3) in a VL, e.g., a mammalian VL, e.g., a human VL. Under IMGT the CDR amino acid residues in the VH are numbered approximately 26-35 (CDR1), 51-57 (CDR2) and 93-102 (CDR3), and the CDR amino acid residues in the VL are numbered approximately 27-32 (CDR1), 50-52 (CDR2), and 89-97 (CDR3) (numbering according to “Kabat”). Under IMGT, the CDR regions of an antibody can be determined using the program IMGT/DomainGap Align. IMGT tools are available at world wide web (www).imgt.org.

In an embodiment, an antibody comprises an “antigen-binding fragment” of an antibody. Examples of such fragments include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al., (1988) Science 242:423-426; and Huston et al., (1988) PNAS USA 85:5879-5883); (viii) a single domain antibody; (ix) diabodies (Dab) (bivalent and bispecific), and (x) chimeric (e.g., humanized) antibodies which may be produced by the modification of whole antibodies or those synthesized de novo using recombinant DNA technologies. These functional antibody fragments retain the ability to selectively bind with their respective antigen or receptor. These antibody fragments are obtained using conventional techniques known to those with skill in the art, and the fragments are screened for utility in the same manner as are intact antibodies.

In mammals there are two types of immunoglobulin light chain, which are called lambda (λ) and kappa (κ). Each antibody contains two light chains that are always identical; only one type of light chain, κ or λ, is present per antibody in mammals. The approximate length of a light chain is 211 to 217 amino acids and each light chain has two domains, one constant domain and one variable domain.

There are five types of mammalian Ig heavy chains denoted α, δ, ε, γ, and μ and the type of heavy chain present in the antibody defines the class or isotype of the antibody: IgM, IgG, IgA, IgD, IgE, respectively. The heavy chains vary in physiochemical, structural, and immunological properties but each heavy chain has two domains, a variable domain and a constant domain. The variable domain comprises a single Ig domain (approximately 110 amino acids long) and determines antibody binding specificity. The constant domain is identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains (Woof & Burton, supra). The term “immunoglobulin” (Ig) is used interchangeably with the term “antibody” herein. In an embodiment, a “fragment thereof” of an immunoglobulin can be an Fc region or one or more Fc domains.

IgG is the most abundant antibody isotype in the blood (plasma), accounting for 70-75% of human immunoglobulins. IgG detoxifies harmful substances and is important in the recognition of antigen-antibody complexes by leukocytes and macrophages. IgG is further divided into 4 subclasses in humans: IgG1, IgG2, IgG3 and IgG4. IgM usually circulates in the blood, accounting for about 10% of human immunoglobulins. IgM has a pentameric structure in which five basic Y-shaped molecules are linked together. B cells produce IgM first in response to microbial infection/antigen invasion. Although IgM has a lower affinity for antigens than IgG, it has higher avidity for antigens because of its pentameric/hexameric structure. IgM, by binding to the cell surface receptor, also activates cell signaling pathways. IgA is abundant in serum, nasal mucus, saliva, breast milk, and intestinal fluid, accounting for 25% of human immunoglobulins. IgA forms dimers (i.e., two IgA monomers joined together). IgA in breast milk protects the gastrointestinal tract of neonates from pathogens. IgA is divided into 2 subclasses: IgA1 and IgA2. IgD accounts for less than 1% of human immunoglobulins and may be involved in the induction of antibody production in B cells, but its exact function remains unknown. IgE is present in minute amounts, accounting for no more than 0.001% of human immunoglobulins. Its original role is to protect against parasites. In regions where parasitic infection is rare, IgE is primarily involved in allergy.

Immune cell activity is modulated by a region of an antibody known as the fragment crystallisable region or “Fc region”. The Fc Region is composed of two polypeptide chains or Fc domains, which in IgG comprises the CH2 and CH3 constant domains or ‘CH2 domain’ and ‘CH3 domain’ respectively, of the heavy chain. IgM and IgE Fc regions contain three heavy chain constant domains (CH domains 2-4) in each polypeptide chain. The amino acid residues in the CH2 and CH3 domains can be numbered according to the EU numbering system (Edelman et al., (1969) PNAS. USA, 63, 78-85), “Kabat” numbering (Kabat et al., supra) or alternatively using the IMGT numbering for C domains. IMGT tools are available at world wide web (www).imgt.org.

The Fc region binds to cell surface receptors, “Fc receptors” and complement proteins mediating physiological effects of antibodies. Fc receptors are found on may cells of the immune system including: B lymphocytes, follicular dendritic cells, natural killer cells, macrophages, neutrophils, eosinophils, basophils, human platelets and mast cells. Binding of antibody Fc region to Fc receptors stimulates phagocytic or cytotoxic cells to destroy microbes, or infected cells by the mechanism of antibody-dependent cell-mediated cytotoxicity (ADCC). There are several different types of Fc receptors (FcR), which are classified based on the type of antibody that they recognize. For example, those that bind IgG are called Fc-gamma receptors (FcγR), those that bind IgA are called Fc-alpha receptors (FcαR) and those that bind IgE are called Fc-epsilon receptors (FccR). The classes of FcRs are also distinguished by the cells that express them (macrophages, granulocytes, natural killer cells, T and B cells) and the signaling properties of each receptor (Owen J et al., (2009) Immunology (7th ed.). New York: W.H. Freeman and Company. p 423). The following table (Table 1) summarizes the different Fc receptors, their ligands, cell distribution and binding effects.

TABLE 1
Summary of Fc receptors and their properties

| | Principal | | | |
|---|---|---|---|---|
| Receptor | antibody | | | |
| name | ligand | Affinity for ligand | Cell distribution | Effect following binding to antibody |
| FcγRI | IgG1 and | High (Kd~10⁻⁹ M) | Macrophages | Phagocytosis |
| (CD64) | IgG3 | | Neutrophils | Cell activation |
| | | | Eosinophils | Activation of respiratory burst |
| | | | Dendritic cells | Induction of microbe killing |
| FcγRIIA | IgG | Low (Kd > 10⁻⁷ M) | Macrophages | Phagocytosis |
| (CD32) | | | Neutrophils | Degranulation (eosinophils) |
| | | | Eosinophils | |
| | | | Platelets | |
| | | | Langerhans cells | |
| FcγRIIB1 | IgG | Low (Kd > 10⁻⁷ M) | B Cells | No phagocytosis |
| (CD32) | | | Mast cells | Inhibition of cell activity |
| FcγRIIB2 | IgG | Low (Kd > 10⁻⁷ M) | Macrophages | Phagocytosis |
| (CD32) | | | Neutrophils | Inhibition of cell activity |
| | | | Eosinophils | |
| FcγRIIIA | IgG | Low (Kd > 10⁻⁶ M) | NK cells | Induction of antibody-dependent cell- |
| (CD16a) | | | Macrophages | mediated cytotoxicity (ADCC) |
| | | | (certain tissues) | Induction of cytokine release by |
| | | | | macrophages |
| FcγRIIIB | IgG | Low (Kd > 10⁻⁶ M) | Eosinophils | Induction of microbe killing |
| (CD16b) | | | Macrophages | |
| | | | Neutrophils | |
| | | | Mast cells | |
| | | | Follicular dendritic | |
| | | | cells | |
| FcεRI | IgE | High | Mast cells | Degranulation |
| | | (Kd~10⁻¹⁰ M) | Eosinophils | Phagocytosis |
| | | | Basophils | |
| | | | Langerhans cells | |
| | | | Monocytes | |
| FcεRII | IgE | Low (Kd > 10⁻⁷ M) | B cells | Possible adhesion molecule |
| (CD23) | | | Eosinophils | IgE transport across human intestinal |
| | | | Langerhans cells | epithelium |
| | | | | Positive-feedback mechanism to |
| | | | | enhance allergic sensitization (B cells) |
| FcαRI | IgA | Low (Kd > 10⁻⁶ M) | Monocytes | Phagocytosis |
| (CD89) | | | Macrophages | Induction of microbe killing |
| | | | Neutrophils | |
| | | | Eosinophils | |
| Fcα/μR | IgA and | High for IgM, | B cells | Endocytosis |
| | IgM | Mid for IgA | Mesangial cells | Induction of microbe killing |
| | | | Macrophages | |
| FcRn | IgG | | Monocytes | Transfers IgG from a mother to fetus |
| | | | Macrophages | through the placenta |
| | | | Dendritic cells | Transfers IgG from a mother to infant |
| | | | Epithelial cells | in milk |
| | | | Endothelial cells | Protects IgG from degradation |
| | | | Hepatocytes | |

In an embodiment, an antibody comprises a full length antibody, or a full length immunoglobulin chain. In an embodiment, an antibody comprises an antigen binding or functional fragment of a full length antibody, or a full length immunoglobulin chain. The preparation of an antibody can be monoclonal or polyclonal. An antibody can also be a human, humanized, CDR-grafted, or in vitro generated antibody.

In one embodiment, the antibody or immunoglobulin can be recombinantly produced, e.g., produced by phage display or by combinatorial methods. Phage display and combinatorial methods for generating antibodies are known in the art (as described in, e.g., Ladner et al., U.S. Pat. No. 5,223,409; Kang et al., WO 92/18619; Dower et al., WO 91/17271; Winter et al., WO 92/20791; Markland et al., WO 92/15679; Breitling et al., WO 93/01288; McCafferty et al., WO 92/01047; Garrard et al., WO 92/09690; Ladner et al., WO 90/02809; Fuchs et al., (1991) Bio/Technology, 9:1370-1372; Hay et al., (1992) Hum Antibody Hybridomas, 3:81-85; Huse et al., (1989) Science 246:1275-1281; Griffths et al., (1993) EMBO J., 12:725-734; Hawkins et al., (1992) J Mol Biol., 226:889-896; Clackson et al., (1991) Nature, 352:624-628; Gram et al., (1992) PNAS, 89:3576-3580; Garrard et al., (1991) Bio/Technology, 9:1373-1377; Hoogenboom et al., (1991) Nuc Acid Res., 19:4133-4137; and Barbas et al., (1991) PNAS, 88:7978-7982; the contents of all of which are incorporated by reference herein).

In one embodiment, the antibody or immunoglobulin is a fully human antibody (e.g., an antibody made in a mouse which has been genetically engineered to produce an antibody from a human immunoglobulin sequence or an antibody isolated from a human), or a non-human antibody, e.g., a rodent (mouse or rat), goat, primate (e.g., monkey), camel antibody. Human monoclonal antibodies can be generated using transgenic mice carrying the human immunoglobulin genes rather than the mouse system. Splenocytes from these transgenic mice immunized with the antigen of interest are used to produce hybridomas that secrete human monoclonal antibodies with specific affinities for epitopes from a human protein (see, e.g., Wood et al., WO 91/00906, Kucherlapati et al., WO 91/10741; Lonberg et al., WO 92/03918; Kay et al., WO 92/03917; Lonberg, N. et al., (1994) Nature 368:856-859; Green, L. L. et al., (1994) Nature Genet. 7:13-21; Morrison, S. L. et al., (1994) PNAS USA 81:6851-6855; Bruggeman et al., (1993) Year Immunol 7:33-40; Tuaillon et al., (1993) PNAS 90:3720-3724; Bruggeman et al., (1991) Eur J Immunol 21:1323-1326).

An antibody or immunoglobulin can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention. Chimeric antibodies can be produced by recombinant DNA techniques known in the art (see Robinson et al., WO 87/002671; Akira et al., EP184187A1; Taniguchi, M., EP171496A1; Morrison et al., EP173494A1; Neuberger et al., WO 86/01533; Cabilly et al., U.S. Pat. No. 4,816,567; Cabilly et al., EP125023A1; Better et al., (1988) Science 240:1041-1043; Liu et al., (1987) PNAS 84:3439-3443; Liu et al., (1987), J. Immunol. 139:3521-3526; Sun et al., (1987) PNAS 84:214-218; Nishimura et al., (1987), Canc. Res. 47:999-1005; Wood et al., (1985) Nature 314:446-449; and Shaw et al., (1988), J. Natl Cancer Inst. 80:1553-1559).

A humanized or CDR-grafted antibody will have at least one or two but generally all three recipient CDRs (of heavy and or light immunoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to the target antigen. Preferably, the donor will be a rodent antibody, e.g., a rat or mouse antibody, and the recipient will be a human framework or a human consensus framework. Typically, the immunoglobulin providing the CDRs is called the ‘donor’ and the immunoglobulin providing the framework is called the ‘acceptor’. In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, (1987) From Genes to Clones (Verlagsgesellschaft, Weinheim, Germany)). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art (see e.g., Morrison, S. L., (1985), Science 229:1202-1207; Oi et al., (1986), BioTechniques 4:214, and Queen et al., U.S. Pat. Nos. 5,585,089, 5,693,761 and 5,693,762, the contents of all of which are hereby incorporated by reference). Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al., (1986) Nature 321:552-525; Verhoeyan et al., (1988) Science 239:1534; Beidler et al., (1988) J. Immunol. 141:4053-4060 and Winter U.S. Pat. No. 5,225,539, the contents of all of which are hereby expressly incorporated by reference. Also within the scope of the invention are humanized antibodies in which specific amino acids have been substituted, deleted or added. Criteria for selecting amino acids from the donor are described in U.S. Pat. No. 5,585,089, e.g., columns 12-16 of U.S. Pat. No. 5,585,089, the contents of which are hereby incorporated by reference. Other techniques for humanizing antibodies are described in Padlan et al., EP 519596 A1.

Methods for altering an antibody constant region are known in the art. Antibodies with altered function, e.g., altered affinity for an effector ligand, such as FcR on a cell, or the C1 component of complement can be produced by replacing at least one amino acid residue in the constant portion of the antibody with a different residue (see e.g., EP388151A1, U.S. Pat. Nos. 5,624,821 and 5,648,260).

A “modification” or “mutation” of an amino acid residue(s)/position(s), as used herein, refers to a change of a primary amino acid sequence as compared to a starting amino acid sequence, wherein the change results from a sequence alteration involving said one or more amino acid residue/positions. For example, typical modifications include substitution of the one or more residue(s) (or at said position(s)) with another amino acid(s) (e.g., a conservative or non-conservative substitution), insertion of one or more amino acids adjacent to said one or more residue(s)/position(s), and deletion of said one or more residue(s)/position(s), inversion of said one or more residue(s)/position(s), and duplication of said one or more residue(s)/position(s).

An “amino acid substitution” or “substitution”, refers to the replacement of an one or more existing amino acid residue(s) in a predetermined (starting or parent) amino acid sequence with a one or more different amino acid residue(s). For example, the substitution 1332E refers to a variant polypeptide, in this case a constant heavy chain variant, in which the isoleucine at position 332 is replaced with glutamic acid (EU numbering). Alternatively, the position of the substitution in the CH2 or CH3 domain can be given, for example, CH2.97 indicates a substitution at position 97 in a CH2 domain with the numbering according to IMGT numbering for C-domain. The exact substitution can also be indicated by, for example, L_CH2.97_Y, which indicates that the leucine at position 97 in a CH2 domain is replaced by tyrosine.

By ‘amino acid insertion’ or ‘insertion’ as used herein is meant the addition of an amino acid at a particular position in a parent polypeptide sequence. An insertion as described herein is designated by the symbol “\hat{ }”, followed by the position, followed by the amino acid that is inserted. For example, “\hat{ }236R” designates an insertion of arginine after position 236; “\hat{ }236RR” depicts the insertion of two arginines after position 236, etc. For ease of reference, the original numbering after an insertion is not changed; therefore in a molecule containing an insertion, the amino acid normally found following the insertion site is still numbered as if the insertion did not occur, unless noted otherwise.

By “amino acid deletion” or “deletion” as used herein is meant the removal of an amino acid at a particular position in a parent polypeptide sequence. A deletion as described herein is designated by the symbol “#”, preceded by the amino acid and position that are to be deleted. For example, G237 #designates the deletion of glycine at position 237. For ease of reference, the original numbering after a deletion is not changed; therefore in a molecule containing a deletion, the amino acid normally found following the deletion site is still numbered as if the deletion did not occur, unless stated otherwise.

Generally and preferably, the modification results in alteration in at least one physicobiochemical activity of the variant polypeptide compared to a polypeptide comprising the starting (or “wild-type”) amino acid sequence. For example, in the case of an antibody or a multi-specific binding molecule, a physicobiochemical activity that is altered can be binding affinity, binding capability and/or binding effect upon a target molecule.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine (K), arginine (R), histidine (H)), acidic side chains (e.g., aspartic acid (D), glutamic acid (E)), uncharged polar side chains (e.g., glycine (G), asparagine (N), glutamine (Q), serine (S), threonine (T), tyrosine (Y), cysteine (C)), nonpolar side chains (e.g., alanine (A), valine (V), leucine (L), isoleucine (I), proline (P), phenylalanine (F), methionine (M), tryptophan (W)), beta-branched side chains (e.g., threonine (T), valine (V), isoleucine (I)) and aromatic side chains (e.g., tyrosine (Y), phenylalanine (F), tryptophan (W), histidine (H)).

The terms “percent identical” or “percent identity” in the context of two or more nucleic acids or polypeptide sequences, refers to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length. A “percentage identity” or “percentage sequence identity” of the present disclosure can be calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present disclosure, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

The term “comparison window” as used herein includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are known in the art. Optimal alignment of sequences for comparison can be conducted, e.g., by the local homology algorithm of Smith & Waterman (1970) Adv. Appl. Math. 2:482c, by the homology alignment algorithm of Needleman & Wunsch (1970) J. Mol. Biol., 48: 443, by the search for similarity method of Pearson & Lipman (1988) PNAS. USA, 85: 2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology).

Two examples of algorithms that are suitable for determining percent sequence identity and sequence similarity are the BLAST and BLAST 2.0 algorithms, which are described in Altschul et al., (1977) Nuc. Acids Res., 25: 3389-3402; and Altschul et al., (1990) J. Mol. Biol., 215: 403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighbourhood word score threshold (Altschul et al., (1990) supra). These initial neighbourhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff & Henikoff, (1989) PNAS. USA, 89: 10915) alignments (B) of 50, expectation (E) of 10, M=5, N=−4, and a comparison of both strands.

The BLAST algorithm also performs a statistical analysis of the similarity between two sequences (see, e.g., Karlin & Altschul (1993) PNAS. USA, 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller (Comput. Appl. Biosci. 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman & Wunsch supra algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

Other than percentage of sequence identity noted above, another indication that two nucleic acid sequences or polypeptides are substantially identical is that the polypeptide encoded by the first nucleic acid is immunologically cross reactive with antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., (1991) Nucleic Acid Res., 19: 5081; Ohtsuka et al., (1985) J Biol Chem., 260: 2605-2608; and Rossolini et al., (1994) Mol Cell Probes, 8: 91-98). As used herein, the term, “optimized nucleotide sequence” means that the nucleotide sequence has been altered to encode an amino acid sequence using codons that are preferred in the production cell, in this case a Chinese Hamster Ovary cell (CHO). The optimized nucleotide sequence is engineered to retain completely the amino acid sequence originally encoded by the starting nucleotide sequence, which is also known as the “parental” sequence. In particular embodiments, the optimized sequences herein have been engineered to have codons that are preferred in CHO mammalian cells.

As used herein, “C-terminus” refers to the carboxyl terminal amino acid of a polypeptide chain having a free carboxyl group (—COOH). As used herein, “N-terminus” refers to the amino terminal amino acid of a polypeptide chain having a free amine group (—NH₂).

The term “operably linked” or “functionally linked”, as used herein, refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The terms “polypeptide” and “protein” are used interchangeably herein to refer to a polymer of amino acid residues. The phrases also apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The term “in vivo half-life”, as used herein, refers to the half-life of the molecule of interest or variants thereof circulating in the blood of a given mammal.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

As used herein, phrases such as “a patient in need of treatment” or “a subject in need of treatment” includes subjects, such as mammalian subjects, that would benefit from administration of molecule or pharmaceutical composition of the present disclosure used, e.g., for detection, for a diagnostic procedure and/or for treatment.

As used herein, the terms term “treatment” or “treat” is herein defined as the application or administration of a multi-specific binding molecule according to the disclosure, or a pharmaceutical composition comprising said multi-specific binding molecule, to a subject or to an isolated tissue or cell line from a subject, where the subject has a particular disease (e.g., arthritis), a symptom associated with the disease, or a predisposition towards development of the disease (if applicable), where the purpose is to cure (if applicable), prevent (if applicable), delay the onset of, reduce the severity of, alleviate, ameliorate one or more symptoms of the disease, improve the disease, reduce or improve any associated symptoms of the disease or the predisposition toward the development of the disease. The term “treatment” or “treat” includes treating a patient suspected to have the disease as well as patients who are ill or who have been diagnosed as suffering from the disease or medical condition, and includes suppression of clinical relapse. The phrase “reducing the likelihood” refers to delaying the onset or development or progression of a disease, infection or disorder.

The term “therapeutically acceptable amount” or “therapeutically effective amount” or “therapeutically effective dose” interchangeably refer to an amount sufficient to effect the desired result (i.e., a reduction disease activity, reduction in disease progression, reduction in disease signs and/or symptoms, etc.). In some aspects, a therapeutically acceptable amount does not induce or cause undesirable side effects. A therapeutically acceptable amount can be determined by first administering a low dose, and then incrementally increasing that dose until the desired effect is achieved. A “prophylactically effective dosage” and a “therapeutically effective dosage” of the molecules of the present disclosure can prevent the onset of (if applicable), or result in a decrease in severity of, respectively, disease symptoms.

As used herein, “selecting” and “selected” in reference to a patient is used to mean that a particular patient is specifically chosen from a larger group of patients due to the particular patient having a predetermined criterion. Similarly, “selectively treating a patient” refers to providing treatment to a patient that is specifically chosen from a larger group of patients due to the particular patient having a predetermined criteria. Similarly, “selectively administering” refers to administering a drug to a patient that is specifically chosen from a larger group of patients due to the particular patient having a predetermined criterion.

Unless otherwise specifically stated or clear from context, as used herein, the term “about” in relation to a numerical value is understood as being within the normal tolerance in the art, e.g., within two standard deviations of the mean. Thus, “about” can be within +/−10%, 9%, 8%, 7%, 6%, 5%, 4%, 3%, 2%, 1%, 0.1%, 0.05%, or 0.01% of the stated value, preferably +/−10% of the stated value. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

The word “substantially” does not exclude “completely,” e.g., a composition which is “substantially free” from Y may be completely free from Y. Where necessary, the word “substantially” may be omitted from the definition of the disclosure.

The term “co-administer” refers to the simultaneous presence of two active agents in the blood of an individual. Active agents (e.g., additional therapeutic agents) that are co-administered with the disclosed antibodies and antigen-binding fragments can be concurrently or sequentially delivered.

Various aspects of the disclosure are described in further detail in the following sections and subsections.

Engineered Immunoglobulins

Besides the ability of antibodies to bind antigen, an important feature of antibodies is their ability to recruit immune effector function. Engagement of the humoral immune response is mainly governed by interactions with Clq and the initiation of the complement cascade (Meyer et al., (2014) MABS, 6(5):1133-44). The cellular immune response occurs mostly due to the interactions between the antibody and Fc gamma receptors (FcγRs). There are five activating FcγRs: the high affinity FcγRI (CD64) that can bind to monovalent antibody, and the lower affinity FcγRII*a and IIc (CD32), and FcγRIIIa (CD16a) and IIIb (CD16b) that require avidity-based interactions. There is one inhibitory receptor: FcγRIIb (CD32). Intracellular signalling through the activating receptors is modulated through the phosphorylation of immunoreceptor tyrosine-based activation motifs (ITAMs), which leads to effector functions such as antibody-dependent cell-mediated cytotoxicity (ADCC), antibody-dependent cellular phagocytosis (ADCP), and inflammation via the induction of cytokine secretion. In contrast, intracellular signalling through the inhibitory FcγRIIb is modulated through the phosphorylation of immunoreceptor tyrosine-based inhibitory motifs (ITIMs), which recruit phosphatases that counter-balance activating signalling pathways (Nimmerjahn & Ravetch, (2008) Nat Rev Immunol., 8: 34-47). Antibody interactions with FcγRs and Clq are dependent on the hinge and proximal CH2 amino acid sequence as well as glycosylation in the CH2 region (Edelman et al., (1969) PNAS USA, 63: 78-85).

Although many antibody-based therapies have shown clinical and commercial success, these therapeutics are often only effective in a subset of patients. To date, all commercial antibodies are of the IgG class, predominantly IgG1, that recruit immune effector function via FcγRIII (CD16). The typical reaction involves activation of natural killer (NK) cells that display FcγRIII on their cell surface by the Fc portion of an IgG antibody triggering ADCC. However NK cells are only one component of the innate immune system and activation of other forms of leukocytes could be used to enhance the ADCC response triggered by IgG. The IgA class of antibodies engage FcαRI, which is widely expressed on neutrophils. Neutrophils comprise the highest percentage of innate effector cells found in the circulation and their activation triggers both ADCC and ADCP. In addition they have been shown to infiltrate many solid tumors (Gregory & Houghton (2011) supra). However, since IgG antibodies do not bind to the FcαRI, most commercial antibody-based therapeutics cannot activate neutrophils. IgA based therapeutic antibodies have not been developed commercially due to various drawbacks and these class of antibodies also lack the favourable therapeutic properties of IgG antibodies. For example, The hinge region of IgA is heavily glycosylated, and producing proteins with multiple complex glycans can be problematic for bioprocessing and quality control during drug development (Woof & Kerr (2006) J. Pathol., 208: 270-82), IgA does not bind to C1q and therefore it cannot mediate CDC through the classical complement pathway (van Egmond et al., (2001) Trends Immunol., 22: 205-11), IgA also does not bind to Fcγ receptors and hence cannot take advantage of the diverse mechanisms for immune cell activation that are relevant to IgG1 therapeutics and IgA exhibits a shorter circulation half-life than IgG because of a lack of binding to the FcRn receptor, which is important for intracellular recycling. Combination therapy of IgG with IgA antibodies has not been developed due to regulatory and cost concerns and potential unwanted therapeutic effects.

Since the immunoglobulin-like domains of the human IgG and IgA classes share a high degree of structural homology, a number of groups have examined the possibility of transfer of properties of one class to the other. For example, single domains of IgA2 were appended to the end of the gamma 1 constant region creating a four-domain constant region (CH_{1g}-CH_{2g}-CH_{3g}-CH₃ₐ) (Chintalacharuvu et al., (2001) Clin Immunol., 101: 21-31) in an attempt to engage FcγRs and FcαRI. To make the constant region more similar to the alpha constant region the CH1 domain of gamma 1 was substituted for the alpha 1 constant region domain (CH₁ₐ-CH_{2g}-CH_{3g}-CH₃ₐ). These four-domain cross-isotype IgG/A chimeric antibodies form polymers with the J chain in a manner similar to natural IgA2 but had reduced transport by polymeric Ig receptors. The four-domain, cross-isotype antibodies were capable of mediating complement-dependent lysis of sheep red blood cells and appeared to be more pH-resistant than IgG1; however they had a 3-fold to 5-fold decrease in FcγRI affinity and possessed the short serum half-life of IgA2 (Chintalacharuvu et al., supra).

A Tandem IgG1/IgA2 antibody format was generated by Borrok et al whereby the Fc region of a trastuzumab binding IgG1 antibody was replaced with the Fc region of an IgA2 or the IgA2 Fc region was appended to the C-terminus of the full length IgG1 antibody (Borrok et al., (2015) MABS, 7(4): 743-51). These constructs exhibited enhanced ADCC and ADCP capabilities, with the IgG1/IgA2 tandem Fc format retaining IgG1 FcγR binding plus FcRn-mediated serum persistence, as well as having myeloid cell-mediated effector functions via FcαRI/IgA Fc interactions. Anti-human epidermal growth factor receptor-2 antibodies with the tandem IgG1/IgA2 Fc were shown to better recruit and engage cytotoxic PMN cells than either the parental IgG1 or IgA2. Pharmacokinetics of IgG1/IgA2 in BALB/c mice are similar to the parental IgG and far surpass the poor serum persistence of IgA2.

Early structural biology work was used to determine which part of the Fc domain of an IgG1 interacts with the Fcγ receptor (Woof et al., (1986) Molecular Immunology, 23(3): 319-330) and which part of the Fc domain of an IgA interacts with the Fcα receptor (Woof et al., (2011) Mucosal Immunology, 4(6): 590-7). With this knowledge, immunoglobulins have been generated that bind an Fcα receptor and an Fcγ receptor. For example, antigen-binding proteins with modified heavy chains were constructed in which a CH3 domain of the Fc region of a modified heavy chain was of the IgG1 isotype and a CH3 domain of the Fc region of another modified heavy chain was of the IgA isotype (WO 12/116926 A1, Bossenmaier & Kettenberger). A chimeric IgG-IgA antibody termed ‘cross-isotype’ antibody was described by Kelton et al, whereby the Fc region of the antibody was engineered to comprises a chimeric IgG CH2 domain having an α1 and/or α2 loop from IgA and a CH3 domain from IgA. FcRn binding activity could also be conferred to the chimeric molecule by adding a FcRn binding peptide to the C-terminus of the chimeric antibody (Kelton et al., (2014) Chem. Biol., 21(12): 1603-9; WO 14/065945 A1).

Whilst the above described chimeric IgG/IgA immunoglobulins do have the property of binding to both Fcα and Fcγ receptors; the Fc region still comprises a high proportion of IgA amino acid residues as it contains complete IgA Fc domains (e.g. CH2 and or CH3 domains) and therefore the Fc domain has greater than 50% IgA residues (WO 14/065945 A1). Therefore, these chimeric proteins are subject to the same drawbacks that have hampered the production of therapeutic IgA antibodies as discussed above, namely poor development properties. The chimeric proteins are also associated with lower affinity measured by SPR for FcγRs and for the FcαRI compared to wild-type, full length IgG1 and IgA2, respectively (Jung et al., (2010) PNAS USA., 107(2): 604-9; WO 14/065945 A1 (pages 27-28)).

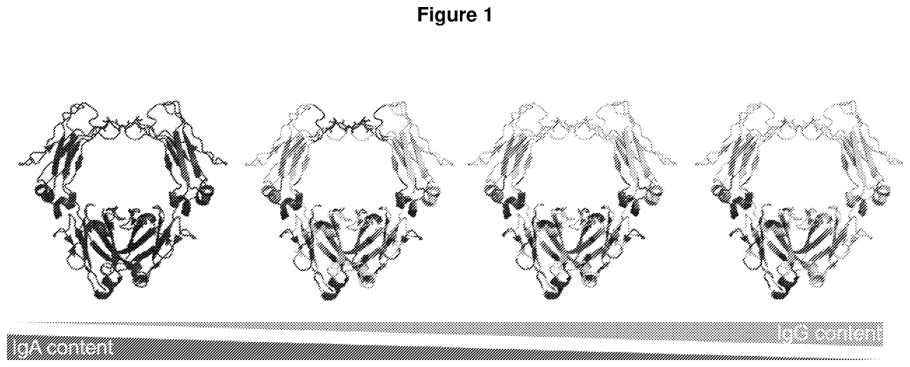

In contrast, in the present invention, we have engineered an immunoglobulin Fc region of the IgG1 isotype that confers IgA effector function but with a minimal number of amino acid modifications. This is shown schematically in FIG. 1. Surprisingly, the desirable expression and manufacturing properties of an IgG1 were maintained with the additional benefit of neutrophil activation. Using rational design, the CH2 and CH3 domains of human IgA2 and human IgG1 were superimposed and length adjusted to determine the amino acid residues for modification. Using this method, the hIgA2 Fc/FcαRI interface was transferred into hIgG1 Fc, creating an IgG1-like molecule recruiting Fcα effector function. In addition, transfer of alpha effector functions was combined with further modifications to the Fc region to restore the capacity to bind FcRn, thereby extending half-life. In addition, to restore IgG effector function, the Fc region was further modified to introduce modifications that resulted in FcγR binding. An anti-HER2 binding antibody comprising the modified Fc sequences was used to exemplify the protein engineering with homodimeric and heterodimeric IgG1-like molecules generated.

1. Engineered Homodimeric Immunoglobulins Recruiting FcαR Function

In a first aspect, the generation of engineered immunoglobulins of the human IgG1 isotype that recruit FcαR function was achieved by the transfer of human IgA2 Fc residues that interface with the human FcαRI to the Fc region of an IgG1 immunoglobulin using both rational and semi-rational design. As IgA naturally exists in a number of different forms (monomeric, dimeric, secretory), initial modifications to the IgA2 structure were made to generate a monomeric IgA2, which was the preferred form for the protein engineering. The tailpiece was removed from the CH3 domain and the proline residue at position 124 (IMGT numbering for C-domain) in the CH1 domain was substituted with arginine to reduce IgA2 heterogeneity. These modifications were followed by replacing the CH1 domain and hinge region of the IgA2 construct with the corresponding CH1 domain and hinge region from IgG1. These replacements had limited impact on FcαRI binding (see Example 1).

In silico analysis was then used to superimpose the Fc regions of IgG1 and IgA1 to identify the IgG1 residues that were structurally equivalent to IgA1 residues involved in the interaction of IgA1 with the FcαRI. The crystal structure of IgA2 Fc is not yet known; however the Fc regions of IgA1 and IgA2 are structurally very similar since they share 96.2% of identity/98.6% of similarity (alignment of CH2-CH3 sequences comprised in SEQ ID NO: 2 (amino acids CH2-1.2 to CH3-125 (IMGT numbering for C-domain) and SEQ ID NO: 254. These identified residues were then replaced in the IgG1 construct with the equivalent IgA2 residues. Whilst IgG1 and IgA2 share structural homology, the sequence length and angles between the CH2 and CH3 domains are different. This CH2-CH3 interface, comprising approximately the last three residues of the CH2 domain and the first six residues of the CH3 domain of IgA2, and termed herein as the “CH2-CH3 elbow”, was modified in IgG1 to shorten the lengths of the CH2 and CH3 domains in this region, to achieve a similar angle between these domains in the IgG1 3D structure as is found in IgA2. The beta-sheet structure of the IgA2 construct was then examined in the context of binding to FcαRI. One side of the sheet turns residue side chains towards FcαRI and the other side turns residue side chains towards the CH3-CH3 core interface. The CH3-CH3 core residues can affect the positioning of residue side chains in their interaction with FcαRI depending on their properties and steric hindrance. Since the beta sheets of the CH2 and CH3 domains of IgA2 and IgG1 share a high degree of structural homology, it was therefore possible to exchange the CH3 residues at the CH3-CH3 interface in the IgG1 construct with the corresponding residues from IgA2 CH3 to correctly orientate the residues interacting with FcαRI.

The use of rational design as described above, enabled the identification of important IgA2 residues and their subsequent transfer to an IgG1 construct. However, to fully complete the engineering campaign, a semi-rational approach was then performed to fine tune the CH2 and CH3 engineering to improve binding to FcαRI. As mentioned above, the beta sheets of IgG1 and IgA2 share a high degree of structural homology. The anti-parallel β-strands A, B, C, D, E, F and G (IMGT nomenclature) of IgA2 CH2 and CH3 were individually scanned by sequential substitution with the equivalent IgG1 β-strands. Following this, the β-strands of IgG1 were replaced two-by-two and three-by-three etc. with the equivalent IgA2 β-strand depending on their role in the IgA2-FcαRI interaction.

The IgG1 and IgA2 CH2 and CH3 domains can effectively be described as ‘building blocks’, and it is therefore possible to cut these domains into pieces. Two different types of sections were cut following the transverse plane (top-down section) and then the frontal plane (front-side section). This gave constructs that contained IgG1/IgA2 hybrid CH2 or CH3 domains comprising 50% IgA2 and 50% IgG1.

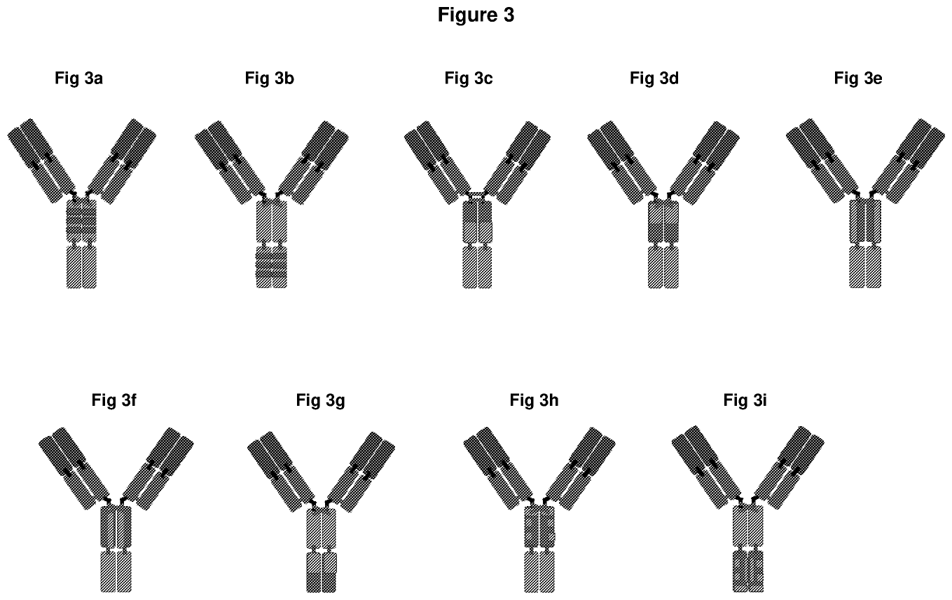

Semi-rational design was also used to determine which parts of the IgA2 Fc region are necessary for its interaction with the FcαRI, despite an apparent unrelated role or location at a long distance from the FcαRI interface. It was found that disulfide bonds and loops that connect the beta sheets, that are spatially located on top of IgA2 CH2 (see FIG. 3) play an important role in the correct positioning of CH2 residues at the FcαRI binding site. In addition, the α-helices located in CH3 that are solvent exposed were also shown to have an impact on IgA2-FcαRI interactions. It was surprising to discover the importance of these regions in FcαRI interactions given their distance from the IgA2-FcαRI binding site.

The work described above, enabled the generation of an engineered homodimeric immunoglobulins comprising an IgG1 Fc region in which a number of residues were substituted with IgA residues to confer FcαRI binding properties. In one embodiment, the present disclosure provides an engineered IgG1 immunoglobulin comprising a number of substitutions wherein the IgG1 immunoglobulin is capable of recognizing and binding to FcαRI. In one embodiment, the present disclosure provides an engineered IgG1 immunoglobulin wherein less than 35% of IgG1 residues in the Fc domain are replaced with the corresponding residue from IgA. In one embodiment, the present disclosure provides an engineered IgG1 immunoglobulin wherein less than about 35%, 30%, 25%, 20%, 15%, 10% of residues in the Fc domain are replaced with corresponding residues from IgA. In one embodiment, the present disclosure provides an engineered IgG1 immunoglobulin comprising a modified Fc domain wherein residues in the Fc domain have been replaced with corresponding residues from IgA, and wherein the modified Fc domain has an amino acid sequence identity to an Fc domain from wild-type IgG1 (SEQ ID NO:1) of at least about 65%, 70%, 75%, 80%. 90% or 95%. In one embodiment, the Fc domain has an amino acid sequence identity to an Fc domain from wild-type IgG1 of at least 70%. In a preferred embodiment, the Fc domain has an amino acid sequence identity to an Fc domain from wild-type IgG1 of at least 75%

In one embodiment, the present disclosure provides an engineered IgG1 immunoglobulin comprising a number of substitutions at certain positions in the Fc domain. The numbering of all substitution positions is according to IMGT numbering for C-domain. In one embodiment, the present disclosure provides an Fc domain wherein the amino acids at positions in the CH2 domain, for example, as listed in Table 6, Table 10, Table 14 and Table 18, have been substituted with the corresponding amino acid from IgA2. In one embodiment, the present disclosure provides an Fc domain wherein the amino acids at positions in the CH3 domain, for example, as listed in Table 8, Table 12, Table 16 and Table 20, have been substituted with the corresponding amino acid from IgA2. In one embodiment, the present disclosure provides an engineered IgG1 immunoglobulin wherein the amino acid at positions in the CH2 and CH3 domains of the Fc domain, for example, as listed in Table 22, have been substituted with the corresponding amino acid from IgA2.

The modified Fc domains were incorporated into a full-length anti-HER2 antibody (VH-CH 1-hinge of amino acid sequence SEQ ID NO: 1 and light chain of SEQ ID NO: 124) and expressed in a mammalian HEK cell line. The binding affinity of the expressed immunoglobulin variants was determined using surface plasmon resonance using a BIAcore® T200 instrument (GE Healthcare). In one embodiment, the present disclosure provides an engineered IgG1 immunoglobulin comprising amino acid substitutions in the CH2 domain, wherein the amino acids have been substituted with the corresponding amino acid from IgA2, and wherein the engineered immunoglobulin binds to human FcαRI with a binding affinity as listed in Table 7, Table 11, Table 15 and Table 19. In one embodiment, the present disclosure provides an engineered IgG1 immunoglobulin comprising amino acid substitutions in the CH3 domain, wherein the amino acids have been substituted with the corresponding amino acid from IgA2, and wherein the engineered immunoglobulin binds to human FcαRI with a binding affinity as listed in Table 9, Table 13, Table 17 and Table 21. In one embodiment, the present disclosure provides an engineered IgG1 immunoglobulin comprising amino acid substitutions in the CH2 and CH3 domains of the Fc domain, wherein the amino acids have been substituted with the corresponding amino acid from IgA2, and wherein the engineered immunoglobulin binds to human FcαRI with a binding affinity as listed in Table 23 and Table 24.

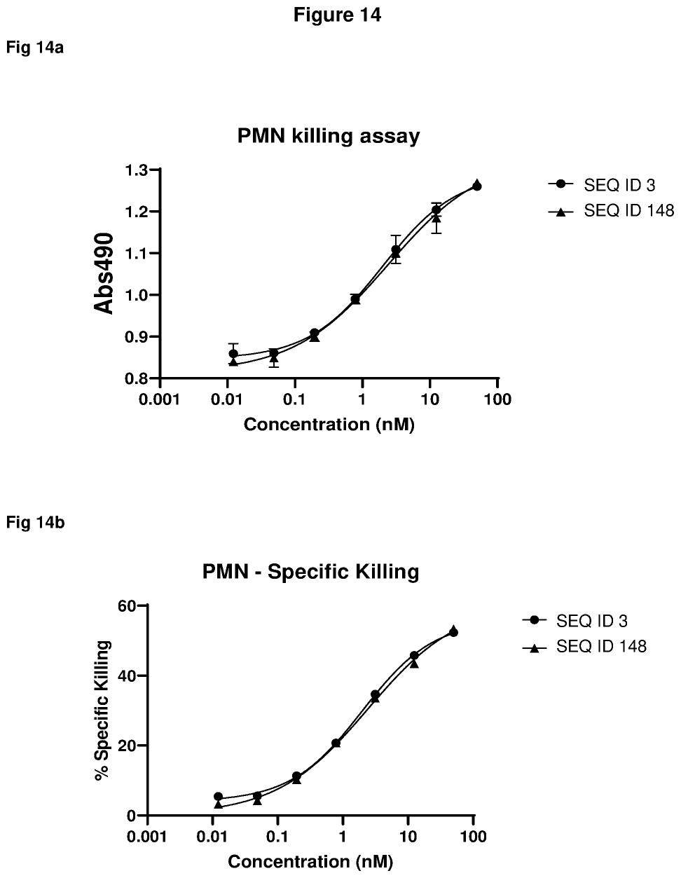

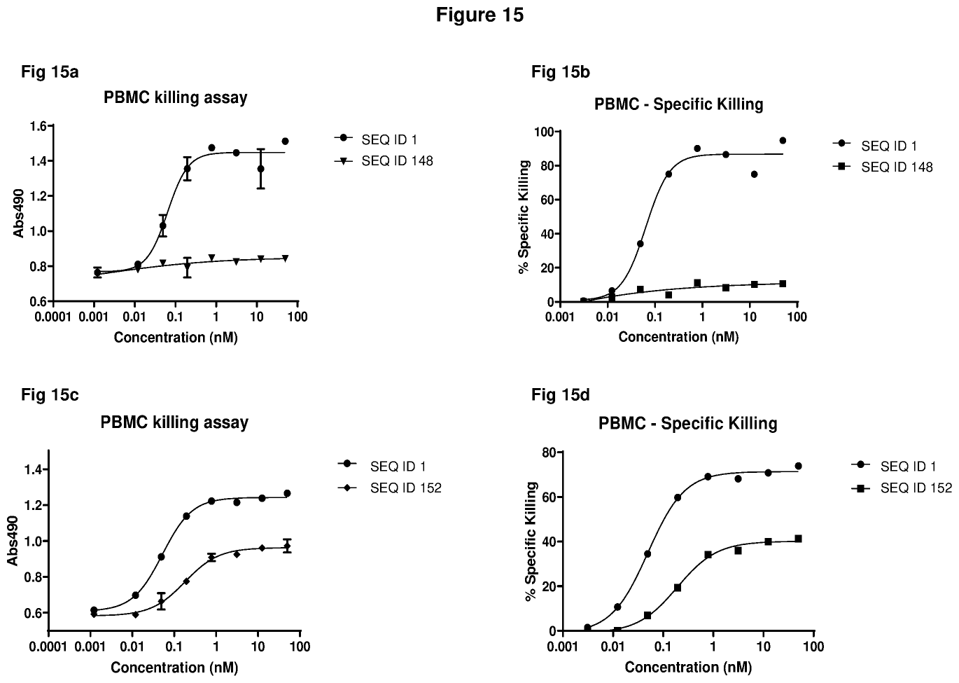

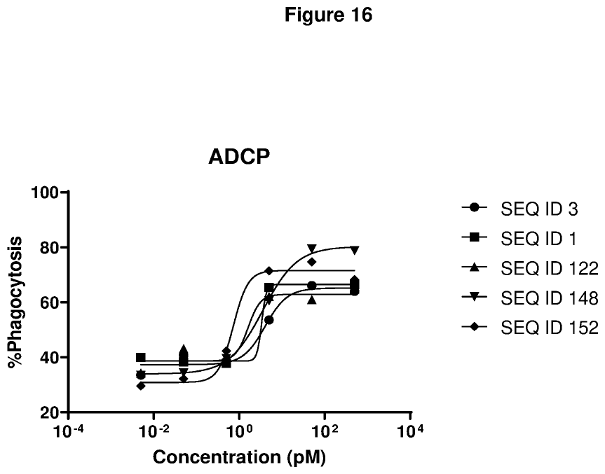

A selection of the engineered IgG1 immunoglobulins with modifications to the CH2 and CH3 domains of the Fc domain were tested in ADCC and ADCP cell killing assays following the procedure described in Examples 6 and 7, respectively. The results are shown in FIG. 10, FIG. 11 and FIG. 12 and indicate that the engineered IgG1 immunoglobulins performed as well as the wild-type immunoglobulins.

As described above we have generated engineered immunoglobulins in which the FcαRI binding properties of an IgA2 immunoglobulin have been transferred to an IgG1 immunoglobulin. However, the substitution of amino acids in the Fc region of IgG1 resulted in the loss of the constitutive FcRn binding properties with a resulting decrease in half-life of the IgG1 immunoglobulin. The critical residues for FcRn binding were identified and the CH3 substitutions A_CH3.15_H, F_CH3.116_Y or P_CH3.113_H, L_CH3.114_N, A_CH3.115_H, F_CH3.116_Y (IMGT numbering for C-domain) were made in an IgA2 immunoglobulin. With these mutations, FcRn binding was restored; however since key residues of IgG1 CH3 that interact with FcRn are located in the corresponding region of IgA2 CH3 that is responsible for FcαRI interactions it was not possible to engineer these residues back to those from IgG1 without losing some of the FcαRI binding as shown in Table 25.

Furthermore, the substitution of amino acids in the Fc region of IgG1 to confer FcαRI binding resulted in the loss of the constitutive FcγR binding properties of the engineered IgG1 immunoglobulin. Amino acid modifications were introduced in the CH2 domain of the engineered IgG1 immunoglobulin to restore FcγR effector function. Mutations to the CH2 domain, for example the ‘SDIE’ mutation of S239D and 1332E/S_CH2.3_D and I_CH2.117_E or the triple mutation S239D, 1332E and A330L/S_CH2.3_D, l_CH2.117_E and A_CH2.115_L (EU/IMGT numbering for C-domain; Lazar et al (2006) PNAS USA 103(11): 4005-10) have been described for improving FcγR effector function. In an embodiment of the present disclosure, the SDIE mutation was made in the CH2 domain of an engineered IgG1 immunoglobulin comprising mutations to confer binding to FcαRI, as set out in Table 26. In combination, these mutations were sufficient to partially restore FcγR effector function.

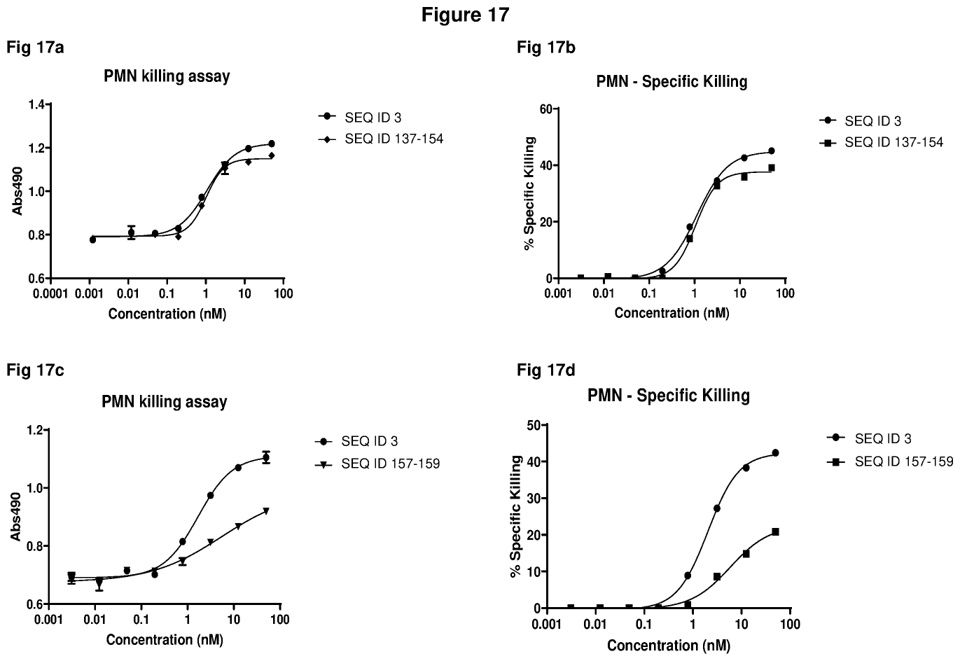

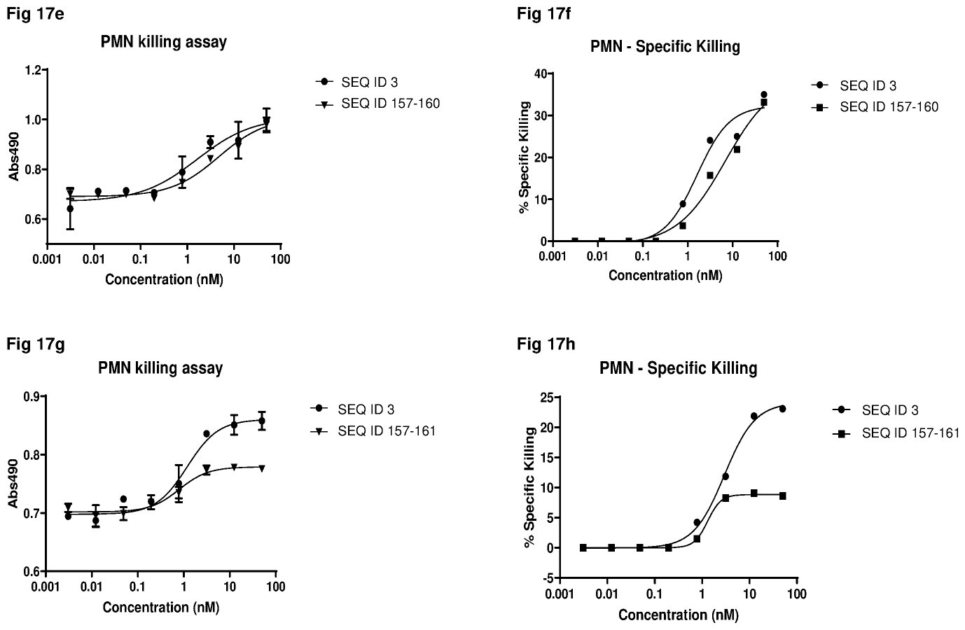

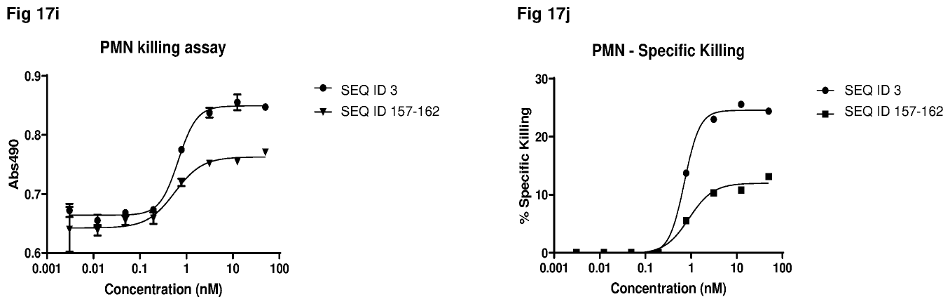

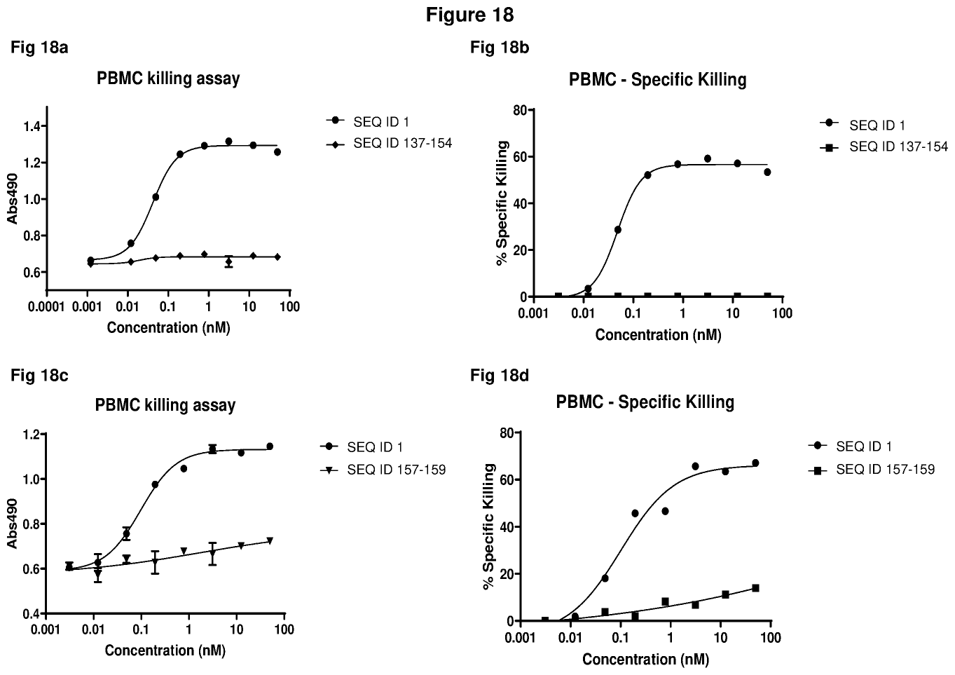

The binding affinity of the engineered IgG1 immunoglobulins comprising amino acid modifications to restore binding to human FcαRI and binding to human FcγRI and FcγRIIIa was determined by SPR. In one embodiment, the present disclosure provides an engineered IgG1 immunoglobulin comprising amino acid substitutions in the Fc domain, wherein the engineered immunoglobulin binds to human FcαRI, FcγRI and FcγRIIIa with a binding affinity as listed in Table 27, Table 28 and Table 29, respectively. Effector function of a number of engineered IgG1 immunoglobulins was tested in an in vitro PMN/PBMC killing assay and an ADCP assay with the results shown in FIGS. 13-16.

2. Engineered Heterodimeric Immunoglobulins Recruiting FcαR Function, FcγR Function and FcRn Binding

The protein engineering work described above resulted in the generation of engineered homodimeric immunoglobulins with the properties of binding FcαRs, FcγRs and FcRn. However, binding to one class of Fc receptors was often at the expense of binding to another class and further optimization work was required. Therefore, to generate an optimized engineered immunoglobulin, a heterodimeric Fc region was generated in which a first Fc domain of an IgG1 Fc region was engineered to recruit FcαRI and a second Fc domain of an IgG1 Fc region remained unchanged. A second aspect of the present invention provides an engineered IgG1 immunoglobulin comprising a heterodimeric Fc region that can recruit both FcαRI and FcγR function and bind to FcRn. When residues spatially located on top of CH2 of the engineered immunoglobulin were derived from IgG1, no further amino acid modifications were required in the heterodimeric Fc region to restore FcRn binding. Therefore, in one embodiment, an engineered IgG1 immunoglobulin is provided comprising residues from IgG1 spatially located on top of CH2. In another embodiment, to fully restore FcγR effector function, the double mutation S239D/I332E (EU numbering)/S_CH2.3_D/I_CH2.117_E (IMGT numbering for C-domain) was made in the CH2 domain of the second Fc domain derived from IgG1. In another embodiment, when residues spatially located on top of the CH2 were derived from IgA2, for example, packed loops and disulfide bonds, the addition of the ‘LS’ or ‘YTE’ mutation was required to restore FcRn binding; however binding to FcγR was lost.

To ensure adequate heterodimerization of the two Fc domains of the Fc region of the engineered immunoglobulins of the present disclosure, a variety of approaches can be used in to enhance dimerization, as described in e.g. EP1870459; U.S. Pat. Nos. 5,582,996; 5,731,168; 5,910,573; 5,932,448; 6,833,441; 7,183,076; US2006204493A1; WO 09/089004A1. In one aspect, one or more mutations to a first Fc domain of the engineered immunoglobulin comprising a heavy chain constant domain creates a “knob” and the one or more mutations to a second Fc domain of the engineered immunoglobulin comprising a heavy chain constant domain creates a “hole,” such that heterodimerization of the first and second Fc domains causes the “knob” to interface (e.g., interact, e.g., a CH2 domain of a first Fc domain interacting with a CH2 domain of a second Fc domain, or a CH3 domain of a first Fc domain interacting with a CH3 domain of a second Fc domain) with the “hole”. As the term is used herein, a “knob” refers to at least one amino acid side chain which projects from the interface of a first Fc domain of the engineered immunoglobulin comprising a heavy chain constant domain and is therefore positionable in a compensatory “hole” in the interface with a second Fc domain of the engineered immunoglobulin comprising a heavy chain constant domain so as to stabilize the heterodimer, and thereby favour heterodimeric formation over homodimeric formation, for example. The preferred import residues for the formation of a knob are generally naturally occurring amino acid residues and are preferably selected from arginine (R), phenylalanine (F), tyrosine (Y) and tryptophan (\A). Most preferred are tryptophan and tyrosine. In the preferred embodiment, the original residue for the formation of the protuberance has a small side chain volume, such as alanine, asparagine, aspartic acid, glycine, serine, threonine or valine.

A “hole” refers to at least one amino acid side chain which is recessed from the interface of a second Fc domain of the engineered immunoglobulin comprising a heavy chain constant domain and therefore accommodates a corresponding knob on the adjacent interfacing surface of a first Fc domain of the engineered immunoglobulin comprising a heavy chain constant domain. The preferred import residues for the formation of a hole are usually naturally occurring amino acid residues and are preferably selected from alanine (A), serine (S), threonine (T) and valine (V). Most preferred are serine, alanine or threonine. In the preferred embodiment, the original residue for the formation of the hole has a large side chain volume, such as tyrosine, arginine, phenylalanine or tryptophan.

In one embodiment, a first CH3 domain is mutated at residue 366, 405 or 407 (EU numbering)/, CH3.22, CH3.85.1, CH3.86 (IMGT numbering for C-domain) to create either a “knob” or a hole” (as described above), and the second CH3 domain that heterodimerizes with the first CH3 domain is mutated at: residue 407/CH3.86 if residue 366/CH3.22 is mutated in the first CH3 domain, residue 394/CH3.81 if residue 405/CH3.85.1 is mutated in the first CH3 domain, or residue 366/CH3.22 if residue 407/CH3.86 is mutated in the first CH3 domain (EU/IMGT numbering for C-domain), to create a “hole” or “knob” complementary to the “knob” or “hole” of the first CH3 domain.

In another embodiment, a first CH3 domain is mutated at residue 366/CH3.22 to create either a “knob” or a “hole” (as described above), and the second CH3 domain that heterodimerizes with the first CH3 domain is mutated at residues 366/CH3.22, 368/CH3.24 and/or 407/CH3.86 (EU/IMGT numbering for C-domain), to create a “hole” or “knob” complementary to the “knob” or “hole” of the first CH3 domain. In one embodiment, the mutation to the first CH3 domain introduces a tyrosine (Y) residue at position 366/CH3.22. In an embodiment, the mutation to the first CH3 is T366Y/T_CH3.22_Y. In one embodiment, the mutation to the first CH3 domain introduces a tryptophan (W) residue at position 366/CH3.22. In an embodiment, the mutation to the first CH3 is T366W/T_CH3.22_W. In embodiments, the mutation to the second CH3 domain that heterodimerizes with the first CH3 domain mutated at position 366/CH3.22 (e.g., has a tyrosine (Y) or tryptophan (N) introduced at position 366/CH3.22, e.g., comprises the mutation T366Y/T_CH3.22_Y or T366W/T_CH3.22_VV), comprises a mutation at position 366/CH3.22, a mutation at position 368/CH3.24 and a mutation at position 407/CH3.86 (EU/IMGT numbering for C-domain). In embodiments, the mutation at position 366/CH3.22 introduces a serine (S) residue, the mutation at position 368/CH3.22 introduces an alanine (A), and the mutation at position 407/CH3.86 introduces a valine (V). In embodiments, the mutations comprise T366S, L368A and Y407V/T_CH3.22_S, L_CH3.24_A and Y_CH3.86_V (EU/IMGT numbering)). In one embodiment the first CH3 domain of the multi-specific binding molecule comprises the mutation T366Y/T_CH3.22_Y, and the second CH3 domain that heterodimerizes with the first CH3 domain comprises the mutations T366S, L368A and Y407V (T_CH3.22_S, L_CH3.24_A and Y_CH3.86_V), or vice versa. In one embodiment the first CH3 domain of the multi-specific binding molecule comprises the mutation T366W/T_CH3.22_W, and the second CH3 domain that heterodimerizes with the first CH3 domain comprises the mutations T366S, L368A and Y407V/T_CH3.22_S, L_CH3.24_A and Y_CH3.86_V, or vice versa.

In a preferred embodiment, the CH3 domain that is mutated to create a “hole” is in a first Fc domain of the Fc region of an engineered IgG1 immunoglobulin that comprises amino acid modifications for recruiting FcαRI effector function and the CH3 domain that is mutated to create a “knob” is in a second Fc domain of the Fc region of an engineered IgG1 immunoglobulin that does not comprise amino acid modifications for recruiting FcαRI effector function. In one embodiment, the present disclosure provides an engineered IgG1 immunoglobulin comprising an Fc ‘hole’ domain with an amino acid sequence comprised within SEQ ID NO: 137 or 157. In another embodiment, the present disclosure provides an engineered IgG1 immunoglobulin comprising an Fc ‘knob’ domain with an amino acid sequence comprised within SEQ ID NO: 154, 159, 160, 161 or 162. In a preferred embodiment the present disclosure provides an engineered IgG1 immunoglobulin with a first and second Fc domain from amino acid sequences selected from: SEQ ID NO: 137 or 157 with SEQ ID NO: 154, 159, 160, 161 or 162. The engineered IgG1 immunoglobulin with a first and second Fc domain from amino acid sequences SEQ ID NO: 157 and 159, respectively was preferred if gamma effector function (i.e. binding to FcγRs) was not required. The engineered IgG1 immunoglobulin with a first and second Fc domain from amino acid sequences SEQ ID NO: 157 and 161, respectively was preferred if gamma effector function (i.e. binding to FcγRs) was required. The second Fc domain of this engineered IgG1 immunoglobulin (SEQ ID NO: 161) also comprised the SDIE mutations. Furthermore, the first Fc domain of the engineered IgG1 immunoglobulins comprised within SEQ ID NO: 157 comprised mutations derived from the affinity maturation campaign to improve binding to FcαRI.

Additional knob in hole mutation pairs suitable for use in any of the engineered immunoglobulins of the present disclosure are further described in, for example, WO1996/027011, and Merchant et al., (1998) supra, the contents of which are hereby incorporated by reference in their entirety.

Further Modifications to Engineered Immunoglobulins

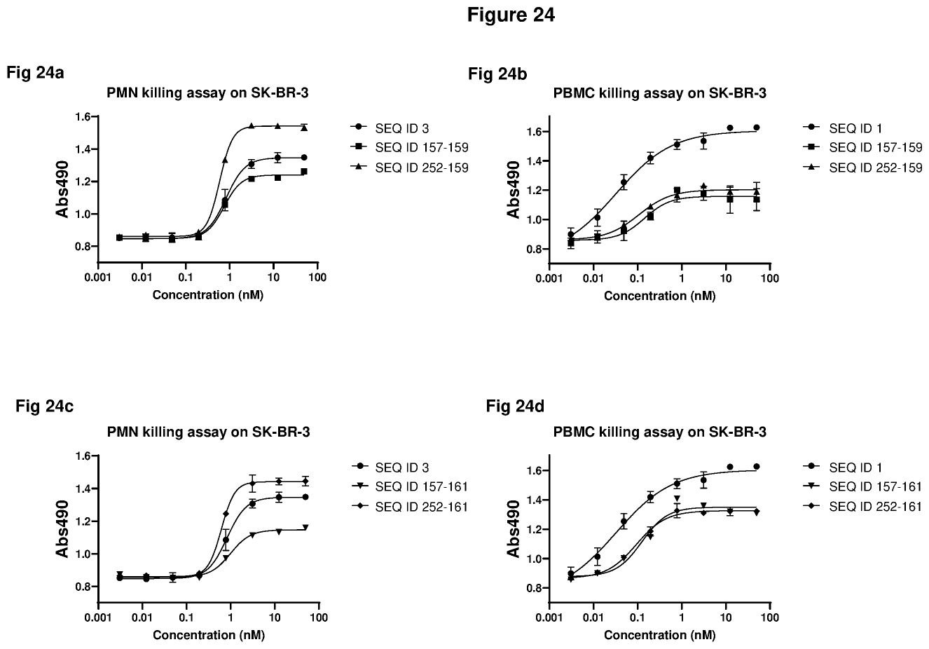

To further enhance binding affinity of the engineered IgG1 immunoglobulins to FcαRI, an affinity maturation campaign was carried out using yeast display to identify amino acids mutations that enhanced IgA2 affinity for FcαRI. In one embodiment, the present disclosure provides an engineered IgG1 immunoglobulin comprising an amino acid mutation in the CH2 domain and/or CH3 domain at one or more of the following positions: CH2.10, CH2.89, CH2.91, CH2.94, CH2.97, CH2.99, CH3.45, CH3.105, CH3.109, CH3.118 and/or CH3.124, wherein numbering is according to IMGT numbering for C-domain. In a one embodiment, the present disclosure provides an engineered IgG1 immunoglobulin comprising an amino acid substitution in the CH2 domain and/or CH3 domain selected from the group consisting of: A_CH2.10_S, L_CH2.89_I, G_CH2.91_Q, G_CH2.91_V, Q_CH2.94_E, N_CH2.97_H, N_CH2.97_Y, G_CH2.99_W, S_CH3.45_D, M_CH3.105_Y, E_CH3.109_D, Q_CH3.118_Y and L_CH3.124_F, wherein numbering is according to IMGT numbering for C-domains. In another embodiment, the present disclosure provides an engineered IgG1 immunoglobulin comprising an amino acid substitution in the CH2 domain and/or CH3 domain selected from one of the mutation sets listed in Table 39. In one embodiment, the present disclosure provides an engineered IgG1 immunoglobulin comprising the following amino acid substitutions: Q_CH2.94_E, N_CH2.97_Y, S_CH3.45_D, M_CH3.105_Y, Q_CH3.118_Y (IMGT numbering for C-domain). In a preferred embodiment, the present disclosure provides an engineered IgG1 immunoglobulin comprising the following amino acid substitutions: Q_CH2.94_E, L_CH2.97_Y and S_CH3.45_D (IMGT numbering for C-domain). This mutation set was applied to the ‘hole’ arm of a heterodimeric Fc comprised within SEQ ID NO: 157 resulting in SEQ ID NO: 252. The engineered IgG1 immunoglobulins with a first and second Fc domain from amino acid sequences selected from and comprised within SEQ ID 252 and 159 or SEQ ID 252 and 161 were shown to have better killing properties on SK-BR-3 cells in PMN killing assays, compared to their parental immunoglobulins and IgA2 (FIG. 24). The engineered IgG1 immunoglobulin with a first and second Fc domain from amino acid sequences SEQ ID NO: 252 and 159, respectively was preferred if gamma effector function (i.e. binding to FcγRs) was not required. The engineered IgG1 immunoglobulin with a first and second Fc domain from amino acid sequences SEQ ID NO: 252 and 161, respectively was preferred if gamma effector function (i.e. binding to FcγRs) was required. The second Fc domain of the engineered IgG1 immunoglobulin comprised within SEQ ID NO: 161 also comprised the SDIE mutations.

In any of the embodiments described herein, the CH3 domains may be additionally mutated to introduce a pair of cysteine residues. Without being bound by theory, it is believed that the introduction of a pair of cysteine residues capable of forming a disulfide bond provides stability to the heterodimeric engineered immunoglobulins. In embodiments, a first CH3 domain comprises a cysteine at position 354/CH3.10 (EU/IMGT numbering for C-domain), and a second CH3 domain that heterodimerizes with the first CH3 domain comprises a cysteine at position 349/CH3.5 (EU/IMGT numbering for C-domain).

In another aspect, heterodimerization of the Fc domains of the engineered immunoglobulins is increased by introducing mutations based on the “polar-bridging” rational, which causes residues at the binding interface of the two Fc domains to interact with residues of similar (or complimentary) physical property in the heterodimer configuration. In particular, these mutations are designed so that, in the heterodimer formation, polar residues interact with polar residues, while hydrophobic residues interact with hydrophobic residues. In contrast, in the homodimer formation, residues are mutated so that polar residues interact with hydrophobic residues. The favorable interactions in the heterodimer configuration and the unfavorable interactions in the homodimer configuration work together to make it more likely for CH3 domains to form heterodimers than to form homodimers.

In an exemplary embodiment, the above mutations are generated at one or more positions of residues 364, 366, 368, 399, 405, 407, 409, and 411 in a CH3 domain (IMGT numbering for C-domain)/CH3.20, CH3.22, CH3.24, CH3.84.2, CH3.85.1, CH3.86, CH3.88, CH3.90 (IMGT numbering for C-domain). In one aspect, one CH3 domain has one or more mutations selected from a group consisting of: S364L, T366V, L368Q, D399L, F405S, K409F and T411K/S_CH3.20_L, T_CH3.22_V, L_CH3.24_Q, D_CH3.84.2_L, F_CH3.85.1_S, K_CH3.88_F, T_CH3.90_K (EU/IMGT numbering for C-domain), while the other CH3 domain has one or more mutations selected from a group consisting of: Y407F, K409Q and T411D/Y_CH3.86_F, K_CH3.88_Q and T_CH3.90_D (EU/IMGT numbering). The polar bridge strategy is described in, for example, WO2006/106905, WO2009/089004 and Gunasekaran K et al., (2010) J Biol Chem., 285: 19637-19646, the contents of which are hereby incorporated by reference in their entirety.

The amino acid replacements described herein are introduced into the CH3 domains using techniques which are known in the art. Normally the DNA encoding the heavy chain(s) is genetically engineered using the techniques described in Mutagenesis: a Practical Approach. Oligonucleotide-mediated mutagenesis is a preferred method for preparing substitution variants of the DNA encoding the two hybrid heavy chains. This technique is known in the art as described by Adelman et al., (1983) DNA, 2:183.

Conjugates

The present disclosure includes engineered immunoglobulins (e.g., antibodies) or fragments thereof recombinantly fused or chemically conjugated (including both covalent and non-covalent conjugations) to a heterologous protein or polypeptide (or fragment thereof, preferably to a polypeptide of at least 10, at least 20, at least 30, at least 40, at least 50, at least 60, at least 70, at least 80, at least 90 or at least 100 amino acids) to generate fusion proteins. Methods for fusing or conjugating proteins, polypeptides, or peptides to an antibody or an antibody fragment are known in the art. See, e.g., U.S. Pat. Nos. 5,336,603, 5,622,929, 5,359,046, 5,349,053, 5,447,851, and 5,112,946; EP 307434 and EP 367166; WO 96/04388 and WO 91/06570; Ashkenazi et al., (1991) PNAS. USA 88:10535-10539; Zheng et al., (1995) J. Immunol. 154: 5590-5600; and Vil et al., (1992) PNAS. USA 89:11337-11341.

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of molecules of the disclosure or fragments thereof (e.g., molecules or fragments thereof with higher affinities and lower dissociation rates). See, generally, U.S. Pat. Nos. 5,605,793, 5,811,238, 5,830,721, 5,834,252, and 5,837,458; Patten et al., (1997) Curr. Opinion Biotechnol. 8:724-33; Harayama (1998) Trends Biotechnol. 16(2):76-82; Hansson et al., (1999) J. Mol. Biol. 287: 265-76; and Lorenzo & Blasco (1998) Biotechniques, 24(2):308-313 (each of these patents and publications are hereby incorporated by reference in its entirety). The molecules described herein or fragments thereof may be altered by being subjected to random mutagenesis by error-prone PCR, random nucleotide insertion or other methods prior to recombination. A polynucleotide encoding a fragment of the present molecule may be recombined with one or more components, motifs, sections, parts, domains, fragments, etc. of one or more heterologous molecules.

Moreover, the engineered immunoglobulins of the present disclosure can be fused to marker sequences, such as a peptide to facilitate purification. In preferred embodiments, the marker amino acid sequence is a hexa-histidine peptide (SEQ ID NO: 255), such as the tag provided in a pQE vector (QIAGEN, Inc., 9259 Eton Avenue, Chatsworth, Calif., 91311), among others, many of which are commercially available. As described in Gentz et al., (1989) PNAS. USA 86:821-824, for instance, hexa-histidine (SEQ ID NO: 255) provides for convenient purification of the fusion protein. Other peptide tags useful for purification include, but are not limited to, the hemagglutinin (“HA”) tag, which corresponds to an epitope derived from the influenza hemagglutinin protein (Wilson et al., (1984) Cell 37:767), and the “flag” tag.

In other embodiments, the engineered immunoglobulins of the present disclosure are conjugated to a diagnostic or detectable agent. Such molecules can be useful for monitoring or prognosing the onset, development, progression and/or severity of a disease or disorder as part of a clinical testing procedure, such as determining the efficacy of a particular therapy. Such diagnosis and detection can accomplished by coupling the molecules to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (131I, 125I, 123I, and 121I,), carbon (14C), sulfur (35S), tritium (3H), indium (115In, 113In, 112In, and 111In,), technetium (99Tc), thallium (201Ti), gallium (68Ga, 67Ga), palladium (103Pd), molybdenum (99Mo), xenon (133Xe), fluorine (18F), 153Sm, 177Lu, 159Gd, 149Pm, 140La, 175Yb, 166Ho, 90Y, 47Sc, 186Re, 188Re, 142 Pr, 105Rh, 97Ru, 68Ge, 57Co, 65Zn, 85Sr, 32P, 153Gd, 169Yb, 51Cr, 54Mn, 75Se, 113Sn, and 117Tin; and positron emitting metals using various positron emission tomographies, and nonradioactive paramagnetic metal ions.

The present application further encompasses uses of the engineered immunoglobulins of the present disclosure conjugated to a therapeutic moiety. For example, the therapeutic moiety may be a cytotoxin, e.g., a cytostatic or cytocidal agent, a therapeutic agent or a radioactive metal ion, e.g., alpha-emitters. A cytotoxin or cytotoxic agent includes any agent that is detrimental to cells.

Furthermore, the engineered immunoglobulin may be conjugated to a therapeutic moiety or drug moiety that modifies a given biological response. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, an anti-angiogenic agent; or, a biological response modifier such as, for example, a lymphokine.

For further discussion of types of cytotoxins, linkers and methods for conjugating therapeutic agents to the engineered immunoglobulin, see also Saito et al., (2003) Adv. Drug Deliv. Rev. 55:199-215; Trail et al., (2003) Cancer Immunol. Immunother. 52: 328-337; Payne (2003) Cancer Cell 3: 207-212; Allen (2002) Nat. Rev. Cancer, 2:750-763; Pastan & Kreitman (2002) Curr. Opin. Investig. Drugs, 3: 1089-1091; Senter & Springer (2001) Adv. Drug Deliv. Rev. 53: 247-264.

The engineered immunoglobulins of the present disclosure also can be conjugated to a radioactive isotope to generate cytotoxic radiopharmaceuticals, also referred to as radioimmunoconjugates. Examples of radioactive isotopes that can be conjugated to engineered immunoglobulins for use diagnostically or therapeutically include, but are not limited to, iodinel3l, indium111, yttrium90, and lutetium177. Method for preparing radioimmunconjugates are established in the art. See, e.g., Denardo et al., (1998) Clin Cancer Res. 4(10): 2483-90; Peterson et al., (1999) Bioconjug. Chem. 10(4):553-7; and Zimmerman et al., (1999) Nucl. Med. Biol. 26(8): 943-50, each incorporated by reference in their entireties.

Techniques for conjugating therapeutic moieties to engineered immunoglobulins such as antibodies or antibody-like molecules are known, see, e.g., Arnon et al., “Monoclonal Antibodies for Immunotargeting of Drugs in Cancer Therapy”, in Monoclonal Antibodies and Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers of Cytotoxic Agents in Cancer Therapy: A Review”, in Monoclonal Antibodies 84: Biological and Clinical Applications, Pinchera et al. (eds.), pp. 475-506 (1985); “Analysis, Results, and Future Prospective of the Therapeutic Use of Radiolabeled Antibody in Cancer Therapy”, in Monoclonal Antibodies for Cancer Detection and Therapy, Baldwin et al. (eds.), pp. 303-16 (Academic Press 1985), and Thorpe et al., (1982) Immunol. Rev. 62:119-58.

The engineered immunoglobulins may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Methods of Making the Engineered Immunoglobulins

Preparing Polypeptide Chains

Antibodies or immunoglobulins and fragments thereof can be produced by a variety of techniques, including conventional monoclonal antibody methodology e.g., the standard somatic cell hybridization technique of Kohler and Milstein, (1975) Nature 256: 495. Many techniques for producing monoclonal antibody can be employed e.g., viral or oncogenic transformation of B lymphocytes.

An animal system for preparing hybridomas is the murine system. Hybridoma production in the mouse is an established procedure. Immunization protocols and techniques for isolation of immunized splenocytes for fusion are known in the art. Fusion partners (e.g., murine myeloma cells) and fusion procedures are also known.

Chimeric or humanized antibodies can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art. See e.g., U.S. Pat. No. 5,225,539 to Winter, and U.S. Pat. Nos. 5,530,101; 5,585,089; 5,693,762 and U.S. Pat. No. 6,180,370 to Queen et al.

In a certain embodiment, the antibody or immunoglobulins of the disclosure are human monoclonal antibodies. Such human monoclonal antibodies can be generated using transgenic or transchromosomic mice carrying parts of the human immune system rather than the mouse system. These transgenic and transchromosomic mice include mice referred to herein as HUMAB mice and KM mice, respectively, and are collectively referred to herein as “human Ig mice.”

The HUMAB mouse (Medarex, Inc.) contains human immunoglobulin gene miniloci that encode un-rearranged human heavy (μ and γ) and K light chain immunoglobulin sequences, together with targeted mutations that inactivate the endogenous μ and κ chain loci (see e.g., Lonberg, et al., (1994) Nature 368(6474): 856-859). Accordingly, the mice exhibit reduced expression of mouse IgM or κ, and in response to immunization, the introduced human heavy and light chain transgenes undergo class switching and somatic mutation to generate high affinity human IgGK monoclonal (Lonberg et al., (1994) supra; reviewed in Lonberg, (1994) Handbook of Experimental Pharmacology 113:49-101; Lonberg & Huszar, (1995) Intern. Rev. Immunol. 13: 65-93, and Harding & Lonberg, (1995) Ann. N. Y. Acad. Sci. 764:536-546). The preparation and use of HUMAB mice, and the genomic modifications carried by such mice, is further described in Taylor et al., (1992) Nucleic Acids Research 20:6287-6295; Chen Y., (1993) International Immunology 5: 647-656; Tuaillon Y., (1993) PNAS USA 94:3720-3724; Choi Y., (1993) Nature Genetics 4:117-123; Chen Y., (1993) EMBO J. 12:821-830; Tuaillon et al., (1994) J. Immunol. 152:2912-2920; Taylor Y., (1994) International Immunology 579-591; and Fishwild Y., (1996) Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg & Kay; U.S. Pat. No. 5,545,807 to Surani et al.; WO 92/103918, WO 93/12227, WO 94/25585, WO 97/113852, WO 98/24884 and WO 99/45962, all to Lonberg & Kay; and WO 01/14424 to Korman et al.

In another embodiment, human antibodies or immunoglobulins used in the present disclosure can be raised using a mouse that carries human immunoglobulin sequences on transgenes and transchomosomes such as a mouse that carries a human heavy chain transgene and a human light chain transchromosome. Such mice, referred to herein as “KM mice”, are described in detail in WO 02/43478 (Ishida et al).

Still further, alternative transgenic animal systems expressing human immunoglobulin genes are available in the art and can be used to raise human antibodies. For example, an alternative transgenic system referred to as the Xenomouse (Abgenix, Inc.) can be used. Such mice are described in, e.g., U.S. Pat. Nos. 5,939,598; 6,075,181; 6,114,598; 6,150,584 and 6,162,963 (Kucherlapati et al).

Moreover, alternative transchromosomic animal systems expressing human immunoglobulin genes are available in the art and can be used to raise the human antibodies or immunoglobulins of the disclosure. For example, mice carrying both a human heavy chain transchromosome and a human light chain tranchromosome, referred to as “TC mice” can be used; such mice are described in Tomizuka et al., (2000) PNAS USA 97:722-727. Furthermore, cows carrying human heavy and light chain transchromosomes have been described in the art (Kuroiwa (2002) Nature Biotechnology 20:889-894) and can be used to raise human antibodies used in the present application.

Human monoclonal antibodies can also be prepared using phage display methods for screening libraries of human immunoglobulin genes. Such phage display methods for isolating human antibodies are established in the art or described in the examples below. See for example: U.S. Pat. Nos. 5,223,409; 5,403,484; and U.S. Pat. No. 5,571,698 (Ladner et al); U.S. Pat. Nos. 5,427,908 and 5,580,717 (Dower et al); U.S. Pat. Nos. 5,969,108 and 6,172,197 (McCafferty et al); and U.S. Pat. Nos. 5,885,793; 6,521,404; 6,544,731; 6,555,313; 6,582,915 and U.S. Pat. No. 6,593,081 (Griffiths et al).

Human monoclonal antibodies used in the disclosure can also be prepared using SCID mice into which human immune cells have been reconstituted such that a human antibody response can be generated upon immunization. Such mice are described in, for example, U.S. Pat. Nos. 5,476,996 and 5,698,767 (Wilson et al).

In one embodiment, the present disclosure provides an antibody that has be generated by any of the aforementioned methods comprising at least one modified Fc domain as described herein.

Nucleic Acids and Expression Systems

The present invention also encompasses nucleic acids encoding the polypeptide chains, Fc domains or Fc regions of the engineered immunoglobulins described herein. Nucleic acid molecules of the disclosure include DNA and RNA in both single-stranded and double-stranded form, as well as the corresponding complementary sequences. The nucleic acid molecules of the disclosure include full-length genes or cDNA molecules as well as a combination of fragments thereof. The nucleic acids of the disclosure are derived from human sources but can include those derived from non-human species.

An “isolated nucleic acid” is a nucleic acid that has been separated from adjacent genetic sequences present in the genome of the organism from which the nucleic acid was isolated, in the case of nucleic acids isolated from naturally-occurring sources. In the case of nucleic acids synthesized enzymatically from a template or chemically, such as PCR products, cDNA molecules, or oligonucleotides for example, it is understood that the nucleic acids resulting from such processes are isolated nucleic acids. An isolated nucleic acid molecule refers to a nucleic acid molecule in the form of a separate fragment or as a component of a larger nucleic acid construct. In one preferred embodiment, the nucleic acids are substantially free from contaminating endogenous material. The nucleic acid molecule has preferably been derived from DNA or RNA isolated at least once in substantially pure form and in a quantity or concentration enabling identification, manipulation, and recovery of its component nucleotide sequences by standard biochemical methods (such as those outlined in Sambrook et al., Molecular Cloning: A Laboratory Manual, 2nd ed., Cold Spring Harbor Laboratory, Cold Spring Harbor, N.Y. (1989)). Such sequences are preferably provided and/or constructed in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region.

Variant sequences can be prepared by site specific mutagenesis of nucleotides in the DNA encoding the polypeptide, using cassette or PCR mutagenesis or other techniques well known in the art, to produce DNA encoding the variant, and thereafter expressing the recombinant DNA in cell culture as outlined herein.

As “optimized nucleotide sequence” means a nucleotide sequence has been altered to encode an amino acid sequence using codons that are preferred in the production cell, for example, a Chinese Hamster Ovary cell (CHO). The optimized nucleotide sequence is engineered to retain completely the amino acid sequence originally encoded by the starting nucleotide sequence, which is also known as the “parental” sequence.

The present disclosure also provides expression systems and constructs in the form of plasmids, expression vectors, transcription or expression cassettes which comprise at least one polynucleotide as above. In addition, the disclosure provides host cells comprising such expression systems or constructs. The heavy and light chains of an engineered IgG1 immunoglobulin or fragment thereof can be encoded by a single nucleic acid (e.g., inserted into a single vector), or can be encoded by multiple nucleic acid molecules, e.g., two nucleic acid molecules (also referred to as a “set”), which can be inserted into multiple vectors (e.g., two vectors, i.e., a set of vectors).

In one embodiment, the present invention provides a method of preparing an engineered IgG1 immunoglobulin or fragment thereof comprising an Fc region comprising modified first and second Fc domains, the method comprising the steps of: (a) culturing a host cell comprising a nucleic acid encoding a heavy chain comprising the engineered Fc domain polypeptide and a nucleic acid comprising a light chain polypeptide, wherein the cultured host cell expresses the engineered polypeptides; and (b) recovering the engineered IgG1 immunoglobulin from the host cell culture.

Expression vectors of use in the present disclosure may be constructed from a starting vector such as a commercially available vector. After the vector has been constructed and a nucleic acid molecule encoding polypeptide chains of the engineered immunoglobulin has been inserted into the proper site of the vector, the completed vector may be inserted into a suitable host cell for amplification and/or polypeptide expression. The transformation of an expression vector into a selected host cell may be accomplished by known methods including transfection, infection, calcium phosphate co-precipitation, electroporation, microinjection, lipofection, DEAE-dextran mediated transfection, or other known techniques. The method selected will in part be a function of the type of host cell to be used. These methods and other suitable methods are well known to the skilled artisan, and are set forth, for example, in Sambrook et al., 2001, supra.

Typically, expression vectors used in the host cells will contain sequences for plasmid maintenance and for cloning and expression of exogenous nucleotide sequences. Such sequences, collectively referred to as ‘flanking sequences’, in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element.

A host cell, when cultured under appropriate conditions, can be used to express bispecific antibody that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule. A host cell may be eukaryotic or prokaryotic.

Mammalian cell lines available as hosts for expression are well known in the art and include, but are not limited to, immortalized cell lines available from the American Type Culture Collection (ATCC) and any cell lines used in an expression system known in the art can be used to make polypeptides comprising the engineered immunoglobulins of the present disclosure. In general, host cells are transformed with a recombinant expression vector that comprises DNA encoding a desired engineered immunoglobulin. Among the host cells that may be employed are prokaryotes, yeast or higher eukaryotic cells. Prokaryotes include gram negative or gram positive organisms, for example E. coli or bacilli. Higher eukaryotic cells include insect cells and established cell lines of mammalian origin. Examples of suitable mammalian host cell lines include the COS-7 cells, L cells, C127 cells, 3T3 cells, Chinese hamster ovary (CHO) cells, or their derivatives and related cell lines which grow in serum free media, HeLa cells, BHK cell lines, the CVIIEBNA cell line, human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Optionally, mammalian cell lines such as HepG2/3B, KB, NIH 3T3 or S49, for example, can be used for expression of the polypeptide when it is desirable to use the polypeptide in various signal transduction or reporter assays. Alternatively, it is possible to produce the polypeptide in lower eukaryotes such as yeast or in prokaryotes such as bacteria. Suitable yeasts include S. cerevisiae, S. pombe, Kluyveromyces strains, Candida, or any yeast strain capable of expressing heterologous polypeptides. Suitable bacterial strains include E. coli, B. subtilis, S. typhimurium, or any bacterial strain capable of expressing heterologous polypeptides. If the engineered immunoglobulin is made in yeast or bacteria, it may be desirable to modify the product produced therein, for example by phosphorylation or glycosylation of the appropriate sites, in order to obtain a functional product. Such covalent attachments can be accomplished using known chemical or enzymatic methods.

Pharmaceutical Compositions and Dosing

Provided herein are pharmaceutical compositions comprising the engineered immunoglobulins of the present disclosure. The engineered immunoglobulin can be in combination with one or more pharmaceutically acceptable excipients, diluents or carriers.

To prepare pharmaceutical or sterile compositions comprising an engineered immunoglobulin of the present disclosure, the immunoglobulin is mixed with a pharmaceutically acceptable carrier or excipient. The phrase “pharmaceutically acceptable” means approved by a regulatory agency of a federal or a state government, or listed in the U.S. Pharmacopeia or other generally recognized pharmacopeia for use in animals, and more particularly, in humans. The term “pharmaceutical composition” refers to a mixture of at least one active ingredient (e.g., an engineered immunoglobulins of the disclosure) and at least one pharmaceutically-acceptable excipient, diluent or carrier. A “medicament” refers to a substance used for medical treatment.

Pharmaceutical compositions of therapeutic and diagnostic agents can be prepared by mixing with physiologically acceptable carriers, excipients, or stabilizers in the form of, e.g., lyophilized powders, slurries, aqueous solutions, lotions, or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Oral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

Selecting an administration regimen for a therapeutic depends on several factors, including the serum or tissue turnover rate of the entity, the level of symptoms, the immunogenicity of the entity, and the accessibility of the target cells in the biological matrix. In certain embodiments, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert, et al. (2003) New Engl. J. Med. 348:601-608; Milgrom, et al. (1999) New Engl. J. Med. 341:1966-1973; Slamon, et al. (2001) New Engl. J. Med. 344:783-792; Beniaminovitz, et al. (2000) New Engl. J. Med. 342:613-619; Ghosh, et al. (2003) New Engl. J. Med. 348:24-32; Lipsky, et al. (2000) New Engl. J. Med.

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

Actual dosage levels of the active ingredients in the pharmaceutical compositions of the present disclosure may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present disclosure employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors known in the medical arts.

Pharmaceutical compositions comprising the engineered immunoglobulins of the present disclosure can be provided by continuous infusion, or by doses at intervals of, e.g., one day, one week, or 1-7 times per week. Doses may be provided intravenously, subcutaneously, topically, orally, nasally, rectally, intramuscular, intracerebrally, or by inhalation.

The desired dose of a therapeutic comprising the engineered immunoglobulins of the present disclosure is about the same as for an antibody or polypeptide, on a moles/kg body weight basis. The doses administered to a subject may number at least 1, 2, 3, 4, 5, 6, 7, 8, 9, 10, 11, or 12, or more.

For a therapeutic comprising the engineered immunoglobulin of the present disclosure, the dosage administered to a patient may be about 0.0001 mg/kg to about 100 mg/kg of the patient's body weight.

Where a series of doses are administered, these may, for example, be administered approximately every day, approximately every week, approximately every month. The doses may, for example, continue to be administered until disease progression, adverse event, or other time as determined by the physician.

An effective amount for a particular patient may vary depending on factors such as the condition being treated, the overall health of the patient, the method route and dose of administration and the severity of side effects (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

Where necessary, the therapeutic comprising the engineered immunoglobulin of the present disclosure may be incorporated into a composition that includes a solubilizing agent and a local anesthetic such as lidocaine to ease pain at the site of the injection. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and WO 92/19244, WO 97/32572, WO 97/44013, WO 98/31346, and WO 99/66903, each of which is incorporated herein by reference their entirety.

A therapeutic comprising an engineered immunoglobulin of the present disclosure can also be administered via one or more routes of administration using one or more of a variety of methods known in the art. As will be appreciated by the skilled artisan, the route and/or mode of administration will vary depending upon the desired results. Selected routes of administration for the antibodies include intravenous, intramuscular, intradermal, intraperitoneal, subcutaneous, spinal or other parenteral routes of administration, for example by injection or infusion. Parenteral administration can represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the present disclosure can be administered via a non-parenteral route, such as a topical, epidermal or mucosal route of administration, for example, intranasally, orally, vaginally, rectally, sublingually or topically.

The therapeutic comprising an engineered immunoglobulin of the present disclosure may be administered via any of the above routes using, e.g., an injection device, an injection pen, a vial and syringe, pre-filled syringe, autoinjector, an infusion pump, a patch pump, an infusion bag and needle, etc. If the molecules or fragments thereof of the disclosure are administered in a controlled release or sustained release system, a pump may be used to achieve controlled or sustained release (see Langer, supra; Sefton, 1987, CRC Crit. Ref Biomed. Eng. 14:20; Buchwald et al., 1980, Surgery 88:507; Saudek et al., 1989, N. Engl. J. Med. 321:574). Polymeric materials can be used to achieve controlled or sustained release of the therapies of the disclosure (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas (1983) J. Macromol. Sci. Rev. Macromol. Chem. 23:61; see also Levy et al., (1985) Science 228:190; During et al., (1989) Ann. Neurol. 25:351; Howard et al., (1989) J. Neurosurg., 7(1):105; U.S. Pat. Nos. 5,679,377; 5,916,597; 5,912,015; 5,989,463; 5,128,326; WO 99/15154; and WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. A controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (Science (1990) 249:1527-1533). Any technique known to one of skill in the art can be used to produce sustained release formulations comprising one or more molecules or fragments thereof of the present application. See, e.g., U.S. Pat. No. 4,526,938, WO 91/05548, WO 96/20698, Ning et al., (1996) Radiotherapy & Oncology 39: 179-189; Song et al., (1995) PDA Journal of Pharm Sci & Tech., 50: 372-397; Cleek et al., (1997) Pro. Intl Symp. Control. Rel. Bioact. Mater. 24: 853-854; Lam et al., (1997) Proc. Intl Symp. Control Rel. Bioact. Mater., 24: 759-760, each of which is incorporated herein by reference in their entirety.

If a pharmaceutical composition comprising an engineered immunoglobulin of the present disclosure is administered topically, it can be formulated in the form of an ointment, cream, transdermal patch, lotion, gel, shampoo, spray, aerosol, solution, emulsion, or other forms known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity, in some instances, greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, in some instances, in combination with a solid or liquid inert carrier, is packaged in a mixture with a pressurized volatile (e.g., a gaseous propellant, such as Freon) or in a squeeze bottle. Moisturizers or humectants can also be added to pharmaceutical compositions and dosage forms if desired. Examples of such additional ingredients are known in the art.

If a pharmaceutical composition comprising an engineered immunoglobulin of the present disclosure is administered intranasally, it can be formulated in an aerosol form, spray, mist or in the form of drops. In particular, prophylactic or therapeutic agents for use according to the present disclosure can be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

A pharmaceutical composition comprising an engineered immunoglobulin of the present disclosure can also be cyclically administered to a patient.

In certain embodiments, pharmaceutical compositions comprising an engineered immunoglobulin of the present disclosure can be formulated to ensure proper distribution in vivo. For example, the blood-brain barrier (BBB) excludes many highly hydrophilic compounds. To ensure that the therapeutic compounds of the disclosure cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., Ranade V V (1989) J. Clin. Pharmacol. 29:685). Exemplary targeting moieties include folate or biotin (see, e.g., U.S. Pat. No. 5,416,016 to Low et al); mannosides (Umezawa et al., (1988) Biochem. Biophys. Res. Commun. 153:1038); antibodies (P. G. Bloeman et al., (1995) FEBS Lett., 357: 140; M. Owais et al. (1995) Antimicrob. Agents Chemother., 39: 180); surfactant protein A receptor (Briscoe et al., (1995) Am. J. Physiol. 1233:134); p 120 (Schreier et al (1994) J. Biol. Chem. 269:9090); see also Keinanen & Laukkanen (1994) FEBS Lett., 346:123-6; Killion & Fidler (1994) Immunomethods, 4: 273.

The present application also provides protocols for the co-administration or treatment of patients using a pharmaceutical composition comprising an engineered immunoglobulin of the present disclosure in combination with other therapies or therapeutic agent(s). Methods for co-administration or treatment with an additional therapeutic agent, e.g., a cytokine, steroid, chemotherapeutic agent, antibiotic, or radiation, are known in the art (see, e.g., Hardman et al., (eds.) (2001) Goodman and Gilman's The Pharmacological Basis of Therapeutics, 10.sup.th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.). An effective amount of therapeutic may decrease the symptoms by at least 10%, by at least 20%, at least about 30%, at least 40%, or at least 50%.

In some embodiments, a pharmaceutical composition of the disclosure further comprises one or more additional therapeutic agents.

In addition to the above therapeutic regimens, the patient may be subjected to surgery and other forms of physical therapy.

Therapeutic Application

Therapeutic or pharmaceutical compositions comprising an engineered immunoglobulin of the present disclosure, whilst not being limited to, are useful for the treatment, prevention, or amelioration of cell proliferative disorders or conditions in which there is an abnormal proliferation of cells, termed herein as “cell proliferative disorders or conditions”. In one aspect, the disclosure provides methods for treating a cell proliferative disorder or condition. In one aspect, the subject of treatment is a human.

Examples of cell proliferative disorders or conditions that may be treated, prevented, or ameliorated using therapeutic or pharmaceutical compositions comprising an engineered immunoglobulin of the present disclosure include but are not limited to fibrosis and cancer. As used herein, the term “cancer” is meant to include all types of cancerous growths or oncogenic processes, metastatic tissues or malignantly transformed cells, tissues, or organs, irrespective of histopathologic type or stage of invasiveness.

In one embodiment, the engineered immunoglobulin binds to a target antigen that is selected from the list consisting of: fibronectin EDA, HER2, EGFR, CD20, CD30, EpCAM, GD2 and solid tumor antigens.

In specific embodiments, the administration of a therapeutic or pharmaceutical composition comprising an engineered immunoglobulin of the present disclosure to a subject in accordance with the methods described herein achieves one, two, or three or more results: (1) a reduction in the growth of a tumor or neoplasm; (2) a reduction in the formation of a tumor; (3) an eradication, removal, or control of primary, regional and/or metastatic cancer; (4) a reduction in metastatic spread; (5) a reduction in mortality; (6) an increase in survival rate; (7) an increase in length of survival; (8) an increase in the number of patients in remission; (9) a decrease in hospitalization rate; (10) a decrease in hospitalization lengths; and (11) the maintenance in the size of the tumor so that it does not increase by more than about 10%, or by more than about 8%, or by more than about 6%, or by more than about 4%; preferably the size of the tumor does not increase by more than about 2%.

In a specific embodiment, the administration of a therapeutic or pharmaceutical composition comprising an engineered immunoglobulin of the present disclosure to a subject with cancer (in some embodiments, an animal model for cancer) in accordance with the methods described herein inhibits or reduces the growth of a tumor by at least about 2-fold, preferably at least about 2.5-fold, at least about-3 fold, at least about 4-fold, at least about 5-fold, at least about 7-fold, or at least about 10-fold relative to the growth of a tumor in a subject with cancer (in some embodiments, in the same animal model for cancer) administered a negative control as measured using assays well known in the art. In another embodiment, the administration of a therapeutic or pharmaceutical composition comprising an engineered immunoglobulin of the present disclosure to a subject with cancer (in some embodiments, an animal model for cancer) in accordance with the methods described herein inhibits or reduces the growth of a tumor by at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% relative to the growth of a tumor in a subject with cancer (in some embodiments, in the same animal model for cancer) administered a negative control, as measured using assays well known in the art.

Examples of cancerous disorders include, but are not limited to, solid tumors, hematological cancers, soft tissue tumors, and metastatic lesions.

In a specific embodiment, the cancer is breast cancer, neuroblastoma, lymphoma, colon cancers, pancreatic ductal adenocarcinoma, melanoma, renal cell carcinoma, bladder cancer, colorectal cancer, non-small cell lung cancer, non-Hodgkins lymphoma or multiple myeloma.

Combination Therapies

Administered “in combination”, in reference to an additional therapeutic agent, means that two (or more) different treatments are delivered to the subject during the course of the subject's affliction with the disorder. In some embodiments, the delivery of one treatment is still occurring when the delivery of the second begins, so that there is overlap in terms of administration. This is referred to as “simultaneous” or “concurrent delivery”. In other embodiments, the delivery of one treatment ends before the delivery of the other treatment begins. This is referred to as “sequential delivery”. In some embodiments of either case, the treatment is more effective because of combined administration. The additional therapeutic agent(s) of the combination therapies of the present disclosure can also be cyclically administered. Combination cycling therapy involves the administration of a first therapy for a period of time, followed by the administration of a second for a period of time and repeating this sequential administration.

A therapeutic or pharmaceutical composition comprising an engineered immunoglobulin as described herein can be administered together with one or more other therapies, e.g., anti-cancer agents, cytokines or anti-hormonal agents, to treat and/or manage cancer. Other therapies that can be used in combination with a therapeutic or pharmaceutical composition comprising an engineered immunoglobulin as described herein, include, but are not limited to, small molecules, synthetic drugs, peptides (including cyclic peptides), polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides), antibodies, synthetic or natural inorganic molecules, mimetic agents, and synthetic or natural organic molecules.

Non-limiting examples of one or more other therapies that can be used in addition to a therapeutic or pharmaceutical composition comprising an engineered immunoglobulin as described herein include, but not limited to, chemotherapy, radiotherapy, cytotoxic agents, chemotherapeutic agents, cytokines, kinase inhibitors, low dose gemcitabine, 5-fluorouracil and cytokine modulators. In particular, one or more other therapies that can be used in addition to a therapeutic or pharmaceutical composition comprising an engineered immunoglobulin of the present disclosure include in particular immune oncology approaches that would perturb the tumor microenvironment, for example, recombinant IL-2, recombinant IL-15, recombinant IL-12, recombinant IL-21, anti-IL1β, anti-TGFβ, anti-CD39, anti-CD73, anti-CTLA4, anti-PD(L)1, anti-TIM3, HDAC inhibitors, HIF1a inhibitors and anti-angiogenics such as anti-VEGF.

Kits

The disclosure also encompasses kits for treating a patient having a cell proliferative disorder. Such kits comprise a therapeutically effective amount of a therapeutic or pharmaceutical composition comprising an engineered immunoglobulin as described herein. Additionally, such kits may comprise means for administering the therapeutic or pharmaceutical composition comprising an engineered immunoglobulin as described herein (e.g., an autoinjector, a syringe and vial, a prefilled syringe, a prefilled pen) and instructions for use. These kits may contain additional therapeutic agents (described infra) for treating a cell proliferative disorder. Such kits may also comprise instructions for administration of the therapeutic or pharmaceutical composition comprising an engineered immunoglobulin as described herein, to treat the patient. Such instructions can provide the dose, route of administration, regimen, and total treatment duration for use with the therapeutic or pharmaceutical composition comprising an engineered immunoglobulin as described herein.

The phrase “means for administering” is used to indicate any available implement for systemically administering a drug to a patient, including, but not limited to, a pre-filled syringe, a vial and syringe, an injection pen, an auto-injector, an IV drip and bag, an infusion pump, a patch, an infusion bag and needle, etc. With such items, a patient may self-administer the drug (i.e., administer the drug without the assistance of a physician) or a medical practitioner may administer the drug.

### EXAMPLES

The following examples are provided to further illustrate the disclosure but not to limit its scope. Other variants of the disclosure will be readily apparent to one of ordinary skill in the art and are encompassed by the appended claims.

All constructs derived from amino acid sequences generated according to Examples 1, 2 and 3, were expressed in mammalian systems and purified (Example 4) to be assessed for binding to FcαRI, FcRn, FcγRIa and FcγRIIIa using surface plasmon resonance (SPR) (Example 5). Functionality of the engineered immunoglobulins was assessed by a cell-based assay using human freshly isolated PMN, PBMC and monocytes-derived macrophages (Examples 6 and 7). Thermal stability was investigated with differential scanning calorimetry (DSC) measurement (Example 8). All Examples were performed using the engineered immunoglobulins in an antibody format comprising VH and VL domains recognizing the antigen HER2 and engineered hinge and Fc regions based on either IgA2 or IgG1. SEQ ID NO: 1 is the full length heavy chain sequence of an anti-HER2 binding antibody having a VH domain that binds HER2 and a hinge and constant domains from IgG1. SEQ ID NO: 3 is a full length heavy chain sequence of an anti-HER2 binding antibody having a VH domain that binds HER2 and a hinge and constant domains from the m2 allotype of IgA2 (Lombana et al., (2019) MABS, 11(6): 1122-38). SEQ ID NO: 124 is the light chain sequence of an anti-HER2-binding antibody having a VL domain that binds HER2 and a constant domain (CL; kappa) from IgG1.

### Example 1: IgA/IgG Protein Engineering

1.1 Transfer of IgA Fc/hFcαRI Interface from Human IgA to Human IgG Immunoglobulin

Initial experimental work focused on the stepwise transfer of constant domains from an IgG1 isotype antibody to an IgA2 isotype antibody comprising VH and VL domains binding to HER2.

1.1.1. Alpha Tail-Piece Removal and P_CH1.124_R (IMGT Numbering for C-Domain) for Monomeric IgA2 Generation

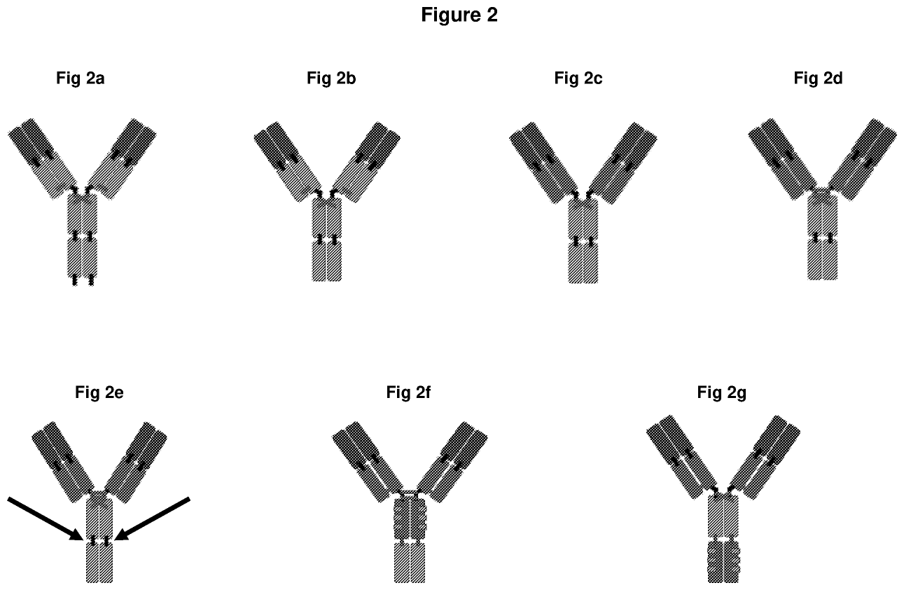

IgA exists in three different forms, a monomeric form in circulating in the blood, a dimeric form found in the mucosae compartment and a secretory form found in association with the secretory compartment that undergoes transcytosis. For the present work, using the monomeric form of IgA2, it was necessary to generate a monomeric form of IgA2 that was less susceptible to degradation by proteolytic enzymes. Therefore the tail piece, found at the C-terminal of the IgA2 CH3 domain, as present in SEQ ID NO: 130 (FIG. 2a), was removed by standard cloning and PCR methods, using modified sequences synthesized by Geneart (Regensburg, Del.). The resulting monomeric form of IgA2 lacked the tail piece and is shown in SEQ ID NO: 2 (FIG. 2b). Furthermore, the presence of the amino acid residue proline at position 124 (IMGT numbering for C-domain) in the CH1 domain and directly preceding the hinge region, impacts how the IgA2 light chain pairs with its heavy chain (Lombana et al., (2019) MABS, 11(1): 75-93). A substitution of proline at position 124 with arginine (P124R) reduced considerably the heterogeneity of IgA2 (i.e. light chain-heavy chain mispairing) without affecting the ability of IgA2 to interact with hFcαRI (Table 2), as determined by SDS-PAGE analysis (results not shown). This modification was made in IgA2 sequences with the tail piece, as shown in SEQ ID NO: 131, and in IgA2 sequences without the tail piece, as shown in SEQ ID NO: 3.

TABLE 2
Affinity and maximum response of
engineered immunoglobulin towards hFcαRI
determined by surface plasmon resonance,
following the procedure described in Example 5a.

| SEQ ID | Ref | | Maximum response at |
|---|---|---|---|
| NO | number | KD (1:1) (M) | 1000 nM hFcαRI (RU) |
| 2 | 2258 | 2.59E−07 | 263 |
| 3 | 2737 | 2.38E−07 | 182 |

1.1.2. IgG1/IgA2 CH1 Replacement (IMGT Numbering for C-Domain)

The CH1 domain of an anti-HER2 antibody comprising IgA2 constant domains was replaced by the corresponding CH1 domain of IgG1 (FIG. 2c). When the engineered antibody was tested for hFcαRI binding, the substitution of the CH1 domain, as shown in SEQ ID NO: 4 was found to have limited impact on hFcαRI binding, as determined using surface plasmon resonance (Table 3).

TABLE 3
Affinity and maximum response of
engineered immunoglobulin towards hFcαRI
determined by surface plasmon resonance,
following the procedure described in Example 5a.

| | | | Maximum response |
|---|---|---|---|
| SEQ ID | Ref | | at 1000 nM hFcαRI |
| NO | number | KD (1:1) (M) | (RU) |
| 2 | 2258 | 2.59E−07 | 263 |
| 4 | 2259 | 4.17E−07 | 294 |
| 5 | 2260 | 4.91E−07 | 210 |
| 6 | 2261 | 4.73E−07 | 270 |

1.1.3. IgG1/IgA2 Hinge Replacement (IMGT Numbering for C-Domain)

The CH1 and hinge domains of the heavy chain of an IgA2 anti-HER2 antibody were replaced with the corresponding CH1 and hinge domains from IgG1 (FIG. 2d). When the engineered antibody was tested for hFcαRI binding, the substitution of the CH1 and hinge domains as shown in SEQ ID NOs: 7, 8 and 9) was found to have little impact on hFcαRI binding, as determined using surface plasmon resonance (Table 4).

TABLE 4
Affinity and maximum response of
engineered immunoglobulin towards hFcαRI
determined by SPR experiment, following
procedure described in Example 5a.

| | | | Maximum response |
|---|---|---|---|
| SEQ ID | Ref | | at 1000 nM hFcαRI |
| NO | number | KD (1:1) (M) | (RU) |
| 2 | 2258 | 2.59E−07 | 263 |
| 7 | 2262 | 3.23E−07 | 290 |
| 8 | 2263 | 6.42E−07 | 202 |
| 9 | 2264 | 3.49E−07 | 300 |

1.1.4. IgA2 Fc/hFcαRI Complex In-Silico Analysis (Yasara and PyMol)

In-silico analysis was performed on a complex of IgA1 Fc with hFcαRI using the crystal structure PDB 1D 1OWO. The computer program YASARA for molecular visualizing (www.yasara.org) and the open source molecular visualization system PyMOL (https://pymol.org) were used to display and work on the 3D complex structure. All solvent exposed residues and residues located in proximity to hFcαRI (distance d<7 Angstrom) were considered to be potentially involved in the interaction of IgA1 Fc with hFcαRI.

1.1.5. Superimposition IgG1/IgA1 Fcs (PyMOL)

In-silico superimposition of IgG1 and IgA1 Fcs was performed using the IgA1 Fc/hFcαRI crystal structure (PDB ID 1OWO) and the IgG1 Fc crystal structure PDB 1FC1. Based on this superimposition and “necklace” representation (IMGT resources), IgG1 residues structurally equivalent to IgA1 residues involved in IgA1 Fc/hFcαRI interactions were identified.

1.1.6. IgA/hFcαRI Interaction Site and Secondary Structure Elements Extraction (IMGT Numbering for C-Domain)

Previously identified IgG1 residues (see section 1.1.4) were replaced by corresponding residues from IgA2. Also, residues responsible for secondary structures (alpha helixes, beta strands and turns holding or related to residues described in 1.1.4 above) were transferred from IgA2 into IgG1 since they were observed from the crystal structure to be holding and orientating residues that interact directly with hFcαRI.

1.1.7. IgG/IgA CH2, CH3 and CH2/CH3 Elbow Length Adjustment (IMGT Numbering for C-Domain)

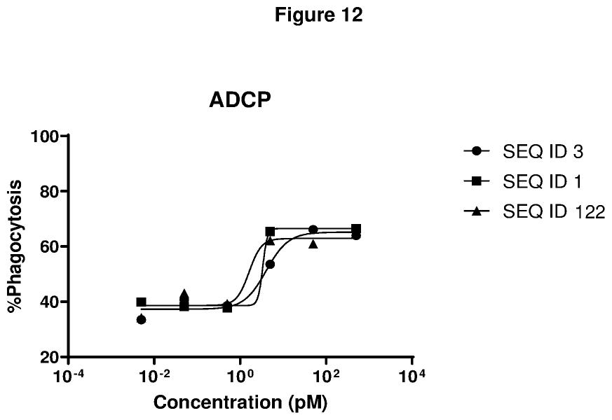

Although IgG1 and IgA2 share the same structural homology, the amino acid sequence length and angles between CH2 and CH3 holding residues interacting with hFcαRI are different. For this reason, the length of IgG1 CH2 and CH3 domains was adjusted in a region at the CH2/CH3 interface, termed herein as the “CH2/CH3 elbow” (FIG. 2e). This region, comprising approximately the last three residues of the CH2 domain and the first six residues of the CH3 domain of IgA2 (shown schematically in FIG. 8), was shortened, based on the structural IgG1/IgA1 comparison and “necklace” display (IMGT resources) as shown in SEQ ID NOs: 4, 5, 6, 7, 8 and 9. Influence of the length of this elbow on binding to hFcαRI is shown in Table 5.

TABLE 5
Affinity and maximum response of
engineered immunoglobulin towards hFcαRI
determined by SPR experiment, following
procedure described in Example 5a.

| | | | Maximum response |
|---|---|---|---|
| SEQ ID | Ref | | at 1000 nM hFcαRI |
| NO | number | KD (1:1) (M) | (RU) |
| 2 | 2258 | 2.59E−07 | 263 |
| 4 | 2259 | 4.17E−07 | 294 |
| 5 | 2260 | 4.91E−07 | 210 |
| 6 | 2261 | 4.73E−07 | 270 |
| 7 | 2262 | 3.23E−07 | 290 |
| 8 | 2263 | 6.42E−07 | 202 |
| 9 | 2264 | 3.49E−07 | 300 |

1.1.8. IgG/IgA CH2/CH2 Modifications Based on Rational Design

In the engineered anti-HER2 antibody already comprising a CH1 domain and hinge region from IgG1, the CH2 domain was then replaced with a CH2 domain from IgG1 (FIG. 2f). Using rational design, various amino acid modifications were made at positions in the CH2 domain based on the superimposition work as described in section 1.1.5 above to substitute the IgG1 residue with the corresponding residue from IgA2 (as shown in SEQ ID NOs: 10 to 12, 14 to 23). These CH2 mutations are summarized in Table 6 below compared to wild-type IgG1 (SEQ ID NO: 1) and their impact on binding on hFcαRI is presented in Table 7.

TABLE 6
Summary of CH2 mutations (based on sequence SEQ ID NO: 1)

| SEQ | |
|---|---|
| ID NO | CH2 Mutation set (IMGT numbering for C-domain) |
| 10 | P8R, K10A, P11L, L12E, T14L, M15.1L, S16G, R17S, T18E, T90P, V91G, L92C, H93A, |
| | D95P, L97N, N98H, S122T, A124S, K125# |
| 11 | P8R, K10A, P11L, L12E, T14L, M15.1L, S16G, R17S, T18E, P19A, E20N, T90P, V91G, |
| | L92C, H93A, D95P, L97N, N98H, S122T, A124S, K125# |
| 12 | P8R, K10A, P11L, L12E, T14L, M15.1L, S16G, R17S, T18E, P19A, E20N, T90P, V91G, |
| | H93A, D95P, L97N, N98H, S122T, A124S, K125# |
| 14 | P8R, K10A, P11L, L12E, T14L, M15.1L, S16G, R17S, T18E, P19A, E20N, N84.4A, T90P, |
| | V91G, H93A, D95P, L97N, K125# |
| 15 | P8R, K10A, P11L, L12E, T14L, M15.1L, S16G, R17S, T18E, P19A, E20N, D34#, H45.5#, |
| | N77#, S85.4G, T90P, V91G, H93A, D95P, L97N, N98H, K125# |
| 16 | A1.6#, P1.5#, E1.4#, L1.3#, L1.2#, G1.1#, P8R, K10A, P11L, L12E, T14L, M15.1L, S16G, |
| | R17S, T18E, P19A, E20N, D34#, H45.5#, N77#, T90P, V91G, H93A, D95P, L97N, N98H, |
| | K125# |
| 17 | L1.2C, G1.1C, P8R, K10A, P11L, L12E, T14L, M15.1L, S16G, R17S, T18E, P19A, E20N, |
| | D34#, H45.5#, N77#, N84.4C, S85.4G, T85.3C, T90P, V91G, H93A, D95P, L97N, N98H, |
| | K125# |
| 18 | A1.6#, P1.5#, E1.4#, L1.3#, L1.2C, G1.1C, P8R, K10A, P11L, L12E, T14L, M15.1L, S16G, |
| | R17S, T18E, P19A, E20N, D34#, H45.5#, N77#, N84.4C, S85.4G, T85.3C, T90P, V91G, |
| | H93A, D95P, L97N, N98H, K125# |
| 19 | P8R, K10A, P11L, L12E, T14L, M15.1L, S16G, R17S, T18E, P19A, E20N, N84.4A, T90P, |
| | V91G, L92C, H93A, D95P, L97N, N98H, K125# |
| 20 | P8R, K10A, P11L, L12E, T14L, M15.1L, S16G, R17S, T18E, P19A, E20N, D34#, H45.5#, |
| | N77#, S85.4G, T90P, V91G, L92C, H93A, D95P, L97N, N98H, K125# |
| 21 | A1.6#, P1.5#, E1.4#, L1.3#, L1.2#, G1.1#, P8R, K10A, P11L, L12E, T14L, M15.1L, S16G, |
| | R17S, T18E, P19A, E20N, D34#, H45.5#, N77#, S85.4G, T90P, V91G, L92C, H93A, D95P, |
| | L97N, N98H, K125# |
| 22 | L1.2C, G1.1C, P8R, K10A, P11L, L12E, T14L, M15.1L, S16G, R17S, T18E, P19A, E20N, |
| | D34#, H45.5#, N77#, N84.4C, S85.4G, T85.3C, T90P, V91G, L92C, H93A, D95P, L97N, |
| | N98H, K125# |
| 23 | A1.6#, P1.5#, E1.4#, L1.3#, L1.2C, G1.1C, P8R, K10A, P11L, L12E, T14L, M15.1L, S16G, |
| | R17S, T18E, P19A, E20N, D34#, H45.5#, N77#, N84.4C, S85.4G, T85.3C, T90P, V91G, |
| | L92C, H93A, D95P, L97N, N98H, K125# |

TABLE 7
Affinity and maximum response of engineered
immunoglobulin towards hFcαRI
determined by surface plasmon resonance,
following the procedure described in Example 5a.

| SEQ ID | Ref | | Maximum response at |
|---|---|---|---|
| NO | number | KD (1:1) (M) | 1000 nM hFcαRI (RU) |
| 2 | 2258 | 2.59E−07 | 263 |
| 10 | 2265 | 0.00E+00 | 0 |
| 11 | 2266 | 0.00E+00 | 0 |
| 12 | 2267 | 0.00E+00 | 10 |
| 14 | 2343 | 0.00E+00 | 0 |
| 15 | 2344 | 0.00E+00 | 0 |
| 16 | 2345 | 0.00E+00 | 0 |
| 17 | 2346 | 0.00E+00 | 0 |
| 18 | 2347 | 0.00E+00 | 0 |
| 19 | 2348 | 0.00E+00 | 0 |
| 20 | 2349 | 0.00E+00 | 0 |
| 21 | 2350 | 0.00E+00 | 0 |
| 22 | 2351 | 0.00E+00 | 0 |
| 23 | 2352 | 0.00E+00 | 0 |

1.1.9 IgG/IgA CH3/CH3 Core Interface Replacement Using Rational Design

The above described CH2 modifications were not sufficient to restore hFcαRI binding to the engineered antibody. Therefore to investigate whether modifications to the CH3 domain could contribute to hFcαRI binding, an engineered anti-HER2 antibody was generated comprising a CH1 domain and a CH3 domain from IgG1 but retaining the hinge and CH2 domain from IgA2, and with a CH2/CH3 elbow region from IgG1. In addition, further modifications were introduced to CH3 domain to replace IgG1 residues with IgA2 residues interacting with hFcαRI held by the beta-sheet. From structural analysis, it is known that one side of the beta sheet turns the side chain of the IgA2 CH3 domain amino acid residues towards hFcαRI, whereas the other side of the beta sheet turns the side chains of the IgA2 CH3 residues towards the CH3/CH3 core interface. The CH3/CH3 core residues can affect the positioning of the residue side chains in their interactions with hFcαRI depending of their properties and steric hindrance. For this reason, IgG1 CH3 core residues at the CH3/CH3 interface were exchanged with IgA2 residues in order to correctly orientate interacting residues with hFcαRI, located on the other side of the beta-sheet (FIG. 2g; as shown in SEQ ID NOs: 13, 24 to 27, 46 to 48 and 87 to 94). These CH2 mutations are summarized in Table 8 below and their effect on hFcαRI binding is presented in Table 9.

TABLE 8
Summary of CH3 mutations (based on sequence SEQ ID NO: 1)

| SEQ | |
|---|---|
| ID NO | CH3 Mutation set (IMGT numbering for C-domain) |
| 87 | R1.1F, T6L, S10P, R11S, D12E, T15A, \hat{ }15L, K16N, N17E, Q18L, V25A, Y29S, S35K, |
| | I37V, A38L, E40R, E42L, S43Q, N44G, G45S, P45.2E, E45.3L, N45.4P, \hat{ }45.4R,E, P82A, |
| | P83S, L84.1Q, D84.2E, S84.3P, D84.4S, \hat{ }84.4Q,G,T, G85.4T, L85V, D92A, K93A, S94E, |
| | R95D, Q97K, Q98K, N100D, V101T, S105M, M107G, H113P, N114L, H115A, Y116F, |
| | L121I, L123R |
| 88 | R1.1F, T6L, V25A, Y29S, S35K, I37V, A38L, E40R, E42L, S43Q, N44G, G45S, P45.2E, |
| | E45.3L, N45.4P, \hat{ }45.4R,E, P82A, P83S, L84.1Q, D84.2E, S84.3P, D84.4S, \hat{ }84.4Q,G,T, |
| | G85.4T, L85V, D92A, K93A, S94E, R95D, Q97K, Q98K, N100D, V101T, S105M, M107G, |
| | H113P, N114L, H115A, Y116F, L121I, L123R |
| 89 | T6L, V25A, S35K, I37V, A38L, E40R, E42L, S43Q, N44G, G45S, P45.2E, E45.3L, N45.4P, |
| | \hat{ }45.4R,E, P82A, P83S, L85V, D92A, K93A, S94E, R95D, Q97K, Q98K, N100D, V101T, |
| | S105M, M107G, H113P, N114L, H115A, Y116F, L121I, L123R |
| 90 | T6L, S10P, R11S, D12E, T15A, \hat{ }15L, K16N, N17E, Q18L, V25A, S35K, I37V, A38L, E40R, |
| | E42L, S43Q, N44G, G45S, P45.2E, E45.3L, N45.4P, \hat{ }45.4R,E, P82A, P83S, L85V, D92A, |
| | K93A, S94E, R95D, Q97K, Q98K, N100D, V101T, S105M, M107G, H113P, N114L, H115A, |
| | Y116F, L121I, L123R |
| 91 | T6L, S10P, R11S, D12E, T15A, \hat{ }15L, K16N, N17E, Q18L, V25A, S35K, I37V, A38L, E40R, |
| | E42L, S43Q, N44G, G45S, P45.2E, E45.3L, N45.4P, \hat{ }45.4R,E, P82A, P83S, L85V, |
| | S105M, M107G, H113P, N114L, H115A, Y116F, L121I |
| 92 | R1.1F, T6L, S10P, R11S, D12E, T15A, \hat{ }15L, K16N, N17E, Q18L, V25A, Y29S, S35K, |
| | I37V, A38L, E40R, E42L, S43Q, N44G, G45S, P45.2E, E45.3L, N45.4P, \hat{ }45.4R,E, P82A, |
| | P83S, L84.1Q, D84.2E, S84.3P, D84.4S, \hat{ }84.4Q,G,T, G85.4T, L85V, S105M, M107G, |
| | H113P, N114L, H115A, Y116F, L121I |
| 93 | R1.1F, T6L, V25A, Y29S, S35K, I37V, A38L, E40R, E42L, S43Q, N44G, G45S, P45.2E, |
| | E45.3L, N45.4P, \hat{ }45.4R,E, P82A, P83S, L84.1Q, D84.2E, S84.3P, D84.4S, \hat{ }84.4Q,G,T, |
| | G85.4T, L85V, S105M, M107G, H113P, N114L, H115A, Y116F, L121I |
| 94 | T6L, V25A, S35K, I37V, A38L, E40R, E42L, S43Q, N44G, G45S, P45.2E, E45.3L, N45.4P, |
| | \hat{ }45.4R,E, P82A, P83S, L85V, S105M, M107G, H113P, N114L, H115A, Y116F, L121I |
| 13 | Q1.3N, P1.2T, R1.1F, E1R, A38L, E40R, E42L, S43Q, N44G, G45S, P45.2E, E45.3L, |
| | V101T, S105M, M107G, H113P, N114L, H115A, Y116F, S120T |
| 24 | A38L, E40R, E42L, S43Q, N44G, G45S, P45.2E, E45.3L, V101T, S105M, M107G, H113P, |
| | N114L, H115A, Y116F, S120T |
| 25 | A38L, E40R, E42L, S43Q, N44G, G45S, P45.2E, E45.3L, N45.4P, \hat{ }45.4R,E, V101T, |
| | S105M, M107G, H113P, N114L, H115A, Y116F, S120T |
| 26 | A38L, E40R, E42L, S43Q, N44G, G45S, P45.2E, E45.3L, N45.4P, \hat{ }45.4R,E, \hat{ }84.4Q,G,T, |
| | V101T, S105M, M107G, H113P, N114L, H115A, Y116F, S120T |
| 27 | \hat{ }15L, A38L, E40R, E42L, S43Q, N44G, G45S, P45.2E, E45.3L, N45.4P, \hat{ }45.4R,E, |
| | \hat{ }84.4Q,G,T, V101T, S105M, M107G, H113P, N114L, H115A, Y116F, S120T |
| 46 | E1R, \hat{ }15L, A38L, E40R, E42L, S43Q, N44G, G45S, P45.2E, E45.3L, N45.4P, \hat{ }45.4R,E, |
| | \hat{ }84.4Q,G,T, V101T, S105M, M107G, H113P, N114L, H115A, Y116F, S120T |
| 47 | Q3E, \hat{ }15L, A38L, E40R, E42L, S43Q, N44G, G45S, P45.2E, E45.3L, N45.4P, \hat{ }45.4R,E, |
| | \hat{ }84.4Q,G,T, V101T, S105M, M107G, H113P, N114L, H115A, Y116F, S120T |
| 48 | E1R, Q3E, \hat{ }15L, A38L, E40R, E42L, S43Q, N44G, G45S, P45.2E, E45.3L, N45.4P, |
| | \hat{ }45.4R,E, \hat{ }84.4Q,G,T, V101T, S105M, M107G, H113P, N114L, H115A, Y116F, S120T |

TABLE 9
Affinity and maximum response of engineered
immunoglobulin towards hFcαRI
determined by surface plasmon resonance,
following the procedure described in Example 5a.

| | | | Maximum response |
|---|---|---|---|
| SEQ ID | Ref | | at 1000nM hFcαRI |
| NO | number | KD (1:1) (M) | (RU) |
| 2 | 2258 | 2.59E−07 | 263 |
| 13 | 2268 | 7.33E−06 | 67 |
| 24 | 2353 | 4.43E−06 | 46 |
| 25 | 2354 | 2.73E−06 | 92 |
| 26 | 2355 | 3.05E−06 | 120 |
| 27 | 2356 | 2.23E−06 | 104 |
| 46 | 2459 | 5.64E−06 | 54 |
| 47 | 2460 | 2.47E−06 | 70 |
| 48 | 2461 | 3.10E−06 | 89 |
| 87 | 2703 | 5.89E−07 | 103 |
| 88 | 2704 | 5.50E−07 | 225 |
| 89 | 2705 | 6.60E−07 | 271 |
| 90 | 2706 | 6.08E−07 | 288 |
| 91 | 2707 | 8.44E−07 | 200 |
| 92 | 2708 | 8.43E−07 | 200 |
| 93 | 2709 | 1.14E−06 | 171 |
| 94 | 2710 | 1.04E−06 | 213 |

1.2 Semi-Rational Design—from IgA2 to IgG1

Pure rational design as described in Section 1.1 above offered the possibility to identify residues involved in the IgA2/hFcαRI interaction for subsequent transfer of these residues into an IgG1 Fc. The transfer of interacting residues and structural elements, length adjustment and engineering of CH3/CH3 core residues was successful for hinge, CH2/CH3 elbow and CH3 engineering. However, for fine tuning of the CH2 and CH3 engineering, it was necessary to complete the campaign using a semi-rational approach.

1.2.1. ABCDEFG Beta-Strands Scan

As previously discussed, IgG1 and IgA2 share a high degree of structural homology and the CH2 and CH3 domains are both comprised of beta-sheets made from anti-parallel β-strands A, B, C, D, E, F and G (IMGT nomenclature). Each of the IgA2 CH2 and CH3 β-strands were individually scanned by sequential substitution with equivalent IgG1 β-strands. Furthermore, β-strands were replaced depending on their role in the IgA2/hFcαRI interaction, since some strands are known to hold hey residues that interact directly with hFcαRI. The replacements were: A+B, E+F, C+D, C+D+G, A+B+E+F, C+D+E+F+G, A+B+C+D+G, with up to five IgA2 beta strands replaced by IgG1 beta strands. The amino acid sequences depicted in SEQ ID NOs: 28 to 41 exemplify the modifications made to the IgA2 CH2 domain by substituting with residues from the IgG1 CH2 domain (FIG. 3a). The amino acid sequences depicted in SEQ ID NOs: 58 to 68 exemplify the modifications made to the IgA2 CH3 domain by substituting with residues from the IgG1 CH3 domain (FIG. 3b). These CH2 and CH3 mutations are summarized in Table 10 and 12 below and their effect on hFcαRI binding is presented in Table 11 and 13.

TABLE 10
Summary of CH2 mutations (based on sequence SEQ ID NO: 1)

| SEQ | |
|---|---|
| ID NO | CH2 Mutation set (IMGT numbering for C-domain) |
| 28 | P8R, K10A, P11L, K12E, T14L, M15.1L, I15.2#, S16G, R17S, T18E, P19A, E20N, V21L, |
| | V24T, V25L, V26T, D27G, V28L, S29R, H30D, E31A, D34#, P35S, E36G, V37A, K38T, |
| | N40T, Y42T, V43P, D44S, G45S, V45.1G, E45.2K, V45.3S, H45.4#, N77#, K79V, T80Q, |
| | K81G, R83P, E84.1R, Q84.2D, Y84.3L, N84.4C, S85.4G, T85.3C, R85.1S, V86S, T90P, |
| | V91G, L92C, H93A, D95P, L97N, N98H, K100E, E101T, Y102F, K103T, K105T, V106A, |
| | S107A, N108H, K109P, A110E, P114K, A115T, P116P, I117L, E118T, K119A, T120N, |
| | S122T, A124S, K125# |
| 29 | A1.6#, P1.5#, E1.4#, L1.3#, L1.2C, G1.1C, G1H, S3R, V4L, F5S, F7H, P8R, K10A, P11L, |
| | K12E, T14L, M15.1L, I15.2#, S16G, R17S, T18E, P19A, E20N, E31A, D34#, P35S, E36G, |
| | V37A, K38T, N40T, Y42T, V43P, D44S, G45S, V45.1G, E45.2K, V45.3S, H45.4#, N77#, |
| | K79V, T80Q, K81G, R83P, E84.1R, Q84.2D, Y84.3L, N84.4C, S85.4G, T85.3C, R85.1S, |
| | V86S, T90P, V91G, L92C, H93A, D95P, L97N, N98H, K100E, E101T, Y102F, K103T, |
| | K105T, V106A, S107A, N108H, K109P, A110E, P114K, A115T, P116P, I117L, E118T, |
| | K119A, T120N, S122T, A124S, K125# |
| 30 | A1.6#, P1.5#, E1.4#, L1.3#, L1.2C, G1.1C, G1H, S3R, V4L, F5S, F7H, P8R, K10A, P11L, |
| | K12E, T14L, M15.1L, I15.2#, S16G, R17S, T18E, P19A, E20N, V21L, V24T, V25L, V26T, |
| | D27G, V28L, S29R, H30D, V45.1G, E45.2K, V45.3S, H45.4#, N77#, K79V, T80Q, K81G, |
| | R83P, E84.1R, Q84.2D, Y84.3L, N84.4C, S85.4G, T85.3C, R85.1S, V86S, T90P, V91G, |
| | L92C, H93A, D95P, L97N, N98H, K100E, E101T, Y102F, K103T, K105T, V106A, S107A, |
| | N108H, K109P, A110E, P114K, A115T, P116P, I117L, E118T, K119A, T120N, S122T, |
| | A124S, K125# |
| 31 | A1.6#, P1.5#, E1.4#, L1.3#, L1.2C, G1.1C, G1H, S3R, V4L, F5S, F7H, P8R, K10A, P11L, |
| | K12E, T14L, M15.1L, I15.2#, S16G, R17S, T18E, P19A, E20N, V21L, V24T, V25L, V26T, |
| | D27G, V28L, S29R, H30D, E31A, D34#, P35S, E36G, V37A, K38T, N40T, Y42T, V43P, |
| | D44S, G45S, H45.4#, N77#, N84.4C, S85.4G, T85.3C, R85.1S, V86S, T90P, V91G, L92C, |
| | H93A, D95P, L97N, N98H, K100E, E101T, Y102F, K103T, K105T, V106A, S107A, N108H, |
| | K109P, A110E, P114K, A115T, P116P, I117L, E118T, K119A, T120N, S122T, A124S, |
| | K125# |
| 32 | A1.6#, P1.5#, E1.4#, L1.3#, L1.2#, G1.1#, G1H, S3R, V4L, F5S, F7H, P8R, K10A, P11L, |
| | K12E, T14L, M15.1L, I15.2#, S16G, R17S, T18E, P19A, E20N, V21L, V24T, V25L, V26T, |
| | D27G, V28L, S29R, H30D, E31A, D34#, P35S, E36G, V37A, K38T, N40T, Y42T, V43P, |
| | D44S, G45S, V45.1G, E45.2K, V45.3S, H45.4#, N77#, K79V, T80Q, K81G, R83P, E84.1R, |
| | Q84.2D, Y84.3L, T90P, V91G, L92C, H93A, D95P, L97N, N98H, K100E, E101T, Y102F, |
| | K103T, K105T, V106A, S107A, N108H, K109P, A110E, P114K, A115T, P116P, I117L, |
| | E118T, K119A, T120N, S122T, A124S, K125# |
| 33 | A1.6#, P1.5#, E1.4#, L1.3#, L1.2C, G1.1C, G1H, S3R, V4L, F5S, F7H, P8R, K10A, P11L, |
| | K12E, T14L, M15.1L, I15.2#, S16G, R17S, T18E, P19A, E20N, V21L, V24T, V25L, V26T, |
| | D27G, V28L, S29R, H30D, E31A, D34#, P35S, E36G, V37A, K38T, N40T, Y42T, V43P, |
| | D44S, G45S, V45.1G, E45.2K, V45.3S, H45.4#, N77#, K79V, T80Q, K81G, R83P, E84.1R, |
| | Q84.2D, Y84.3L, N84.4C, S85.4G, T85.3C, R85.1S, V86S, T90P, V91G, L92C, H93A, |
| | D95P, L97N, N98H, P114K, A115T, P116P, I117L, E118T, K119A, T120N, S122T, A124S, |
| | K125# |
| 34 | A1.6#, P1.5#, E1.4#, L1.3#, L1.2C, G1.1C, G1H, S3R, V4L, F5S, F7H, P8R, K10A, P11L, |
| | K12E, T14L, M15.1L, I15.2#, S16G, R17S, T18E, P19A, E20N, V21L, V24T, V25L, V26T, |
| | D27G, V28L, S29R, H30D, E31A, D34#, P35S, E36G, V37A, K38T, N40T, Y42T, V43P, |
| | D44S, G45S, V45.1G, E45.2K, V45.3S, H45.4#, N77#, K79V, T80Q, K81G, R83P, E84.1R, |
| | Q84.2D, Y84.3L, N84.4C, S85.4G, T85.3C, R85.1S, V86S, T90P, V91G, L92C, H93A, |
| | D95P, L97N, N98H, K100E, E101T, Y102F, K103T, K105T, V106A, S107A, N108H, |
| | K109P, A110E, S122T, A124S, K125# |
| 35 | A1.6#, P1.5#, E1.4#, L1.3#, L1.2C, G1.1C, G1H, S3R, V4L, F5S, F7H, P8R, K10A, P11L, |
| | K12E, T14L, M15.1L, I15.2#, S16G, R17S, T18E, P19A, E20N, V21L, V24T, V25L, V26T, |
| | D27G, V28L, S29R, H30D, N84.4C, S85.4G, T85.3C, R85.1S, V86S, T90P, V91G, L92C, |
| | H93A, D95P, L97N, N98H, K100E, E101T, Y102F, K103T, K105T, V106A, S107A, N108H, |
| | K109P, A110E, P114K, A115T, P116P, I117L, E118T, K119A, T120N, S122T, A124S, |
| | K125# |
| 36 | A1.6#, P1.5#, E1.4#, L1.3#, L1.2C, G1.1C, G1H, S3R, V4L, F5S, F7H, P8R, K10A, P11L, |
| | K12E, T14L, M15.1L, I15.2#, S16G, R17S, T18E, P19A, E20N, V21L, V24T, V25L, V26T, |
| | D27G, V28L, S29R, H30D, N84.4C, S85.4G, T85.3C, R85.1S, V86S, T90P, V91G, L92C, |
| | H93A, D95P, L97N, N98H, K100E, E101T, Y102F, K103T, K105T, V106A, S107A, N108H, |
| | K109P, A11OE, S122T, A124S, K125# |
| 37 | A1.6#, P1.5#, E1.4#, L1.3#, L1.2#, G1.1#, G1H, S3R, V4L, F5S, F7H, P8R, K10A, P11L, |
| | K12E, T14L, M15.1L, I15.2 delete, S16G, R17S, T18E, P19A, E20N, V21L, V24T, V25L, |
| | V26T, D27G, V28L, S29R, H30D, T90P, V91G, L92C, H93A, D95P, L97N, N98H, S122T, |
| | A124S, K125# |
| 38 | P8R, K10A, P11L, K12E, T14L, M15.1L, I15.2#, S16G, R17S, T18E, P19A, E20N, N84.4C, |
| | S85.4G, T85.3C, R85.1S, V86S, T90P, V91G, L92C, H93A, D95P, L97N, N98H, K100E, |
| | E101T, Y102F, K103T, K105T, V106A, S107A, N108H, K109P, A110E, S122T, A124S, |
| | K125# |
| 39 | P8R, K10A, P11L, K12E, T14L, M15.1L, I15.2#, S16G, R17S, T18E, P19A, E20N, E31A, |
| | D34#, P35S, E36G, V37A, K38T, N40T, Y42T, V43P, D44S, G45S, V45.1G, E45.2K, |
| | V45.3S, H45.4#, N77#, K79V, T80Q, K81G, R83P, E84.1R, Q84.2D, Y84.3L, T90P, V91G, |
| | L92C, H93A, D95P, L97N, N98H, P114K, A115T, P116P, I117L, E118T, K119A, T120N, |
| | S122T, A124S, K125# |
| 40 | P8R, K10A, P11L, K12E, T14L, M15.1L, I15.2#, S16G, R17S, T18E, P19A, E20N, E31A, |
| | D34#, P35S, E36G, V37A, K38T, N40T, Y42T, V43P, D44S, G45S, V45.1G, E45.2K, |
| | V45.3S, H45.4#, N77#, K79V, T80Q, K81G, R83P, E84.1R, Q84.2D, Y84.3L, N84.4C, |
| | S85.4G, T85.3C, R85.1S, V86S, T90P, V91G, L92C, H93A, D95P, L97N, N98H, K100E, |
| | E101T, Y102F, K103T, K105T, V106A, S107A, N108H, K109P, A110E, P114K, A115T, |
| | P116P, I117L, E118T, K119A, T120N, S122T, A124S, K125# |
| 41 | A1.6#, P1.5#, E1.4#, L1.3#, L1.2#, G1.1#, G1H, S3R, V4L, F5S, F7H, P8R, K10A, P11L, |
| | K12E, T14L, M15.1L, I15.2#, S16G, R17S, T18E, P19A, E20N, V21L, V24T, V25L, V26T, |
| | D27G, V28L, S29R, H30D, E31A, D34#, P35S, E36G, V37A, K38T, N40T, Y42T, V43P, |
| | D44S, G45S, V45.1G, E45.2K, V45.3S, H45.4#, N77#, K79V, T80Q, K81G, R83P, E84.1R, |
| | Q84.2D, Y84.3L, T90P, V91G, L92C, H93A, D95P, L97N, N98H, P114K, A115T, P116P, |
| | I117L, E118T, K119A, T120N, S122T, A124S, K125# |

TABLE 11
Affinity and maximum response of engineered
immunoglobulin towards hFcαRI
determined by surface plasmon resonance, following
the procedure described in Example 5a.

| | | | Maximum response |
|---|---|---|---|
| SEQ ID | Ref | | at 1000 nM hFcαRI |
| NO | number | KD (1:1) (M) | (RU) |
| 2 | 2258 | 2.59E−07 | 263 |
| 28 | 2462 | 4.41E−07 | 114 |
| 29 | 2463 | 0.00E+00 | 10 |
| 30 | 2464 | 3.71E−07 | 250 |
| 31 | 2465 | 3.36E−07 | 156 |
| 32 | 2466 | 3.17E−07 | 25 |
| 33 | 2467 | 0.00E+00 | 0 |
| 34 | 2468 | 3.64E−07 | 300 |
| 35 | 2469 | 3.65E−07 | 229 |
| 36 | 2470 | 3.15E−07 | 267 |
| 37 | 2471 | 0.00E+00 | 0 |
| 38 | 2472 | 0.00E+00 | 0 |
| 39 | 2473 | 0.00E+00 | 0 |
| 40 | 2474 | 3.57E−07 | 24 |
| 41 | 2475 | 0.00E+00 | 0 |

TABLE 12
Summary of CH3 mutations (based on sequence SEQ ID NO: 1)

| SEQ | |
|---|---|
| ID NO | CH3 Mutation set (IMGT numbering for C-domain) |
| 58 | \hat{ }15L, K16N, N17E, O18L, S20T, V25A, K26R, Y29S, S31K, I37V, A38L, E40R, E42L, |
| | S43Q, N44G, G45S, P45.2E, E45.3L, N45.4P, \hat{ }45.4RE, N77K, K79L, P82A, P83S, V84R, |
| | L84.1Q, D84.2E, S84.3P, D84.4S, \hat{ }84.4QGT, G85.4T, S85.3T, F85.1A, L85V, Y86T, K88I, |
| | T90R, D92A, K93A, S94E, R95D, Q97K, Q98K, N100D, V101T, S105M, M107G, H113P, |
| | N114L, H115A, Y116F, S120T, L121I, S122D, L123R, S124L, P125A |
| 59 | Q3E, Y5H, T6L, S10P, R11S, D12E, T15A, S31K, I37V, A38L, E40R, E42L, S43Q, N44G, |
| | G45S, P45.2E, E45.3L, N45.4P, \hat{ }45.4RE, N77K, K79L, P82A, P83S, V84R, L84.1Q, |
| | D84.2E, S84.3P, D84.4S, \hat{ }84.4QGT, G85.4T, S85.3T, F85.1A, L85V, Y86T, K88I, T90R, |
| | D92A, K93A, S94E, R95D, Q97K, Q98K, N100D, V101T, S105M, M107G, H113P, N114L, |
| | H115A, Y116F, S120T, L121I, S122D, L123R, S124L, P125A |
| 60 | Q3E, Y5H, T6L, S10P, R11S, D12E, T15A, \hat{ }15L, K16N, N17E, O18L, S20T, V25A, K26R, |
| | Y29S, N77K, K79L, P82A, P83S, V84R, L84.1Q, D84.2E, S84.3P, D84.4S, \hat{ }84.4QGT, |
| | G85.4T, S85.3T, F85.1A, L85V, Y86T, K88I, T90R, D92A, K93A, S94E, R95D, Q97K, |
| | Q98K, N100D, V101T, S105M, M107G, H113P, N114L, H115A, Y116F, S120T, L121I, |
| | S122D, L123R, S124L, P125A |
| 61 | Q3E, Y5H, T6L, S10P, R11S, D12E, T15A, \hat{ }15L, K16N, N17E, O18L, S20T, V25A, K26R, |
| | Y29S, E42L, S43Q, N44G, G45S, P45.2E, E45.3L, N45.4P, A45.4RE, N77K, K79L, P82A, |
| | P83S, V84R, L84.1Q, D84.2E, S84.3P, D84.4S, \hat{ }84.4QGT, G85.4T, S85.3T, F85.1A, |
| | L85V, Y86T, K88I, T90R, D92A, K93A, S94E, R95D, Q97K, Q98K, N100D, V101T, S105M, |
| | M107G, H113P, N114L, H115A, Y116F, S120T, L121I, S122D, L123R, S124L, P125A |
| 62 | Q3E, Y5H, T6L, S10P, R11S, D12E, T15A, \hat{ }15L, K16N, N17E, O18L, S20T, V25A, K26R, |
| | Y29S, S31K, I37V, A38L, E40R, N77K, K79L, P82A, P83S, V84R, L84.1Q, D84.2E, |
| | S84.3P, D84.4S, \hat{ }84.4QGT, G85.4T, S85.3T, F85.1A, L85V, Y86T, K88I, T90R, D92A, |
| | K93A, S94E, R95D, Q97K, Q98K, N100D, V101T, S105M, M107G, H113P, N114L, H115A, |
| | Y116F, S120T, L121I, S122D, L123R, S124L, P125A |
| 63 | Q3E, Y5H, T6L, S10P, R11S, D12E, T15A, \hat{ }15L, K16N, N17E, O18L, S20T, V25A, K26R, |
| | Y29S, S31K, I37V, A38L, E40R, E42L, S43Q, N44G, G45S, P45.2E, E45.3L, N45.4P, |
| | \hat{ }45.4RE, \hat{ }84.4QGT, G85.4T, S85.3T, F85.1A, L85V, Y86T, K88I, T90R, D92A, K93A, |
| | S94E, R95D, Q97K, Q98K, N100D, V101T, S105M, M107G, H113P, N114L, H115A, |
| | Y116F, S120T, L121I, S122D, L123R, S124L, P125A |
| 64 | Q3E, Y5H, T6L, S10P, R11S, D12E, T15A, \hat{ }15L, K16N, N17E, O18L, S20T, V25A, K26R, |
| | Y29S, S31K, I37V, A38L, E40R, E42L, S43Q, N44G, G45S, P45.2E, E45.3L, N45.4P, |
| | \hat{ }45.4RE, N77K, K79L, P82A, P83S, V84R, L84.1Q, D84.2E, S84.3P, D84.4S, \hat{ }84.4QGT, |
| | K93A, S94E, R95D, Q97K, Q98K, N100D, V101T, S105M, M107G, H113P, N114L, H115A, |
| | Y116F, S120T, L121I, S122D, L123R, S124L, P125A |
| 65 | Q3E, Y5H, T6L, S10P, R11S, D12E, T15A, \hat{ }15L, K16N, N17E, O18L, S20T, V25A, K26R, |
| | Y29S, S31K, I37V, A38L, E40R, E42L, S43Q, N44G, G45S, P45.2E, E45.3L, N45.4P, |
| | \hat{ }45.4RE, N77K, K79L, P82A, P83S, V84R, L84.1Q, D84.2E, S84.3P, D84.4S, \hat{ }84.4QGT, |
| | G85.4T, S85.3T, F85.1A, L85V, Y86T, K88I, T90R, D92A, V101T, S105M, M107G, H113P, |
| | N114L, H115A, Y116F, S120T, L121I, S122D, L123R, S124L, P125A |
| 66 | Q3E, Y5H, T6L, S10P, R11S, D12E, T15A, \hat{ }15L, K16N, N17E, O18L, S20T, V25A, K26R, |
| | Y29S, S31K, I37V, A38L, E40R, E42L, S43Q, N44G, G45S, P45.2E, E45.3L, N45.4P, |
| | \hat{ }45.4RE, N77K, K79L, P82A, P83S, V84R, L84.1Q, D84.2E, S84.3P, D84.4S, \hat{ }84.4QGT, |
| | G85.4T, S85.3T, F85.1A, L85V, Y86T, K88I, T90R, D92A, K93A, S94E, R95D, Q97K, |
| | Q98K, N100D, H113P, N114L, H115A, Y116F, S120T, L121I, S122D, L123R, S124L, |
| | P125A |
| 67 | Q3E, Y5H, T6L, S10P, R11S, D12E, T15A, \hat{ }15L, K16N, N17E, O18L, S20T, V25A, K26R, |
| | Y29S, S31K, I37V, A38L, E40R, E42L, S43Q, N44G, G45S, P45.2E, E45.3L, N45.4P, |
| | \hat{ }45.4RE, N77K, K79L, P82A, P83S, V84R, L84.1Q, D84.2E, S84.3P, D84.4S, \hat{ }84.4QGT, |
| | G85.4T, S85.3T, F85.1A, L85V, Y86T, K88I, T90R, D92A, K93A, S94E, R95D, Q97K, |
| | Q98K, N100D, V101T, S105M, M107G, S120T, L121I, S122D, L123R, S124L, P125A |
| 68 | Q3E, Y5H, T6L, S10P, R11S, D12E, T15A, \hat{ }15L, K16N, N17E, O18L, S20T, V25A, K26R, |
| | Y29S, S31K, I37V, A38L, E40R, E42L, S43Q, N44G, G45S, P45.2E, E45.3L, N45.4P, |
| | \hat{ }45.4RE, N77K, K79L, P82A, P83S, V84R, L84.1Q, D84.2E, S84.3P, D84.4S, \hat{ }84.4QGT, |
| | G85.4T, S85.3T, F85.1A, L85V, Y86T, K88I, T90R, D92A, K93A, S94E, R95D, Q97K, |
| | Q98K, N100D, V101T, S105M, M107G, H113P, N114L, H115A, Y116F |

TABLE 13
Affinity and maximum response of engineered
immunoglobulin towards hFcαRI
determined by SPR experiment, following
procedure described in Example 5a.

| SEQ ID | Ref | | Maximum response at |
|---|---|---|---|
| NO | number | KD (1:1) (M) | 1000 nM hFcαRI (RU) |
| 2 | 2258 | 2.59E−07 | 263 |
| 58 | 2576 | 4.33E−07 | 160 |
| 59 | 2577 | 6.26E−07 | 62 |
| 60 | 2578 | 0.00E+00 | 0 |
| 61 | 2579 | 1.75E−06 | 25 |
| 62 | 2580 | 2.56E−07 | 17 |
| 63 | 2581 | 1.23E−06 | 155 |
| 64 | 2582 | 3.70E−07 | 12 |
| 65 | 2583 | 9.88E−07 | 148 |
| 66 | 2584 | 0.00E+00 | 0 |
| 67 | 2585 | 0.00E+00 | 0 |
| 68 | 2586 | 5.99E−07 | 233 |

1.2.2. Domain Cutting (Top/Down, Front/Side)

Given their structural similarity, IgG1 and IgA2 CH2 and CH3 can be defined as “building blocks”, with the possibility to cut these domains into pieces. Two different types of sections were cut: (i) a section following the transverse plane (top/down section), and (ii) a section following the frontal plane (front/side section). Final constructs based on an anti-HER2 antibody with a CH1 domain from IgG1, contained IgG1/IgA2 hybrid CH2 or CH3 domains, made of 50% IgA2 and 50% IgG1. The amino acid sequences depicted in SEQ ID NOs: 42 to 45 exemplify the modifications made to the CH2 domain and the amino acid sequences depicted in SEQ ID NOs: 69 and 70 exemplify the modifications made to the CH3 domain by the transverse or frontal plane sectional substitutions. These CH2 and CH3 mutations are shown schematically in FIG. 3c-FIG. 3g, are summarized in Tables 14 and 16 below and their effect on hFcαRI binding is presented in Tables 15 and 17.

TABLE 14
Summary of CH2 mutations (based on sequence SEQ ID NO: 1)

| SEQ | |
|---|---|
| ID NO | CH2 Mutation set (IMGT numbering for C-domain) |
| 42 | P8R, K10A, P11L, K12E, T14L, M15.1L, I15.2#, S16G, R17S, T18E, P19A, E20N, E31A, |
| | D32#, P35S, E36G, V37A, K38T, N40T, Y42T, V43P, D44S, G45S, T90P, V91G, L92C, |
| | H93A, D95P, L97N, N98H, K100E, E101T, Y102F, K103T, K105T, V106A, S107A, N108H, |
| | K109P, A110E, P114K, A115T, I117L, E118T, K119A, T120N, S122T, A124S |
| 43 | A1.6#, P1.5#, E1.4#, L1.3#, L1.2C, G1.1C, G1H, S3R, V4L, F5S, F7H, P8R, K10A, P11L, |
| | K12E, T14L, M15.1L, I15.2#, S16G, R17S, T18E, P19A, E20N, V21L, V24T, V25L, V26T, |
| | D27G, V28L, S29R, H30D, V45.1G, E45.2K, V45.3S, H45.4#, N77#, K79V, T80Q, K81G, |
| | R83P, E84.1R, Q84.2D, Y84.3L, N84.4C, S85.4G, T85.3C, R85.1S, V86S, ET90P, V91G, |
| | L92C, H93A, D95P, L97N, N98H, S122T, A124S |
| 44 | V4L, F5S, F7H, P8R, K10A, P11L, K12E, T14L, M15.1L, I15.2#, S16G, R17S, T18E, P19A, |
| | E20N, V21L, V24T, V25L, N40T, Y42T, V43P, D44S, G45S, V45.1G, E45.2K, V45.3S, |
| | H45.4#, N77#, K79V, V86S, ET90P, V91G, L92C, H93A, D95P, L97N, N98H, K100E, |
| | E101T, Y102F, K103T, K105T, I117L, E118T, K119A, T120N, S122T, A124S |
| 45 | A1.6#, P1.5#, E1.4#, L1.3#, L1.2C, G1.1C, G1H, S3R, P8R, K10A, P11L, K12E, T14L, |
| | M15.1L, I15.2#, S16G, R17S, T18E, P19A, E20N, D27G, V28L, S29R, H30D, E31A, D34#, |
| | P35S, E36G, V37A, K38T, T80Q, K81G, R83P, E84.1R, Q84.2D, Y84.3L, N84.4C, S85.4G, |
| | T85.3C, R85.1S, V86S, ET90P, V91G, L92C, H93A, D95P, L97N, N98H, V106A, S107A, |
| | N108H, K109P, A110E, P114K, A115T S122T, A124S |

TABLE 15
Affinity and maximum response of engineered immunoglobulin
towards hFcαRI determined by surface plasmon resonance, following
the procedure described in Example 5a.

| | | | Maximum response |
|---|---|---|---|
| | | | at 1000 nM hFcαRI |
| SEQ ID NO | Ref number | KD (1:1) (M) | (RU) |
| 2 | 2258 | 2.59E−07 | 263 |
| 42 | 2476 | 0.00E+00 | 0 |
| 43 | 2477 | 0.00E+00 | 0 |
| 44 | 2478 | 0.00E+00 | 0 |
| 45 | 2479 | 1.91E−07 | 233 |
| 45 | 2479 | 2.97E−07 | 187 |

TABLE 16
Summary of CH3 mutations (based on sequence SEQ ID NO: 1)

| SEQ | |
|---|---|
| ID NO | CH3 Mutation set (IMGT numbering for C-domain) |
| 69 | Q3E, Y5H, T6L, V25A, K26R, Y29S, S35K, I37V, A38L, E40R, E42L, S43Q, N44G, G45S, |
| | P45.2E, E45.3L, N45.4P, \hat{ }45.4RE, T81W, P82A, P83S, V84R, L84.1Q, D84.2E, S85.3P, |
| | D84.4S, \hat{ }84.4QGT, G85.4T, S85.3T, F85.1A, L85V, Y86T, S105M, M107G, H113P, |
| | N114L, H115A, Y116F, S120T |
| 70 | S10P, R11S, D12E, T15A, \hat{ }15L, K16N, N17E, Q18L, S20T, A38L, E40R, E42L, S43Q, |
| | N44G, G45S, P45.2E, E45.3L, N45.4P, \hat{ }45.4RE, N77K, K79L, K88I, T90R, D92A, K93A, |
| | S94E, R95D, Q97K, Q98K, N100D, V101T, S105M, M107G, H113P, N114L, H115A, |
| | Y116F, S120T, L121I, S122D, L123R, S124L, P125A |

TABLE 17
Affinity and maximum response of engineered immunoglobulin
towards hFcαRI determined by surface plasmon resonance,
following the procedure described in Example 5a.

| | | | Maximum response |
|---|---|---|---|
| | | | at 1000 nM hFcαRI |
| SEQ ID NO | Ref number | KD (1:1) (M) | (RU) |
| 2 | 2258 | 2.59E−07 | 263 |
| 69 | 2587 | 0.00E+00 | 0 |
| 70 | 2588 | 1.92E−06 | 94 |

1.2.3. Top-CH2 Disulfide Node, CH3-Exposed Alpha Helix

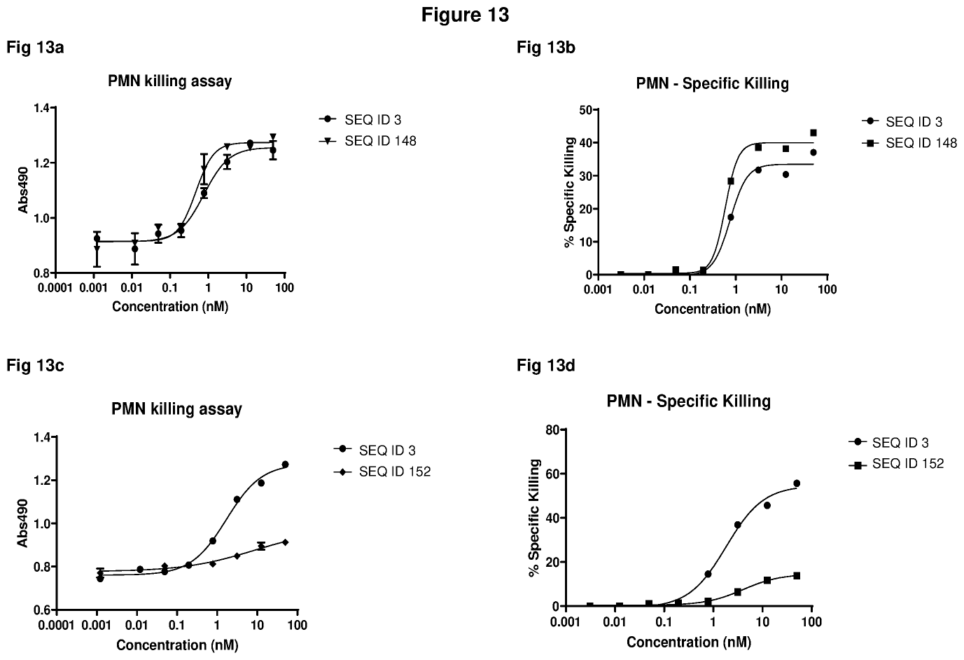

Semi-rational design was used to determine which amino acid residues of the Fc region of IgA2 are necessary to interact with hFcαRI, despite their apparent unrelated role in such an interaction, or the long distance separating these residues from the hFcαRI interface. It was found that disulfide bonds and packed loops spatially located on top of the CH2 domain of IgA2 (shown in FIG. 9) play an important role in the correct positioning of CH2 domain residues at the hFcαRI binding site. Such an effect was very surprising to discover since this part of the CH2 domain is found distant to the binding site when IgA2 binds to hFcαRI. It was also found that alpha-helices located on the CH3 domain of IgA2, that are exposed by solvent and located far from hFcαRI, have an impact on IgA2 Fc/hFcαRI interactions. The amino acid sequences depicted in SEQ ID NOs: 49 to 57 and 73 to 79 exemplify the modifications made to the CH2 domain to determine residues involved in indirect hFcαRI binding. The amino acid sequences depicted in SEQ ID NOs: 80 to 99 exemplify the modifications made to the CH3 domain to determine residues involved in indirect hFcαRI binding. These CH2 and CH3 mutations are shown schematically in FIG. 3h and FIG. 3i, are summarized in Tables 18 and 20 below and their effect on hFcαRI binding is presented in Tables 19 and 21.

TABLE 18
Summary of CH2 mutations (based on sequence SEQ ID NO: 1)

| SEQ | |
|---|---|
| ID NO | CH2 Mutation set (IMGT numbering for C-domain) |
| 49 | P8R, K10A, P11L, K12E, T14L, M15.1L, I15.2#, S16G, R17S, T18E, P19A, E20N, V26T, |
| | D27D, V28L, S29R, H30D, E31A, D34#, P35S, E36G, V37A, K38T, T80Q, K81G, R83P, |
| | E84.1R, Q84.2D, Y84.3L, N84.4C, S85.4G, T85.3C, R85.1S, T90P, V91G, L92C, H93A, |
| | D95P, L97N, N98H, V106A, S107A, N108H, K109P, A110E, P114K, A115T, S122T, A124S |
| 50 | A1.6#, P1.5#, E1.4#, L1.3#, L1.2C, G1.1C, G1H, S3R, P8R, K10A, P11L, K12E, T14L, |
| | M15.1L, I15.2#, S16G, R17S, T18E, P19A, E20N, V26T, D27D, V28L, S29R, H30D, T80Q, |
| | K81G, R83P, E84.1R, Q84.2D, Y84.3L, N84.4C, S85.4G, T85.3C, R85.1S, T90P, V91G, |
| | L92C, H93A, D95P, L97N, N98H, V106A, S107A, N108H, K109P, A110E, P114K, A115T, |
| | S122T, A124S |
| 51 | A1.6#, P1.5#, E1.4#, L1.3#, L1.2C, G1.1C, G1H, S3R, P8R, K10A, P11L, K12E, T14L, |
| | M15.1L, I15.2#, S16G, R17S, T18E, P19A, E20N, V26T, D27D, V28L, S29R, H30D, E31A, |
| | D34#, P35S, E36G, V37A, K38T, N84.4C, S85.4G, T85.3C, R85.1S, T90P, V91G, L92C, |
| | H93A, D95P, L97N, N98H, V106A, S107A, N108H, K109P, A110E, P114K, A115T, S122T, |
| | A124S |
| 52 | A1.6#, P1.5#, E1.4#, L1.3#, L1.2C, G1.1C, G1H, S3R, P8R, K10A, P11L, K12E, T14L, |
| | M15.1L, I15.2#, S16G, R17S, T18E, P19A, E20N, V26T, D27D, V28L, S29R, H30D, E31A, |
| | D34#, P35S, E36G, V37A, K38T, T80Q, K81G, R83P, E84.1R, Q84.2D, Y84.3L, N84.4C, |
| | S85.4G, T85.3C, R85.1S, T90P, V91G, L92C, H93A, D95P, L97N, N98H, P114K, A115T, |
| | S122T, A124S |
| 53 | A1.6#, P1.5#, E1.4#, L1.3#, L1.2C, G1.1C, G1H, S3R, P8R, K10A, P11L, K12E, T14L, |
| | M15.1L, I15.2#, S16G, R17S, T18E, P19A, E20N, V26T, D27D, V28L, S29R, H30D, E31A, |
| | D34#, P35S, E36G, V37A, K38T, T80Q, K81G, R83P, E84.1R, Q84.2D, Y84.3L, N84.4C, |
| | S85.4G, T85.3C, R85.1S, T90P, V91G, L92C, H93A, D95P, L97N, N98H, V106A, S107A, |
| | N108H, K109P, A110E, S122T, A124S |
| 54 | P8R, K10A, P11L, K12E, T14L, M15.1L, I15.2#, S16G, R17S, T18E, P19A, E20N, V26T, |
| | D27D, V28L, S29R, H30D, N84.4C, S85.4G, T85.3C, R85.1S, T90P, V91G, L92C, H93A, |
| | D95P, L97N, N98H, V106A, S107A, N108H, K109P, A110E, S122T, A124S |
| 55 | P8R, K10A, P11L, K12E, T14L, M15.1L, I15.2#, S16G, R17S, T18E, P19A, E20N, V26T, |
| | D27D, V28L, S29R, H30D, N84.4C, S85.4G, T85.3C, R85.1S, T90P, V91G, L92C, H93A, |
| | D95P, L97N, N98H, S122T, A124S |
| 56 | A1.6#, P1.5#, E1.4#, L1.3#, L1.2C, G1.1C, G1H, S3R, P8R, K10A, P11L, K12E, T14L, |
| | M15.1L, I15.2#, S16G, R17S, T18E, P19A, E20N, V26T, D27D, V28L, S29R, H30D, |
| | N84.4C, S85.4G, T85.3C, R85.1S, T90P, V91G, L92C, H93A, D95P, L97N, N98H, V106A, |
| | S107A, N108H, K109P, A110E, S122T, A124S |
| 57 | A1.6#, P1.5#, E1.4#, L1.3#, L1.2C, G1.1C, G1H, S3R, P8R, K10A, P11L, K12E, T14L, |
| | M15.1L, I15.2#, S16G, R17S, T18E, P19A, E20N, V26T, D27D, V28L, S29R, H30D, |
| | N84.4C, S85.4G, T85.3C, R85.1S, T90P, V91G, L92C, H93A, D95P, L97N, N98H, S122T, |
| | A124S |
| 73 | A1.6#, P1.5#, E1.4#, L1.3#, L1.2C, G1.1C, G1H, S3R, P8R, K10A, P11L, K12E, T14L, |
| | M15.1L, I15.2#, S16G, R17S, T18E, P19A, E20N, N84.4C, S85.4G, T85.3C, R85.1S, |
| | T90P, V91G, L92C, H93A, D95P, L97N, N98H, V106A, S107A, N108H, K109P, A110E, |
| | S122T, A124S |
| 74 | A1.6#, P1.5#, E1.4#, L1.3#, L1.2C, G1.1C, G1H, S3R, P8R, K10A, P11L, K12E, T14L, |
| | M15.1L, I15.2#, S16G, R17S, T18E, P19A, E20N, N84.4C, S85.4G, T85.3C, R85.1S, |
| | T90P, V91G, L92C, H93A, D95P, L97N, N98H, S122T, A124S |
| 75 | A1.6#, P1.5#, E1.4#, L1.3#, L1.2C, G1.1C, G1H, S3R, P8R, K10A, P11L, K12E, T14L, |
| | M15.1L, I15.2#, S16G, R17S, T18E, P19A, E20N, V26T, D27D, V28L, S29R, H30D, T90P, |
| | V91G, L92C, H93A, D95P, L97N, N98H, V106A, S107A, N108H, K109P, A110E, S122T, |
| | A124S |
| 76 | A1.6#, P1.5#, E1.4#, L1.3#, L1.2C, G1.1C, G1H, S3R, P8R, K10A, P11L, K12E, T14L, |
| | M15.1L, I15.2#, S16G, R17S, T18E, P19A, E20N, V26T, D27D, V28L, S29R, H30D, T90P, |
| | V91G, L92C, H93A, D95P, L97N, N98H, S122T, A124S |
| 77 | P8R, K10A, P11L, K12E, T14L, M15.1L, I15.2#, S16G, R17S, T18E, P19A, E20N, V26T, |
| | D27D, V28L, S29R, H30D, T90P, V91G, L92C, H93A, D95P, L97N, N98H, V106A, S107A, |
| | N108H, K109P, A110E, S122T, A124S |
| 78 | P8R, K10A, P11L, K12E, T14L, M15.1L, I15.2#, S16G, R17S, T18E, P19A, E20N, V26T, |
| | D27D, V28L, S29R, H30D, T90P, V91G, L92C, H93A, D95P, L97N, N98H, S122T, A124S |
| 79 | A1.6#, P1.5#, E1.4#, L1.3#, L1.2C, G1.1C, G1H, S3R, P8R, K10A, P11L, K12E, T14L, |
| | M15.1L, I15.2#, S16G, R17S, T18E, P19A, E20N, V26T, D27D, V28L, S29R, H30D, |
| | N84.4C, S85.4G, T85.3C, R85.1S, V106A, S107A, N108H, K109P, A110E, S122T, A124S |

TABLE 19
Affinity and maximum response of engineered immunoglobulin
towards hFcαRI determined by surface plasmon resonance,
following the procedure described in Example 5a.

| | | | Maximum response |
|---|---|---|---|
| | | | at 1000 nM hFcαRI |
| SEQ ID NO | Ref number | KD (1:1) (M) | (RU) |
| 2 | 2258 | 2.59E−07 | 263 |
| 49 | 2564 | 1.51E−07 | 179 |
| 50 | 2565 | 1.44E−07 | 213 |
| 51 | 2566 | 2.10E−07 | 229 |
| 52 | 2567 | 2.71E−07 | 214 |
| 53 | 2568 | 1.43E−07 | 231 |
| 54 | 2569 | 7.05E−07 | 20 |
| 55 | 2570 | 1.67E−07 | 129 |
| 56 | 2571 | 2.26E−07 | 209 |
| 57 | 2572 | 2.90E−07 | 220 |
| 73 | 2683 | 4.84E−07 | 54 |
| 74 | 2684 | 4.11E−07 | 54 |
| 75 | 2685 | 3.72E−07 | 20 |
| 76 | 2686 | 6.61E−07 | 22 |
| 77 | 2687 | 0.00E+00 | 0 |
| 78 | 2688 | 4.44E−07 | 20 |
| 79 | 2689 | 5.67E−07 | 200 |

TABLE 20
Summary of CH3 mutations (based on sequence SEQ ID NO: 1)

| SEQ | |
|---|---|
| ID NO | CH3 Mutation set (IMGT numbering for C-domain) |
| 80 | S35K, I37V, A38L, E40R, E42L, S43Q, N44G, G45S, P45.2E, E45.3L, N45.4P, \hat{ }45.4RE, |
| | \hat{ }84.4QGT, G85.4T, S85.3T, F85.1A, L85V, Y86T, K88I, T90R, D92A, V101T, S105M, |
| | M107G, H113P, N114L, H115A, Y116F, S120T |
| 81 | A38L, E40R, E42L, S43Q, N44G, G45S, P45.2E, E45.3L, N45.4P, \hat{ }45.4RE, \hat{ }84.4QGT, |
| | G85.4T, S85.3T, F85.1A, L85V, Y86T, K88I, T90R, D92A, V101T, S105M, M107G, H113P, |
| | N114L, H115A, Y116F, S120T |
| 82 | E40R, E42L, S43Q, N44G, G45S, P45.2E, E45.3L, N45.4P, \hat{ }45.4RE, \hat{ }84.4QGT, G85.4T, |
| | S85.3T, F85.1A, L85V, Y86T, K88I, T90R, D92A, V101T, S105M, M107G, H113P, N114L, |
| | H115A, Y116F, S120T |
| 83 | S35K, I37V, A38L, E40R, E42L, S43Q, N44G, G45S, P45.2E, E45.3L, N45.4P, \hat{ }45.4RE, |
| | G85.4T, S85.3T, F85.1A, L85V, Y86T, K88I, T90R, D92A, V101T, S105M, M107G, H113P, |
| | N114L, H115A, Y116F, S120T |
| 84 | S35K, I37V, A38L, E40R, E42L, S43Q, N44G, G45S, P45.2E, E45.3L, N45.4P, \hat{ }45.4RE, |
| | K88I, T90R, D92A, V101T, S105M, M107G, H113P, N114L, H115A, Y116F, S120T |
| 85 | E40R, E42L, S43Q, N44G, G45S, P45.2E, E45.3L, N45.4P, \hat{ }45.4RE, K88I, T90R, D92A, |
| | V101T, S105M, M107G, H113P, N114L, H115A, Y116F, S120T |
| 86 | S35K, I37V, A38L, E40R, E42L, S43Q, N44G, G45S, P45.2E, E45.3L, N45.4P, \hat{ }45.4RE, |
| | V101T, S105M, M107G, H113P, N114L, H115A, Y116F, S120T |
| 87 | R1.1F, T6L, S10P, D12E, T15A, \hat{ }15L, K16N, N17E, Q18L, V25A, Y29S, S35K, I37V, |
| | A38L, E40R, E42L, S43Q, N44G, G45S, P45.2E, E45.3L, N45.4P, \hat{ }45.4RE, P82A, P83S, |
| | L84.1Q, D84.2E, S84.3P, D84.4S, \hat{ }84.4QGT, G85.4T, L85V, D92A, K93A, S94E, R95D, |
| | Q97K, Q98K, N100D, V101T, S105M, M107G, H113P, N114L, H115A, Y116F, L121I, |
| | L123R |
| 88 | R1.1F, T6L, V25A, Y29S, S35K, I37V, A38L, E40R, E42L, S43Q, N44G, G45S, P45.2E, |
| | E45.3L, N45.4P, \hat{ }45.4RE, P82A, P83S, L84.1Q, D84.2E, S84.3P, D84.4S, \hat{ }84.4QGT, |
| | G85.4T, L85V, D92A, K93A, S94E, R95D, Q97K, Q98K, N100D, V101T, S105M, M107G, |
| | H113P, N114L, H115A, Y116F, L121I, L123R |
| 89 | T6L, V25A, S35K, I37V, A38L, E40R, E42L, S43Q, N44G, G45S, P45.2E, E45.3L, N45.4P, |
| | \hat{ }45.4RE, P82A, P83S, L85V, D92A, K93A, S94E, R95D, Q97K, Q98K, N100D, V101T, |
| | S105M, M107G, H113P, N114L, H115A, Y116F, L121I, L123R |
| 90 | T6L, S10P, D12E, T15A, \hat{ }15L, K16N, N17E, Q18L, V25A, S35K, I37V, A38L, E40R, E42L, |
| | S43Q, N44G, G45S, P45.2E, E45.3L, N45.4P, \hat{ }45.4RE, P82A, P83S, L85V, D92A, K93A, |
| | S94E, R95D, Q97K, Q98K, N100D, V101T, S105M, M107G, H113P, N114L, H115A, |
| | Y116F, L121I, L123R |
| 91 | T6L, S10P, D12E, T15A, \hat{ }15L, K16N, N17E, Q18L, V25A, S35K, I37V, A38L, E40R, E42L, |
| | S43Q, N44G, G45S, P45.2E, E45.3L, N45.4P, \hat{ }45.4RE, P82A, P83S, L85V, S105M, |
| | M107G, H113P, N114L, H115A, Y116F, L121I |
| 92 | R1.1F, T6L, S10P, D12E, T15A, \hat{ }15L, K16N, N17E, Q18L, V25A, Y29S, S35K, I37V, |
| | A38L, E40R, E42L, S43Q, N44G, G45S, P45.2E, E45.3L, N45.4P, \hat{ }45.4RE, P82A, P83S, |
| | L84.1Q, D84.2E, S84.3P, D84.4S, \hat{ }84.4QGT, G85.4T, L85V, S105M, M107G, H113P, |
| | N114L, H115A, Y116F, L121I |
| 93 | R1.1F, T6L, V25A, Y29S, S35K, I37V, A38L, E40R, E42L, S43Q, N44G, G45S, P45.2E, |
| | E45.3L, N45.4P, \hat{ }45.4RE, P82A, P83S, L84.1Q, D84.2E, S84.3P, D84.4S, \hat{ }84.4QGT, |
| | G85.4T, L85V, S105M, M107G, H113P, N114L, H115A, Y116F, L121I |
| 94 | T6L, V25A, S35K, I37V, A38L, E40R, E42L, S43Q, N44G, G45S, P45.2E, E45.3L, N45.4P, |
| | \hat{ }45.4RE, P82A, P83S, L85V, S105M, M107G, H113P, N114L, H115A, Y116F, L121I |
| 95 | Q1.3N, P1.2T, R1.1F, E1R, T6L, V25A, Y29S, S35K, I37V, A38L, E40R, E42L, S43Q, |
| | N44G, G45S, P45.2E, E45.3L, N45.4P, \hat{ }45.4RE, P82A, P83S, L84.1Q, D84.2E, S84.3P, |
| | D84.4S, \hat{ }84.4QGT, G85.4T, S85.3T, L85V, S105M, M107G, H113P, N114L, H115A, |
| | Y116F, L121I |
| 96 | Q1.3N, P1.2T, R1.1F, E1R, T6L, V25A, Y29S, S35K, I37V, A38L, E40R, E42L, S43Q, |
| | N44G, G45S, P45.2E, E45.3L, N45.4P, \hat{ }45.4RE, P82A, P83S, L84.1Q, D84.2E, S84.3P, |
| | D84.4S, \hat{ }84.4QGT, G85.4T, S85.3T, L85V, D92A, K93A, S94E, R95D, Q97K, Q98K, |
| | N100D, V101T, S105M, M107G, H113P, N114L, H115A, Y116F, L121I, L123R |
| 97 | Q1.3N, P1.2T, R1.1F, E1R, T6L, S10P, D12E, T15A, \hat{ }15L, K16N, N17E, Q18L, V25A, |
| | Y29S, S35K, I37V, A38L, E40R, E42L, S43Q, N44G, G45S, P45.2E, E45.3L, N45.4P, |
| | \hat{ }45.4RE, P82A, P83S, L84.1Q, D84.2E, S84.3P, D84.4S, \hat{ }84.4QGT, G85.4T, S85.3T, |
| | L85V, S105M, M107G, H113P, N114L, H115A, Y116F, L121I |
| 98 | Q1.3N, P1.2T, R1.1F, E1R, T6L, S10P, D12E, T15A, \hat{ }15L, K16N, N17E, Q18L, V25A, |
| | Y29S, S35K, I37V, A38L, E40R, E42L, S43Q, N44G, G45S, P45.2E, E45.3L, N45.4P, |
| | \hat{ }45.4RE, P82A, P83S, L84.1Q, D84.2E, S84.3P, D84.4S, \hat{ }84.4QGT, G85.4T, S85.3T, |
| | L85V, D92A, K93A, S94E, R95D, Q97K, Q98K, N100D, V101T, S105M, M107G, H113P, |
| | N114L, H115A, Y116F, L121I, L123R |
| 99 | Q1.3N, P1.2T, R1.1F, E1R, T6L, S10P, D12E, T15A, \hat{ }15L, K16N, N17E, Q18L, V25A, |
| | Y29S, S35K, I37V, A38L, E40R, E42L, S43Q, N44G, G45S, P45.2E, E45.3L, N45.4P, |
| | \hat{ }45.4RE, P82A, P83S, L84.1Q, D84.2E, S84.3P, D84.4S, A84.4QGT, G85.4T, S85.3T, |
| | F85.1A, L85V, Y86T, K88I, T90R, D92A, K93A, S94E, R95D, Q97K, Q98K, N100D, V101T, |
| | S105M, M107G, H113P, N114L, H115A, Y116F, L121I, L123R |

TABLE 21
Affinity and maximum response of engineered immunoglobulin
towards hFcαRI determined by surface plasmon resonance,
following the procedure described in Example 5a.

| | | | Maximum response |
|---|---|---|---|
| | | | at 1000 nM hFcαRI |
| SEQ ID NO | Ref number | KD (1:1) (M) | (RU) |
| 2 | 2258 | 2.59E−07 | 263 |
| 80 | 2696 | 3.56E−06 | 50 |
| 81 | 2697 | 6.32E−06 | 32 |
| 82 | 2698 | 0.00E+00 | 6 |
| 83 | 2699 | 4.65E−06 | 43 |
| 84 | 2700 | 3.66E−06 | 43 |
| 85 | 2701 | 1.62E−06 | 20 |
| 86 | 2702 | 1.68E−06 | 89 |
| 87 | 2703 | 5.89E−07 | 103 |
| 88 | 2704 | 5.50E−07 | 225 |
| 89 | 2705 | 6.60E−07 | 271 |
| 90 | 2706 | 6.08E−07 | 288 |
| 91 | 2707 | 8.44E−07 | 200 |
| 92 | 2708 | 8.43E−07 | 200 |
| 93 | 2709 | 1.14E−06 | 171 |
| 94 | 2710 | 1.04E−06 | 213 |
| 95 | 2711 | 1.30E−06 | 164 |
| 96 | 2712 | 8.98E−07 | 240 |
| 97 | 2713 | 1.30E−06 | 167 |
| 98 | 2714 | 7.34E−07 | 257 |
| 99 | 2715 | 2.14E−06 | 117 |

1.3 Summary of Rational and Semi Rational Design to Generate Homodimers

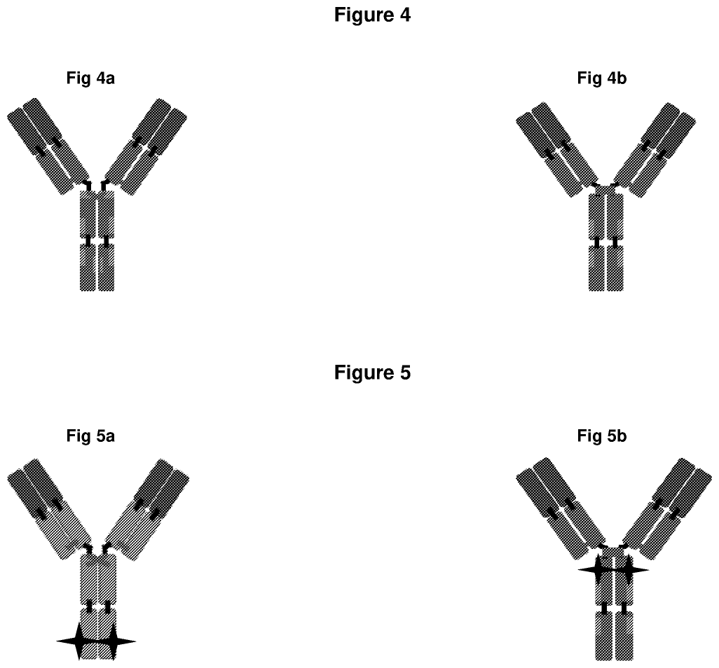

The preferred engineered sequences from IgG1 CH2, CH3 and CH2/CH3 elbows identified as described above, were assembled together to generate an engineered IgG1 that was able to retain the hFcαRI binding capacity of an IgA immunoglobulin. The preferred structures depicting the positions of the CH2 and CH3 mutations are shown schematically in FIG. 4a and FIG. 4b. The CH2 and CH3 mutations of the engineered sequences are listed in Table 22. The binding to hFcαRI was characterized by surface plasmon resonance, as shown in Table 23 and Table 24. The resulting engineered immunoglobulins had hFcαRI binding in the range from parental IgA2-like binding to about 10-fold lower binding than parental IgA2.

Lead candidates selected from this round of engineering comprised SEQ ID NOs: 119, 120, 122 and 123 and the corresponding binding data towards hFcαRI are presented in Table 24. These candidates were then tested in an ADCC and ADCP assay following the procedure described in Examples 6 and 7, respectively. The results are shown in FIGS. 10, 11 and 12. Engineered IgG1 immunoglobulins comprising SEQ ID NOs: 119, 120, 122 and 123 were shown to have an improved efficacy compared to parental IgA2 in a SK-BR-3 cell PMN killing assay (FIG. 10). As shown in FIG. 11, the engineered IgG1 immunoglobulin comprising SEQ ID NO: 122 was shown to have an efficacy comparable to parental IgA2 in a Calu-3 cell PMN killing assay. The phagocytosis activity of the engineered IgG1 immunoglobulin comprising SEQ ID NO: 122 was determined in an ADCP assay and is shown in FIG. 12. The phagocytosis activity of this immunoglobulin was shown to be comparable to both wild-type IgG1 (SEQ ID NO: 1) and parental IgA2 (SEQ ID NO: 3).

TABLE 22
Summary of CH2 and CH3 mutations (based on sequence SEQ ID NO: 1)

| SEQ | |
|---|---|
| ID NO | CH2 and CH3 Mutation set (IMGT numbering for C-domain) |
| 100 | CH2: L1.2C, G1.1C, G1H, S3R, P8R, K10A, P11L, K12E, T14L, M15.1L, I15.2#, S16G, |
| | R17S, T18E, P19A, E20N, V26T, D27G, V28L, S29R, H30D, N84.4C, S85.4G, T85.3C, |
| | R85.1S, T90P, V91G, L92C, H93A, D95P, L97N, N98H, V106A, S107A, N108H, K109P, |
| | A110E |
| | CH3: A38L, E40R, E42L, S43Q, N44G, G45S, P45.2E, E45.3L, N45.4P, \hat{ }45.4RE, V101T, |
| | S105M, M107G, H113P, N114L, H115A, Y116F, S120T |
| 101 | CH2: L1.2C, G1.1C, G1H, S3R, P8R, K10A, P11L, K12E, T14L, M15.1L, I15.2#, S16G, |
| | R17S, T18E, P19A, E20N, V26T, D27G, V28L, S29R, H30D, N84.4C, S85.4G, T85.3C, |
| | R85.1S, T90P, V91G, L92C, H93A, D95P, L97N, N98H, V106A, S107A, N108H, K109P, |
| | A110E |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 102 | CH2: L1.2C, G1.1C, G1H, S3R, P8R, K10A, P11L, K12E, T14L, M15.1L, I15.2#, S16G, |
| | R17S, T18E, P19A, E20N, V26T, D27G, V28L, S29R, H30D, N84.4C, S85.4G, T85.3C, |
| | R85.1S, T90P, V91G, L92C, H93A, D95P, L97N, N98H, V106A, S107A, N108H, K109P, |
| | A110E |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, S105M, M107G, H113P, N114L, H115A, Y116F, |
| | L121I |
| 103 | CH2: L1.2C, G1.1C, G1H, S3R, P8R, K10A, P11L, K12E, T14L, M15.1L, I15.2#, S16G, |
| | R17S, T18E, P19A, E20N, V26T, D27G, V28L, S29R, H30D, N84.4C, S85.4G, T85.3C, |
| | R85.1S, V106A, S107A, N108H, K109P, A110E |
| | CH3: A38L, E40R, E42L, S43Q, N44G, G45S, P45.2E, E45.3L, N45.4P, \hat{ }45.4RE, V101T, |
| | S105M, M107G, H113P, N114L, H115A, Y116F, S120T |
| 104 | CH2: L1.2C, G1.1C, G1H, S3R, P8R, K10A, P11L, K12E, T14L, M15.1L, I15.2#, S16G, |
| | R17S, T18E, P19A, E20N, V26T, D27G, V28L, S29R, H30D, N84.4C, S85.4G, T85.3C, |
| | R85.1S, V106A, S107A, N108H, K109P, A110E |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 105 | CH2: L1.2C, G1.1C, G1H, S3R, P8R, K10A, P11L, K12E, T14L, M15.1L, I15.2#, S16G, |
| | R17S, T18E, P19A, E20N, V26T, D27G, V28L, S29R, H30D, N84.4C, S85.4G, T85.3C, |
| | R85.1S, V106A, S107A, N108H, K109P, A110E |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, S105M, M107G, H113P, N114L, H115A, Y116F, |
| | L121I |
| 106 | CH2: L1.2C, G1.1C, G1H, S3R, P8R, K10A, P11L, K12E, T14L, M15.1L, I15.2#, S16G, |
| | R17S, T18E, P19A, E20N, V26T, D27G, V28L, S29R, H30D, N84.4C, S85.4G, T85.3C, |
| | R85.1S, T90P, V91G, L92C, H93A, D95P, L97N, N98H |
| | CH3: A38L, E40R, E42L, S43Q, N44G, G45S, P45.2E, E45.3L, N45.4P, \hat{ }45.4RE, V101T, |
| | S105M, M107G, H113P, N114L, H115A, Y116F, S120T |
| 107 | CH2: L1.2C, G1.1C, G1H, S3R, P8R, K10A, P11L, K12E, T14L, M15.1L, I15.2#, S16G, |
| | R17S, T18E, P19A, E20N, V26T, D27G, V28L, S29R, H30D, N84.4C, S85.4G, T85.3C, |
| | R85.1S, T90P, V91G, L92C, H93A, D95P, L97N, N98H |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 108 | CH2: A1.6#, P1.5#, E1.4#, L1.3#, L1.2C, G1.1C, G1H, S3R, P8R, K10A, P11L, K12E, |
| | T14L, M15.1L, I15.2#, S16G, R17S, T18E, P19A, E20N, V26T, D27G, V28L, S29R, H30D, |
| | N84.4C, S85.4G, T85.3C, R85.1S, T90P, V91G, L92C, H93A, D95P, L97N, N98H |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, S105M, M107G, H113P, N114L, H115A, Y116F, |
| | L121I |
| 109 | CH2: A1.6#, P1.5#, E1.4#, L1.3#, L1.2C, G1.1C, G1H, S3R, P8R, K10A, P11L, K12E, |
| | T14L, M15.1L, I15.2#, S16G, R17S, T18E, P19A, E20N, V26T, D27G, V28L, S29R, H30D, |
| | N84.4C, S85.4G, T85.3C, R85.1S |
| | CH3: A38L, E40R, E42L, S43Q, N44G, G45S, P45.2E, E45.3L, N45.4P, \hat{ }45.4RE, V101T, |
| | S105M, M107G, H113P, N114L, H115A, Y116F, S120T |
| 110 | CH2: A1.6#, P1.5#, E1.4#, L1.3#, L1.2C, G1.1C, G1H, S3R, P8R, K10A, P11L, K12E, |
| | T14L, M15.1L, I15.2#, S16G, R17S, T18E, P19A, E20N, V26T, D27G, V28L, S29R, H30D, |
| | N84.4C, S85.4G, T85.3C, R85.1S |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 111 | CH2: A1.6#, P1.5#, E1.4#, L1.3#, L1.2C, G1.1C, G1H, S3R, P8R, K10A, P11L, K12E, |
| | T14L, M15.1L, I15.2#, S16G, R17S, T18E, P19A, E20N, V26T, D27G, V28L, S29R, H30D, |
| | N84.4C, S85.4G, T85.3C, R85.1S |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, S105M, M107G, H113P, N114L, H115A, Y116F, |
| | L121I |
| 112 | CH2: A1.6#, P1.5#, E1.4#, L1.3#, L1.2C, G1.1C, G1H, S3R, P8R, K10A, P11L, K12E, |
| | T14L, M15.1L, I15.2#, S16G, R17S, T18E, P19A, E20N, V26T, D27G, V28L, S29R, H30D, |
| | N84.4C, S85.4G, T85.3C, R85.1S, T90P, V91G, L92C, H93A, D95P, L97N, N98H, V106A, |
| | S107A, N108H, K109P, A110E, K125# |
| | CH3: A38L, E40R, E42L, S43Q, N44G, G45S, P45.2E, E45.3L, N45.4P, \hat{ }45.4RE, V101T, |
| | S105M, M107G, H113P, N114L, H115A, Y116F, S120T |
| 113 | CH2: A1.6#, P1.5#, E1.4#, L1.3#, L1.2C, G1.1C, G1H, S3R, P8R, K10A, P11L, K12E, |
| | T14L, M15.1L, I15.2#, S16G, R17S, T18E, P19A, E20N, V26T, D27G, V28L, S29R, H30D, |
| | N84.4C, S85.4G, T85.3C, R85.1S, T90P, V91G, L92C, H93A, D95P, L97N, N98H, V106A, |
| | S107A, N108H, K109P, A110E, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 114 | CH2: A1.6#, P1.5#, E1.4#, L1.3#, L1.2C, G1.1C, G1H, S3R, P8R, K10A, P11L, K12E, |
| | T14L, M15.1L, I15.2#, S16G, R17S, T18E, P19A, E20N, V26T, D27G, V28L, S29R, H30D, |
| | N84.4C, S85.4G, T85.3C, R85.1S, T90P, V91G, L92C, H93A, D95P, L97N, N98H, V106A, |
| | S107A, N108H, K109P, A110E, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, S105M, M107G, H113P, N114L, H115A, Y116F, |
| | L121I |
| 115 | CH2: A1.6#, P1.5#, E1.4#, L1.3#, L1.2C, G1.1C, G1H, S3R, P8R, K10A, P11L, K12E, |
| | T14L, M15.1L, I15.2#, S16G, R17S, T18E, P19A, E20N, V26T, D27G, V28L, S29R, H30D, |
| | N84.4C, S85.4G, T85.3C, R85.1S, K125# |
| | CH3: A38L, E40R, E42L, S43Q, N44G, G45S, P45.2E, E45.3L, N45.4P, \hat{ }45.4RE, V101T, |
| | S105M, M107G, H113P, N114L, H115A, Y116F, S120T |
| 116 | CH2: A1.6#, P1.5#, E1.4#, L1.3#, L1.2C, G1.1C, G1H, S3R, P8R, K10A, P11L, K12E, |
| | T14L, M15.1L, I15.2#, S16G, R17S, T18E, P19A, E20N, V26T, D27G, V28L, S29R, H30D, |
| | N84.4C, S85.4G, T85.3C, R85.1S, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 117 | CH2: A1.6#, P1.5#, E1.4#, L1.3#, L1.2C, G1.1C, G1H, S3R, P8R, K10A, P11L, K12E, |
| | T14L, M15.1L, I15.2#, S16G, R17S, T18E, P19A, E20N, V26T, D27G, V28L, S29R, H30D, |
| | N84.4C, S85.4G, T85.3C, R85.1S, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, S105M, M107G, H113P, N114L, H115A, Y116F, |
| | L121I |
| 118 | CH2: A1.6#, P1.5#, E1.4#, L1.3#, L1.2C, G1.1C, G1H, S3R, P8R, K10A, P11L, K12E, |
| | T14L, M15.1L, I15.2#, S16G, R17S, T18E, P19A, E20N, V26T, D27G, V28L, S29R, H30D, |
| | N84.4C, S85.4G, T85.3C, R85.1S, T90P, V91G, L92C, H93A, D95P, L97N, N98H, K125# |
| | CH3: A38L, E40R, E42L, S43Q, N44G, G45S, P45.2E, E45.3L, N45.4P, \hat{ }45.4RE, V101T, |
| | S105M, M107G, H113P, N114L, H115A, Y116F, S120T |
| 119 | CH2: L1.2C, G1.1C, G1H, S3R, P8R, K10A, P11L, K12E, T14L, M15.1L, I15.2#, S16G, |
| | R17S, T18E, P19A, E20N, V26T, D27G, V28L, S29R, H30D, N84.4C, S85.4G, T85.3C, |
| | R85.1S, T90P, V91G, L92C, H93A, D95P, L97N, N98H, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 120 | CH2: A1.6#, P1.5#, E1.4#, L1.3#, L1.2C, G1.1C, G1H, S3R, P8R, K10A, P11L, K12E, |
| | T14L, M15.1L, I15.2#, S16G, R17S, T18E, P19A, E20N, V26T, D27G, V28L, S29R, H30D, |
| | N84.4C, S85.4G, T85.3C, R85.1S, T90P, V91G, L92C, H93A, D95P, L97N, N98H, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, S105M, M107G, H113P, N114L, H115A, Y116F, |
| | L121I |
| 121 | CH2: A1.6#, P1.5#, E1.4#, L1.3#, L1.2C, G1.1C, G1H, S3R, P8R, K10A, P11L, K12E, |
| | T14L, M15.1L, I15.2#, S16G, R17S, T18E, P19A, E20N, V26T, D27G, V28L, S29R, H30D, |
| | N84.4C, S85.4G, T85.3C, R85.1S, K125# |
| | CH3: A38L, E40R, E42L, S43Q, N44G, G45S, P45.2E, E45.3L, N45.4P, \hat{ }45.4RE, V101T, |
| | S105M, M107G, H113P, N114L, H115A, Y116F, S120T |
| 122 | CH2: A1.6#, P1.5#, E1.4#, L1.3#, L1.2C, G1.1C, G1H, S3R, P8R, K10A, P11L, K12E, |
| | T14L, M15.1L, I15.2#, S16G, R17S, T18E, P19A, E20N, V26T, D27G, V28L, S29R, H30D, |
| | N84.4C, S85.4G, T85.3C, R85.1S, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 123 | CH2: A1.6#, P1.5#, E1.4#, L1.3#, L1.2C, G1.1C, G1H, S3R, P8R, K10A, P11L, K12E, |
| | T14L, M15.1L, I15.2#, S16G, R17S, T18E, P19A, E20N, V26T, D27G, V28L, S29R, H30D, |
| | N84.4C, S85.4G, T85.3C, R85.1S, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, S105M, M107G, H113P, N114L, H115A, Y116F, |
| | L1211 |
| 146 | CH2: P8R, K10A, P11L, K12E, T14L, M15.1L, I15.2#, S16G, R17S, T18E, P19A, E20N, |
| | T90P, V91G, L92C, H93A, D95P, L97N, N98H, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 147 | CH2: P8R, K10A, P11L, K12E, T14L, M15.1L, I15.2#, S16G, R17S, T18E, P19A, E20N, |
| | T90P, V91G, L92C, H93A, D95P, L97N, N98H, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, S105M, M107G, H113P, N114L, H115A, Y116F, |
| | L121I |
| 148 | CH2: P8R, K10A, P11L, K12E, T14L, M15.1L, I15.2#, S16G, R17S, T18E, P19A, E20N, |
| | K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 149 | CH2: P8R, K10A, P11L, K12E, T14L, M15.1L, I15.2#, S16G, R17S, T18E, P19A, E20N, |
| | K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, S105M, M107G, H113P, N114L, H115A, Y116F, |
| | L121I |
| 165 | CH2: A1.3L, L1.2C, G1.1C, G1H, S3R, K10A, P11L, K12E, T14L, M15.1L, I15.2#, S16G, |
| | R17S, T18E, P19A, E20N, V26T, D27G, V28L, S29R, H30D, N84.4C, S85.4G, T85.3C, |
| | R85.1S, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 166 | CH2: A1.3L, L1.2C, G1.1C, G1H, S3R, K10A, P11L, K12E, T14L, M15.1L, I15.2#, S16G, |
| | R17S, T18E, P19A, E20N, V26T, D27G, V28L, S29R, H30D, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 167 | CH2: A1.3L, L1.2C, G1.1C, G1H, S3R, K10A, P11L, K12E, T14L, M15.1L, I15.2#, S16G, |
| | R17S, T18E, P19A, E20N, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 168 | CH2: K10A, P11L, K12E, T14L, M15.1L, I15.2#, S16G, R17S, T18E, P19A, E20N, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 169 | CH2: K10A, P11L, K12E, T14L, M15.1L, I15.2#, S16G, R17S, T18E, P19A, E20N, V26T, |
| | D27G, V28L, S29R, H30D, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 170 | CH2: K10A, P11L, K12E, T14L, M15.1L, I15.2#, S16G, R17S, T18E, P19A, E20N, V26T, |
| | D27G, V28L, S29R, H30D, N84.4C, S85.4G, T85.3C, R85.1S, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 171 | CH2: K10A, P11L, K12E, T14L, M15.1L, I15.2#, S16G, R17S, T18E, P19A, E20N, N84.4C, |
| | S85.4G, T85.3C, R85.1S, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 172 | CH2: A1.6#, P1.5#, E1.4#, L1.3#, K10A, P11L, K12E, T14L, M15.1L, I15.2#, S16G, R17S, |
| | T18E, P19A, E20N, N84.4C, S85.4G, T85.3C, R85.1S |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, |
| 173 | CH2: A1.6#, P1.5#, E1.4#, L1.3#, K10A, P11L, K12E, T14L, M15.1L, I15.2#, S16G, R17S, |
| | T18E, P19A, E20N, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 174 | CH2: A1.6#, P1.5#, E1.4#, L1.3#, L1.2C, G1.1C, G1H, S3R, P8R, K10A, P11L, K12E, |
| | T14L, M15.1L, I15.2#, S16G, R17S, T18E, P19A, E20N, V26T, D27G, V28L, S29R, H30D, |
| | N84.4C, S85.4G, T85.3C, R85.1S, K125# |
| | CH3: E1R, Q3E, T6L, V25A, S35K, I37V, A38L, E40R, E42L, S43Q, N44G, G45S, P45.2E, |
| | E45.3L, N45.4P, \hat{ }45.4RE, P82A, P83S, L85V, D92A, K93A, S94E, R95D, Q97K, Q98K, |
| | N100D, V101T, S105M, M107G, H113P, N114L, H115A, Y116F, L121I, L123R |
| 175 | CH2: A1.6#, P1.5#, E1.4#, L1.3#, L1.2C, G1.1C, G1H, S3R, K10A, P11L, K12E, T14L, |
| | M15.1L, I15.2#, S16G, R17S, T18E, P19A, E20N, V26T, D27G, V28L, S29R, H30D, |
| | N84.4C, S85.4G, T85.3C, R85.1S, K125# |
| | CH3: E1R, Q3E, T6L, V25A, S35K, I37V, A38L, E40R, E42L, S43Q, N44G, G45S, P45.2E, |
| | E45.3L, N45.4P, \hat{ }45.4RE, P82A, P83S, L85V, D92A, K93A, S94E, R95D, Q97K, Q98K, |
| | N100D, V101T, S105M, M107G, H113P, N114L, H115A, Y116F, L121I, L123R |
| 176 | CH2: A1.6#, P1.5#, E1.4#, L1.3#, L1.2C, G1.1C, G1H, S3R, P8R, K10A, P11L, K12E, |
| | T14L, M15.1L, I15.2#, S16G, R17S, T18E, P19A, E20N, V26T, D27G, V28L, S29R, H30D, |
| | E31A, D34#, P35S, E36G, V37A, K38T, T80Q, K81G, R83P, E84.1R, Q84.2D, Y84.3L, |
| | N84.4C, S85.4G, T85.3C, R85.1S, T90P, V91G, L92C, H93A, D95P, L97N, N98H, V106A, |
| | S107A, N108H, K109P, A110E, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 177 | CH2: A1.6#, P1.5#, E1.4#, L1.3#, L1.2C, G1.1C, G1H, S3R, P8R, K10A, P11L, K12E, |
| | T14L, M15.1L, I15.2#, S16G, R17S, T18E, P19A, E20N, V26T, D27G, V28L, S29R, H30D, |
| | E31A, D34#, P35S, E36G, V37A, K38T, T80Q, K81G, R83P, E84.1R, Q84.2D, Y84.3L, |
| | N84.4C, S85.4G, T85.3C, R85.1S, V106A, S107A, N108H, K109P, A110E, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 178 | CH2: A1.3L, L1.2C, G1.1C, G1H, S3R, P8R, K10A, P11L, K12E, T14L, M15.1L, I15.2#, |
| | S16G, R17S, T18E, P19A, E20N, V26T, D27G, V28L, S29R, H30D, E31A, D34#, P35S, |
| | E36G, V37A, K38T, T80Q, K81G, R83P, E84.1R, Q84.2D, Y84.3L, N84.4C, S85.4G, |
| | T85.3C, R85.1S, T90P, V91G, L92C, H93A, D95P, L97N, N98H, V106A, S107A, N108H, |
| | K109P, A110E, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 179 | CH2: A1.3L, L1.2C, G1.1C, G1H, S3R, P8R, K10A, P11L, K12E, T14L, M15.1L, I15.2#, |
| | S16G, R17S, T18E, P19A, E20N, V26T, D27G, V28L, S29R, H30D, E31A, D34#, P35S, |
| | E36G, V37A, K38T, T80Q, K81G, R83P, E84.1R, Q84.2D, Y84.3L, N84.4C, S85.4G, |
| | T85.3C, R85.1S, V106A, S107A, N108H, K109P, A110E, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 180 | CH2: P8R, K10A, P11L, K12E, T14L, M15.1L, I15.2#, S16G, R17S, T18E, P19A, E20N, |
| | V26T, D27G, V28L, S29R, H30D, E31A, D34#, P35S, E36G, V37A, K38T, T80Q, K81G, |
| | R83P, E84.1R, Q84.2D, Y84.3L, N84.4C, S85.4G, T85.3C, R85.1S, |
| | T90P, V91G, L92C, H93A, D95P, L97N, N98H, V106A, S107A, N108H, K109P, A110E, |
| | K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 181 | CH2: P8R, K10A, P11L, K12E, T14L, M15.1L, I15.2#, S16G, R17S, T18E, P19A, E20N, |
| | V26T, D27G, V28L, S29R, H30D, E31A, D34#, P35S, E36G, V37A, K38T, T80Q, K81G, |
| | R83P, E84.1R, Q84.2D, Y84.3L, N84.4C, S85.4G, T85.3C, R85.1S, V106A, S107A, |
| | N108H, K109P, A110E, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |

TABLE 23
Affinity and maximum response of engineered immunoglobulin
towards hFcαRI determined by surface plasmon resonance,
following the procedure described in Example 5a.

| | | | Maximum response at |
|---|---|---|---|
| SEQ ID NO | Ref number | KD (1:1) (M) | 1000 nM hFcαRI (RU) |
| 2 | 2258 | 5.66E−07 | 80 |
| 100 | 2749 | 1.21E−06 | 27 |
| 101 | 2750 | 1.80E−06 | 24 |
| 102 | 2751 | 1.20E−06 | 38 |
| 103 | 2752 | 8.24E−07 | 34 |
| 104 | 2753 | 2.81E−06 | 75 |
| 105 | 2754 | 1.73E−06 | 70 |
| 106 | 2755 | 9.54E−07 | 28 |
| 107 | 2756 | 2.24E−06 | 76 |
| 108 | 2757 | 2.23E−06 | 68 |
| 109 | 2758 | 8.02E−07 | 27 |
| 110 | 2759 | 3.29E−06 | 80 |
| 111 | 2760 | 2.47E−06 | 74 |
| 112 | 2761 | 1.65E−06 | 52 |
| 113 | 2762 | 7.87E−07 | 118 |
| 114 | 2763 | 1.19E−06 | 76 |
| 115 | 2764 | 1.26E−06 | 55 |
| 116 | 2765 | 1.51E−06 | 137 |
| 117 | 2766 | 1.65E−06 | 92 |
| 118 | 2767 | 2.07E−06 | 62 |
| 119 | 2768 | 8.22E−07 | 156 |
| 120 | 2769 | 1.30E−06 | 142 |
| 121 | 2770 | 2.08E−06 | 71 |
| 122 | 2771 | 1.31E−06 | 150 |
| 123 | 2772 | 1.77E−06 | 120 |

TABLE 24
Affinity and maximum response of engineered immunoglobulin
towards hFcαRI determined by surface plasmon resonance,
following the procedure described in Example 5b.

| | | | Maximum response at |
|---|---|---|---|
| SEQ ID NO | Ref number | KD (1:1) (M) | 1000 nM hFcαRI (RU) |
| 3 | 2737 | 5.20E−07 | 168 |
| 146 | 3082 | 0.00E+00 | 18 |
| 147 | 3083 | 0.00E+00 | 11 |
| 148 | 3084 | 5.09E−07 | 146 |
| 149 | 3085 | 2.22E−07 | 100 |
| 165 | 3014 | 3.04E−07 | 178 |
| 166 | 3015 | 6.36E−07 | 100 |
| 167 | 3016 | 7.17E−07 | 88 |
| 168 | 3017 | 2.54E−07 | 116 |
| 169 | 3018 | 2.26E−07 | 105 |
| 170 | 3019 | 3.51E−07 | 120 |
| 171 | 3020 | 4.00E−07 | 98 |
| 172 | 3021 | 4.74E−07 | 101 |
| 173 | 3022 | 2.28E−07 | 146 |
| 174 | 3023 | 3.42E−07 | 177 |
| 175 | 3024 | 4.40E−07 | 128 |
| 176 | 3025 | 1.64E−07 | 155 |
| 177 | 3026 | 2.85E−07 | 165 |
| 178 | 3027 | 1.40E−07 | 190 |
| 179 | 3028 | 2.85E−07 | 162 |
| 180 | 3029 | 1.92E−07 | 146 |
| 181 | 3030 | 3.58E−07 | 143 |
| 119 | 2768 | 2.22E−07 | 191 |
| 120 | 2769 | 3.18E−07 | 147 |
| 122 | 2771 | 2.70E−07 | 197 |
| 123 | 2772 | 3.72E−07 | 154 |

### Example 2: Restoration of hFcRn Binding, hFcγR Binding and Generation of Heterodimers Combining Alpha/Gamma Effector Functions and hFcRn Binding

2.1 hFcRn Binding Restoration for Half-Life Extension

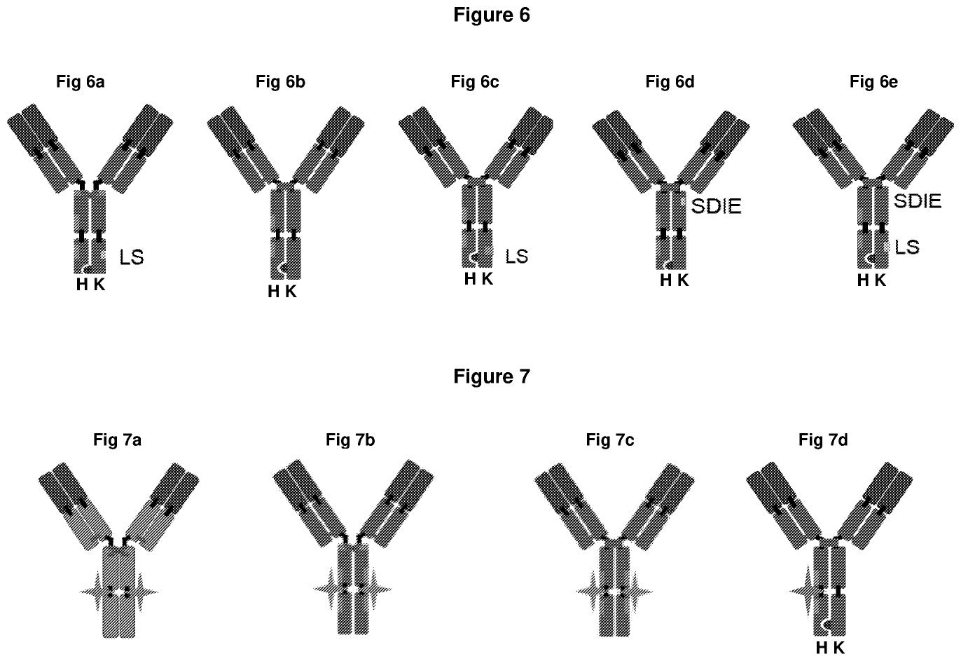

The key residues of IgG1 Fc that interact with hFcRn are located in the corresponding region of IgA2 Fc (CH3) that is responsible for hFcαRI interactions. For this reason, it was not possible to transfer the complete hFcαRI interaction site from IgA2 CH3 to IgG1 CH3 without losing hFcRn binding capacity. To determine whether hFcRn binding could be conferred to an engineered immunoglobulin comprising CH2 and CH3 domains from IgA2, substitutions to the CH3 domain were made at with the corresponding amino acid residue from IgG1 (shown schematically in FIG. 5a; see Table 25). With these mutations in the engineered immunoglobulin, it was possible to restore hFcRn binding; however this was to the detriment of hFcαR binding and therefore there was some loss of response of the engineered immunoglobulins towards hFcαRI, as shown in Table 25.

TABLE 25
Summary of CH3 mutations (based on parental IgA2 (SEQ ID NO: 3)
and numbered according to IMGT numbering for C-domain), and
affinity and maximum response of engineered immunoglobulins
towards hFcαRI determined by surface plasmon resonance,
following the procedure described in Example 5b

| | | | | Maximum response |
|---|---|---|---|---|
| SEQ ID | CH3 | Ref | KD (1:1) | at 1000 nM |
| NO | Mutation set | number | (M) | hFcαRI (RU) |
| 3 | — | 2737 | 5.20E−07 | 168 |
| 71 | A115H, F116Y | 2591 | 0.00E+00 | 7 |
| 72 | P113H, L114N, | 2592 | 0.00E+00 | 0 |
| | A115H, F116Y | | | |

2.2 Gamma Response Restoration

The engineered immunoglobulins based on an IgG1 Fc region and having mutations to restore IgA binding lacked the ability to recruit gamma effector function by binding to hFcγRs. Therefore, it was necessary to replace IgA residues located in top-CH2 disulfide node region by IgG1 residues. Enhancement of binding to hFcγRs was possible by introducing further mutations to the homodimer. These mutations S239D and 1332E (“SDIE”; EU Numbering; Lazar et al (2006) supra) were introduced into the CH2 domain of both Fc domains of the homodimer (shown schematically in FIG. 5b) and were effective in restoring IgG effector function to the engineered homodimeric immunoglobulin. The amino acid sequences shown in SEQ ID NOs: 150 to 153 include the SDIE mutations and additional mutations which are shown in Table 26 below.

However, restoration of hFcγRIa and hFcγRIIIa interaction to the engineered homodimeric immunoglobulins reduced binding to hFcαRI, as shown Tables 27, 28 and 29. As can be seen in Table 27, the engineered immunoglobulins comprising a higher content of residues derived from IgA had an improved binding affinity to hFcαRI over engineered immunoglobulins with a lower content of IgA residues. As can be seen in Table 28, engineered immunoglobulins comprising a lower content of residues derived from IgA had an improved binding affinity to hFcγRIa compared to engineered immunoglobulins with a higher content of IgA residues. Binding to hFcγRIa was only observed when the residues spatially located on top of the CH2 were fully derived from IgG1. As can be seen in Table 29, engineered immunoglobulins comprising a lower content of residues derived from IgA had an improved binding affinity to hFcγRIIIa compared to engineered immunoglobulins with a higher content of IgA residues.

Therefore, it was difficult to maintain both alpha and gamma effector functions at IgA2 or IgG1 level using a homodimeric Fc format. An in vitro PMN/PBMC killing assay and an ADCP assay confirm these observations, as shown in FIGS. 13-16 for the engineered immunoglobulins comprising SEQ ID NOs: 122, 148 and 152.

Preferred homodimeric engineered IgG1 immunoglobulin candidates having hFcαRI, hFcγRIa and hFcγRIIIa binding properties comprised Fc domains comprised within SEQ ID NOs: 148 and 152.

TABLE 26
Summary of CH2 and CH3 mutations (based on sequence SEQ ID NO: 1)

| SEQ | |
|---|---|
| ID NO | CH2 and CH3 Mutation set (IMGT numbering for C-domain) |
| 150 | CH2: S3D, P8R, K10A, P11L, K12E, T14L, M15.1L, I15.2#, S16G, R17S, T18E, P19A, |
| | E20N, T90P, V91G, L92C, H93A, D95P, L97N, N98H, I117E, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 151 | CH2: S3D, P8R, K10A, P11L, K12E, T14L, M15.1L, I15.2#, S16G, R17S, T18E, P19A, |
| | E20N, T90P, V91G, L92C, H93A, D95P, L97N, N98H, I117E, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, S105M, M107G, H113P, N114L, H115A, Y116F, |
| | L121I, |
| 152 | CH2: S3D, P8R, K10A, P11L, K12E, T14L, M15.1L, I15.2#, S16G, R17S, T18E, P19A, |
| | E20N, H17E, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, D92A, K93A, S94E, R95D, Q97K, Q98K, N100D, |
| | V101T, S105M, M107G, H113P, N114L, H115A, Y116F, L121I, L123R |
| 153 | CH2: S3D, P8R, K10A, P11L, K12E, T14L, M15.1L, I15.2#, S16G, R17S, T18E, P19A, |
| | E20N, H17E, K125# |
| | CH3: T6L, V25A, S35K, I37V, A38L, E40R, E42L, S43Q, N44G, G45S, P45.2E, E45.3L, |
| | N45.4P, \hat{ }45.4RE, P82A, P83S, L85V, S105M, M107G, H113P, N114L, H115A, Y116F, |
| | L121I, |

TABLE 27
Affinity and maximum response of engineered immunoglobulins
towards hFcαRI as determined by surface plasmon resonance,
following the procedure described in Example 5b.

| | | | Maximum response |
|---|---|---|---|
| | | | at 1000 nM hFcαRI |
| SEQ ID NO | Ref number | KD (1:1) (M) | (RU) |
| 3 | 2737 | 2.38E−07 | 182 |
| 122 | 2771 | 2.70E−07 | 197 |
| 165 | 3014 | 3.04E−07 | 178 |
| 166 | 3015 | 6.36E−07 | 100 |
| 167 | 3016 | 7.17E−07 | 88 |
| 168 | 3017 | 2.54E−07 | 116 |
| 169 | 3018 | 2.26E−07 | 105 |
| 170 | 3019 | 3.51E−07 | 120 |
| 171 | 3020 | 4.00E−07 | 98 |
| 172 | 3021 | 4.74E−07 | 101 |
| 173 | 3022 | 2.28E−07 | 146 |
| 146 | 3082 | 0.00E+00 | 18 |
| 147 | 3083 | 0.00E+00 | 10 |
| 148 | 3084 | 5.09E−07 | 146 |
| 149 | 3085 | 2.22E−07 | 100 |
| 150 | 3086 | 0.00E+00 | 0 |
| 151 | 3087 | 0.00E+00 | 7 |
| 152 | 3088 | 1.99E−07 | 85 |
| 153 | 3089 | 3.00E−07 | 56 |

TABLE 28
Affinity and maximum response of engineered immunoglobulins
towards hFcγRIa as determined by surface plasmon resonance,
following the procedure described in Example 5b.

| | | | Maximum response at |
|---|---|---|---|
| SEQ ID NO | Ref number | KD (1:1) (M) | 1000 nM hFcγRIa (RU) |
| 1 | 2257 | 3.50E−11 | 208 |
| 3 | 2737 | 0 | 0 |
| 122 | 2771 | 0.00E+00 | 0 |
| 165 | 3014 | 0.00E+00 | 0 |
| 166 | 3015 | 0.00E+00 | 0 |
| 167 | 3016 | 0.00E+00 | 0 |
| 168 | 3017 | 7.96E−10 | 266 |
| 169 | 3018 | 0.00E+00 | 0 |
| 170 | 3019 | 0.00E+00 | 0 |
| 171 | 3020 | 0.00E+00 | 0 |
| 172 | 3021 | 0.00E+00 | 0 |
| 173 | 3022 | 0.00E+00 | 0 |
| 146 | 3082 | 0.00E+00 | 0 |
| 147 | 3083 | 0.00E+00 | 0 |
| 148 | 3084 | 1.26E−09 | 211 |
| 149 | 3085 | 1.38E−09 | 199 |
| 150 | 3086 | 0.00E+00 | 0 |
| 151 | 3087 | 0.00E+00 | 13 |
| 152 | 3088 | 1.06E−10 | 218 |
| 153 | 3089 | 1.40E−10 | 203 |

TABLE 29
Maximum response of engineered immunoglobulins toward
hFcγRIIIa as determined by surface plasmon resonance,
following the procedure described in Example 5b. Maximum
response only was determined to speed up the screening process.

| | | | Maximum response at |
|---|---|---|---|
| SEQ ID NO | Ref number | KD (1:1) (M) | 1000 nM hFcγRIIIa (RU) |
| 1 | 2257 | ND | 164 |
| 3 | 2737 | ND | 0 |
| 122 | 2771 | ND | 0 |
| 168 | 3017 | ND | 0 |
| 146 | 3082 | ND | 0 |
| 147 | 3083 | ND | 0 |
| 148 | 3084 | ND | 23 |
| 149 | 3085 | ND | 16 |
| 150 | 3086 | ND | 0 |
| 151 | 3087 | ND | 0 |
| 152 | 3088 | ND | 62 |
| 153 | 3089 | ND | 53 |

2.3 Heterodimeric Fc (Knob into Hole)

The challenge of trying to maintain both alpha and gamma effector functions at IgA2 or IgG1 level was therefore addressed by using an engineered heterodimeric immunoglobulin Fc region, based on “knob into hole” technology (Merchant et al., (1998) supra). In such a molecule, binding to hFcRn was restored when one half of the IgG1 Fc was engineered to bind to and recruit hFcαRI, and the second half of the IgG1 Fc was kept unchanged. It was also found that when the second half of the IgG1 Fc comprised residues spatially located on top of CH2 that were derived from IgA2, for example, packed loops and disulfide bonds, the amino acid mutations “LS” (M428L, N434S (EU numbering)/M_CH3.107_L/N_CH3.114_S (IMGT numbering for C-domain); Zalevsky et al., (2010) supra) and the “YTE” mutation (M252Y, S254T, T256E (EU Numbering)/M_CH2.15.1_Y/S_CH3.16_T/T_CH2.18_E (IMGT numbering for C-domain); Dall'acqua et al., (2002) supra), were required in the second half of the IgG1 Fc to restore binding to hFcRn. Fc domains comprised within SEQ ID NOs: 132, 134, 136, 138, 140, 142, 144, 154, 159, 160, 161, 162, 163 and 164 include the mutations S354C/S_CH3.10_C and T366W/T_CH3.22_W (EU/IMGT numbering for C-domain) to introduce a “knob”. Fc domains comprised within SEQ ID NOs: 133, 135, 137, 139, 141, 143, 145, 155, 156, 157 and 158 include the mutations Y349C/Y_CH3.5_C, T366S/T_CH3.22_S, L368A/L_CH3.24_S and Y407V/Y_CH3.86_V (EU/IMGT numbering for C-domain) to introduce a “hole”. Fc domains comprised within SEQ ID NOs: 154, 160 and 162 also include the LS mutation, and the Fc domain comprised within SEQ ID NO: 163 also includes the YTE mutation. Details of the complete mutations sets for the Fc domains comprising the “knob” mutations and additional mutations are shown in Table 30 below. Details of the complete mutations sets for the Fc domains comprising the “hole” mutations and additional mutations are shown in Table 31 below.

Important for the functioning of this “knob into hole” structure was that the “hole” mutations were made in the half of the Fc comprising the residues recruiting hFcαRI whereas the “knob” mutations were placed on the other half of the Fc. It was found that making the “knob” mutations in the half of the Fc recruiting hFcαRI lead to a high loss of affinity of the engineered IgG1 immunoglobulin for hFcαRI.

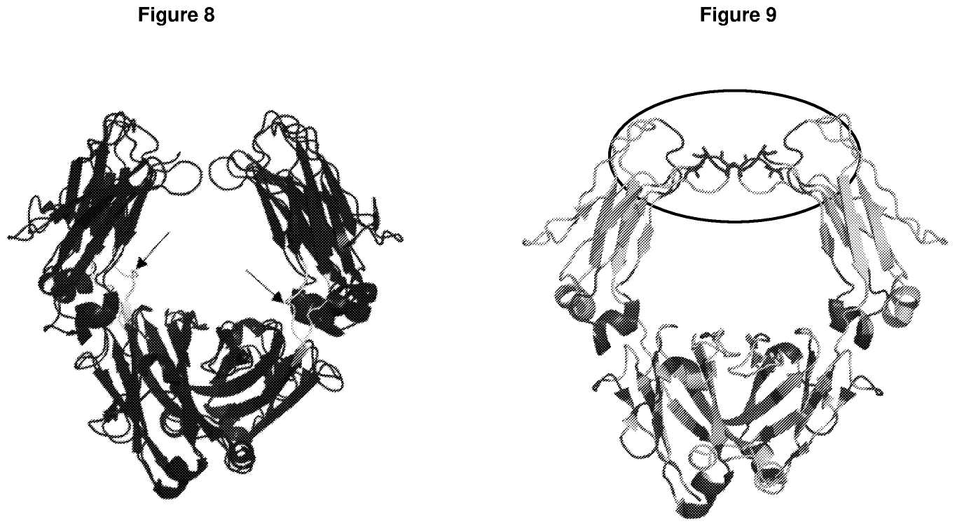

For the heterodimeric engineered IgG1 immunoglobulins comprising a first Fc domain with mutations to restore IgA effector function and a second Fc domain from IgG1, both Fc domains having “hole” and “knob” mutations respectively, as described in Example 1.3, full FcγR effector function was restored with the addition of the SDIE mutations. However, it was necessary to introduce the SDIE mutations into the Fc domain containing the “knob” mutation only, so as not to interfere with FcαRI binding. The Fc domains comprised within SEQ ID NOs: 161, 162 and 164 include the SDIE mutations as well as mutations to generate a “knob” for heterodimer stabilization. The Fc domain comprised within SEQ ID NO: 162 also includes the LS mutations to restore hFcRn binding. The generated heterodimeric formats are shown schematically in FIGS. 6a-e.

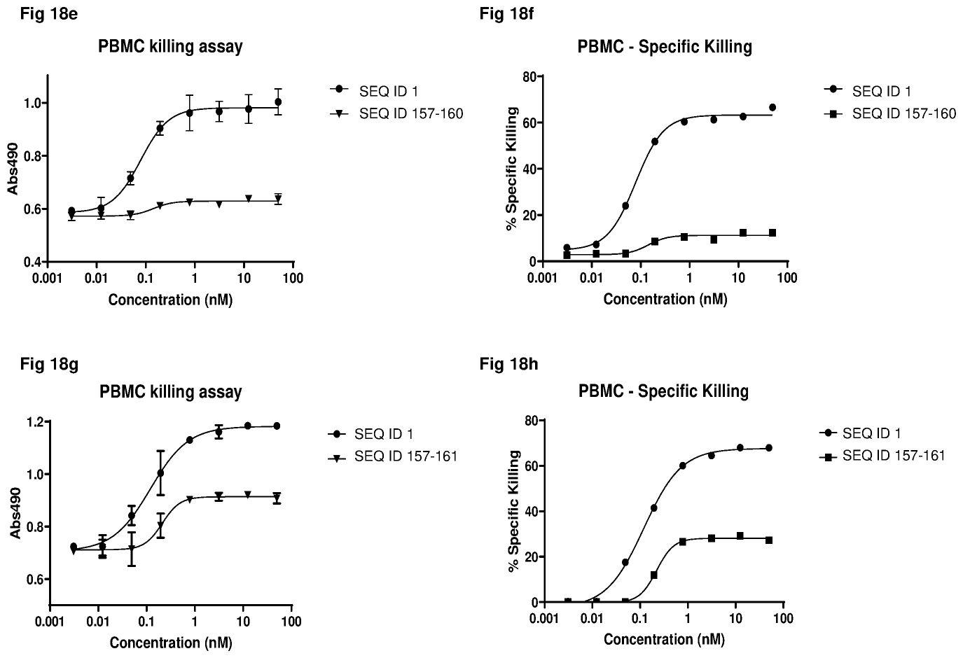

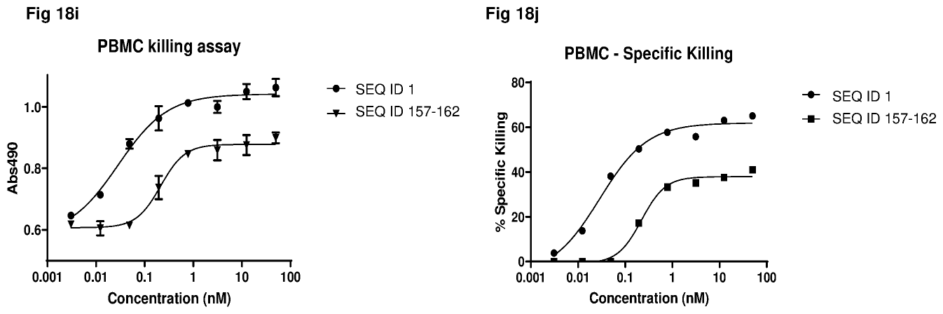

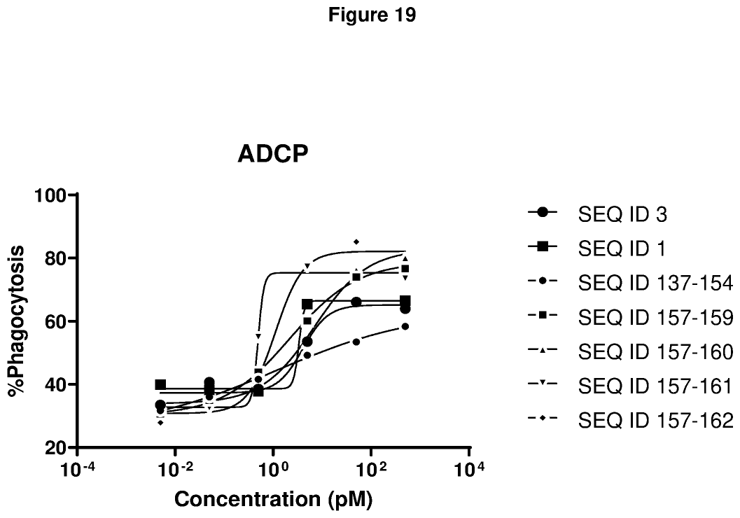

Binding of the engineered heterodimeric immunoglobulins to hFcαRI, hFcγRIa, hFcγRIIIa and hFcRn was characterized by surface plasmon resonance. The results are shown in Tables 32-36. The heterodimeric lead candidates (shown schematically in FIG. 6a and FIG. 6b) are listed in Table 37, and were tested in an in vitro ADCC and ADCP assay, as described in Examples 6 and 7, respectively. The results are shown in FIG. 17, FIG. 18 and FIG. 19. The resulting engineered immunoglobulins were constructed to achieve the following binding properties (see Table 37):
a. hFcαRI binding in a range from parental IgA2-like binding to about 10-fold lower than parental IgA2;
b. hFcαRI binding in a range from parental IgA2-like binding to about 10-fold lower, and wild-type IgG1-like binding towards hFcγ receptors;
c. hFcαRI binding in a range from parental IgA2-like binding to about 10-fold lower, and wild-type IgG1-like binding towards hFcRn; and
d. hFcαRI binding in a range from parental IgA2-like binding to about 10-fold lower, and wild-type IgG1-like binding towards hFcγRIa, hFcγRIIIa and hFcRn.

In an ADCP assay to assess phagocytosis, all heterodimeric candidates except for the SEQ ID NO: 137-154 pair, showed improved phagocytosis with increasing concentration compared to parental IgA2 (SEQ ID NO: 3) and wild-type IgG1 (SEQ ID NO: 1), as shown in FIG. 19.

TABLE 30
Summary of CH2 and CH3 mutations (based on sequence SEQ ID NO: 1)

| SEQ ID | |
|---|---|
| NO | CH2 and CH3 Mutation set ‘knob’ (IMGT numbering for C-domain) |
| 132 | CH2: A1.6#, P1.5#, E1.4#, L1.3#, L1.2C, G1.1C, G1H, S3R, P8R, K10A, P11L, K12E, |
| | T14L, M15.1L, 115.2#, S16G, R17S, T18E, P19A, E20N, V26T, D27G, V28L, S29R, |
| | H30D, N84.4C, S85.4G, T85.3C, R85.1S, T90P, V91G, L92C, H93A, D95P, L97N, N98H, |
| | K125# |
| | CH3: T6L, S10C, T22W, V25A, S35K, I37V, A38L, E40R, E42L, S43Q, N44G, G45S, |
| | P45.2E, E45.3L, N45.4P, A45.4RE, P82A, P83S, L85V, D92A, K93A, S94E, R95D, Q97K, |
| | Q98K, N100D, V101T, S105M, M107G, H113P, N114L, H115A, Y116F, L121I, L123R |
| 134 | CH2: A1.6#, P1.5#, E1.4#, L1.3#, L1.2C, G1.1C, G1H, S3R, P8R, K10A, P11L, K12E, |
| | T14L, M15.1L, 115.2#, S16G, R17S, T18E, P19A, E20N, V26T, D27G, V28L, S29R, |
| | H30D, N84.4C, S85.4G, T85.3C, R85.1S, T90P, V91G, L92C, H93A, D95P, L97N, N98H, |
| | K125# |
| | CH3: T6L, S10C, T22W, V25A, S35K, I37V, A38L, E40R, E42L, S43Q, N44G, G45S, |
| | P45.2E, E45.3L, N45.4P, A45.4RE, P82A, P83S, L85V, S105M, M107G, H113P, N114L, |
| | H115A, Y116F, L1211 |
| 136 | CH2: A1.6#, P1.5#, E1.4#, L1.3#, L1.2C, G1.1C, G1H, S3R, P8R, K10A, P11L, K12E, |
| | T14L, M15.1L, 115.2#, S16G, R17S, T18E, P19A, E20N, V26T, D27G, V28L, S29R, |
| | H30D, N84.4C, S85.4G, T85.3C, R85.1S, K125# |
| | CH3: T6L, S10C, T22W, V25A, S35K, I37V, A38L, E40R, E42L, S43Q, N44G, G45S, |
| | P45.2E, E45.3L, N45.4P, A45.4RE, P82A, P83S, L85V, D92A, K93A, S94E, R95D, Q97K, |
| | Q98K, N100D, V101T, S105M, M107G, H113P, N114L, H115A, Y116F, L121I, L123R |
| 138 | CH2: A1.6#, P1.5#, E1.4#, L1.3#, L1.2C, G1.1C, G1H, S3R, P8R, K10A, P11L, K12E, |
| | T14L, M15.1L, 115.2#, S16G, R17S, T18E, P19A, E20N, V26T, D27G, V28L, S29R, |
| | H30D, N84.4C, S85.4G, T85.3C, R85.1S, K125# |
| | CH3: T6L, S10C, T22W, V25A, S35K, I37V, A38L, E40R, E42L, S43Q, N44G, G45S, |
| | P45.2E, E45.3L, N45.4P, A45.4RE, P82A, P83S, L85V, S105M, M107G, H113P, N114L, |
| | H115A, Y116F, L1211 |
| 140 | CH2: A1.6#, P1.5#, E1.4#, L1.3#, L1.2C, G1.1C, G1H, S3R, P8R, K10A, P11L, K12E, |
| | T14L, M15.1L, 115.2#, S16G, R17S, T18E, P19A, E20N, V26T, D27G, V28L, S29R, |
| | H30D, N84.4C, S85.4G, T85.3C, R85.1S, T90P, V91G, L92C, H93A, D95P, L97N, N98H, |
| | K125# |
| | CH3: S10C, T22W, A45.4RE |
| 142 | CH2: A1.6#, P1.5#, E1.4#, L1.3#, L1.2C, G1.1C, G1H, S3R, P8R, K10A, P11L, K12E, |
| | T14L, M15.1L, 115.2#, S16G, R17S, T18E, P19A, E20N, V26T, D27G, V28L, S29R, |
| | H30D, N84.4C, S85.4G, T85.3C, R85.1S, K125# |
| | CH3: S10C, T22W, A45.4RE |
| 144 | CH2: A1.6#, P1.5#, E1.4#, L1.3#, L1.2C, G1.1C, G1H, S3R, V26T, D27G, V28L, S29R, |
| | H30D, N84.4C, S85.4G, T85.3C, R85.1S, K125# |
| | CH3: S10C, T22W, A45.4RE |
| 154 | CH2: A1.6#, P1.5#, E1.4#, L1.3#, L1.2C, G1.1C, G1H, S3R, V26T, D27G, V28L, S29R, |
| | H30D, N84.4C, S85.4G, T85.3C, R85.1S, K125# |
| | CH3: S10C, T22W, A45.4RE, M107L, N114S |
| 159 | CH2: K125# |
| | CH3: S10C, T22W, A45.4RE |
| 160 | CH2: K125# |
| | CH3: S10C, T22W, A45.4RE, M107L, N114S |
| 161 | CH2: S3D, H17E, K125# |
| | CH3: S10C, T22W, A45.4RE |
| 162 | CH2: S3D, H17E, K125# |
| | CH3: S10C, T22W, A45.4RE, M107L, N114S |
| 163 | CH2: A1.6#, P1.5#, E1.4#, L1.3#, L1.2C, G1.1C, G1H, S3R, M15.1Y, S16T, T18E, V26T, |
| | D27G, V28L, S29R, H30D, N84.4C, S85.4G, T85.3C, R85.1S, K125# |
| | CH3: S10C, T22W, A45.4RE |
| 164 | CH2: A1.3L, L1.2C, G1.1C, G1H, S3D, V26T, D27G, V28L, S29R, H30D, N84.4C, |
| | S85.4G, T85.3C, R85.1S, 1117E, K125# |
| | CH3: S10C, T22W, A45.4RE |

TABLE 31
Summary of CH2 and CH3 mutations (based on sequence SEQ ID NO: 1)

| SEQ ID | |
|---|---|
| NO | CH2 and CH3 Mutation set ‘hole’ (IMGT numbering for C-domain) |
| 133 | CH2: A1.6#, P1.5#, E1.4#, L1.3#, L1.2C, G1.1C, G1H, S3R, P8R, K10A, P11L, K12E, |
| | T14L, M15.1L, 115.2#, S16G, R17S, T18E, P19A, E20N, V26T, D27G, V28L, S29R, |
| | H30D, N84.4C, S85.4G, T85.3C, R85.1S, T90P, V91G, L92C, H93A, D95P, L97N, N98H, |
| | K125# |
| | CH3: T6L, T22S, L24A, V25A, S35K, I37V, A38L, E40R, E42L, S43Q, N44G, G45S, |
| | P45.2E, E45.3L, N45.4P, A45.4RE, P82A, P83S, L85V, Y86V, D92A, K93A, S94E, R95D, |
| | Q97K, Q98K, N100D, V101T, S105M, M107G, H113P, N114L, H115A, Y116F, L121I, |
| | L123R |
| 135 | CH2: A1.6#, P1.5#, E1.4#, L1.3#, L1.2C, G1.1C, G1H, S3R, P8R, K10A, P11L, K12E, |
| | T14L, M15.1L, 115.2#, S16G, R17S, T18E, P19A, E20N, V26T, D27G, V28L, S29R, |
| | H30D, N84.4C, S85.4G, T85.3C, R85.1S, T90P, V91G, L92C, H93A, D95P, L97N, N98H, |
| | K125# |
| | CH3: T6L, T22S, L24A, V25A, S35K, I37V, A38L, E40R, E42L, S43Q, N44G, G45S, |
| | P45.2E, E45.3L, N45.4P, A45.4RE, P82A, P83S, L85V, Y86V, S105M, M107G, H113P, |
| | N114L, H115A, Y116F, L1211 |
| 137 | CH2: A1.6#, P1.5#, E1.4#, L1.3#, L1.2C, G1.1C, G1H, S3R, P8R, K10A, P11L, K12E, |
| | T14L, M15.1L, 115.2#, S16G, R17S, T18E, P19A, E20N, V26T, D27G, V28L, S29R, |
| | H30D, N84.4C, S85.4G, T85.3C, R85.1S, K125# |
| | CH3: T6L, T22S, L24A, V25A, S35K, I37V, A38L, E40R, E42L, S43Q, N44G, G45S, |
| | P45.2E, E45.3L, N45.4P, A45.4RE, P82A, P83S, L85V, Y86V, D92A, K93A, S94E, R95D, |
| | Q97K, Q98K, N100D, V101T, S105M, M107G, H113P, N114L, H115A, Y116F, L121I, |
| | L123R |
| 139 | CH2: A1.6#, P1.5#, E1.4#, L1.3#, L1.2C, G1.1C, G1H, S3R, P8R, K10A, P11L, K12E, |
| | T14L, M15.1L, 115.2#, S16G, R17S, T18E, P19A, E20N, V26T, D27G, V28L, S29R, |
| | H30D, N84.4C, S85.4G, T85.3C, R85.1S, K125# |
| | CH3: T6L, T22S, L24A, V25A, S35K, I37V, A38L, E40R, E42L, S43Q, N44G, G45S, |
| | P45.2E, E45.3L, N45.4P, A45.4RE, P82A, P83S, L85V, Y86V, S105M, M107G, H113P, |
| | N114L, H115A, Y116F, L1211 |
| 141 | CH2: A1.6#, P1.5#, E1.4#, L1.3#, L1.2C, G1.1C, G1H, S3R, P8R, K10A, P11L, K12E, |
| | T14L, M15.1L, 115.2#, S16G, R17S, T18E, P19A, E20N, V26T, D27G, V28L, S29R, |
| | H30D, N84.4C, S85.4G, T85.3C, R85.1S, T90P, V91G, L92C, H93A, D95P, L97N, N98H, |
| | K125# |
| | CH3: T22S, L24A |
| 143 | CH2: A1.6#, P1.5#, E1.4#, L1.3#, L1.2C, G1.1C, G1H, S3R, P8R, K10A, P11L, K12E, |
| | T14L, M15.1L, 115.2#, S16G, R17S, T18E, P19A, E20N, V26T, D27G, V28L, S29R, |
| | H30D, N84.4C, S85.4G, T85.3C, R85.1S, K125# |
| | CH3: T22S, L24A |
| 145 | CH2: A1.6#, P1.5#, E1.4#, L1.3#, L1.2C, G1.1C, G1H, S3R, V26T, D27G, V28L, S29R, |
| | H30D, N84.4C, S85.4G, T85.3C, R85.1S, K125# |
| | CH3: T22S, L24A |
| 155 | CH2: P8R, K10A, P11L, K12E, T14L, M15.1L, 115.2#, S16G, R17S, T18E, P19A, E2ON, |
| | T90P, V91G, L92C, H93A, D95P, L97N, N98H, K125# |
| | CH3: T6L, T22S, L24A, V25A, S35K, I37V, A38L, E40R, E42L, S43Q, N44G, G45S, |
| | P45.2E, E45.3L, N45.4P, A45.4RE, P82A, P83S, L85V, Y86V, D92A, K93A, S94E, R95D, |
| | Q97K, Q98K, N100D, V101T, S105M, M107G, H113P, N114L, H115A, Y116F, L121I, |
| | L123R |
| 156 | CH2: P8R, K10A, P11L, K12E, T14L, M15.1L, 115.2#, S16G, R17S, T18E, P19A, E2ON, |
| | T90P, V91G, L92C, H93A, D95P, L97N, N98H, K125# |
| | CH3: T6L, T22S, L24A, V25A, S35K, I37V, A38L, E40R, E42L, S43Q, N44G, G45S, |
| | P45.2E, E45.3L, N45.4P, A45.4RE, P82A, P83S, L85V, Y86V, S105M, M107G, H113P, |
| | N114L, H115A, Y116F, L1211 |
| 157 | CH2: P8R, K10A, P11L, K12E, T14L, M15.1L, 115.2#, S16G, R17S, T18E, P19A, E2ON, |
| | K125# |
| | CH3: T6L, T22S, L24A, V25A, S35K, I37V, A38L, E40R, E42L, S43Q, N44G, G45S, |
| | P45.2E, E45.3L, N45.4P, A45.4RE, P82A, P83S, L85V, Y86V, D92A, K93A, S94E, R95D, |
| | Q97K, Q98K, N100D, V101T, S105M, M107G, H113P, N114L, H115A, Y116F, L121I, |
| | L123R |
| 158 | CH2: P8R, K10A, P11L, K12E, T14L, M15.1L, 115.2#, S16G, R17S, T18E, P19A, E20N, |
| | K125# |
| | CH3: T6L, T22S, L24A, V25A, S35K, I37V, A38L, E40R, E42L, S43Q, N44G, G45S, |
| | P45.2E, E45.3L, N45.4P, A45.4RE, P82A, P83S, L85V, Y86V, S105M, M107G, H113P, |
| | N114L, H115A, Y116F, L1211 |

TABLE 32
Affinity and maximum response of engineered immunoglobulins
toward hFcαRI determined by SPR experiment, following procedure
described in Example 5b.

| | | | Monovalent maximum |
|---|---|---|---|
| | | KD (1:1) | response at 1000 nM |
| SEQ ID NO | Ref number | (M) | hFcαRI (RU) |
| 3 | 2737 | 5.20E−07 | 168 |
| 119 | 2768 | 5.79E−07 | 133 |
| 120 | 2769 | 9.73E−07 | 95 |
| 122 | 2771 | 9.24E−07 | 122 |
| 123 | 2772 | 1.19E−06 | 88 |
| 132-141 | 2840-2851 | 3.34E−06 | 38 |
| 136-143 | 2841-2852 | 3.05E−07 | 68 |
| 134-141 | 2842-2851 | 4.82E−07 | 34 |
| 138-143 | 2843-2852 | 1.85E−06 | 33 |
| 132-145 | 2840-2853 | 8.57E−07 | 48 |
| 136-145 | 2841-2853 | 1.29E−06 | 34 |
| 134-145 | 2842-2853 | 2.01E−06 | 28 |
| 138-145 | 2843-2853 | 8.50E−07 | 28 |
| 133-140 | 3090-2844 | 7.01E−07 | 45 |
| 137-142 | 3092-2845 | 1.16E−06 | 57 |
| 135-140 | 3091-2844 | 7.60E−07 | 40 |
| 139-142 | 3093-2845 | 1.09E−06 | 49 |
| 133-144 | 3090-2846 | 9.19E−07 | 45 |
| 137-144 | 3092-2846 | 1.05E−06 | 56 |
| 135-144 | 3091-2846 | 9.17E−07 | 46 |
| 139-144 | 3093-2846 | 1.82E−06 | 31 |
| 137-163 | 3011-3092 | 4.36E−07 | 59 |
| 133-154 | 3090-3094 | 2.32E−07 | 64 |
| 135-154 | 3091-3094 | 2.90E−07 | 61 |
| 137-154 | 3092-3094 | 3.78E−07 | 77 |
| 139-154 | 3093-3094 | 4.53E−07 | 75 |
| 155-159 | 3095-3099 | 4.27E−07 | 63 |
| 156-159 | 3096-3099 | 5.00E−07 | 52 |
| 157-159 | 3097-3099 | 4.63E−07 | 86 |
| 158-159 | 3098-3099 | 6.07E−07 | 72 |
| 155-160 | 3095-3100 | 3.86E−07 | 67 |
| 156-160 | 3096-3100 | 4.61E−07 | 63 |
| 157-160 | 3097-3100 | 6.62E−07 | 83 |
| 158-160 | 3098-3100 | 6.99E−07 | 68 |
| 157-161 | 3097-3101 | 5.25E−07 | 70 |
| 158-161 | 3098-3101 | 7.45E−07 | 66 |
| 157-162 | 3097-3102 | 6.90E−07 | 66 |
| 158-162 | 3098-3102 | 8.56E−07 | 64 |

TABLE 33
Affinity and maximum response of engineered immunoglobulins
toward hFcγRIa determined by SPR experiment, following procedure
described in Example 5b.

| | | | Maximum response at |
|---|---|---|---|
| SEQ ID NO | Ref number | KD (1:1) (M) | 1000 nM hFcγRIa (RU) |
| 1 | 2257 | 3.50E−11 | 208 |
| 3 | 2737 | 0.00E+00 | 0 |
| 122 | 2771 | 0.00E+00 | 0 |
| 137-163 | 3011-3092 | 0.00E+00 | 0 |
| 133-154 | 3090-3094 | 0.00E+00 | 0 |
| 135-154 | 3091-3094 | 0.00E+00 | 0 |
| 137-154 | 3092-3094 | 0.00E+00 | 0 |
| 139-154 | 3093-3094 | 0.00E+00 | 0 |
| 155-159 | 3095-3099 | 6.32E−10 | 126 |
| 156-159 | 3096-3099 | 5.87E−10 | 110 |
| 157-159 | 3097-3099 | 7.37E−10 | 178 |
| 158-159 | 3098-3099 | 7.72E−10 | 182 |
| 155-160 | 3095-3100 | 1.54E−09 | 83 |
| 156-160 | 3096-3100 | 1.77E−09 | 80 |
| 157-160 | 3097-3100 | 1.91E−09 | 98 |
| 158-160 | 3098-3100 | 1.81E−09 | 92 |
| 157-161 | 3097-3101 | 5.55E−11 | 373 |
| 158-161 | 3098-3101 | 4.65E−11 | 417 |
| 155-162 | 3095-3102 | 1.25E−08 | 341 |
| 157-162 | 3097-3102 | 8.75E−10 | 150 |
| 158-162 | 3098-3102 | 7.85E−10 | 157 |

TABLE 34
Maximum response of engineered immunoglobulins toward hFcγRIIIa
determined by SPR experiment, following procedure described in
Example 5b. Maximum response only was determined to drive
selection and speed up screening.

| | | | Maximum response |
|---|---|---|---|
| | | | at 500 nM hFcγRIIIa |
| SEQ ID NO | Ref number | KD (1:1) (M) | (RU) |
| 1 | 2257 | ND | 164 |
| 3 | 2737 | ND | 0 |
| 133-154 | 3090-3094 | ND | 0 |
| 135-154 | 3091-3094 | ND | 0 |
| 137-154 | 3092-3094 | ND | 0 |
| 139-154 | 3093-3094 | ND | 0 |
| 155-159 | 3095-3099 | ND | 15 |
| 156-159 | 3096-3099 | ND | 13 |
| 157-159 | 3097-3099 | ND | 28 |
| 158-159 | 3098-3099 | ND | 25 |
| 155-160 | 3095-3100 | ND | 0 |
| 156-160 | 3096-3100 | ND | 0 |
| 157-160 | 3097-3100 | ND | 0 |
| 158-160 | 3098-3100 | ND | 0 |
| 155-161 | 3095-3101 | ND | ND |
| 156-161 | 3096-3101 | ND | ND |
| 157-161 | 3097-3101 | ND | 53 |
| 158-161 | 3098-3101 | ND | 51 |
| 155-162 | 3095-3102 | ND | ND |
| 156-162 | 3096-3102 | ND | ND |
| 157-162 | 3097-3102 | ND | 16 |
| 158-162 | 3098-3102 | ND | 10 |

TABLE 35
Maximum response of engineered immunoglobulins toward hFcRn
determined by SPR experiment, following procedure described in
Example 5b. Maximum response only was determined
to drive selection and speed up screening.

| | | | Maximum response |
|---|---|---|---|
| | | KD (1:1) | at 2500 nM hFcRn |
| SEQ ID NO | Ref number | (M) | (RU) |
| 1 | 2257 | ND | 177 |
| 132-141 | 2840-2851 | ND | 0 |
| 136-143 | 2841-2852 | ND | 0 |
| 134-141 | 2842-2851 | ND | 0 |
| 138-143 | 2843-2852 | ND | 0 |
| 132-145 | 2840-2853 | ND | 22 |
| 136-145 | 2841-2853 | ND | 24 |
| 134-145 | 2842-2853 | ND | 21 |
| 138-145 | 2843-2853 | ND | 28 |
| 133-140 | 3090-2844 | ND | 0 |
| 137-142 | 3092-2845 | ND | 0 |
| 135-140 | 3091-2844 | ND | 0 |
| 139-142 | 3093-2845 | ND | 0 |
| 133-144 | 3090-2846 | ND | 13 |
| 137-144 | 3092-2846 | ND | 23 |
| 135-144 | 3091-2846 | ND | 15 |
| 139-144 | 3093-2846 | ND | 22 |

TABLE 36
Affinity and maximum response of engineered immunoglobulins
toward hFcRn determined by SPR experiment, following
procedure described in Example 5b.

| | | | Maximum response |
|---|---|---|---|
| | | | at 5000nM hFcRn |
| SEQ ID NO | Ref number | KD (1:1) (M) | (RU) |
| 1 | 2257 | 3.00E−07 | 332 |
| 136-145 | 2841-2853 | 1.51E−06 | 47 |
| 133-144 | 3090-2846 | 2.58E−08 | 14 |
| 137-144 | 3092-2846 | 1.39E−07 | 34 |
| 135-144 | 3091-2846 | 1.83E−06 | 33 |
| 139-144 | 3093-2846 | 1.79E−07 | 31 |
| 137-163 | 3011-3092 | 4.87E−07 | 83 |
| 137-154 | 3092-3094 | 2.19E−07 | 97 |
| 157-159 | 3097-3099 | 1.42E−07 | 131 |
| 157-160 | 3097-3100 | 5.37E−08 | 76 |
| 157-161 | 3097-3101 | 1.63E−07 | 101 |
| 157-162 | 3097-3102 | 9.76E−08 | 90 |

### SUMMARY

The protein engineering campaign described in Examples 1 and 2 led to the generation of engineered IgG1 immunoglobulins having both alpha and gamma effector function. These engineered immunoglobulins could be constructed with or without hFcRn binding for half-life extension and the binding properties and cellular effector function of the lead candidates are shown in Table 37.

TABLE 37
Lead candidates selected through engineering campaign described in Examples 1
and 2, and description of their ability to mediate alpha and gamma effector functions.

| | | SPR data | | | | Cellular effector function | | |
|---|---|---|---|---|---|---|---|---|
| | | Binding to | Binding to | Binding to | Binding | PMN | PBMC | |
| SEQ ID NO | Ref number | FcαRI | FegRIa | FcgRIIIa | to FeRn | ADCC | ADCC | ADCP |
| 1 | 2257 | | + | + | + | | + | + |
| 3 | 2737 | + | | | | + | | + |
| 122 | 2771 | + | | | | + | | + |
| 148 | 3084 | + | + | + | | + | | + |
| 152 | 3088 | + | + | + | | + | + | + |
| 137-154 | 3092-3094 | + | | | + | + | | + |
| 157-159 | 3097-3099 | + | + | + | + | + | | + |
| 157-160 | 3097-3100 | + | + | | + | + | | + |
| 157-161 | 3097-3101 | + | + | + | + | + | + | + |
| 157-162 | 3097-3102 | + | + | + | + | + | + | + |

### Example 3: IgA2 Fc Affinity Maturation for hFcαRI

3.1 IgA2 Fc Library Design

In-silico analysis was performed on the IgA1 Fc/hFcαRI complex using the crystal structure PDB 1OWO, since IgA1 has a similar structure to IgA2, differing only in the hinge region. All residues located in proximity to hFcαRI were considered to be potentially involved in the IgA1 Fc/hFcαRI interaction and were classified into two categories: (i) residues from the “core” interface region (LCH2.15, LCH2.15.1, MCH3.105, ECH3.109, PCH3.113, LCH3.114, ACH3.115, FCH3.116, QCH3.118, using IMGT numbering for C-domain) and (ii) residues from the “shell” region surrounding the core (QCH2.94, NCH2.97, HCH2.98, RCH3.1, ECH3.3, RCH3.40, LCH3.42, SCH3.45, ECH3.45.2, using IMGT numbering for C-domain). Both groups of residues were diversified using a trinucleotide-directed mutagenesis (TRIM) based approach (Virnekäs et al, (1994) Nucleic Acids Res., 22: 5600-5607; Knappik et al., (2000) J Mol Biol., 296: 57-86) to generate two libraries L1 and L2, corresponding to the ‘shell’ region and the ‘core’ region, respectively.

A third library was generated using error prone PCR of the IgA2 Fc domain (EP library) (Gram et al., (1992) PNAS USA, 89: 3576-3580).

3.2 Library Screening by Yeast Display

All three IgA2 Fc libraries were screened using yeast display technology (Boder et al., (1997) Nature Biot., 15: 553-557). Briefly, IgA Fcs were displayed on yeast cell membranes via the α-agglutinin (Aga1/Aga2) protein heterodimer, with IgA2 Fc fused to the N-terminus of the Aga2 protein. Four rounds of sorting were performed:

During the first round of sorting, all three libraries were grown for two days at 20° C. with shaking in selective medium containing 1% raffinose and 2% galactose to induce IgA2 Fc expression on yeast cell surface. Each culture was pelleted, the supernatant removed, and the pellet washed once with PBSM (PBS (Gibco, Waltham, Mass.) with 1% BSA (bovine serum albumin) and 2 mM EDTA. After resuspension of pellets in 20 ml PBSM, 100 μl each streptavidin and anti-biotin microbeads (Miltenyi Biotec, Bergisch Gladbach, Germany) were added. Cells were incubated for 1 hr at room temperature with rotation. The beads were then removed using a MACS LS column (Miltenyi). The libraries were then pelleted and resuspended in 20 ml PBSM+50 nM biotinylated FcαRI (CD89) (SEQ ID NO: 253) and incubated for 1 hr at room temperature with rotation. The cells were then pelleted, the supernatant removed, washed once in PBSM, then resuspended in 20 ml PBSM+100 μl streptavidin microspheres (Miltenyi). The libraries were incubated for 5 min on ice with occasional shaking, then the cells were pelleted, the supernatant removed, resuspended in 20 ml PBSM, then separated on MACS LS columns (Miltenyi). The columns were washed once with 5 ml PBSM, then the bound cells were eluted with selective medium, brought to 10 ml final in selective medium, and grown at 30° C. with shaking overnight.

(ii) For the second round of sorting, the first round output from each of the three libraries was grown 24 hrs at 20° C. in selective medium containing 1% raffinose and 2% galactose to induce IgA expression. The libraries were pelleted, washed once in PBSF (PBS (Gibco)+0.1% bovine serum albumin), and resuspended in PBSF. Each library was divided in to two samples; the first sample was brought to 25 nM biotinylated FcαRI in PBSF, and the second sample was brought to 10 nM biotinylated FcαRI in PBSF. To each, rabbit anti myc-tag Dylight 488 (Rockland, Limerick, Pa.) was added ata 1:100 final dilution, and the samples were incubated for 1.5 hrs at room temperature with rotation. The samples were pelleted, washed once with PBSF, then incubated with PBSF+1:100 final streptavidin Dylight 633 (Invitrogen, Waltham, Mass.) for 5 min with rotation. The samples were then pelleted, washed once, resuspended in PBSF, filtered through a 40 μm strainer, then analyzed and sorted using flow cytometry on a FACS Aria cell sorter (Becton Dickinson Biosciences, San Jose, Calif.). For the L1 and L2 libraries, the 10 nM FcαRI sample was sorted, and for the EP library, the 25 nM FcαRI sample was sorted. In each case, the yeast showing the top 1-2% of signal were gated, collected, and grown overnight at 30° C. in selective medium.

(iii) For the third round of sorting, the cultures from the second round of sorting were inoculated in to selective medium+1% raffinose+2% galactose, and grown overnight at 20° to induce IgA expression. Cells from each of the three libraries were prepared and sorted as was done in the second round, except with the use of chicken anti myc tag FITC (Genetex, Irvine, Calif.) and Neutravidin Dylight 633 (Invitrogen), as detection reagents. Biotinylated FcαRI was used at 5 nM for the EP library, 2 nM for the L1 library, and 1 nM for the L2 library. In each case, the yeast showing the top 1-2% of signal were gated, collected, and grown overnight at 30° C. in selective medium.

(iv) The fourth round of sorting was completed on the EP and L1 libraries only. The cultures from the third round of sorting were inoculated in to selective medium+1% raffinose+2% galactose, and grown overnight at 20° to induce IgA expression. Cells from each of the libraries were prepared and sorted as was done in the second round, except with the use of mouse anti c myc Dylight 488 (Invitrogen) and Streptavidin cy5 (Invitrogen), as detection reagents. Biotinylated FcαRI was used at 2 nM for the EP library, and 1 nM for the L1 library. In each case, the yeast showing the top 1-2% of signal were gated, collected, and grown overnight at 30° C. in selective medium.

3.3 Hot Spot Identification and Translation in Full-Length Immunoglobulins

Plasmids were purified from the third (L2 library) and fourth (EP and L1 libraries) round cultures, transformed in to E. coli, plated on selective agar plates, grown overnight at 37°, and submitted to Genewiz (South Plainfield, N.J.) for Sanger sequencing (Sanger et al (1975) J Mol Biol., 94(3): 441-8; Sanger et al (1977) PNAS USA., 74(12): 5463-7). The top clones were selected based on their frequency of appearance and were used to identify mutations enhancing the IgA2/FcαRI interaction. The IgA2 residue positions are presented Table 38.

TABLE 38
Mutations determined by yeast display for enhancing IgA2
affinity towards hFcαRI.

| IgA2 sequence position (IMGT | Initial residue | |
|---|---|---|
| numbering for C-domain) | in IgA2 | Mutation |
| CH2.10 | A | S |
| CH2.89 | L | I |
| CH2.91 | G | V |
| CH2.94 | Q | E |
| CH2.97 | N | Y, H |
| CH2.99 | G | W |
| CH3.105 | M | Y |
| CH3.109 | E | D |
| CH3.118 | Q | Y |
| CH3.124 | L | F |

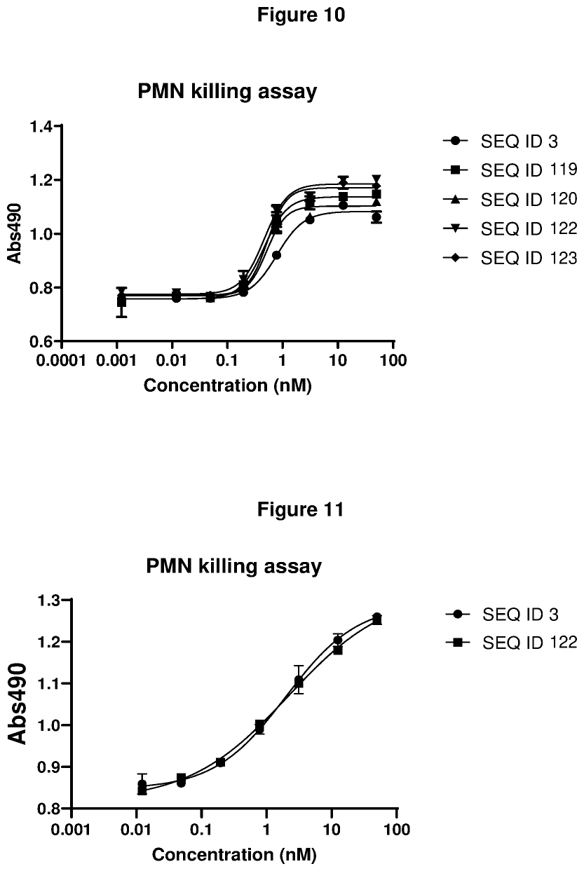

Identified mutations were incorporated into the full-length IgA2 immunoglobulin having SEQ ID NO: 3 (FIG. 7a), as single point mutation or in combination, and expressed transiently in HEK293 cells (as described in Example 4). The same mutations were also incorporated into IgG1 isotype immunoglobulins having SEQ ID NOs: 122 and 148 and containing an engineered IgG Fc that was capable of binding to hFcαRI (FIG. 7b and FIG. 7c, respectively). The tested mutation sets are presented in Table 39 (based on SEQ ID NO: 3), Table 41 (based on SEQ ID NO: 122) and Table 43 (based on SEQ ID NO: 148).

The Fc variants were purified and assessed using surface plasmon resonance (SPR), measured against hFcαRI, to evaluate the effect of specific mutations on immunoglobulin affinity to hFcαRI. Interestingly, all mutations had a limited effect or no real effect on expression yield and aggregation propensity of the respective immunoglobulin. SPR data and aggregation content after capture are shown in Table 40, Table 42 and Table 44.

Finally, lead candidates were selected based on SPR and aggregation data and SPR experiments were repeated using a broader range of hFcαRI concentrations. The use of the adapted concentration range enabled a more precise measurement of the interaction between engineered immunoglobulin and hFcαRI. Results are shown in Table 45, Table 46 and Table 47.

TABLE 39
Tested mutation sets, based on parental IgA2 immunoglobulin SEQ ID 3.

| SEQ | Ref | |
|---|---|---|
| ID NO | number | Mutation set |
| 3 | 2737 | — |
| 182 | 3240 | Q_CH2.94_E |
| 183 | 3241 | N_CH2.97_Y |
| 184 | 3242 | S_CH3.45_D |
| 185 | 3243 | M_CH3.105_Y |
| 186 | 3244 | Q_CH3.118_Y |
| 187 | 3245 | Q_CH2.94_E, N_CH2.97_Y |
| 225 | 3309 | Q_CH2.94_E, S_CH3.45_D |
| 226 | 3310 | Q_CH2.94_E, M_CH3.105_Y |
| 227 | 3311 | N_CH2.97_Y, S_CH3.45_D |
| 228 | 3312 | N_CH2.97_Y, M_CH3.105_Y |
| 229 | 3313 | S_CH3.45_D, M_CH3.105_Y |
| 200 | 3258 | M_CH3.105_Y, Q_CH3.118_Y |
| 230 | 3314 | Q_CH2.94_E, N_CH2.97_Y, M_CH3.105_Y |
| 231 | 3315 | N_CH2.97_Y, S_CH3.45_D, M_CH3.105_Y |
| 232 | 3316 | Q_CH2.94_E, S_CH3.45_D, M_CH3.105_Y |
| 201 | 3259 | M_CH3.105_Y, Q_CH3.118_Y, S_CH3.45_D |
| 202 | 3260 | Q_CH2.94_E, N_CH2.97_Y, S_CH3.45_D |
| 233 | 3317 | Q_CH2.94_E, N_CH2.97_Y, S_CH3.45_D, M_CH3.105_Y |
| 203 | 3261 | Q_CH2.94_E, N_CH2.97_Y, M_CH3.105_Y, Q_CH3.118_Y |
| 204 | 3262 | Q_CH2.94_E, N_CH2.97_Y, S_CH3.45_D, M_CH3.105_Y, Q_CH3.118_Y |
| 215 | 3273 | A_CH2.10_S |
| 216 | 3274 | L_CH2.89_I |
| 217 | 3275 | G_CH2.91_V |
| 222 | 3280 | N_CH2.97_H |
| 218 | 3276 | G_CH2.99_W |
| 219 | 3277 | E_CH3.109_D |
| 220 | 3278 | L_CH3.124_F |
| 221 | 3279 | L_CH2.89_I, G_CH2.91_V, Q_CH2.94_E, N_CH2.97_Y, G_CH2.99_W |

TABLE 40
Biophysical characterization of the Fc variants based on parental
IgA2 SEQ ID No: 3.

| | | | Maximum response¹ | |
|---|---|---|---|---|
| SEQ ID | Ref | KD (1:1) | at 1000 nM | Aggregation² |
| NO | number | (M)¹ | hFCaRI (RU) | (%) |
| 3 | 2737 | 5.20E−07 | 168 | 11.5 |
| 182 | 3240 | 1.26E−07 | 253 | 12.40 |
| 183 | 3241 | 8.69E−08 | 235 | 10.60 |
| 184 | 3242 | 6.67E−08 | 261 | 9.93 |
| 185 | 3243 | 2.63E−08 | 278 | 11.30 |
| 186 | 3244 | 1.46E−07 | 194 | 8.60 |
| 187 | 3245 | 6.73E−08 | 250 | 14.00 |
| 225 | 3309 | 5.20E−08 | 242 | 8.30 |
| 226 | 3310 | 1.70E−08 | 213 | 8.10 |
| 227 | 3311 | 1.94E−08 | 246 | 9.60 |
| 228 | 3312 | 6.77E−09 | 251 | 7.50 |
| 229 | 3313 | 9.81E−09 | 194 | 8.90 |
| 200 | 3258 | 1.30E−08 | 278 | 10.20 |
| 230 | 3314 | 5.65E−09 | 247 | 8.40 |
| 231 | 3315 | 3.96E−09 | 223 | 9.90 |
| 232 | 3316 | 9.80E−09 | 226 | 7.40 |
| 201 | 3259 | 7.43E−09 | 289 | 10.40 |
| 202 | 3260 | 4.10E−08 | 306 | 13.10 |
| 233 | 3317 | 3.81E−09 | 244 | 11.90 |
| 203 | 3261 | 5.66E−09 | 289 | 13.60 |
| 204 | 3262 | 5.99E−09 | 294 | 16.70 |
| 215 | 3273 | 1.09E−07 | 244 | 13.50 |
| 216 | 3274 | 1.24E−07 | 189 | 11.00 |
| 217 | 3275 | 1.38E−07 | 247 | 10.30 |
| 222 | 3280 | 1.00E−07 | 188 | nd |
| 218 | 3276 | 1.43E−07 | 225 | 9.90 |
| 219 | 3277 | 9.20E−08 | 220 | 11.40 |
| 220 | 3278 | 1.60E−07 | 231 | 12.80 |
| 221 | 3279 | nd | nd | nd |

¹ Affinity and maximum response of engineered immunoglobulins towards hFcαRI were determined by SPR experiments, following the procedure described in Example 5b.
² Aggregation propensity was measured following the procedure described in Example 4.
nd: not determined

TABLE 41
Tested mutation sets, based on parental

| | | IgG1 engineered immunoglobulin SEQ ID 122. |
|---|---|---|
| SEQ | Ref | |
| ID NO | number | Mutation set |
| 122 | 2771 | — |
| 188 | 3246 | Q_CH2.94_E |
| 189 | 3247 | L_CH2.97_Y |
| 190 | 3248 | S_CH3.45_D |
| 191 | 3249 | M_CH3.105_Y |
| 192 | 3250 | Q_CH3.118_Y |
| 193 | 3251 | Q_CH2.94_E, L_CH2.97_Y |
| 234 | 3318 | Q_CH2.94_E, S_CH3.45_D |
| 235 | 3319 | Q_CH2.94_E, M_CH3.105_Y |
| 236 | 3320 | L_CH2.97_Y, S_CH3.45_D |
| 237 | 3321 | L_CH2.97_Y, M_CH3.105_Y |
| 238 | 3322 | S_CH3.45_D, M_CH3.105_Y |
| 205 | 3263 | M_CH3.105_Y, Q_CH3.118_Y |
| 239 | 3323 | Q_CH2.94_E, L_CH2.97_Y, M_CH3.105_Y |
| 240 | 3324 | L_CH2.97_Y, S_CH3.45_D, M_CH3.105_Y |
| 241 | 3325 | Q_CH2.94_E, S_CH3.45_D, M_CH3.105_Y |
| 206 | 3264 | M_CH3.105_Y, Q_CH3.118_Y, S_CH3.45_D |
| 207 | 3265 | Q_CH2.94_E, L_CH2.97_Y, S_CH3.45_D |
| 242 | 3326 | Q_CH2.94_E, L_CH2.97_Y, S_CH3.45_D, M_CH3.105_Y |
| 208 | 3266 | Q_CH2.94_E, L_CH2.97_Y, M_CH3.105_Y, Q_CH3.118_Y |
| 209 | 3267 | Q_CH2.94_E, L_CH2.97_Y, S_CH3.45_D, M_CH3.105_Y, Q_CH3.118_Y |
| 223 | 3281 | L_CH2.89_I, V_CH2.91, Q_CH2.94_E, L_CH2.97_Y, G_CH2.99_W |

TABLE 42
Biophysical characterization of Fc variants based on parental engineered
immunoglobulin SEQ ID NO: 122

| | | | Maximum response¹ | |
|---|---|---|---|---|
| SEQ ID | Ref | KD (1:1) | at 1000 nM hFcαRI | |
| NO | number | (M)1 | (RU) | Aggregation² (%) |
| 3 | 2737 | 5.20E−07 | 168 | 11.5 |
| 122 | 2771 | 2.70E−07 | 197 | 9.20 |
| 188 | 3246 | 8.72E−08 | 267 | 14.80 |
| 189 | 3247 | 1.45E−08 | 372 | 12.00 |
| 190 | 3248 | 5.75E−08 | 344 | 9.80 |
| 191 | 3249 | 2.06E−08 | 339 | 6.80 |
| 192 | 3250 | 1.04E−07 | 240 | 10.00 |
| 193 | 3251 | 1.22E−08 | 344 | 9.00 |
| 234 | 3318 | 4.38E−08 | 342 | 10.10 |
| 235 | 3319 | 1.51E−08 | 352 | 9.40 |
| 236 | 3320 | 4.81E−09 | 326 | 7.90 |
| 237 | 3321 | 1.64E−09 | 289 | 8.30 |
| 238 | 3322 | 1.00E−08 | 327 | 8.70 |
| 205 | 3263 | 9.56E−09 | 322 | 12.70 |
| 239 | 3323 | 9.56E−10 | 319 | 9.00 |
| 240 | 3324 | 7.81E−10 | 304 | 7.50 |
| 241 | 3325 | 8.47E−09 | 325 | 7.80 |
| 206 | 3264 | 3.81E−09 | 289 | 11.40 |
| 207 | 3265 | 5.02E−09 | 278 | 13.90 |
| 242 | 3326 | 5.84E−10 | 307 | 8.50 |
| 208 | 3266 | 5.57E−10 | 333 | 12.05 |
| 209 | 3267 | 3.84E−10 | 305 | 13.70 |
| 223 | 3281 | 1.87E−08 | 228 | nd |

¹ Affinity and maximum response of engineered immunoglobulins toward hFcαRI were determined by SPR experiment, following the procedure described in Example 5b.
² Aggregation propensity was measured following the procedure described in Example 4.
nd: not determined

TABLE 43
Tested mutation sets, based on parental
IgG1 engineered immunoglobulin SEQ ID 148

| SEQ | Ref | |
|---|---|---|
| ID NO | number | Mutation set |
| 148 | 3084 | — |
| 194 | 3252 | Q_CH2.94_E |
| 195 | 3253 | L_CH2.97_Y |
| 196 | 3254 | S_CH3.45_D |
| 197 | 3255 | M_CH3.105_Y |
| 198 | 3256 | Q_CH3.118_Y |
| 199 | 3257 | Q_CH2.94_E, L_CH2.97_Y |
| 243 | 3327 | Q_CH2.94_E, S_CH3.45_D |
| 244 | 3328 | Q_CH2.94_E, M_CH3.105_Y |
| 245 | 3329 | L_CH2.97_Y, S_CH3.45_D |
| 246 | 3330 | L_CH2.97_Y, M_CH3.105_Y |
| 247 | 3331 | S_CH3.45_D, M_CH3.105_Y |
| 210 | 3268 | M_CH3.105_Y, Q_CH3.118_Y |
| 248 | 3332 | Q_CH2.94_E, L_CH2.97_Y, M_CH3.105_Y |
| 249 | 3333 | L_CH2.97_Y, S_CH3.45_D, M_CH3.105_Y |
| 250 | 3334 | Q_CH2.94_E, S_CH3.45_D, M_CH3.105_Y |
| 211 | 3269 | M_CH3.105_Y, Q_CH3.118_Y, S_CH3.45_D |
| 212 | 3270 | Q_CH2.94_E, L_CH2.97_Y, S_CH3.45_D |
| 251 | 3335 | Q_CH2.94_E, L_CH2.97_Y, S_CH3.45_D, M_CH3.105_Y |
| 213 | 3271 | Q_CH2.94_E, L_CH2.97_Y, M_CH3.105_Y, Q_CH3.118_Y |
| 214 | 3272 | Q_CH2.94_E, L_CH2.97_Y, S_CH3.45_D, M_CH3.105_Y, Q_CH3.118_Y |
| 224 | 3282 | L_CH2.89_I, V_CH2.91, Q_CH2.94_E, L_CH2.97_Y, G_CH2.99_W |

TABLE 44
Biophysical characterization of Fc variants based on parental engineered
immunoglobulin SEQ ID NO: 148

| | | | Maximum response¹ | |
|---|---|---|---|---|
| SEQ ID | Ref | KD (1:1) | at1000nM hFcαRI | |
| NO | number | (M)¹ | (RU) | Aggregation² (%) |
| 3 | 2737 | 5.20E−07 | 168 | 11.5 |
| 148 | 3084 | 5.09E−07 | 146 | 10.30 |
| 194 | 3252 | 1.15E−07 | 212 | 9.20 |
| 195 | 3253 | 3.52E−08 | 359 | 11.30 |
| 196 | 3254 | 1.32E−07 | 276 | 12.00 |
| 197 | 3255 | 5.38E−08 | 300 | 13.90 |
| 198 | 3256 | 1.33E−07 | 194 | 11.70 |
| 199 | 3257 | 2.77E−08 | 376 | 9.80 |
| 243 | 3327 | 7.53E−08 | 303 | 9.30 |
| 244 | 3328 | 3.88E−08 | 342 | 8.70 |
| 245 | 3329 | 1.12E−08 | 369 | 12.70 |
| 246 | 3330 | 4.32E−09 | 275 | 13.80 |
| 247 | 3331 | 2.80E−08 | 342 | 14.60 |
| 210 | 3268 | 2.29E−08 | 341 | 13.60 |
| 248 | 3332 | 2.39E−09 | 294 | 11.70 |
| 249 | 3333 | 1.96E−09 | 238 | 9.10 |
| 250 | 3334 | 2.23E−08 | 353 | 7.50 |
| 211 | 3269 | 1.05E−08 | 353 | 12.00 |
| 212 | 3270 | 1.04E−08 | 365 | 11.70 |
| 251 | 3335 | 1.27E−09 | 282 | 8.30 |
| 213 | 3271 | 1.13E−09 | 371 | 15.20 |
| 214 | 3272 | 4.86E−10 | 371 | 14.70 |
| 224 | 3282 | 4.71E−08 | 235 | nd |

¹ Affinity and maximum response of engineered immunoglobulins toward hFcαRI were determined by SPR experiment, following the procedure described in Example 5b.
² Aggregation propensity was measured following the procedure described in Example 4.
nd: not determined

TABLE 45
Affinity and maximum response of Fc variants, based on
parental IgA2 (SEQ ID NO: 3), towards hFcαRI as
determined by SPR experiment described in Example 5b.

| SEQ ID | Ref | KD (1:1) | Max response at 31.25 |
|---|---|---|---|
| NO | number | (M) | nM hFcαRI (RU) |
| 185 | 3243 | 8.45E−09 | 157 |
| 200 | 3258 | 4.02E−09 | 179 |
| 231 | 3315 | 1.45E−09 | 190 |
| 202 | 3260 | 8.34E−09 | 185 |
| 233 | 3317 | 1.06E−09 | 201 |
| 204 | 3262 | 1.13E−09 | 191 |

TABLE 46
Affinity and maximum response of Fc variants, based on parental
IgG1 engineered immunoglobulin (SEQ ID NO: 122), towards
hFcαRI as determined by SPR experiment described in Example 5b.

| SEQ ID | Ref | KD (1:1) | Max response at 31.25 |
|---|---|---|---|
| NO | number | (M) | nM hFcαRI (RU) |
| 189 | 3247 | 9.34E−09 | 259 |
| 236 | 3320 | 2.56E−09 | 252 |
| 238 | 3322 | 5.40E−09 | 220 |
| 240 | 3324 | 3.24E−10 | 249 |
| 242 | 3326 | 2.95E−10 | 248 |
| 209 | 3267 | 2.19E−10 | 268 |

TABLE 47
Affinity and maximum response of Fc variants, based on parental IgG1
engineered immunoglobulin (SEQ ID NO: 148), towards hFcαRI as
determined by SPR experiment described in Example 5b.

| SEQ ID | Ref | KD (1:1) | Max response at 31.25 |
|---|---|---|---|
| NO | number | (M) | nM hFcαRI (RU) |
| 195 | 3253 | 2.25E−08 | 187 |
| 197 | 3255 | 2.92E−08 | 139 |
| 199 | 3257 | 1.49E−08 | 239 |
| 245 | 3329 | 9.11E−09 | 229 |
| 248 | 3332 | 1.20E−09 | 237 |
| 249 | 3333 | 8.80E−10 | 189 |
| 212 | 3270 | 5.17E−09 | 290 |
| 251 | 3335 | 6.51E−10 | 240 |
| 213 | 3271 | 3.82E−10 | 322 |
| 214 | 3272 | 1.96E−10 | 313 |

3.4 Translation of Affinity Maturation to Enhancement of Alpha Effector Function in In Vitro Assays

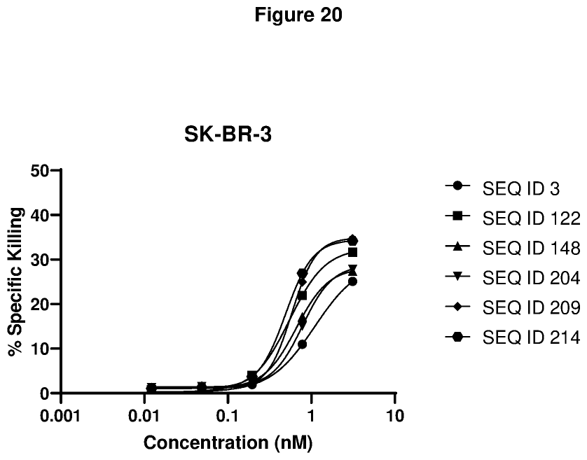

Lead candidates having a homodimeric Fc comprised within SEQ ID NOs: 204, 209 and 214 were selected for their improved binding capacities towards hFcαRI. They were next tested in a PMN killing assay following the procedure described in Example 6. Potency (EC₅₀; concentration of the immunoglobulin required to produce 50% of its maximal effect) and efficacy (Emax; maximum effect expected of the immunoglobulin) results are shown in FIG. 20 to FIG. 24.

All tested candidates were active and were shown to have an improved efficacy compared to the parental IgA2 in a SK-BR-3 PMN killing assay (FIG. 20).

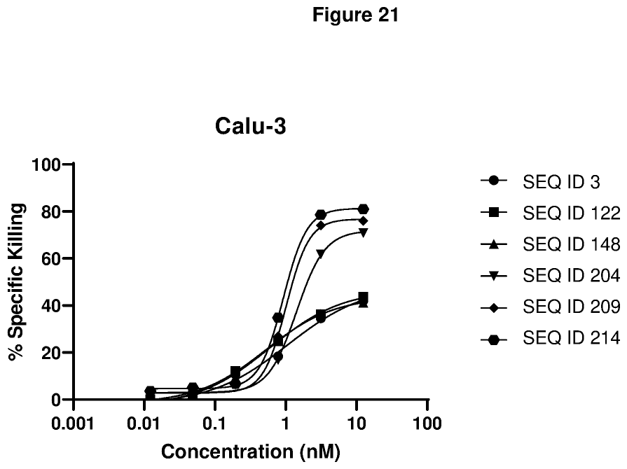

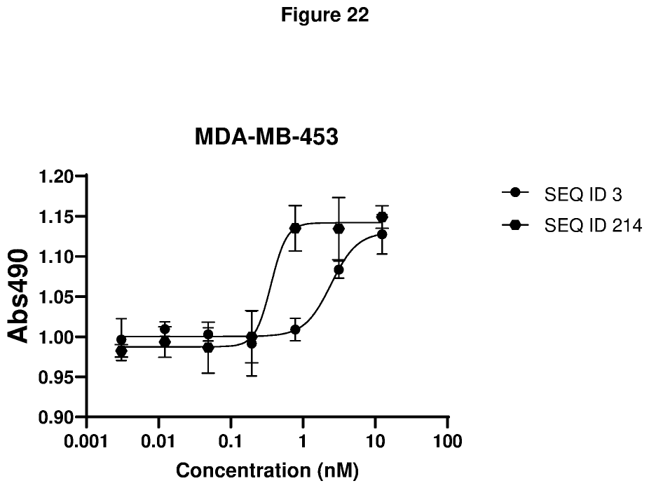

In addition, a substantial improvement in efficacy was shown in a PMN killing assay using Calu-3 cells (FIG. 21) where HER2 receptor density is known to be lower than SK-BR-3 cells (described in Example 6). Moreover, a substantial improvement in potency was shown for the variant having SEQ ID NO: 214 in a PMN killing assay on MDA-MB-453 cells, also known to express a lower level of HER2 receptors (FIG. 22).

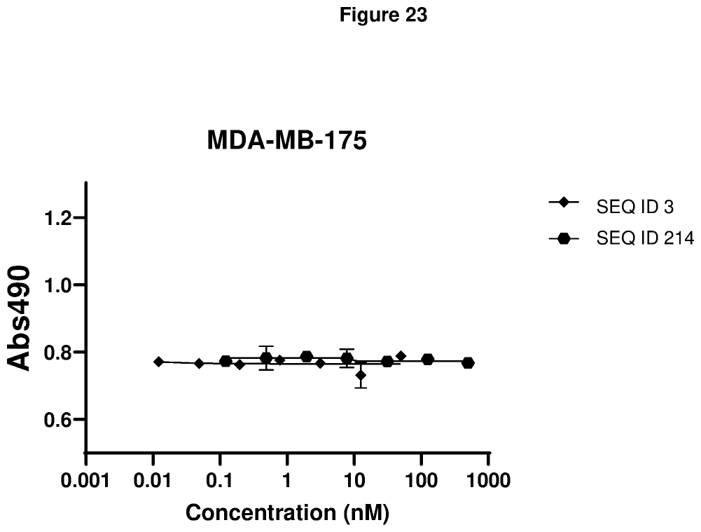

Finally, the affinity matured variant having SEQ ID NO: 214 was tested in a PMN killing assay using MDA-MB-175 cells, known as the lowest HER2 expressing cell line, and showed no killing, even at very high concentrations (FIG. 23). This observation highlights the safe profile of the tested candidates.

The mutation set of Q_CH2.94_E, L_CH2.97_Y and S_CH3.45_D was applied to the ‘hole’ arm of a heterodimeric Fc comprised within SEQ ID NO: 157 resulting in SEQ ID NO: 252. Heterodimeric Fc candidates comprising SEQ ID NO: 252-159 and SEQ ID NO: 252-161 (shown schematically in FIG. 7d) were tested against the lead Fc heterodimers from Example 2 having SEQ ID NO: 157-159 and SEQ ID NO: 157-161. The engineered IgG1 immunoglobulins with Fc domains comprised within SEQ ID NO: 252-159 and SEQ ID NO: 252-161 were shown to have better killing properties on SK-BR-3 cells in PMN killing assays, compared to their parental immunoglobulins and IgA2 (FIGS. 24a and 24c) but showed no effect in a PBMC killing assay to mediate gamma response (FIGS. 24b and 24d). Introducing the mutations derived from affinity maturation into the Fc domain recognizing hFcαRI improves alpha receptor binding but does not affect gamma receptor binding.

### Example 4: Expression and Purification of Engineered Proteins

Nucleic acid sequences coding for heavy and light chains were synthesized at Geneart (LifeTechnologies) and cloned into a mammalian expression vector using restriction enzyme-ligation based cloning techniques. The resulting plasmids were co-transfected into HEK293T cells. In brief, for transient expression of immunoglobulins (IgG, IgA and engineered immunoglobulins), equal quantities of light chain and each engineered heavy chain vectors were co-transfected into suspension-adapted HEK293T cells using Polyethylenimine ((PEI) Ref. cat#24765 Polysciences, Inc.). Typically, 100 ml of cells in suspension at a density of 1-2 Mio cells per ml was transfected with DNA containing 50 μg of expression vector encoding the engineered heavy chain and 50 μg expression vectors encoding the light chain. The recombinant expression vectors were then introduced into the host cells and the construct produced by further culturing of the cells for a period of 7 days to allow for secretion into the culture medium (HEK, serum-fee medium) supplemented with 0.1% pluronic acid, 4 mM glutamine, and 0.25 μg/ml antibiotic.

The produced constructs were then purified from cell-free supernatant using immuno-affinity chromatography. Anti-Kappa LC resin (KappaSelect, GE Healthcare Life Sciences), equilibrated with PBS buffer pH 7.4 was incubated with filtered conditioned media using liquid chromatography system (Aekta pure chromatography system, GE Healthcare Life Sciences). The resin was washed with PBS pH 7.4 before the constructs were eluted with elution buffer (50 mM citrate, 90 mM NaCl, pH 2.7).

After capture, eluted proteins were pH neutralized using 1M TRIS pH 10.0 solution and polished using size exclusion chromatography technique (HiPrep Superdex 200 16/60, GE Healthcare Life Sciences). Purified proteins were finally formulated in PBS buffer pH 7.4.

Aggregation propensity was measured after capture and pH neutralization step using analytical size exclusion chromatography technique (Superdex 200 Increase 3.2/300 GL, GE Healthcare Life Sciences)

### Example 5: SPR Measurement Against Human Fc Receptors

A direct binding assay was performed to characterize the binding of the engineered immunoglobulins (in full antibody format) against hFcαR, hFcγRIa, hFcγRIIIa or hFcRn.

Kinetic binding affinity constants (KD) were measured on a BIAcore® T200 instrument (GE Healthcare, Glattbrugg, Switzerland) at room temperature, with proteins diluted in running buffer 10 mM NaP, 150 mM NaCl, 0.05% Tween 20, pH7.6. A CM5 sensor chip (Sensor Chip SA, GE Healthcare Life Sciences) immobilized with an anti-kappa light chain scFv by amine coupling (Example 5a) or a streptavidin sensor chip (Sensor Chip SA, GE Healthcare Life Sciences) immobilized with a biotinylated anti-kappa light chain scFv (Example 5b) were used to capture engineered immunoglobulins, and recombinant human hFcαR, recombinant human hFcγRIa, recombinant human hFcγRIIIa or recombinant human hFcRn was used as the analyte, as appropriate.

To serve as a reference, one flow cell did not capture any immunoglobulin. Binding data were acquired by subsequent injection of analyte dilution series on the reference and measuring flow cells. Zero concentration samples (running buffer only) were included to allow double referencing during data evaluation. For data evaluation, doubled referenced sensorgrams were analyzed by applying a 1:1 binding model analysis to generate the equilibrium dissociation constant (KD). In addition, the maximum response reached during the experiment was monitored. Maximum response describes the binding capacity of the surface in terms of the response at saturation.

### Example 6: Antibody-Dependent Cell Cytotoxicity (ADCC) Assay

Blood samples were obtained from healthy donors, collected from freshly drawn peripheral blood, according to the Swiss human research act (Basel tissue donor program—Prevomed). After lysis of red blood cells with ACK lysis buffer, Polymorphonuclear cells (PMN) or Peripheral Blood Mononuclear cells (PBMC) were isolated by ficoll-paque gradient. PMN were used to characterize alpha effector function of engineered immunoglobulins, whereas PBMC were used to characterize gamma effector function of the engineered immunoglobulins.

Effector cells (freshly isolated PMN or PBMC cells) were added to HER2 expressing target cells (SK-BR-3, Calu-3, MDA-MB-453 or MDA-MB-175 cells, purchased at the American Type Culture Collection, Rockville Md.) at an effector to target ratio of 20:1. SK-BR-3 is a breast cancer cell line overexpressing HER2. Calu-3 and MDA-MB-453 are lung and breast cancer cell lines respectively, overexpressing HER2 at a lower level compared to SK-BR-3 (Cheung et al., 2019). MDA-MD-175 is a breast cancer cell line expressing the lowest amount of HER2 (Crocker et al., 2005).

The immunoglobulin construct was added at the indicated concentration and the combination was gently mixed and then centrifuged at 260×g for 4 minutes without break to encourage co-localization of target and effector cells. The assay was then incubated for 18 hours at 37° C. in 5% CO₂ in a standard tissue culture incubator. After 18 hours, supernatant was used for LDH release measurements using Cytotox96 reagent (Promega) according to the manufacturer instructions. Absorbance at 490 nm was read on a Biotek Synergy HT plate reader. Data were graphed and analyzed using GraphPad Prism 6.0.

### Example 7: Antibody-Dependent Cell Phagocytosis (ADCP) Assay

Monocytes were freshly isolated from healthy human blood and differentiated for 6 days using human M-CSF. Celltrace violet-labeled monocytes-derived macrophages (effector cells) were added to CFSE-labeled HER2 expressing target cells (SK-BR-3 overexpressing cells) at an effector to target ratio of 8:1. Immunoglobulin was added at the indicated concentration, and the combination was gently mixed. The assay was then incubated for 2 hours at 37° C. in 5% CO₂ in a standard tissue culture incubator. Phagocytosis was quantified by analyzing the double positive population using BD science FACS Fortessa flow cytometer. Data were graphed and analyzed using GraphPad Prism 6.0.

### Example 8: Thermal Stability Assessment by Differential Scanning Calorimetry (DSC)

The thermal stability of engineered immunoglobulins and their parental IgG1 and IgA2 were compared using calorimetric measurements, as described below.

calorimetric measurements were carried out on a differential scanning micro calorimeter (Nano DSC, TA Instrument). The cell volume was 0.3 ml and the heating rate was 1° C./min. All proteins were used at a concentration of 1 mg/ml in PBS (pH 7.4). The molar heat capacity of each protein was estimated by comparison with duplicate samples containing identical buffer from which the protein had been omitted. The partial molar heat capacities and melting curves were analyzed using standard procedure. Thermograms were baseline corrected and concentration normalized.

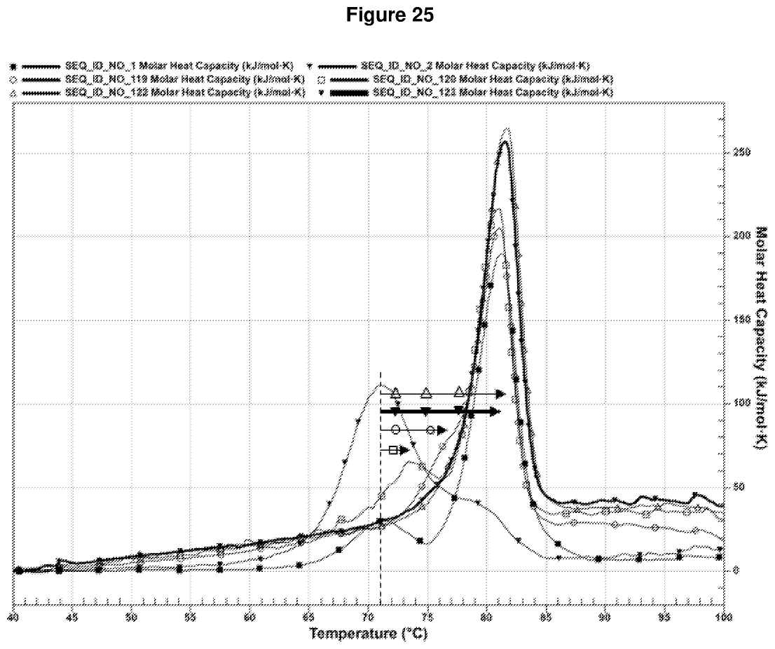

FIG. 25 shows overall improvement of thermal stability of the engineered immunoglobulins compare to both parental IgA2 and wild-type IgG1 demonstrating that immunoglobulin engineering resulted in a stable molecule with improved thermal stability over parental immunoglobulins.

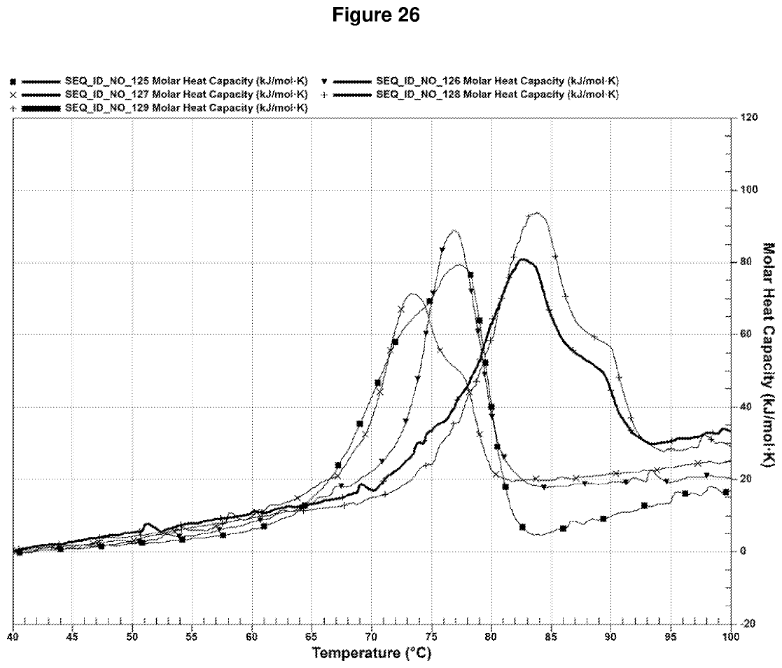

Corresponding recombinant Fcs were generated and their thermal stability compared using same method (FIG. 26), independently of the Fab. Melting temperature (TM) of individual CH2 and CH3 domains could be determined (Table 48), showing how engineered Fcs are all more stable than both parental IgA2 and IgG1. Ranking of Fcs and engineered immunoglobulins regarding their overall thermostability stay the same but it is interesting to see that Fab fusion can affect the Fc thermostability.

TABLE 48
Melting temperature (TM) of individual CH2 and CH3 domains measured on Fc
constructs, free of Fab fragment. Variations of TM compared to parental
IgA2 Fc are shown in parenthesis.

| SEQ ID NO | CH2 TM (° C.) | CH3 TM (° C.) |
|---|---|---|
| SEQ ID NO: 125-IgA_Fc | 73 | 77 |
| SEQ ID NO: 126-2768_Fc | 77 (+4) | 77 |
| SEQ ID NO: 127-2729_Fc | 73 | 77 |
| SEQ ID NO: 128-2771_Fc | 84 (+11) | 89 (+12) |
| SEQ ID NO: 129-2772_Fc | 83 (+10) | 89 (+12) |

### Example 9: Improvement of Engineered Immunoglobulins Pharmacokinetic (PK) Properties Compare to IgA

9.1 Engineered Immunoglobulin Material Production

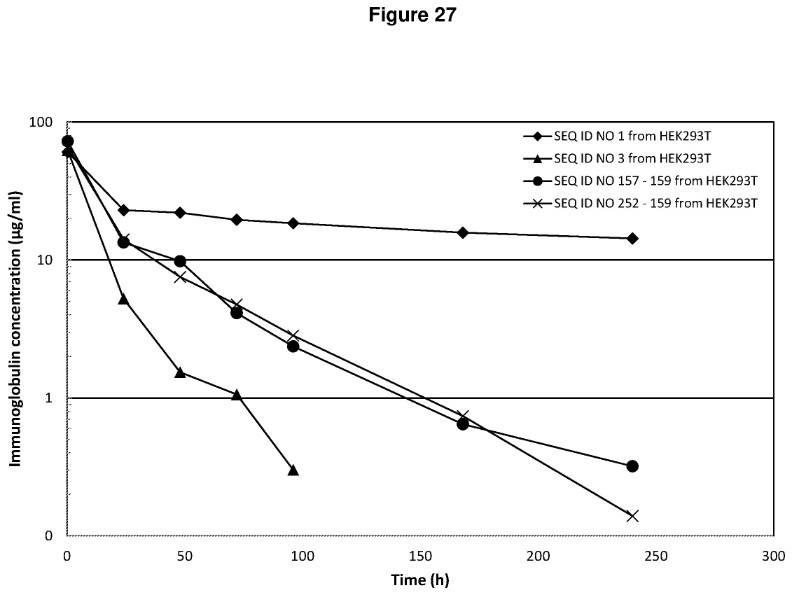

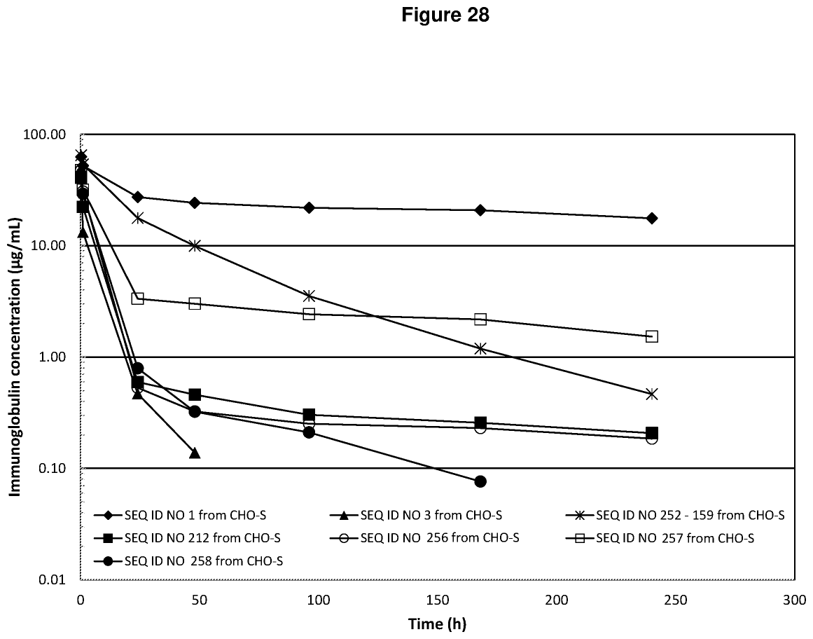

Nucleic acids coding for anti-HER2 engineered immunoglobulin heavy chain variants SEQ ID NOs: 1, 3, 157, 159, 212, 252, 256, 257, 258 were synthesized at Geneart (LifeTechnologies) and cloned into a mammalian expression vector using restriction enzyme-ligation based cloning techniques. Selected N-glycosylation sites were removed by substitution of concerned Asp residues by Ala. The resulting plasmids coding for the heavy chain were co-transfected with the plasmid coding for light chain (SEQ ID NO: 124) into a mammalian expression system. For the HEK293T expression cell line, expression was performed according to the procedure described Example 4. For the CHO-S expression cell line (Thermo), the following procedure was used. In brief, for transient protein expression, the expression vector was transfected into suspension-adapted CHO-S cells using the ExpifectamineCHO transfecting agent (Thermo). Typically, 400 ml of cells in suspension at a density of 6 Mio cells per ml were transfected with DNA containing 400 μg of expression vector encoding the engineered protein. The recombinant expression vector was then introduced into the host cells for further secretion for seven days in culture medium (ExpiCHO expression media, supplemented with ExpiCHO feed and enhancer reagent (Thermo)). The constructs produced were then purified from cell-free supernatant according to the procedure described Example 4. The generated material is described in Tables 49 and 50 and measured immunoglobulins concentrations in serum were plotted in function of time and presented in FIGS. 27 and 28.

TABLE 49
Description of immunoglobulins produced
in HEK293T mammalian system

| | Description, with numbering is |
|---|---|
| SEQ | according to IMGT |
| ID | numbering for C-domain |
| 1 | IgG1 |
| 3 | IgG2 |
| 157- | Heterodimeric engineered IgG1 |
| 159 | recruits CD89 via first half-Fc, |
| | recruits FcγRs and FcRn |
| | via second half-Fc |
| 252- | Heterodimeric engineered IgG1 |
| 159 | recruits CD89 via first |
| | half Fc (affinity matured), |
| | recruits FcγRs and FcRn |
| | via second half-Fc |

TABLE 50
Description of immunoglobulins produced
in CHO mammalian system

| | Description, with numbering |
|---|---|
| SEQ | is according to IMGT |
| ID | numbering for C-domain |
| 1 | IgG1 |
| 3 | IgG2 |
| 252- | Heterodimer engineered IgG1. |
| 159 | recruits CD89 via first |
| | half Fc (affinity matured), |
| | recruits FcγRs and FcRn |
| | via second half-Fc. |
| 212 | Homodimer engineered IgG1 |
| 256 | Homodimer engineered IgG1 |
| | N_CH2.20_A |
| 257 | Homodimer engineered IgG1 |
| | N_CH2.84.4_A |
| 258 | Homodimer engineered IgG1 |
| | N_CH2.20_A |
| | N_CH2.84.4_A |

In line with construct design and previous SPR measurements, engineered immunoglobulins with sequences SEQ ID NOs:157-159 and SEQ ID NOs: 252-159 bound to CD89 while retaining binding to FcRn (as described Example 5) and show improved PK properties and an improved half-life compared to IgA immunoglobulin, as shown in FIG. 27.

Moreover, as shown in FIG. 27, the affinity matured variant with sequence SEQ ID NOs: 252-159 exhibits an identical PK profile as the parental immunoglobulin with sequence SEQ ID NOs:157-159. This indicates that affinity maturation towards CD89 does impair the PK properties of the engineered immunoglobulins.

Data presented in FIG. 28 describe how the N-glycosylation pattern affects immunoglobulin PK. As shown, the PK properties of the engineered immunoglobulins were improved compared to IgA and improved when the individual N-glycosylation site CH2.84.4 was removed (SEQ ID NO: 257).

9.2 Mouse Studies

Male CD1 mice were obtained from Charles River laboratories. Following arrival, all mice were maintained in a pathogen-free animal facility under a standard 12 h light/12 h dark cycle at 21° C. room temperature with access to food and water ad libitum. All mice received a single intravenous (IV) injection of IgG or IgA or engineered immunoglobulin (3 mg/kg) produced and purified as described above. Each compound was injected into three mice. Blood samples were collected into serum separator tubes via saphenous vein at various times post injection. The blood was allowed to clot at ambient temperature for at least 20 min. Clotted samples were maintained at room temperature until centrifuged, commencing within 1 h of the collection time. Each sample was centrifuged at a relative centrifugal force of 1500-2000×g for 5 min at 2-8° C. The serum was separated from the blood sample within 20 min after centrifugation and transferred into labelled 2.0-mL polypropylene, conical-bottom microcentrifuge tubes. Only animals that appeared to be healthy and that were free of obvious abnormalities were used for the study. All animal work performed was reviewed and approved by Novartis' Institutional Animal Care and Use Committee.

9.3 Immunoglobulin ELISA for Pharmacokinetic Studies

Immunoglobulin levels were measured by sequential sandwich ELISA. For IgA dosing, wells of Nunc Maxisorp microtiter plates were coated overnight at 4° C. with goat anti-human IgA (Southern Biotech, Cat #2053-01). For IgG and engineered immunoglobulins dosing, wells of Roche StreptaWell microtiter plates were coated 1 h at room temperature with biotinylated SB goat anti-human IgG (Southern Biotech, Cat #2049-08). After 1 h incubation with blocking buffer (PBS, 0.5% bovine serum albumin (BSA)), samples diluted in same blocking buffer were added to the blocked plates and incubated for 2 h at room temperature. After incubation, horseradish peroxidase-conjugated goat anti-human IgA (SouthernBiotech, Cat #2053-05) or horseradish peroxidase-conjugated goat anti-human IgG (SouthernBiotech, Cat #2049-05) were added and incubated for 1 h at room temperature. The plates were then incubated with substrate solution (BMBlue POD Substrate TMB, Roche, Cat #11484281001), and the reaction was stopped with 0.5M sulfuric acid. Absorbance was measured at 450 nm with a reduction at 650 nm using a plate reader. Between steps, plates were washed 3 times with washing buffer (0.05% Tween-20 in PBS).

Sequence Information

Table 51 lists the amino acid sequences (SEQ ID NOs) of the full length heavy chains comprising the variant Fc regions as described in the examples as well as the light chain used to generate complete antibodies. The engineered IgG1 immunoglobulins, full length heavy chains, light chains or complete antibodies as described herein can be produced using conventional recombinant protein production and purification processes.

All the sequences referred to in this specification (SEQ ID NOs) are found in Table 51. Throughout the text of this application, should there be a discrepancy between the text of the specification (e.g., Table 51) and the sequence listing, the text of the specification shall prevail. Ref No refers to an internal sequence reference number.

TABLE 51
Sequence Table

| SEQ | | Ref |
|---|---|---|
| ID NO: | Sequence | No: |
| 1 | evqlvesggglvqpggslrlscaasgfnikdtyihwvrqapgkglewvariyptngytryadsv | 2257 |
| | kgrftisadtskntaylqmnslraedtavyycsrwggdgfyamdywgqgtlvtvssastkgpsv | |
| | fplapsskstsggtaalgclvkdvfpepvtvswnsgaltsgvhtfpavlqssglvslssvvtvp | |
| | ssslgtqtyicnvnhkpsntkvdkkvepkscdkthtcppcpapellggpsvflfppkpkdtlmi | |
| | srtpevtcvvvdvshedpevkfnwyvdgvevhnaktkpreeqynstyrvvsvltvlhqdwlngk | |
| | eykckvsnkalpapiektiskakgqprepqvytlppsrdeltknqvsltclvkgfypsdiavew | |
| | esngqpennykttppvldsdgsfflyskltvdksrwqqgnvfscsvmhealhnhytqkslslsp | |
| 2 | evqlvesggglvqpggslrlscaasgfnikdtyihwvrqapgkglewvariyptngytryadsv | 2258 |
| | kgrftisadtskntaylqmnslraedtavyycsrwggdgfyamdywgqgtlvtvssasptspkv | |
| | fplsldstpgdgnvvvaclvqqffpgeplsvtwsesggnvtarnfppsqdasqdlyttssqltl | |
| | patqcpdgksvtchvkhytnpsqdvtvpcpvpppppcchprlslhrpaledlllgseanltctl | |
| | tglrdasgatftwtpssgksavqgpperdlcgcysvssvlpgcaqpwnhgetftctaahpelkt | |
| | pltanitksgntfrpevhllpppseelalnelvtltclargfspkdvlvrwlqgsqelprekyl | |
| | twasrqepsqgtttfavtsilrvaaedwkkgdtfscmvghealplaftqktidrla | |
| 3 | evqlvesggglvqpggslrlscaasgfnikdtyihwvrqapgkglewvariyptngytryadsv | 2737 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvlpgcaqpwnhgetftctaahpelkt | |
| | pltanitksgntfrpevhllpppseelalnelvtltclargfspkdvlvrwlqgsqelprekyl | |
| | twasrqepsqgtttfavtsilrvaaedwkkgdtfscmvghealplaftqktidrla | |
| 4 | evqlvesggglvqpggslrlscaasgfnikdtyihwvrqapgkglewvariyptngytryadsv | 2259 |
| | kgrftisadtskntaylqmnslraedtavyycsrwggdgfyamdywgqgtlvtvssastkgpsv | |
| | fplapsskstsggtaalgclvkdvfpepvtvswnsgaltsgvhtfpavlqssglvslssvvtvp | |
| | ssslgtqtyicnvnhkpsntkvdkkvvpppppcchprlslhrpaledlllgseanltctltglr | |
| | dasgatftwtpssgksavqgpperdlcgcysvssvlpgcaqpwnhgetftctaahpelktplta | |
| | nitksgntfrpevhllpppseelalnelvtltclargfspkdvlvrwlqgsqelprekyltwas | |
| | rqepsqgtttfavtsilrvaaedwkkgdtfscmvghealplaftqktidrla | |
| 5 | evqlvesggglvqpggslrlscaasgfnikdtyihwvrqapgkglewvariyptngytryadsv | 2260 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvvpppppcchprlslhrpaledlllgseanltctltglr | |
| | dasgatftwtpssgksavqgpperdlcgcysvssvlpgcaqpwnhgetftctaahpelktplta | |
| | niskakgqprepevhllpppseelalnelvtltclargfspkdvlvrwlqgsqelprekyltwa | |
| | srqepsqgtttfavtsilrvaaedwkkgdtfscmvghealplaftqktidrla | |
| 6 | evqlvesggglvqpggslrlscaasgfnikdtyihwvrqapgkglewvariyptngytryadsv | 2261 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvvpppppcchprlslhrpaledlllgseanltctltglr | |
| | dasgatftwtpssgksavqgpperdlcgcysvssvlpgcaqpwnhgetftctaahpelktplta | |
| | niskagqprepevhllpppseelalnelvtltclargfspkdvlvrwlqgsqelprekyltwas | |
| | rqepsqgtttfavtsilrvaaedwkkgdtfscmvghealplaftqktidrla | |
| 7 | evqlvesggglvqpggslrlscaasgfnikdtyihwvrqapgkglewvariyptngytryadsv | 2262 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdicgcysvssvlpgcaqpwnhgetftetaahpe | |
| | lktpltanitksgntfrpevhllpppseelalnelvtltclargfspkdvlvrwlqgsqelpre | |
| | kyltwasrqepsqgtttfavtsilrvaaedwkkgdtfscmvghealplaftqktidrla | |
| 8 | evqlvesggglvqpggslrlscaasgfnikdtyihwvrqapgkglewvariyptngytryadsv | 2263 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdicgcysvssvlpgcaqpwnhgetftetaahpe | |
| | lktpltaniskakgqprepevhllpppseelalnelvtitelargfspkdvivrwlqgsqelpr | |
| | ekyltwasrqepsqgtttfavtsilrvaaedwkkgdtfscmvghealplaftqktidrla | |
| 9 | evqlvesggglvqpggslrlscaasgfnikdtyihwvrqapgkglewvariyptngytryadsv | 2264 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdicgcysvssvlpgcaqpwnhgetftctaahpe | |
| | iktpltaniskagqprepevhllpppseelalnelvtitclargfspkdvlvrwlqgsqelpre | |
| | kyltwasrqepsqgtttfavtsilrvaaedwkkgdtfscmvghealplaftqktidrla | |
| 10 | evqlvesggglvqpggslrlscaasgfnikdtyihwvrqapgkglewvariyptngytryadsv | 2265 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | sepevtcvvvdvshedpevkfnwyvdgvevhnaktkpreeqynstyrvvsvlpgcaqpwnhgke | |
| | ykckvsnkalpapiektitksgntfrpevhllpppseelalnelvtltclargfspkdvivrwl | |
| | qgsqelprekyltwasrqepsqgtttfavtsilrvaaedwkkgdtfscmvghealplaftqkti | |
| | drla | |
| 11 | evqlvesggglvqpggslrlscaasgfnikdtyihwvrqapgkglewvariyptngytryadsv | 2266 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvlpgcaqpwnhgke | |
| | ykckvsnkalpapiektitksgntfrpevhllpppseelalnelvtltclargfspkdvivrwl | |
| | qgsqelprekyltwasrqepsqgtttfavtsilrvaaedwkkgdtfscmvghealplaftqkti | |
| | drla | |
| 12 | evqlvesggglvqpggslrlscaasgfnikdtyihwvrqapgkglewvariyptngytryadsv | 2267 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvlpglaqpwnhgke | |
| | ykckvsnkalpapiektitksgntfrpevhllpppseelalnelvtltclargfspkdvivrwl | |
| | qgsqelprekyltwasrqepsqgtttfavtsilrvaaedwkkgdtfscmvghealplaftqkti | |
| | drla | |
| 13 | evqlvesggglvqpggslrlscaasgfnikdtyihwvrqapgkglewvariyptngytryadsv | 2268 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdicgcysvssvlpgcaqpwnhgetftetaahpe | |
| | iktpltanitksgntfrpqvytlppsrdeltknqvsltclvkgfypsdilvrwlqgsqelnnyk | |
| | ttppvldsdgsfflyskltvdksrwqqgntfscmvghealplaftqktlslsp | |
| 14 | evqlvesggglvqpggslrlscaasgfnikdtyihwvrqapgkglewvariyptngytryadsv | 2343 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqyastyrvvsvlpglaqpwnhgke | |
| | ykekvsnkaipapiektiskagqprepevhllpppseelalnelvtitclargfspkdvivrwl | |
| | qgsqelprekyltwasrqepsqgtttfavtsilrvaaedwkkgdtfscmvghealplaftqkti | |
| | drla | |
| 15 | evqlvesggglvqpggslrlscaasgfnikdtyihwvrqapgkglewvariyptngytryadsv | 2344 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshepevkfnwyvdgvevaktkpreeqyngtyrvvsvlpglaqpwnhgkeykc | |
| | kvsnkalpapiektiskagqprepevhllpppseelalnelvtltclargfspkdvlvrwlqgs | |
| | qelprekyltwasrqepsqgtttfavtsilrvaaedwkkgdtfscmvghealplaftqktidrl | |
| | a | |
| 16 | evqlvesggglvqpggslrlscaasgfnikdtyihwvrqapgkglewvariyptngytryadsv | 2345 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcgpsvflfrpaledlllgseanvtc | |
| | vvvdvshepevkfnwyvdgvevaktkpreeqyngtyrvvsvlpglaqpwnhgkeykckvsnkal | |
| | papiektiskagqprepevhllpppseelalnelvtitclargfspkdvlvrwlqgsqelprek | |
| | yltwasrqepsqgtttfavtsilrvaaedwkkgdtfscmvghealplaftqktidrla | |
| 17 | evqlvesggglvqpggslrlscaasgfnikdtyihwvrqapgkglewvariyptngytryadsv | 2346 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapelccgpsvflfrpaledlllg | |
| | seanvtcvvvdvshepevkfnwyvdgvevaktkpreeqycgcyrvvsvlpglaqpwnhgkeykc | |
| | kvsnkalpapiektiskagqprepevhllpppseelalnelvtitclargfspkdvlvrwlqgs | |
| | qelprekyltwasrqepsqgtttfavtsilrvaaedwkkgdtfscmvghealplaftqktidrl | |
| | a | |
| 18 | evqlvesggglvqpggslrlscaasgfnikdtyihwvrqapgkglewvariyptngytryadsv | 2347 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpccgpsvflfrpaledlllgsean | |
| | vtcvvvdvshepevkfnwyvdgvevaktkpreeqycgcyrvvsvlpglaqpwnhgkeykckvsn | |
| | kalpapiektiskagqprepevhllpppseelalnelvtitclargfspkdvlvrwlqgsqelp | |
| | rekyltwasrqepsqgtttfavtsilrvaaedwkkgdtfscmvghealplaftqktidrla | |
| 19 | evqlvesggglvqpggslrlscaasgfnikdtyihwvrqapgkglewvariyptngytryadsv | 2348 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqyastyrvvsvlpgcaqpwnhgke | |
| | ykckvsnkalpapiektiskagqprepevhllpppseelalnelvtitclargfspkdvivrwl | |
| | qgsqelprekyltwasrqepsqgtttfavtsilrvaaedwkkgdtfscmvghealplaftqkti | |
| | drla | |
| 20 | evqlvesggglvqpggslrlscaasgfnikdtyihwvrqapgkglewvariyptngytryadsv | 2349 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshepevkfnwyvdgvevaktkpreeqyngtyrvvsvlpgcaqpwnhgkeykc | |
| | kvsnkalpapiektiskagqprepevhllpppseelalnelvtitclargfspkdvlvrwlqgs | |
| | qelprekyltwasrqepsqgtttfavtsilrvaaedwkkgdtfscmvghealplaftqktidrl | |
| | a | |
| 21 | evqlvesggglvqpggslrlscaasgfnikdtyihwvrqapgkglewvariyptngytryadsv | 2350 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcgpsvflfrpaledlllgseanvtc | |
| | vvvdvshepevkfnwyvdgvevaktkpreeqyngtyrvvsvlpgcaqpwnhgkeykckvsnkal | |
| | papiektiskagqprepevhllpppseelalnelvtitclargfspkdvlvrwlqgsqelprek | |
| | yltwasrqepsqgtttfavtsilrvaaedwkkgdtfscmvghealplaftqktidrla | |
| 22 | evqlvesggglvqpggslrlscaasgfnikdtyihwvrqapgkglewvariyptngytryadsv | 2351 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapelccgpsvflfrpaledlllg | |
| | seanvtcvvvdvshepevkfnwyvdgvevaktkpreeqycgcyrvvsvlpgcaqpwnhgkeykc | |
| | kvsnkalpapiektiskagqprepevhllpppseelalnelvtitclargfspkdvlvrwlqgs | |
| | qelprekyltwasrqepsqgtttfavtsilrvaaedwkkgdtfscmvghealplaftqktidrl | |
| | a | |
| 23 | evqlvesggglvqpggslrlscaasgfnikdtyihwvrqapgkglewvariyptngytryadsv | 2352 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpccgpsvflfrpaledlllgsean | |
| | vtcvvvdvshepevkfnwyvdgvevaktkpreeqycgcyrvvsvlpgcaqpwnhgkeykckvsn | |
| | kalpapiektiskagqprepevhllpppseelalnelvtitclargfspkdvlvrwlqgsqelp | |
| | rekyltwasrqepsqgtttfavtsilrvaaedwkkgdtfscmvghealplaftqktidrla | |
| 24 | evqlvesggglvqpggslrlscaasgfnikdtyihwvrqapgkglewvariyptngytryadsv | 2353 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdlcgcysvssvlpgcaqpwnhgetftctaahpe | |
| | lktpltaniskagqprepqvytlppsrdeltknqvsltclvkgfypsdilvrwlqgsqelnnyk | |
| | ttppvldsdgsfflyskltvdksrwqqgntfscmvghealplaftqktlslsp | |
| 25 | evqlvesggglvqpggslrlscaasgfnikdtyihwvrqapgkglewvariyptngytryadsv | 2354 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdicgcysvssvlpgcaqpwnhgetftetaahpe | |
| | iktpltaniskagqprepqvytlppsrdeltknqvsltclvkgfypsdilvrwlqgsqelpren | |
| | ykttppvldsdgsfflyskltvdksrwqqgntfscmvghealplaftqktlslsp | |
| 26 | evqlvesggglvqpggslrlscaasgfnikdtyihwvrqapgkglewvariyptngytryadsv | 2355 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdicgcysvssvlpgcaqpwnhgetftctaahpe | |
| | lktpltaniskagqprepqvytlppsrdeltknqvsltclvkgfypsdilvrwlqgsqelpren | |
| | ykttppvldsdqgtgsfflyskltvdksrwqqgntfscmvghealplaftqktlslsp | |
| 27 | evqlvesggglvqpggslrlscaasgfnikdtyihwvrqapgkglewvariyptngytryadsv | 2356 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdicgcysvssvlpgcaqpwnhgetftetaahpe | |
| | iktpltaniskagqprepqvytlppsrdeltlknqvsltclvkgfypsdilvrwlqgsqelpre | |
| | nykttppvldsdqgtgsfflyskltvdksrwqqgntfscmvghealplaftqktlslsp | |
| 28 | evqlvesggglvqpggslrlscaasgfnikdtyihwvrqapgkglewvariyptngytryadsv | 2462 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanltctltglrdasgatftwtpssgksavqgpperdicgcysvssvlpgcaqpwnhgetftc | |
| | taahpelktpltanitksgntfrpevhllpppseelalnelvtltclargfspkdvlvrwlqgs | |
| | qelprekyltwasrqepsqgtttfavtsilrvaaedwkkgdtfscmvghealplaftqktidrl | |
| | a | |
| 29 | evqlvesggglvqpggslrlscaasgfnikdtyihwvrqapgkglewvariyptngytryadsv | 2463 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | vtcvvvdvshasgatftwtpssgksavqgpperdicgcysvssvlpgcaqpwnhgetftetaah | |
| | pelktpitanitksgntfrpevhllpppseelalnelvtltclargfspkdvlvrwlqgsqelp | |
| | rekyltwasrqepsqgtttfavtsilrvaaedwkkgdtfscmvghealplaftqktidrla | |
| 30 | evqlvesggglvqpggslrlscaasgfnikdtyihwvrqapgkglewvariyptngytryadsv | 2464 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdedpevkfnwyvdggksavqgpperdicgcysvssvlpgcaqpwnhgetftctaa | |
| | hpeiktpltanitksgntfrpevhllpppseelalnelvtltclargfspkdvlvrwlqgsqel | |
| | prekyltwasrqepsqgtttfavtsilrvaaedwkkgdtfscmvghealplaftqktidrla | |
| 31 | evqlvesggglvqpggslrlscaasgfnikdtyihwvrqapgkglewvariyptngytryadsv | 2465 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdasgatftwtpssvevhnaktkpreeqycgcysvssvlpgcaqpwnhgetfteta | |
| | ahpelktpltanitksgntfrpevhllpppseelalnelvtltclargfspkdvlvrwlqgsqe | |
| | lprekyltwasrqepsqgtttfavtsilrvaaedwkkgdtfscmvghealplaftqktidrla | |
| 32 | evqlvesggglvqpggslrlscaasgfnikdtyihwvrqapgkglewvariyptngytryadsv | 2466 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdlnstyrvvsvlpgcaqpwnhgetftetaahpe | |
| | lktpltanitksgntfrpevhllpppseelalnelvtltclargfspkdvlvrwlqgsqelpre | |
| | kyltwasrqepsqgtttfavtsilrvaaedwkkgdtfscmvghealplaftqktidrla | |
| 33 | evqlvesggglvqpggslrlscaasgfnikdtyihwvrqapgkglewvariyptngytryadsv | 2467 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdasgatftwtpssgksavqgpperdicgcysvssvlpgcaqpwnhgkeykckvsn | |
| | kalktpltanitksgntfrpevhllpppseelalnelvtltclargfspkdvlvrwlqgsqelp | |
| | rekyltwasrqepsqgtttfavtsilrvaaedwkkgdtfscmvghealplaftqktidrla | |
| 34 | Evqlvesggglvqpggslrlscaasgfnikdtyihwvrqapgkglewvariyptngytryadsv | 2468 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdasgatftwtpssgksavqgpperdlcgcysvssvlpgcaqpwnhgetftctaah | |
| | pelpapiektitksgntfrpevhllpppseelalnelvtltclargfspkdvlvrwlqgsqelp | |
| | rekyltwasrqepsqgtttfavtsilrvaaedwkkgdtfscmvghealplaftqktidrla | |
| 35 | evqlvesggglvqpggslrlscaasgfnikdtyihwvrqapgkglewvariyptngytryadsv | 2469 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdedpevkfnwyvdgvevhnaktkpreeqycgcysvssvlpgcaqpwnhgetftct | |
| | aahpelktpltanitksgntfrpevhllpppseelalnelvtltclargfspkdvlvrwlqgsq | |
| | elprekyltwasrqepsqgtttfavtsilrvaaedwkkgdtfscmvghealplaftqktidrla | |
| 36 | evqlvesggglvqpggslrlscaasgfnikdtyihwvrqapgkglewvariyptngytryadsv | 2470 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdedpevkfnwyvdgvevhnaktkpreeqycgcysvssvlpgcaqpwnhgetftct | |
| | aahpelpapiektitksgntfrpevhllpppseelalnelvtltclargfspkdvlvrwlqgsq | |
| | elprekyltwasrqepsqgtttfavtsilrvaaedwkkgdtfscmvghealplaftqktidrla | |
| 37 | evqlvesggglvqpggslrlscaasgfnikdtyihwvrqapgkglewvariyptngytryadsv | 2471 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdedpevkfnwyvdgvevhnaktkpreeqynstyrvvsvlpgcaqpwnhgkeykckvs | |
| | nkalpapiektitksgntfrpevhllpppseelalnelvtltclargfspkdvlvrwlqgsqel | |
| | prekyltwasrqepsqgtttfavtsilrvaaedwkkgdtfscmvghealplaftqktidrla | |
| 38 | evqlvesggglvqpggslrlscaasgfnikdtyihwvrqapgkglewvariyptngytryadsv | 2472 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqycgcysvssvlpgcaqpwnhget | |
| | ftctaahpelpapiektitksgntfrpevhllpppseelalnelvtltclargfspkdvivrwl | |
| | qgsqelprekyltwasrqepsqgtttfavtsilrvaaedwkkgdtfscmvghealplaftqkti | |
| | drla | |
| 39 | evqlvesggglvqpggslrlscaasgfnikdtyihwvrqapgkglewvariyptngytryadsv | 2473 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshasgatftwtpssgksavqgpperdlnstyrvvsvlpgcaqpwnhgkeykc | |
| | kvsnkalktpltanitksgntfrpevhllpppseelalnelvtltclargfspkdvlvrwlqgs | |
| | qelprekyltwasrqepsqgtttfavtsilrvaaedwkkgdtfscmvghealplaftqktidrl | |
| | a | |
| 40 | evqlvesggglvqpggslrlscaasgfnikdtyihwvrqapgkglewvariyptngytryadsv | 2474 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshasgatftwtpssgksavqgpperdlcgcysvssvlpgcaqpwnhgetftc | |
| | taahpelktpitanitksgntfrpevhllpppseelalnelvtltclargfspkdvlvrwlqgs | |
| | qelprekyltwasrqepsqgtttfavtsilrvaaedwkkgdtfscmvghealplaftqktidrl | |
| | a | |
| 41 | evqlvesggglvqpggslrlscaasgfnikdtyihwvrqapgkglewvariyptngytryadsv | 2475 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdlnstyrvvsvlpgcaqpwnhgkeykckvsnka | |
| | iktpltanitksgntfrpevhllpppseelalnelvtltclargfspkdvlvrwlqgsqelpre | |
| | kyltwasrqepsqgtttfavtsilrvaaedwkkgdtfscmvghealplaftqktidrla | |
| 42 | evqlvesggglvqpggslrlscaasgfnikdtyihwvrqapgkglewvariyptngytryadsv | 2476 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshasgatftwtpssvevhnaktkpreeqynstyrvvsvlpgcaqpwnhgetf | |
| | tctaahpelktpltanitksgntfrpevhllpppseelalnelvtltclargfspkdvlvrwlq | |
| | gsqelprekyltwasrqepsqgtttfavtsilrvaaedwkkgdtfscmvghealplaftqktid | |
| | rla | |
| 43 | evqlvesggglvqpggslrlscaasgfnikdtyihwvrqapgkglewvariyptngytryadsv | 2477 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdedpevkfnwyvdggksavqgpperdlcgcysvssvlpgcaqpwnhgkeykckvs | |
| | nkalpapiektitksgntfrpevhllpppseelalnelvtltclargfspkdvlvrwlqgsqel | |
| | prekyltwasrqepsqgtttfavtsilrvaaedwkkgdtfscmvghealplaftqktidrla | |
| 44 | evqlvesggglvqpggslrlscaasgfnikdtyihwvrqapgkglewvariyptngytryadsv | 2478 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpslslhrpaledlllg | |
| | seanltctlvdvshedpevkftwtpssgksavtkpreeqynstyrvssvlpgcaqpwnhgetft | |
| | ctvsnkalpapltanitksgntfrpevhllpppseelalnelvtltclargfspkdvlvrwlqg | |
| | sqelprekyltwasrqepsqgtttfavtsilrvaaedwkkgdtfscmvghealplaftqktidr | |
| | la | |
| 45 | evqlvesggglvqpggslrlscaasgfnikdtyihwvrqapgkglewvariyptngytryadsv | 2479 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdasgatfnwyvdgvevhnakqgpperdicgcysvvsvlpgcaqpwnhgkeykcka | |
| | ahpelktpiektitksgntfrpevhllpppseelalnelvtltclargfspkdvlvrwlqgsqe | |
| | lprekyltwasrqepsqgtttfavtsilrvaaedwkkgdtfscmvghealplaftqktidrla | |
| 46 | evqlvesggglvqpggslrlscaasgfnikdtyihwvrqapgkglewvariyptngytryadsv | 2459 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdicgcysvssvlpgcaqpwnhgetftetaahpe | |
| | lktpltaniskagqprrpqvytlppsrdeltlknqvsltclvkgfypsdilvrwlqgsqelpre | |
| | nykttppvldsdqgtgsfflyskltvdksrwqqgntfscmvghealplaftqktlslsp | |
| 47 | evqlvesggglvqpggslrlscaasgfnikdtyihwvrqapgkglewvariyptngytryadsv | 2460 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdicgcysvssvlpgcaqpwnhgetftetaahpe | |
| | iktpltaniskagqprepevytlppsrdeltlknqvsltclvkgfypsdilvrwlqgsqelpre | |
| | nykttppvldsdqgtgsfflyskltvdksrwqqgntfscmvghealplaftqktlslsp | |
| 48 | evqlvesggglvqpggslrlscaasgfnikdtyihwvrqapgkglewvariyptngytryadsv | 2461 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdicgcysvssvlpgcaqpwnhgetftetaahpe | |
| | iktpltaniskagqprrpevytlppsrdeltlknqvsltclvkgfypsdilvrwlqgsqelpre | |
| | nykttppvldsdqgtgsfflyskltvdksrwqqgntfscmvghealplaftqktlslsp | |
| 49 | evqlvesggglvqpggslrlscaasgfnikdtyihwvrqapgkglewvariyptngytryadsv | 2564 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvtglrdasgatfnwyvdgvevhnakqgpperdicgcysvvsvlpgcaqpwnhgkey | |
| | kckaahpelktpiektitksgntfrpevhllpppseelalnelvtltclargfspkdvlvrwlq | |
| | gsqelprekyltwasrqepsqgtttfavtsilrvaaedwkkgdtfscmvghealplaftqktid | |
| | ria | |
| 50 | evqlvesggglvqpggslrlscaasgfnikdtyihwvrqapgkglewvariyptngytryadsv | 2565 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnakqgpperdlcgcysvvsvlpgcaqpwnhgkeykck | |
| | aahpelktpiektitksgntfrpevhllpppseelalnelvtltclargfspkdvlvrwlqgsq | |
| | elprekyltwasrqepsqgtttfavtsilrvaaedwkkgdtfscmvghealplaftqktidrla | |
| 51 | evqlvesggglvqpggslrlscaasgfnikdtyihwvrqapgkglewvariyptngytryadsv | 2566 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdasgatfnwyvdgvevhnaktkpreeqycgcysvvsvlpgcaqpwnhgkeykcka | |
| | ahpelktpiektitksgntfrpevhllpppseelalnelvtltclargfspkdvlvrwlqgsqe | |
| | lprekyltwasrqepsqgtttfavtsilrvaaedwkkgdtfscmvghealplaftqktidrla | |
| 52 | evqlvesggglvqpggslrlscaasgfnikdtyihwvrqapgkglewvariyptngytryadsv | 2567 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdasgatfnwyvdgvevhnakqgpperdlcgcysvvsvlpgcaqpwnhgkeykckv | |
| | snkalktpiektitksgntfrpevhllpppseelalnelvtltclargfspkdvlvrwlqgsqe | |
| | lprekyltwasrqepsqgtttfavtsilrvaaedwkkgdtfscmvghealplaftqktidrla | |
| 53 | evqlvesggglvqpggslrlscaasgfnikdtyihwvrqapgkglewvariyptngytryadsv | 2568 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdasgatfnwyvdgvevhnakqgpperdicgcysvvsvlpgcaqpwnhgkeykcka | |
| | ahpelpaplektitksgntfrpevhllpppseelalnelvtltclargfspkdvlvrwlqgsqe | |
| | lprekyltwasrqepsqgtttfavtsilrvaaedwkkgdtfscmvghealplaftqktidrla | |
| 54 | evqlvesggglvqpggslrlscaasgfnikdtyihwvrqapgkglewvariyptngytryadsv | 2569 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvtglrdedpevkfnwyvdgvevhnaktkpreeqycgcysvvsvlpgcaqpwnhgke | |
| | ykckaahpelpaplektitksgntfrpevhllpppseelalnelvtltclargfspkdvlvrwl | |
| | qgsqelprekyltwasrqepsqgtttfavtsilrvaaedwkkgdtfscmvghealplaftqkti | |
| | drla | |
| 55 | evqlvesggglvqpggslrlscaasgfnikdtyihwvrqapgkglewvariyptngytryadsv | 2570 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvtglrdedpevkfnwyvdgvevhnaktkpreeqycgcysvvsvlpgcaqpwnhgke | |
| | ykckvsnkalpapiektitksgntfrpevhllpppseelalnelvtltclargfspkdvlvrwl | |
| | qgsqelprekyltwasrqepsqgtttfavtsilrvaaedwkkgdtfscmvghealplaftqkti | |
| | drla | |
| 56 | evqlvesggglvqpggslrlscaasgfnikdtyihwvrqapgkglewvariyptngytryadsv | 2571 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvlpgcaqpwnhgkeykck | |
| | aahpelpapiektitksgntfrpevhllpppseelalnelvtltclargfspkdvlvrwlqgsq | |
| | elprekyltwasrqepsqgtttfavtsilrvaaedwkkgdtfscmvghealplaftqktidrla | |
| 57 | evqlvesggglvqpggslrlscaasgfnikdtyihwvrqapgkglewvariyptngytryadsv | 2572 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvlpgcaqpwnhgkeykck | |
| | vsnkalpapiektitksgntfrpevhllpppseelalnelvtltclargfspkdvlvrwlqgsq | |
| | elprekyltwasrqepsqgtttfavtsilrvaaedwkkgdtfscmvghealplaftqktidrla | |
| 58 | evqlvesggglvqpggslrlscaasgfnikdtyihwvrqapgkglewvariyptngytryadsv | 2576 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdicgcysvssvlpgcaqpwnhgetftetaahpe | |
| | iktpltaniskagqprepqvytlppsrdeltlnelvtltclargfspkdvlvrwlqgsqelpre | |
| | kyltwasrqepsqgtttfavtsilrvaaedwkkgdtfscmvghealplaftqktidrla | |
| 59 | evqlvesggglvqpggslrlscaasgfnikdtyihwvrqapgkglewvariyptngytryadsv | 2577 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdicgcysvssvlpgcaqpwnhgetftetaahpe | |
| | iktpltaniskagqprepevhllpppseelaknqvsltclvkgfypkdvlvrwlqgsqelprek | |
| | yltwasrqepsqgtttfavtsilrvaaedwkkgdtfscmvghealplaftqktidrla | |
| 60 | evqlvesggglvqpggslrlscaasgfnikdtyihwvrqapgkglewvariyptngytryadsv | 2578 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdicgcysvssvlpgcaqpwnhgetftctaahpe | |
| | iktpltaniskagqprepevhllpppseelalnelvtltclargfspsdiavewesngqpenky | |
| | ltwasrqepsqgtttfavtsilrvaaedwkkgdtfscmvghealplaftqktidrla | |
| 61 | evqlvesggglvqpggslrlscaasgfnikdtyihwvrqapgkglewvariyptngytryadsv | 2579 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdicgcysvssvlpgcaqpwnhgetftetaahpe | |
| | lktpltaniskagqprepevhllpppseelalnelvtltclargfspsdiavewlqgsqelpre | |
| | kyltwasrqepsqgtttfavtsilrvaaedwkkgdtfscmvghealplaftqktidrla | |
| 62 | evqlvesggglvqpggslrlscaasgfnikdtyihwvrqapgkglewvariyptngytryadsv | 2580 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdlcgcysvssvlpgcaqpwnhgetftctaahpe | |
| | iktpltaniskagqprepevhllpppseelalnelvtltclargfspkdvlvrwesngqpenky | |
| | ltwasrqepsqgtttfavtsilrvaaedwkkgdtfscmvghealplaftqktidrla | |
| 63 | evqlvesggglvqpggslrlscaasgfnikdtyihwvrqapgkglewvariyptngytryadsv | 2581 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdicgcysvssvlpgcaqpwnhgetftetaahpe | |
| | iktpltaniskagqprepevhllpppseelalnelvtitclargfspkdvlvrwlqgsqelpre | |
| | nykttppvldsdqgtttfavtsilrvaaedwkkgdtfscmvghealplaftqktidrla | |
| 64 | evqlvesggglvqpggslrlscaasgfnikdtyihwvrqapgkglewvariyptngytryadsv | 2582 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdicgcysvssvlpgcaqpwnhgetftctaahpe | |
| | lktpltaniskagqprepevhllpppseelalnelvtitclargfspkdvlvrwlqgsqelpre | |
| | kyltwasrqepsqgtgsfflyskltvdaedwkkgdtfscmvghealplaftqktidrla | |
| 65 | evqlvesggglvqpggslrlscaasgfnikdtyihwvrqapgkglewvariyptngytryadsv | 2583 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdicgcysvssvlpgcaqpwnhgetftetaahpe | |
| | lktpltaniskagqprepevhllpppseelalnelvtitclargfspkdvlvrwlqgsqelpre | |
| | kyltwasrqepsqgtttfavtsilrvaksrwqqgntfscmvghealplaftqktidrla | |
| 66 | evqlvesggglvqpggslrlscaasgfnikdtyihwvrqapgkglewvariyptngytryadsv | 2584 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdicgcysvssvlpgcaqpwnhgetftetaahpe | |
| | lktpltaniskagqprepevhllpppseelalnelvtitclargfspkdvlvrwlqgsqelpre | |
| | kyltwasrqepsqgtttfavtsilrvaaedwkkgdvfscsvmhealplaftqktidrla | |
| 67 | evqlvesggglvqpggslrlscaasgfnikdtyihwvrqapgkglewvariyptngytryadsv | 2585 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdicgcysvssvlpgcaqpwnhgetftetaahpe | |
| | lktpltaniskagqprepevhllpppseelalnelvtitclargfspkdvlvrwlqgsqelpre | |
| | kyltwasrqepsqgtttfavtsilrvaaedwkkgdtfscmvghealhnhytqktidrla | |
| 68 | evqlvesggglvqpggslrlscaasgfnikdtyihwvrqapgkglewvariyptngytryadsv | 2586 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdicgcysvssvlpgcaqpwnhgetftetaahpe | |
| | lktpltaniskagqprepevhllpppseelalnelvtitclargfspkdvlvrwlqgsqelpre | |
| | kyltwasrqepsqgtttfavtsilrvaaedwkkgdtfscmvghealplaftqkslslsp | |
| 69 | evqlvesggglvqpggslrlscaasgfnikdtyihwvrqapgkglewvariyptngytryadsv | 2587 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdicgcysvssvlpgcaqpwnhgetftetaahpe | |
| | iktpltaniskagqprepevhllppsrdeltknqvsltclargfspkdvlvrwlqgsqelpren | |
| | yktwasrqepsqgtttfavtskltvdksrwqqgnvfscmvghealplaftqktlslsp | |
| 70 | evqlvesggglvqpggslrlscaasgfnikdtyihwvrqapgkglewvariyptngytryadsv | 2588 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdicgcysvssvlpgcaqpwnhgetftetaahpe | |
| | lktpltaniskagqprepqvytlpppseelalnelvtitclvkgfypsdilvrwlqgsqelpre | |
| | kylttppvldsdgsfflysilrvaaedwkkgdtfscmvghealplaftqktidrla | |
| 71 | evqlvesggglvqpggslrlscaasgfnikdtyihwvrqapgkglewvariyptngytryadsv | 2591 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdlcgcysvssvlpgcaqpwnhgetftctaahpe | |
| | lktpltanitksgntfrpevhllpppseelalnelvtltclargfspkdvlvrwlqgsqelpre | |
| | kyltwasrqepsqgtttfavtsilrvaaedwkkgdtfscmvghealplhytqktidrla | |
| 72 | evqlvesggglvqpggslrlscaasgfnikdtyihwvrqapgkglewvariyptngytryadsv | 2592 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcphprlslhrpaledlllgseanlt | |
| | ctltglrdasgatftwtpssgksavqgpperdicgcysvssvlpgcaqpwnhgetftetaahpe | |
| | iktpltanitksgntfrpevhllpppseelalnelvtltclargfspkdvlvrwlqgsqelpre | |
| | kyltwasrqepsqgtttfavtsilrvaaedwkkgdtfscmvghealhnhytqktidrla | |
| 73 | evqlvesggglvqpggslrlscaasgfnikdtyihwvrqapgkglewvariyptngytryadsv | 2683 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvvdvshedpevkfnwyvdgvevhnaktkpreeqycgcysvvsvlpgcaqpwnhgkeykck | |
| | aahpelpapiektitksgntfrpevhllpppseelalnelvtltclargfspkdvlvrwlqgsq | |
| | elprekyltwasrqepsqgtttfavtsilrvaaedwkkgdtfscmvghealplaftqktidrla | |
| 74 | evqlvesggglvqpggslrlscaasgfnikdtyihwvrqapgkglewvariyptngytryadsv | 2684 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvvdvshedpevkfnwyvdgvevhnaktkpreeqycgcysvvsvlpgcaqpwnhgkeykck | |
| | vsnkalpapiektitksgntfrpevhllpppseelalnelvtltclargfspkdvlvrwlqgsq | |
| | elprekyltwasrqepsqgtttfavtsilrvaaedwkkgdtfscmvghealplaftqktidrla | |
| 75 | evqlvesggglvqpggslrlscaasgfnikdtyihwvrqapgkglewvariyptngytryadsv | 2685 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqynstyrvvsvlpgcaqpwnhgkeykck | |
| | aahpelpapiektitksgntfrpevhllpppseelalnelvtltclargfspkdvlvrwlqgsq | |
| | elprekyltwasrqepsqgtttfavtsilrvaaedwkkgdtfscmvghealplaftqktidrla | |
| 76 | evqlvesggglvqpggslrlscaasgfnikdtyihwvrqapgkglewvariyptngytryadsv | 2686 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqynstyrvvsvlpgcaqpwnhgkeykck | |
| | vsnkalpapiektitksgntfrpevhllpppseelalnelvtltclargfspkdvlvrwlqgsq | |
| | elprekyltwasrqepsqgtttfavtsilrvaaedwkkgdtfscmvghealplaftqktidrla | |
| 77 | evqlvesggglvqpggslrlscaasgfnikdtyihwvrqapgkglewvariyptngytryadsv | 2687 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvtglrdedpevkfnwyvdgvevhnaktkpreeqynstyrvvsvlpgcaqpwnhgke | |
| | ykekaahpelpapiektitksgntfrpevhllpppseelalnelvtltclargfspkdvivrwl | |
| | qgsqelprekyltwasrqepsqgtttfavtsilrvaaedwkkgdtfscmvghealplaftqkti | |
| | drla | |
| 78 | evqlvesggglvqpggslrlscaasgfnikdtyihwvrqapgkglewvariyptngytryadsv | 2688 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvtglrdedpevkfnwyvdgvevhnaktkpreeqynstyrvvsvlpgcaqpwnhgke | |
| | ykckvsnkalpapiektitksgntfrpevhllpppseelalnelvtltclargfspkdvivrwl | |
| | qgsqelprekyltwasrqepsqgtttfavtsilrvaaedwkkgdtfscmvghealplaftqkti | |
| | drla | |
| 79 | evqlvesggglvqpggslrlscaasgfnikdtyihwvrqapgkglewvariyptngytryadsv | 2689 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | aahpelpapiektitksgntfrpevhllpppseelalnelvtltclargfspkdvlvrwlqgsq | |
| | elprekyltwasrqepsqgtttfavtsilrvaaedwkkgdtfscmvghealplaftqktidrla | |
| 80 | evqlvesggglvqpggslrlscaasgfnikdtyihwvrqapgkglewvariyptngytryadsv | 2696 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdasgatftwtpssgksavqgpperdicgcysvssvlpgcaqpwnhgetftctaah | |
| | pelktpltaniskagqprepqvytlppsrdeltknqvsltclvkgfypkdvlvrwlqgsqelpr | |
| | enykttppvldsdqgtttfavtsilrvaksrwqqgntfscmvghealplaftqktlslsp | |
| 81 | evqlvesggglvqpggslrlscaasgfnikdtyihwvrqapgkglewvariyptngytryadsv | 2697 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdasgatftwtpssgksavqgpperdlcgcysvssvlpgcaqpwnhgetftctaah | |
| | pelktpltaniskagqprepqvytlppsrdeltknqvsltclvkgfypsdilvrwlqgsqelpr | |
| | enykttppvldsdqgtttfavtsilrvaksrwqqgntfscmvghealplaftqktlslsp | |
| 82 | evqlvesggglvqpggslrlscaasgfnikdtyihwvrqapgkglewvariyptngytryadsv | 2698 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdasgatftwtpssgksavqgpperdicgcysvssvlpgcaqpwnhgetftetaah | |
| | pelktpltaniskagqprepqvytlppsrdeltknqvsltclvkgfypsdiavrwlqgsqelpr | |
| | enykttppvldsdqgtttfavtsilrvaksrwqqgntfscmvghealplaftqktlslsp | |
| 83 | evqlvesggglvqpggslrlscaasgfnikdtyihwvrqapgkglewvariyptngytryadsv | 2699 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdasgatftwtpssgksavqgpperdicgcysvssvlpgcaqpwnhgetftetaah | |
| | pelktpltaniskagqprepqvytlppsrdeltknqvsltclvkgfypkdvlvrwlqgsqelpr | |
| | enykttppvldsdttfavtsilrvaksrwqqgntfsemvghealplaftqktlslsp | |
| 84 | evqlvesggglvqpggslrlscaasgfnikdtyihwvrqapgkglewvariyptngytryadsv | 2700 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdasgatftwtpssgksavqgpperdicgcysvssvlpgcaqpwnhgetftetaah | |
| | pelktpltaniskagqprepqvytlppsrdeltknqvsltclvkgfypkdvlvrwlqgsqelpr | |
| | enykttppvldsdgsfflysilrvaksrwqqgntfsemvghealplaftqktlslsp | |
| 85 | evqlvesggglvqpggslrlscaasgfnikdtyihwvrqapgkglewvariyptngytryadsv | 2701 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdasgatftwtpssgksavqgpperdicgcysvssvlpgcaqpwnhgetftetaah | |
| | pelktpltaniskagqprepqvytlppsrdeltknqvsltclvkgfypsdiavrwlqgsqelpr | |
| | enykttppvldsdgsfflysilrvaksrwqqgntfsemvghealplaftqktlslsp | |
| 86 | evqlvesggglvqpggslrlscaasgfnikdtyihwvrqapgkglewvariyptngytryadsv | 2702 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdasgatftwtpssgksavqgpperdicgcysvssvlpgcaqpwnhgetftetaah | |
| | pelktpltaniskagqprepqvytlppsrdeltknqvsltclvkgfypkdvlvrwlqgsqelpr | |
| | enykttppvldsdgsfflyskltvdksrwqqgntfsemvghealplaftqktlslsp | |
| 87 | evqlvesggglvqpggslrlscaasgfnikdtyihwvrqapgkglewvariyptngytryadsv | 2703 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdasgatftwtpssgksavqgpperdicgcysvssvlpgcaqpwnhgetftetaah | |
| | pelktpltaniskagqpfepqvyllpppseelalnelvsltclakgfspkdvlvrwlqgsqelp | |
| | renykttasvqepsqgttsffvyskltvaaedwkkgdtfscmvghealplaftqksisrsp | |
| 88 | evqlvesggglvqpggslrlscaasgfnikdtyihwvrqapgkglewvariyptngytryadsv | 2704 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdasgatftwtpssgksavqgpperdicgcysvssvlpgcaqpwnhgetftetaah | |
| | pelktpltaniskagqpfepqvyllppsrdeltknqvsltclakgfspkdvlvrwlqgsqelpr | |
| | enykttasvqepsqgttsffvyskltvaaedwkkgdtfscmvghealplaftqksisrsp | |
| 89 | evqlvesggglvqpggslrlscaasgfnikdtyihwvrqapgkglewvariyptngytryadsv | 2705 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdasgatftwtpssgksavqgpperdicgcysvssvlpgcaqpwnhgetftetaah | |
| | pelktpltaniskagqprepqvyllppsrdeltknqvsltclakgfypkdvlvrwlqgsqelpr | |
| | enykttasvidsdgsffvyskltvaaedwkkgdtfscmvghealplaftqksisrsp | |
| 90 | evqlvesggglvqpggslrlscaasgfnikdtyihwvrqapgkglewvariyptngytryadsv | 2706 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdasgatftwtpssgksavqgpperdlcgcysvssvlpgcaqpwnhgetftctaah | |
| | pelktpltaniskagqprepqvyllpppseelalnelvsltclakgfypkdvlvrwlqgsqelp | |
| | renykttasvldsdgsffvyskltvaaedwkkgdtfscmvghealplaftqksisrsp | |
| 91 | evqlvesggglvqpggslrlscaasgfnikdtyihwvrqapgkglewvariyptngytryadsv | 2707 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdasgatftwtpssgksavqgpperdicgcysvssvlpgcaqpwnhgetftetaah | |
| | pelktpltaniskagqprepqvyllpppseelalnelvsltclakgfypkdvlvrwlqgsqelp | |
| | renykttasvldsdgsffvyskltvdksrwqqgnvfscmvghealplaftqksislsp | |
| 92 | evqlvesggglvqpggslrlscaasgfnikdtyihwvrqapgkglewvariyptngytryadsv | 2708 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdasgatftwtpssgksavqgpperdicgcysvssvlpgcaqpwnhgetftetaah | |
| | pelktpltaniskagqpfepqvyllpppseelalnelvsltclakgfspkdvlvrwlqgsqelp | |
| | renykttasvqepsqgttsffvyskltvdksrwqqgnvfscmvghealplaftqksislsp | |
| 93 | evqlvesggglvqpggslrlscaasgfnikdtyihwvrqapgkglewvariyptngytryadsv | 2709 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdasgatftwtpssgksavqgpperdicgcysvssvlpgcaqpwnhgetftetaah | |
| | pelktpltaniskagqpfepqvylippsrdeltknqvsltclakgfspkdvlvrwlqgsqelpr | |
| | enykttasvqepsqgttsffvyskltvdksrwqqgnvfscmvghealplaftqksislsp | |
| 94 | evqlvesggglvqpggslrlscaasgfnikdtyihwvrqapgkglewvariyptngytryadsv | 2710 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdasgatftwtpssgksavqgpperdicgcysvssvlpgcaqpwnhgetftetaah | |
| | pelktpltaniskagqprepqvyllppsrdeltknqvsltclakgfypkdvlvrwlqgsqelpr | |
| | enykttasvidsdgsffvyskltvdksrwqqgnvfscmvghealplaftqksislsp | |
| 95 | evqlvesggglvqpggslrlscaasgfnikdtyihwvrqapgkglewvariyptngytryadsv | 2711 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdasgatftwtpssgksavqgpperdicgcysvssvlpgcaqpwnhgetftetaah | |
| | pelktpltanitksgntfrpqvyllppsrdeltknqvsitelakgfspkdvlvrwlqgsqelpr | |
| | enykttasvqepsqgtttffvyskltvdksrwqqgnvfscmvghealplaftqksislsp | |
| 96 | evqlvesggglvqpggslrlscaasgfnikdtyihwvrqapgkglewvariyptngytryadsv | 2712 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdasgatftwtpssgksavqgpperdicgcysvssvlpgcaqpwnhgetftetaah | |
| | pelktpltanitksgntfrpqvyllppsrdeltknqvsitelakgfspkdvlvrwlqgsqelpr | |
| | enykttasvqepsqgtttffvyskltvaaedwkkgdtfscmvghealplaftqksisrsp | |
| 97 | evqlvesggglvqpggslrlscaasgfnikdtyihwvrqapgkglewvariyptngytryadsv | 2713 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdasgatftwtpssgksavqgpperdicgcysvssvlpgcaqpwnhgetftetaah | |
| | pelktpltanitksgntfrpqvyllpppseelalnelvsltclakgfspkdvlvrwlqgsqelp | |
| | renykttasvqepsqgtttffvyskltvdksrwqqgnvfscmvghealplaftqksislsp | |
| 98 | evqlvesggglvqpggslrlscaasgfnikdtyihwvrqapgkglewvariyptngytryadsv | 2714 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdasgatftwtpssgksavqgpperdicgcysvssvlpgcaqpwnhgetftetaah | |
| | pelktpltanitksgntfrpqvyllpppseelalnelvsltclakgfspkdvlvrwlqgsqelp | |
| | renykttasvqepsqgtttffvyskltvaaedwkkgdtfscmvghealplaftqksisrsp | |
| 99 | evqlvesggglvqpggslrlscaasgfnikdtyihwvrqapgkglewvariyptngytryadsv | 2715 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprlslhrpaledlllgsean | |
| | ltctltglrdasgatftwtpssgksavqgpperdicgcysvssvlpgcaqpwnhgetftetaah | |
| | pelktpltanitksgntfrpqvyllpppseelalnelvsltclakgfspkdvlvrwlqgsqelp | |
| | renykttasvqepsqgtttfavtsilrvaaedwkkgdtfscmvghealplaftqksisrsp | |
| 100 | evqlvesggglvqpggslrlscaasgfnikdtyihwvrqapgkglewvariyptngytryadsv | 2749 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvlpgcaqpwnhgkeykck | |
| | aahpelpapiektiskakgqprepqvytlppsrdeltknqvsltclvkgfypsdilvrwlqgsq | |
| | elprenykttppvldsdgsfflyskltvdksrwqqgntfscmvghealplaftqktlslsp | |
| 101 | evqlvesggglvqpggslrlscaasgfnikdtyihwvrqapgkglewvariyptngytryadsv | 2750 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvlpgcaqpwnhgkeykck | |
| | aahpelpapiektiskakgqprepqvyllppsrdeltknqvsltclakgfypkdvlvrwlqgsq | |
| | elprenykttasvldsdgsffvyskltvaaedwkkgdtfscmvghealplaftqksisrsp | |
| 102 | evqlvesggglvqpggslrlscaasgfnikdtyihwvrqapgkglewvariyptngytryadsv | 2751 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvlpgcaqpwnhgkeykck | |
| | aahpelpapiektiskakgqprepqvyllppsrdeltknqvsltclakgfypkdvlvrwlqgsq | |
| | elprenykttasvldsdgsffvyskltvdksrwqqgnvfscmvghealplaftqksislsp | |
| 103 | evqlvesggglvqpggslrlscaasgfnikdtyihwvrqapgkglewvariyptngytryadsv | 2752 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | aahpelpapiektiskakgqprepqvytlppsrdeltknqvsltclvkgfypsdilvrwlqgsq | |
| | elprenykttppvldsdgsfflyskltvdksrwqqgntfscmvghealplaftqktlslsp | |
| 104 | Evqlvesggglvqpggslrlscaasgfnikdtyihwvrqapgkglewvariyptngytryadsv | 2753 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | aahpelpapiektiskakgqprepqvyllppsrdeltknqvsltclakgfypkdvlvrwlqgsq | |
| | elprenykttasvldsdgsffvyskltvaaedwkkgdtfscmvghealplaftqksisrsp | |
| 105 | evqlvesggglvqpggslrlscaasgfnikdtyihwvrqapgkglewvariyptngytryadsv | 2754 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | aahpelpapiektiskakgqprepqvyllppsrdeltknqvsltclakgfypkdvlvrwlqgsq | |
| | elprenykttasvldsdgsffvyskltvdksrwqqgnvfscmvghealplaftqksislsp | |
| 106 | evqlvesggglvqpggslrlscaasgfnikdtyihwvrqapgkglewvariyptngytryadsv | 2755 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvlpgcaqpwnhgkeykck | |
| | vsnkalpapiektiskakgqprepqvytlppsrdeltknqvsltclvkgfypsdilvrwlqgsq | |
| | elprenykttppvldsdgsfflyskltvdksrwqqgntfscmvghealplaftqktlslsp | |
| 107 | evqlvesggglvqpggslrlscaasgfnikdtyihwvrqapgkglewvariyptngytryadsv | 2756 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvlpgcaqpwnhgkeykck | |
| | vsnkalpapiektiskakgqprepqvyllppsrdeltknqvsltclakgfypkdvlvrwlqgsq | |
| | elprenykttasvldsdgsffvyskltvaaedwkkgdtfscmvghealplaftqksisrsp | |
| 108 | evqlvesggglvqpggslrlscaasgfnikdtyihwvrqapgkglewvariyptngytryadsv | 2757 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvlpgcaqpwnhgkeykck | |
| | vsnkalpapiektiskakgqprepqvyllppsrdeltknqvsltclakgfypkdvlvrwlqgsq | |
| | elprenykttasvldsdgsffvyskltvdksrwqqgnvfscmvghealplaftqksislsp | |
| 109 | evqlvesggglvqpggslrlscaasgfnikdtyihwvrqapgkglewvariyptngytryadsv | 2758 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | vsnkalpapiektiskakgqprepqvytlppsrdeltknqvsltclvkgfypsdilvrwlqgsq | |
| | elprenykttppvldsdgsfflyskltvdksrwqqgntfscmvghealplaftqktlslsp | |
| 110 | evqlvesggglvqpggslrlscaasgfnikdtyihwvrqapgkglewvariyptngytryadsv | 2759 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | vsnkalpapiektiskakgqprepqvyllppsrdeltknqvsltclakgfypkdvlvrwlqgsq | |
| | elprenykttasvldsdgsffvyskltvaaedwkkgdtfscmvghealplaftqksisrsp | |
| 111 | evqlvesggglvqpggslrlscaasgfnikdtyihwvrqapgkglewvariyptngytryadsv | 2760 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | vsnkalpapiektiskakgqprepqvyllppsrdeltknqvsltclakgfypkdvlvrwlqgsq | |
| | elprenykttasvldsdgsffvyskltvdksrwqqgnvfscmvghealplaftqksislsp | |
| 112 | evqlvesggglvqpggslrlscaasgfnikdtyihwvrqapgkglewvariyptngytryadsv | 2761 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvlpgcaqpwnhgkeykck | |
| | aahpelpapiektiskagqprepqvytlppsrdeltknqvsltclvkgfypsdilvrwlqgsqe | |
| | lprenykttppvldsdgsfflyskltvdksrwqqgntfscmvghealplaftqktlslsp | |
| 113 | evqlvesggglvqpggslrlscaasgfnikdtyihwvrqapgkglewvariyptngytryadsv | 2762 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvlpgcaqpwnhgkeykck | |
| | aahpelpapiektiskagqprepqvyllppsrdeltknqvsltclakgfypkdvlvrwlqgsqe | |
| | lprenykttasvldsdgsffvyskltvaaedwkkgdtfscmvghealplaftqksisrsp | |
| 114 | evqlvesggglvqpggslrlscaasgfnikdtyihwvrqapgkglewvariyptngytryadsv | 2763 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvlpgcaqpwnhgkeykck | |
| | aahpelpapiektiskagqprepqvyllppsrdeltknqvsltclakgfypkdvlvrwlqgsqe | |
| | lprenykttasvldsdgsffvyskltvdksrwqqgnvfscmvghealplaftqksislsp | |
| 115 | evqlvesggglvqpggslrlscaasgfnikdtyihwvrqapgkglewvariyptngytryadsv | 2764 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | aahpelpapiektiskagqprepqvytlppsrdeltknqvsltclvkgfypsdilvrwlqgsqe | |
| | lprenykttppvldsdgsfflyskltvdksrwqqgntfscmvghealplaftqktlslsp | |
| 116 | evqlvesggglvqpggslrlscaasgfnikdtyihwvrqapgkglewvariyptngytryadsv | 2765 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | aahpelpapiektiskagqprepqvyllppsrdeltknqvsltclakgfypkdvlvrwlqgsqe | |
| | lprenykttasvldsdgsffvyskltvaaedwkkgdtfscmvghealplaftqksisrsp | |
| 117 | evqlvesggglvqpggslrlscaasgfnikdtyihwvrqapgkglewvariyptngytryadsv | 2766 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | aahpelpapiektiskagqprepqvyllppsrdeltknqvsltclakgfypkdvlvrwlqgsqe | |
| | lprenykttasvldsdgsffvyskltvdksrwqqgnvfscmvghealplaftqksislsp | |
| 118 | evqlvesggglvqpggslrlscaasgfnikdtyihwvrqapgkglewvariyptngytryadsv | 2767 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvlpgcaqpwnhgkeykck | |
| | vsnkalpapiektiskagqprepqvytlppsrdeltknqvsltclvkgfypsdilvrwlqgsqe | |
| | lprenykttppvldsdgsfflyskltvdksrwqqgntfscmvghealplaftqktlslsp | |
| 119 | evqlvesggglvqpggslrlscaasgfnikdtyihwvrqapgkglewvariyptngytryadsv | 2768 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvlpgcaqpwnhgkeykck | |
| | vsnkalpapiektiskagqprepqvyllppsrdeltknqvsltclakgfypkdvlvrwlqgsqe | |
| | lprenykttasvldsdgsffvyskltvaaedwkkgdtfscmvghealplaftqksisrsp | |
| 120 | evqlvesggglvqpggslrlscaasgfnikdtyihwvrqapgkglewvariyptngytryadsv | 2769 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvlpgcaqpwnhgkeykck | |
| | vsnkalpapiektiskagqprepqvyllppsrdeltknqvsltclakgfypkdvlvrwlqgsqe | |
| | lprenykttasvldsdgsffvyskltvdksrwqqgnvfscmvghealplaftqksislsp | |
| 121 | evqlvesggglvqpggslrlscaasgfnikdtyihwvrqapgkglewvariyptngytryadsv | 2770 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | vsnkalpapiektiskagqprepqvytlppsrdeltknqvsltclvkgfypsdilvrwlqgsqe | |
| | lprenykttppvldsdgsfflyskltvdksrwqqgntfscmvghealplaftqktlslsp | |
| 122 | evqlvesggglvqpggslrlscaasgfnikdtyihwvrqapgkglewvariyptngytryadsv | 2771 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | vsnkalpapiektiskagqprepqvyllppsrdeltknqvsltclakgfypkdvlvrwlqgsqe | |
| | lprenykttasvldsdgsffvyskltvaaedwkkgdtfscmvghealplaftqksisrsp | |
| 123 | evqlvesggglvqpggslrlscaasgfnikdtyihwvrqapgkglewvariyptngytryadsv | 2772 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | vsnkalpapiektiskagqprepqvyllppsrdeltknqvsltclakgfypkdvlvrwlqgsqe | |
| | lprenykttasvldsdgsffvyskltvdksrwqqgnvfscmvghealplaftqksislsp | |
| 124 | diqmtqspsslsasvgdrvtitcrasqdvntavawyqqkpgkapklliysasflysgvpsrfsg | 2156 |
| | srsgtdftltisslqpedfatyycqqhyttpptfgqgtkveikrtvaapsvflfppsdeqlksg | |
| | tasvvcllnnfypreakvqwkvdnalqsgnsqesvteqdskdstyslsstltlskadyekhkvy | |
| | acevthqglsspvtksfnrgec | |
| 125 | hhhhhhepkscdkthtcppcphprlslhrpaledlllgseanltctltglrdasgatftwtpss | 2835 |
| | gksavqgpperdlcgcysvssvlpgcaqpwnhgetftctaahpelktpltanitksgntfrpev | |
| | hllpppseelalnelvtltclargfspkdvlvrwlqgsqelprekyltwasrqepsqgtttfav | |
| | tsilrvaaedwkkgdtfscmvghealplaftqktidrla | |
| 126 | hhhhhhepkscdkthtcppcpcchprvflfrpaledl11gseanvtcvvtglrdedpevkfnwy | 2836 |
| | vdgvevhnaktkpreeqycgcysvvsvlpgcaqpwnhgkeykckvsnkalpapiektiskagqp | |
| | repqvyllppsrdeltknqvsltclakgfypkdvlvrwlqgsqelprenykttasvldsdgsff | |
| | vyskltvaaedwkkgdtfscmvghealplaftqksisrsp | |
| 127 | hhhhhhepkscdkthtcppcpcchprvflfrpaledl11gseanvtcvvtglrdedpevkfnwy | 2837 |
| | vdgvevhnaktkpreeqycgcysvvsvlpgcaqpwnhgkeykckvsnkalpapiektiskagqp | |
| | repqvyllppsrdeltknqvsltclakgfypkdvlvrwlqgsqelprenykttasvldsdgsff | |
| | vyskltvdksrwqqgnvfscmvghealplaftqksislsp | |
| 128 | hhhhhhepkscdkthtcppcpcchprvflfrpaledl11gseanvtcvvtglrdedpevkfnwy | 2838 |
| | vdgvevhnaktkpreeqycgcysvvsvltvlhqdwlngkeykckvsnkalpapiektiskagqp | |
| | repqvyllppsrdeltknqvsltclakgfypkdvlvrwlqgsqelprenykttasvldsdgsff | |
| | vyskltvaaedwkkgdtfscmvghealplaftqksisrsp | |
| 129 | hhhhhhepkscdkthtcppcpcchprvflfrpaledl11gseanvtcvvtglrdedpevkfnwy | 2839 |
| | vdgvevhnaktkpreeqycgcysvvsvltvlhqdwlngkeykckvsnkalpapiektiskagqp | |
| | repqvyllppsrdeltknqvsltclakgfypkdvlvrwlqgsqelprenykttasvldsdgsff | |
| | vyskltvdksrwqqgnvfscmvghealplaftqksislsp | |
| 130 | evqlvesggglvqpggslrlscaasgfnikdtyihwvrqapgkglewvariyptngytryadsv | 2741 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcpvpppppcchprlslhrpaledlllgseanltctl | |
| | tglrdasgatftwtpssgksavqgpperdlcgcysvssvlpgcaqpwnhgetftctaahpelkt | |
| | pltanitksgntfrpevhllpppseelalnelvtltclargfspkdvlvrwlqgsqelprekyl | |
| | twasrqepsqgtttfavtsilrvaaedwkkgdtfscmvghealplaftqktidrlagkpthvnv | |
| | svvmaevdgtcy | |
| 131 | evqlvesggglvqpggslrlscaasgfnikdtyihwvrqapgkglewvariyptngytryadsv | 2742 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvlpgcaqpwnhgetftctaahpelkt | |
| | pltanitksgntfrpevhllpppseelalnelvtltclargfspkdvlvrwlqgsqelprekyl | |
| | twasrqepsqgtttfavtsilrvaaedwkkgdtfscmvghealplaftqktidrlagkpthvnv | |
| | svvmaevdgtcy | |
| 132 | evqlvesggglvqpggslrlscaasgfnikdtyihwvrqapgkglewvariyptngytryadsv | 2840 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvlpgcaqpwnhgkeykck | |
| | vsnkalpapiektiskagqprepqvylipperdeitknqvslwclakgfypkdvlvrwlqgsqe | |
| | lprenykttasvldsdgsffvyskltvaaedwkkgdtfscmvghealplaftqksisrsp | |
| 133 | evqlvesggglvqpggslrlscaasgfnikdtyihwvrqapgkglewvariyptngytryadsv | 3090 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvlpgcaqpwnhgkeykck | |
| | vsnkalpapiektiskagqprepqvcllppsrdeltknqvslscaakgfypkdvlvrwlqgsqe | |
| | lprenykttasvldsdgsffvvskltvaaedwkkgdtfscmvghealplaftqksisrsp | |
| 134 | evqlvesggglvqpggslrlscaasgfnikdtyihwvrqapgkglewvariyptngytryadsv | 2842 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvlpgcaqpwnhgkeykck | |
| | vsnkalpapiektiskagqprepqvylipperdeitknqvslwclakgfypkdvlvrwlqgsqe | |
| | lprenykttasvldsdgsffvyskltvdksrwqqgnvfscmvghealplaftqksislsp | |
| 135 | evqlvesggglvqpggslrlscaasgfnikdtyihwvrqapgkglewvariyptngytryadsv | 3091 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvlpgcaqpwnhgkeykck | |
| | vsnkalpapiektiskagqprepqvcllppsrdeltknqvslscaakgfypkdvlvrwlqgsqe | |
| | lprenykttasvldsdgsffvvskltvdksrwqqgnvfscmvghealplaftqksislsp | |
| 136 | evqlvesggglvqpggslrlscaasgfnikdtyihwvrqapgkglewvariyptngytryadsv | 2841 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | vsnkalpapiektiskagqprepqvylipperdeitknqvslwclakgfypkdvlvrwlqgsqe | |
| | lprenykttasvldsdgsffvyskltvaaedwkkgdtfscmvghealplaftqksisrsp | |
| 137 | evqlvesggglvqpggslrlscaasgfnikdtyihwvrqapgkglewvariyptngytryadsv | 3092 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | vsnkalpapiektiskagqprepqvcllppsrdeltknqvslscaakgfypkdvlvrwlqgsqe | |
| | lprenykttasvldsdgsffvvskltvaaedwkkgdtfscmvghealplaftqksisrsp | |
| 138 | evqlvesggglvqpggslrlscaasgfnikdtyihwvrqapgkglewvariyptngytryadsv | 2843 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | vsnkalpapiektiskagqprepqvylipperdeitknqvslwclakgfypkdvlvrwlqgsqe | |
| | lprenykttasvldsdgsffvyskltvdksrwqqgnvfscmvghealplaftqksislsp | |
| 139 | evqlvesggglvqpggslrlscaasgfnikdtyihwvrqapgkglewvariyptngytryadsv | 3093 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | vsnkalpapiektiskagqprepqvcllppsrdeltknqvslscaakgfypkdvlvrwlqgsqe | |
| | lprenykttasvldsdgsffvvskltvdksrwqqgnvfscmvghealplaftqksislsp | |
| 140 | evqlvesggglvqpggslrlscaasgfnikdtyihwvrqapgkglewvariyptngytryadsv | 2844 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvlpgcaqpwnhgkeykck | |
| | vsnkalpapiektiskagqprepqvytlppcrdeltknqvslwclvkgfypsdiavewesngqp | |
| | ennykttppvldsdgsfflyskltvdksrwqqgnvfscsvmhealhnhytqksislsp | |
| 141 | evqlvesggglvqpggslrlscaasgfnikdtyihwvrqapgkglewvariyptngytryadsv | 2851 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvlpgcaqpwnhgkeykck | |
| | vsnkalpapiektiskagqprepqvctlppsrdeltknqvslscavkgfypsdiavewesngqp | |
| | ennykttppvldsdgsfflvskltvdksrwqqgnvfscsvmhealhnhytqkslslsp | |
| 142 | evqlvesggglvqpggslrlscaasgfnikdtyihwvrqapgkglewvariyptngytryadsv | 2845 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | vsnkalpapiektiskagqprepqvytlppcrdeltknqvslwclvkgfypsdiavewesngqp | |
| | ennykttppvldsdgsfflyskltvdksrwqqgnvfscsvmhealhnhytqkslslsp | |
| 143 | evqlvesggglvqpggslrlscaasgfnikdtyihwvrqapgkglewvariyptngytryadsv | 2852 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | vsnkalpapiektiskagqprepqvctlppsrdeltknqvslscavkgfypsdiavewesngqp | |
| | ennykttppvldsdgsfflvskltvdksrwqqgnvfscsvmhealhnhytqkslslsp | |
| 144 | evqlvesggglvqpggslrlscaasgfnikdtyihwvrqapgkglewvariyptngytryadsv | 2846 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfppkpkdtlmisrtp | |
| | evtcvvtglrdedpevkfnwyvdgvevhnaktkpreeqycgcysvvsvltvlhqdwlngkeykc | |
| | kvsnkalpapiektiskagqprepqvytlppcrdeltknqvslwclvkgfypsdiavewesngq | |
| | pennykttppvldsdgsfflyskltvdksrwqqgnvfscsvmhealhnhytqkslslsp | |
| 145 | evqlvesggglvqpggslrlscaasgfnikdtyihwvrqapgkglewvariyptngytryadsv | 2853 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfppkpkdtlmisrtp | |
| | evtcvvtglrdedpevkfnwyvdgvevhnaktkpreeqycgcysvvsvltvlhqdwlngkeykc | |
| | kvsnkalpapiektiskagqprepqvctlppsrdeltknqvslscavkgfypsdiavewesngq | |
| | pennykttppvldsdgsfflvskltvdksrwqqgnvfscsvmhealhnhytqkslslsp | |
| 146 | evqlvesggglvqpggslrlscaasgfnikdtyihwvrqapgkglewvariyptngytryadsv | 3082 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvlpgcaqpwnhgke | |
| | ykckvsnkalpapiektiskagqprepqvyllppsrdeltknqvsltclakgfypkdvlvrwlq | |
| | gsqelprenykttasvldsdgsffvyskltvaaedwkkgdtfscmvghealplaftqksisrsp | |
| 147 | evqlvesggglvqpggslrlscaasgfnikdtyihwvrqapgkglewvariyptngytryadsv | 3083 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvlpgcaqpwnhgke | |
| | ykckvsnkalpapiektiskagqprepqvyllppsrdeltknqvsltclakgfypkdvlvrwlq | |
| | gsqelprenykttasvldsdgsffvyskltvdksrwqqgnvfscmvghealplaftqkslslsp | |
| 148 | evqlvesggglvqpggslrlscaasgfnikdtyihwvrqapgkglewvariyptngytryadsv | 3084 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhqdwlngke | |
| | ykckvsnkalpapiektiskagqprepqvyllppsrdeltknqvsltclakgfypkdvlvrwlq | |
| | gsqelprenykttasvldsdgsffvyskltvaaedwkkgdtfscmvghealplaftqksisrsp | |
| 149 | evqlvesggglvqpggslrlscaasgfnikdtyihwvrqapgkglewvariyptngytryadsv | 3085 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhqdwlngke | |
| | ykckvsnkalpapiektiskagqprepqvyllppsrdeltknqvsltclakgfypkdvlvrwlq | |
| | gsqelprenykttasvldsdgsffvyskltvdksrwqqgnvfscmvghealplaftqkslslsp | |
| 150 | evqlvesggglvqpggslrlscaasgfnikdtyihwvrqapgkglewvariyptngytryadsv | 3086 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpdvfifrpaledlllg | |
| | seanvtcvvvdvshedpevkfnwyvdgvevhnaktkpreeqynstyrvvsvlpgcaqpwnhgke | |
| | ykckvsnkalpapeektiskagqprepqvyllppsrdeltknqvsltclakgfypkdvlvrwlq | |
| | gsqelprenykttasvldsdgsffvyskltvaaedwkkgdtfscmvghealplaftqksisrsp | |
| 151 | evqlvesggglvqpggslrlscaasgfnikdtyihwvrqapgkglewvariyptngytryadsv | 3087 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpdvfifrpaledlllg | |
| | seanvtcvvvdvshedpevkfnwyvdgvevhnaktkpreeqynstyrvvsvlpgcaqpwnhgke | |
| | ykckvsnkalpapeektiskagqprepqvyllppsrdeltknqvsltclakgfypkdvlvrwlq | |
| | gsqelprenykttasvldsdgsffvyskitvdksrwqqgnvfscmvghealplaftqksislsp | |
| 152 | evqlvesggglvqpggslrlscaasgfnikdtyihwvrqapgkglewvariyptngytryadsv | 3088 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpdvfifrpaledlllg | |
| | seanvtcvvvdvshedpevkfnwyvdgvevhnaktkpreeqynstyrvvsvltvlhqdwlngke | |
| | ykckvsnkalpapeektiskagqprepqvyllppsrdeltknqvsltclakgfypkdvlvrwlq | |
| | gsqelprenykttasvldsdgsffvyskltvaaedwkkgdtfscmvghealplaftqksisrsp | |
| 153 | evqlvesggglvqpggslrlscaasgfnikdtyihwvrqapgkglewvariyptngytryadsv | 3089 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpdvfifrpaledlllg | |
| | seanvtcvvvdvshedpevkfnwyvdgvevhnaktkpreeqynstyrvvsvltvlhqdwlngke | |
| | ykckvsnkalpapeektiskagqprepqvyllppsrdeltknqvsltclakgfypkdvlvrwlq | |
| | gsqelprenykttasvldsdgsffvyskitvdksrwqqgnvfscmvghealplaftqksislsp | |
| 154 | evqlvesggglvqpggslrlscaasgfnikdtyihwvrqapgkglewvariyptngytryadsv | 3094 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfppkpkdtlmisrtp | |
| | evtcvvtglrdedpevkfnwyvdgvevhnaktkpreeqycgcysvvsvltvlhqdwlngkeykc | |
| | kvsnkalpapiektiskagqprepqvytlppcrdeltknqvslwclvkgfypsdiavewesngq | |
| | pennykttppvldsdgsfflyskltvdksrwqqgnvfscsvlhealhshytqkslslsp | |
| 155 | evqlvesggglvqpggslrlscaasgfnikdtyihwvrqapgkglewvariyptngytryadsv | 3095 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvlpgcaqpwnhgke | |
| | ykckvsnkalpapiektiskagqprepqvcllppsrdeltknqvslscaakgfypkdvlvrwlq | |
| | gsqelprenykttasvldsdgsffvvskltvaaedwkkgdtfscmvghealplaftqksisrsp | |
| 156 | evqlvesggglvqpggslrlscaasgfnikdtyihwvrqapgkglewvariyptngytryadsv | 3096 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvlpgcaqpwnhgke | |
| | ykckvsnkalpapiektiskagqprepqvcllppsrdeltknqvslscaakgfypkdvlvrwlq | |
| | gsqelprenykttasvldsdgsffvvskltvdksrwqqgnvfscmvghealplaftqksislsp | |
| 157 | evqlvesggglvqpggslrlscaasgfnikdtyihwvrqapgkglewvariyptngytryadsv | 3097 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhqdwlngke | |
| | ykckvsnkalpapiektiskagqprepqvcllppsrdeltknqvslscaakgfypkdvlvrwlq | |
| | gsqelprenykttasvldsdgsffvvskltvaaedwkkgdtfscmvghealplaftqksisrsp | |
| 158 | evqlvesggglvqpggslrlscaasgfnikdtyihwvrqapgkglewvariyptngytryadsv | 3098 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhqdwlngke | |
| | ykckvsnkalpapiektiskagqprepqvcllppsrdeltknqvslscaakgfypkdvlvrwlq | |
| | gsqelprenykttasvldsdgsffvvskltvdksrwqqgnvfscmvghealplaftqksislsp | |
| 159 | evqlvesggglvqpggslrlscaasgfnikdtyihwvrqapgkglewvariyptngytryadsv | 3099 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfppkpkdtlmi | |
| | srtpevtcvvvdvshedpevkfnwyvdgvevhnaktkpreeqynstyrvvsvltvlhqdwlngk | |
| | eykckvsnkalpapiektiskagqprepqvytlppcrdeltknqvslwclvkgfypsdiavewe | |
| | sngqpennykttppvldsdgsfflyskltvdksrwqqgnvfscsvmhealhnhytqkslslsp | |
| 160 | evqlvesggglvqpggslrlscaasgfnikdtyihwvrqapgkglewvariyptngytryadsv | 3100 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfppkpkdtlmi | |
| | srtpevtcvvvdvshedpevkfnwyvdgvevhnaktkpreeqynstyrvvsvltvlhqdwlngk | |
| | eykckvsnkalpapiektiskagqprepqvytlppcrdeltknqvslwclvkgfypsdiavewe | |
| | sngqpennykttppvldsdgsfflyskltvdksrwqqgnvfscsvlhealhshytqkslslsp | |
| 161 | evqlvesggglvqpggslrlscaasgfnikdtyihwvrqapgkglewvariyptngytryadsv | 3101 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpdvfifppkpkdtlmi | |
| | srtpevtcvvvdvshedpevkfnwyvdgvevhnaktkpreeqynstyrvvsvltvlhqdwlngk | |
| | eykckvsnkalpapeektiskagqprepqvytlppcrdeltknqvslwclvkgfypsdiavewe | |
| | sngqpennykttppvldsdgsfflyskltvdksrwqqgnvfscsvmhealhnhytqkslslsp | |
| 162 | evqlvesggglvqpggslrlscaasgfnikdtyihwvrqapgkglewvariyptngytryadsv | 3102 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpdvfifppkpkdtlmi | |
| | srtpevtcvvvdvshedpevkfnwyvdgvevhnaktkpreeqynstyrvvsvltvlhqdwlngk | |
| | eykckvsnkalpapeektiskagqprepqvytlppcrdeltknqvslwclvkgfypsdiavewe | |
| | sngqpennykttppvldsdgsfflyskltvdksrwqqgnvfscsvlhealhshytqkslslsp | |
| 163 | evqlvesggglvqpggslrlscaasgfnikdtyihwvrqapgkglewvariyptngytryadsv | 3011 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfppkpkdtlyitrep | |
| | evtcvvtglrdedpevkfnwyvdgvevhnaktkpreeqycgcysvvsvltvlhqdwlngkeykc | |
| | kvsnkalpapiektiskagqprepqvytlppcrdeltknqvslwclvkgfypsdiavewesngq | |
| | pennykttppvldsdgsfflyskltvdksrwqqgnvfscsvmhealhnhytqkslslsp | |
| 164 | evqlvesggglvqpggslrlscaasgfnikdtyihwvrqapgkglewvariyptngytryadsv | 3013 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellcchpdvfifppkpkdtlm | |
| | isrtpevtcvvtglrdedpevkfnwyvdgvevhnaktkpreeqycgcysvvsvltvlhqdwlng | |
| | keykckvsnkalpapeektiskagqprepqvytlppcrdeltknqvslwclvkgfypsdiavew | |
| | esngqpennykttppvldsdgsfflyskltvdksrwqqgnvfscsvmhealhnhytqkslslsp | |
| 165 | evqlvesggglvqpggslrlscaasgfnikdtyihwvrqapgkglewvariyptngytryadsv | 3014 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellcchprvflfppaledlll | |
| | gseanvtcvvtglrdedpevkfnwyvdgvevhnaktkpreeqycgcysvvsvltvlhqdwlngk | |
| | eykckvsnkalpapiektiskagqprepqvyllppsrdeltknqvsltclakgfypkdvlvrwl | |
| | qgsqelprenykttasvldsdgsffvyskltvaaedwkkgdtfscmvghealplaftqksisrs | |
| | p | |
| 166 | evqlvesggglvqpggslrlscaasgfnikdtyihwvrqapgkglewvariyptngytryadsv | 3015 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellcchprvflfppaledlll | |
| | gseanvtcvvtglrdedpevkfnwyvdgvevhnaktkpreeqynstyrvvsvltvlhqdwlngk | |
| | eykckvsnkalpapiektiskagqprepqvyllppsrdeltknqvsltclakgfypkdvlvrwl | |
| | qgsqelprenykttasvldsdgsffvyskltvaaedwkkgdtfscmvghealplaftqksisrs | |
| | p | |
| 167 | evqlvesggglvqpggslrlscaasgfnikdtyihwvrqapgkglewvariyptngytryadsv | 3016 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellcchprvflfppaledlll | |
| | gseanvtcvvvdvshedpevkfnwyvdgvevhnaktkpreeqynstyrvvsvltvlhqdwlngk | |
| | eykckvsnkalpapiektiskagqprepqvyllppsrdeltknqvsltclakgfypkdvlvrwl | |
| | qgsqelprenykttasvldsdgsffvyskltvaaedwkkgdtfscmvghealplaftqksisrs | |
| | p | |
| 168 | evqlvesggglvqpggslrlscaasgfnikdtyihwvrqapgkglewvariyptngytryadsv | 3017 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfppaledlllg | |
| | seanvtcvvvdvshedpevkfnwyvdgvevhnaktkpreeqynstyrvvsvltvlhqdwlngke | |
| | ykckvsnkalpapiektiskagqprepqvyllppsrdeltknqvsltclakgfypkdvlvrwlq | |
| | gsqelprenykttasvldsdgsffvyskltvaaedwkkgdtfscmvghealplaftqksisrsp | |
| 169 | evqlvesggglvqpggslrlscaasgfnikdtyihwvrqapgkglewvariyptngytryadsv | 3018 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfppaledlllg | |
| | seanvtcvvtglrdedpevkfnwyvdgvevhnaktkpreeqynstyrvvsvltvlhqdwlngke | |
| | ykckvsnkalpapiektiskagqprepqvyllppsrdeltknqvsltclakgfypkdvlvrwlq | |
| | gsqelprenykttasvldsdgsffvyskltvaaedwkkgdtfscmvghealplaftqksisrsp | |
| 170 | evqlvesggglvqpggslrlscaasgfnikdtyihwvrqapgkglewvariyptngytryadsv | 3019 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfppaledlllg | |
| | seanvtcvvtglrdedpevkfnwyvdgvevhnaktkpreeqycgcysvvsvltvlhqdwlngke | |
| | ykckvsnkalpapiektiskagqprepqvyllppsrdeltknqvsltclakgfypkdvlvrwlq | |
| | gsqelprenykttasvldsdgsffvyskltvaaedwkkgdtfscmvghealplaftqksisrsp | |
| 171 | evqlvesggglvqpggslrlscaasgfnikdtyihwvrqapgkglewvariyptngytryadsv | 3020 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfppaledlllg | |
| | seanvtcvvvdvshedpevkfnwyvdgvevhnaktkpreeqycgcysvvsvltvlhqdwlngke | |
| | ykckvsnkalpapiektiskagqprepqvyllppsrdeltknqvsltclakgfypkdvlvrwlq | |
| | gsqelprenykttasvldsdgsffvyskltvaaedwkkgdtfscmvghealplaftqksisrsp | |
| 172 | evqlvesggglvqpggslrlscaasgfnikdtyihwvrqapgkglewvariyptngytryadsv | 3021 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcplggpsvflfppaledlllgsean | |
| | vtcvvvdvshedpevkfnwyvdgvevhnaktkpreeqycgcysvvsvltvlhqdwlngkeykck | |
| | vsnkalpapiektiskagqprepqvyllppsrdeltknqvsltclakgfypkdvlvrwlqgsqe | |
| | lprenykttasvldsdgsffvyskltvaaedwkkgdtfscmvghealplaftqksisrsp | |
| 173 | evqlvesggglvqpggslrlscaasgfnikdtyihwvrqapgkglewvariyptngytryadsv | 3022 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcplggpsvflfppaledlllgsean | |
| | vtcvvvdvshedpevkfnwyvdgvevhnaktkpreeqynstyrvvsvltvlhqdwlngkeykck | |
| | vsnkalpapiektiskagqprepqvyllppsrdeltknqvsltclakgfypkdvlvrwlqgsqe | |
| | lprenykttasvldsdgsffvyskltvaaedwkkgdtfscmvghealplaftqksisrsp | |
| 174 | evqlvesggglvqpggslrlscaasgfnikdtyihwvrqapgkglewvariyptngytryadsv | 3023 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | vsnkalpapiektiskagqprrpevyllppsrdeltknqvsltclakgfypkdvlvrwlqgsqe | |
| | lprenykttasvldsdgsffvyskltvaaedwkkgdtfscmvghealplaftqksisrsp | |
| 175 | evqlvesggglvqpggslrlscaasgfnikdtyihwvrqapgkglewvariyptngytryadsv | 3024 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfppaledlllgsean | |
| | vtcvvtglrdedpevkfnwyvdgvevhnaktkpreeqycgcysvvsvltvlhqdwlngkeykck | |
| | vsnkalpapiektiskagqprrpevyllppsrdeltknqvsltclakgfypkdvlvrwlqgsqe | |
| | lprenykttasvldsdgsffvyskltvaaedwkkgdtfscmvghealplaftqksisrsp | |
| 176 | evqlvesggglvqpggslrlscaasgfnikdtyihwvrqapgkglewvariyptngytryadsv | 3025 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdasgatfnwyvdgvevhnakqgpperdlcgcysvvsvlpgcaqpwnhgkeykcka | |
| | ahpelktpiektiskagqprepqvyllppsrdeltknqvsltclakgfypkdvlvrwlqgsqel | |
| | prenykttasvidsdgsffvyskltvaaedwkkgdtfscmvghealplaftqksisrsp | |
| 177 | evqlvesggglvqpggslrlscaasgfnikdtyihwvrqapgkglewvariyptngytryadsv | 3026 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdasgatfnwyvdgvevhnakqgpperdlcgcysvvsvltvlhqdwlngkeykcka | |
| | ahpelktpiektiskagqprepqvyllppsrdeltknqvsltclakgfypkdvlvrwlqgsqel | |
| | prenykttasvidsdgsffvyskltvaaedwkkgdtfscmvghealplaftqksisrsp | |
| 178 | evqlvesggglvqpggslrlscaasgfnikdtyihwvrqapgkglewvariyptngytryadsv | 3027 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellcchprvflfrpaledlll | |
| | gseanvtcvvtglrdasgatfnwyvdgvevhnakqgpperdlcgcysvvsvlpgcaqpwnhgke | |
| | ykckaahpelktpiektiskagqprepqvyllppsrdeltknqvsltclakgfypkdvlvrwlq | |
| | gsqelprenykttasvldsdgsffvyskltvaaedwkkgdtfscmvghealplaftqksisrsp | |
| 179 | evqlvesggglvqpggslrlscaasgfnikdtyihwvrqapgkglewvariyptngytryadsv | 3028 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellcchprvflfrpaledlll | |
| | gseanvtcvvtglrdasgatfnwyvdgvevhnakqgpperdlcgcysvvsvltvlhqdwlngke | |
| | ykckaahpelktpiektiskagqprepqvyllppsrdeltknqvsltclakgfypkdvlvrwlq | |
| | gsqelprenykttasvldsdgsffvyskltvaaedwkkgdtfscmvghealplaftqksisrsp | |
| 180 | evqlvesggglvqpggslrlscaasgfnikdtyihwvrqapgkglewvariyptngytryadsv | 3029 |
| | kgrftisadtskntaylqmnslraedtavyycsrwggdgfyamdywgqgtlvtvssastkgpsv | |
| | fplapsskstsggtaalgclvkdvfpepvtvswnsgaltsgvhtfpavlqssglvslssvvtvp | |
| | ssslgtqtyicnvnhkpsntkvdkkvepkscdkthtcppcpapellggpsvflfrpaledlllg | |
| | seanvtcvvtglrdasgatfnwyvdgvevhnakqgpperdlcgcysvvsvlpgcaqpwnhgkey | |
| | kckaahpelktpiektiskagqprepqvyllppsrdeltknqvsltclakgfypkdvlvrwlqg | |
| | sqelprenykttasvldsdgsffvyskltvaaedwkkgdtfscmvghealplaftqksisrsp | |
| 181 | evqlvesggglvqpggslrlscaasgfnikdtyihwvrqapgkglewvariyptngytryadsv | 3030 |
| | kgrftisadtskntaylqmnslraedtavyycsrwggdgfyamdywgqgtlvtvssastkgpsv | |
| | fplapsskstsggtaalgclvkdvfpepvtvswnsgaltsgvhtfpavlqssglvslssvvtvp | |
| | ssslgtqtyicnvnhkpsntkvdkkvepkscdkthtcppcpapellggpsvflfrpaledlllg | |
| | seanvtcvvtglrdasgatfnwyvdgvevhnakqgpperdlcgcysvvsvltvlhqdwlngkey | |
| | kckaahpelktpiektiskagqprepqvyllppsrdeltknqvsltclakgfypkdvlvrwlqg | |
| | sqelprenykttasvldsdgsffvyskltvaaedwkkgdtfscmvghealplaftqksisrsp | |
| 182 | Evqlvesggglvqpggslrlscaasgfnikdtyihwvrqapgkglewvariyptngytryadsv | 3240 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvlpgcaepwnhgetftctaahpelkt | |
| | pltanitksgntfrpevhllpppseelalnelvtltclargfspkdvlvrwlqgsqelprekyl | |
| | twasrqepsqgtttfavtsilrvaaedwkkgdtfscmvghealplaftqktidrla | |
| 183 | evqlvesggglvqpggslrlscaasgfnikdtyihwvrqapgkglewvariyptngytryadsv | 3241 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvlpgcaqpwyhgetftctaahpelkt | |
| | pltanitksgntfrpevhllpppseelalnelvtltclargfspkdvlvrwlqgsqelprekyl | |
| | twasrqepsqgtttfavtsilrvaaedwkkgdtfscmvghealplaftqktidrla | |
| 184 | evqlvesggglvqpggslrlscaasgfnikdtyihwvrqapgkglewvariyptngytryadsv | 3242 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvlpgcaqpwnhgetftctaahpelkt | |
| | pltanitksgntfrpevhllpppseelalnelvtltclargfspkdvlvrwlqgdqelprekyl | |
| | twasrqepsqgtttfavtsilrvaaedwkkgdtfscmvghealplaftqktidrla | |
| 185 | evqlvesggglvqpggslrlscaasgfnikdtyihwvrqapgkglewvariyptngytryadsv | 3243 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvlpgcaqpwnhgetftctaahpelkt | |
| | pltanitksgntfrpevhllpppseelalnelvtltclargfspkdvlvrwlqgsqelprekyl | |
| | twasrqepsqgtttfavtsilrvaaedwkkgdtfscyvghealplaftqktidrla | |
| 186 | evqlvesggglvqpggslrlscaasgfnikdtyihwvrqapgkglewvariyptngytryadsv | 3244 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvlpgcaqpwnhgetftctaahpelkt | |
| | pltanitksgntfrpevhllpppseelalnelvtltclargfspkdvlvrwlqgsqelprekyl | |
| | twasrqepsqgtttfavtsilrvaaedwkkgdtfscmvghealplaftyktidrla | |
| 187 | evqlvesggglvqpggslrlscaasgfnikdtyihwvrqapgkglewvariyptngytryadsv | 3245 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvlpgcaepwyhgetftctaahpelkt | |
| | pltanitksgntfrpevhllpppseelalnelvtltclargfspkdvlvrwlqgsqelprekyl | |
| | twasrqepsqgtttfavtsilrvaaedwkkgdtfscmvghealplaftqktidrla | |
| 188 | evqlvesggglvqpggslrlscaasgfnikdtyihwvrqapgkglewvariyptngytryadsv | 3246 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhedwlngkeykck | |
| | vsnkalpapiektiskagqprepqvyllppsrdeltknqvsltclakgfypkdvlvrwlqgsqe | |
| | lprenykttasvldsdgsffvyskltvaaedwkkgdtfscmvghealplaftqksisrsp | |
| 189 | evqlvesggglvqpggslrlscaasgfnikdtyihwvrqapgkglewvariyptngytryadsv | 3247 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwyngkeykck | |
| | vsnkalpapiektiskagqprepqvyllppsrdeltknqvsltclakgfypkdvlvrwlqgsqe | |
| | lprenykttasvldsdgsffvyskltvaaedwkkgdtfscmvghealplaftqksisrsp | |
| 190 | evqlvesggglvqpggslrlscaasgfnikdtyihwvrqapgkglewvariyptngytryadsv | 3248 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | vsnkalpapiektiskagqprepqvyllppsrdeltknqvsltclakgfypkdvlvrwlqgdqe | |
| | lprenykttasvldsdgsffvyskltvaaedwkkgdtfscmvghealplaftqksisrsp | |
| 191 | Evqlvesggglvqpggslrlscaasgfnikdtyihwvrqapgkglewvariyptngytryadsv | 3249 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | vsnkalpapiektiskagqprepqvyllppsrdeltknqvsltclakgfypkdvlvrwlqgsqe | |
| | lprenykttasvldsdgsffvyskltvaaedwkkgdtfscyvghealplaftqksisrsp | |
| 192 | evqlvesggglvqpggslrlscaasgfnikdtyihwvrqapgkglewvariyptngytryadsv | 3250 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | vsnkalpapiektiskagqprepqvyllppsrdeltknqvsltclakgfypkdvlvrwlqgsqe | |
| | lprenykttasvldsdgsffvyskltvaaedwkkgdtfscmvghealplaftyksisrsp | |
| 193 | evqlvesggglvqpggslrlscaasgfnikdtyihwvrqapgkglewvariyptngytryadsv | 3251 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhedwyngkeykck | |
| | vsnkalpapiektiskagqprepqvyllppsrdeltknqvsltclakgfypkdvlvrwlqgsqe | |
| | lprenykttasvldsdgsffvyskltvaaedwkkgdtfscmvghealplaftqksisrsp | |
| 194 | evqlvesggglvqpggslrlscaasgfnikdtyihwvrqapgkglewvariyptngytryadsv | 3252 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhedwlngke | |
| | ykckvsnkalpapiektiskagqprepqvyllppsrdeltknqvsltclakgfypkdvlvrwlq | |
| | gsqelprenykttasvldsdgsffvyskltvaaedwkkgdtfscmvghealplaftqksisrsp | |
| 195 | evqlvesggglvqpggslrlscaasgfnikdtyihwvrqapgkglewvariyptngytryadsv | 3253 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhqdwyngke | |
| | ykckvsnkalpapiektiskagqprepqvyllppsrdeltknqvsltclakgfypkdvlvrwlq | |
| | gsqelprenykttasvldsdgsffvyskltvaaedwkkgdtfscmvghealplaftqksisrsp | |
| 196 | evqlvesggglvqpggslrlscaasgfnikdtyihwvrqapgkglewvariyptngytryadsv | 3254 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhqdwlngke | |
| | ykckvsnkalpapiektiskagqprepqvyllppsrdeltknqvsltclakgfypkdvlvrwlq | |
| | gdqelprenykttasvldsdgsffvyskltvaaedwkkgdtfscmvghealplaftqksisrsp | |
| 197 | evqlvesggglvqpggslrlscaasgfnikdtyihwvrqapgkglewvariyptngytryadsv | 3255 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhqdwlngke | |
| | ykckvsnkalpapiektiskagqprepqvyllppsrdeltknqvsltclakgfypkdvlvrwlq | |
| | gsqelprenykttasvldsdgsffvyskltvaaedwkkgdtfscyvghealplaftqksisrsp | |
| 198 | evqlvesggglvqpggslrlscaasgfnikdtyihwvrqapgkglewvariyptngytryadsv | 3256 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhqdwlngke | |
| | ykckvsnkalpapiektiskagqprepqvyllppsrdeltknqvsltclakgfypkdvlvrwlq | |
| | gsqelprenykttasvldsdgsffvyskltvaaedwkkgdtfscmvghealplaftyksisrsp | |
| 199 | evqlvesggglvqpggslrlscaasgfnikdtyihwvrqapgkglewvariyptngytryadsv | 3257 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhedwyngke | |
| | ykckvsnkalpapiektiskagqprepqvyllppsrdeltknqvsltclakgfypkdvlvrwlq | |
| | gsqelprenykttasvldsdgsffvyskltvaaedwkkgdtfscmvghealplaftqksisrsp | |
| 200 | evqlvesggglvqpggslrlscaasgfnikdtyihwvrqapgkglewvariyptngytryadsv | 3258 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvlpgcaqpwnhgetftctaahpelkt | |
| | pltanitksgntfrpevhllpppseelalnelvtltclargfspkdvlvrwlqgsqelprekyl | |
| | twasrqepsqgtttfavtsilrvaaedwkkgdtfscyvghealplaftyktidrla | |
| 201 | evqlvesggglvqpggslrlscaasgfnikdtyihwvrqapgkglewvariyptngytryadsv | 3259 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtamfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvlpgcaqpwnhgetftctaahpelkt | |
| | pltanitksgntfrpevhllpppseelalnelvtltclargfspkdvlvrwlqgdqelprekyl | |
| | twasrqepsqgtttfavtsilrvaaedwkkgdtfscyvghealplaftyktidrla | |
| 202 | evqlvesggglvqpggslrlscaasgfnikdtyihwvrqapgkglewvariyptngytryadsv | 3260 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtamfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvipgcaepwyhgetftctaahpelkt | |
| | pltanitksgntfrpevhllpppseelalnelvtltclargfspkdvlvrwlqgdqelprekyl | |
| | twasrqepsqgtttfavtsilrvaaedwkkgdtfscmvghealplaftqktidrla | |
| 203 | evqlvesggglvqpggslrlscaasgfnikdtyihwvrqapgkglewvariyptngytryadsv | 3261 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtamfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvipgcaepwyhgetftctaahpelkt | |
| | pltanitksgntfrpevhllpppseelalnelvtltclargfspkdvlvrwlqgsqelprekyl | |
| | twasrqepsqgtttfavtsilrvaaedwkkgdtfscyvghealplaftyktidrla | |
| 204 | evqlvesggglvqpggslrlscaasgfnikdtyihwvrqapgkglewvariyptngytryadsv | 3262 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtamfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvipgcaepwyhgetftctaahpelkt | |
| | pltanitksgntfrpevhllpppseelalnelvtltclargfspkdvlvrwlqgdqelprekyl | |
| | twasrqepsqgtttfavtsilrvaaedwkkgdtfscyvghealplaftyktidrla | |
| 205 | evqlvesggglvqpggslrlscaasgfnikdtyihwvrqapgkglewvariyptngytryadsv | 3263 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | vsnkalpapiektiskagqprepqvyllppsrdeltknqvsltclakgfypkdvlvrwlqgsqe | |
| | lprenykttasvldsdgsffvyskltvaaedwkkgdtfscyvghealplaftyksisrsp | |
| 206 | evqlvesggglvqpggslrlscaasgfnikdtyihwvrqapgkglewvariyptngytryadsv | 3264 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | vsnkalpapiektiskagqprepqvyllppsrdeltknqvsltclakgfypkdvlvrwlqgdqe | |
| | lprenykttasvldsdgsffvyskltvaaedwkkgdtfscyvghealplaftyksisrsp | |
| 207 | evqlvesggglvqpggslrlscaasgfnikdtyihwvrqapgkglewvariyptngytryadsv | 3265 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhedwyngkeykck | |
| | vsnkalpapiektiskagqprepqvyllppsrdeltknqvsltclakgfypkdvlvrwlqgdqe | |
| | lprenykttasvldsdgsffvyskltvaaedwkkgdtfscmvghealplaftqksisrsp | |
| 208 | evqlvesggglvqpggslrlscaasgfnikdtyihwvrqapgkglewvariyptngytryadsv | 3266 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhedwyngkeykck | |
| | vsnkalpapiektiskagqprepqvyllppsrdeltknqvsltclakgfypkdvlvrwlqgsqe | |
| | lprenykttasvldsdgsffvyskltvaaedwkkgdtfscyvghealplaftyksisrsp | |
| 209 | evqlvesggglvqpggslrlscaasgfnikdtyihwvrqapgkglewvariyptngytryadsv | 3267 |
| | kgrftisadtskntaylqmnslraedtavyycsrwggdgfyamdywgqgtlvtvssastkgpsv | |
| | fplapsskstsqqtaalqclvkdyfpepvtvswnsqaltsqvhtfpavlqssqlyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhedwyngkeykck | |
| | vsnkalpapiektiskagqprepqvyllppsrdeltknqvsltclakgfypkdvlvrwlqgdqe | |
| | lprenykttasvldsdgsffvyskltvaaedwkkgdtfscyvghealplaftyksisrsp | |
| 210 | evqlvesggglvqpggslrlscaasgfnikdtyihwvrqapgkglewvariyptngytryadsv | 3268 |
| | kgrftisadtskntaylqmnslraedtavyycsrwggdgfyamdywgqgtlvtvssastkgpsv | |
| | fplapsskstsggtaalgclvkdvfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhqdwlngke | |
| | ykckvsnkalpapiektiskagqprepqvyllppsrdeltknqvsltclakgfypkdvlvrwlq | |
| | gsqelprenykttasvldsdgsffvyskltvaaedwkkgdtfscyvghealplaftyksisrsp | |
| 211 | evqlvesggglvqpggslrlscaasgfnikdtyihwvrqapgkglewvariyptngytryadsv | 3269 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhqdwlngke | |
| | ykckvsnkalpapiektiskagqprepqvyllppsrdeltknqvsltclakgfypkdvlvrwlq | |
| | gdqelprenykttasvldsdgsffvyskltvaaedwkkgdtfscyvghealplaftyksisrsp | |
| 212 | evqlvesggglvqpggslrlscaasgfnikdtyihwvrqapgkglewvariyptngytryadsv | 3270 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhedwyngke | |
| | ykckvsnkalpapiektiskagqprepqvyllppsrdeltknqvsltclakgfypkdvlvrwlq | |
| | gdqelprenykttasvldsdgsffvyskltvaaedwkkgdtfscmvghealplaftqksisrsp | |
| 213 | evqlvesggglvqpggslrlscaasgfnikdtyihwvrqapgkglewvariyptngytryadsv | 3271 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhedwyngke | |
| | ykckvsnkalpapiektiskagqprepqvyllppsrdeltknqvsltclakgfypkdvlvrwlq | |
| | gsqelprenykttasvldsdgsffvyskltvaaedwkkgdtfscyvghealplaftyksisrsp | |
| 214 | evqlvesggglvqpggslrlscaasgfnikdtyihwvrqapgkglewvariyptngytryadsv | 3272 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhedwyngke | |
| | ykckvsnkalpapiektiskagqprepqvyllppsrdeltknqvsltclakgfypkdvlvrwlq | |
| | gdqelprenykttasvldsdgsffvyskltvaaedwkkgdtfscyvghealplaftyksisrsp | |
| 215 | evqlvesggglvqpggslrlscaasgfnikdtyihwvrqapgkglewvariyptngytryadsv | 3273 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpsledlllgseanltctl | |
| | tglrdasgatftwtpssgksavqgpperdlcgcysvssvipgcaqpwnhgetftctaahpelkt | |
| | pltanitksgntfrpevhllpppseelalnelvtltclargfspkdvlvrwlqgsqelprekyl | |
| | twasrqepsqgtttfavtsilrvaaedwkkgdtfscmvghealplaftqktidrla | |
| 216 | evqlvesggglvqpggslrlscaasgfnikdtyihwvrqapgkglewvariyptngytryadsv | 3274 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvipgcaqpwnhgetftctaahpelkt | |
| | pltanitksgntfrpevhllpppseelalnelvtltclargfspkdvlvrwlqgsqelprekyl | |
| | twasrqepsqgtttfavtsilrvaaedwkkgdtfscmvghealplaftqktidrla | |
| 217 | evqlvesggglvqpggslrlscaasgfnikdtyihwvrqapgkglewvariyptngytryadsv | 3275 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvlpvcaqpwnhgetftctaahpelkt | |
| | pltanitksgntfrpevhllpppseelalnelvtltclargfspkdvlvrwlqgsqelprekyl | |
| | twasrqepsqgtttfavtsilrvaaedwkkgdtfscmvghealplaftqktidrla | |
| 218 | evqlvesggglvqpggslrlscaasgfnikdtyihwvrqapgkglewvariyptngytryadsv | 3276 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvlpgcaqpwnhwetftctaahpelkt | |
| | pltanitksgntfrpevhllpppseelalnelvtltclargfspkdvlvrwlqgsqelprekyl | |
| | twasrqepsqgtttfavtsilrvaaedwkkgdtfscmvghealplaftqktidrla | |
| 219 | evqlvesggglvqpggslrlscaasgfnikdtyihwvrqapgkglewvariyptngytryadsv | 3277 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvlpgcaqpwnhgetftctaahpelkt | |
| | pltanitksgntfrpevhllpppseelalnelvtltclargfspkdvlvrwlqgsqelprekyl | |
| | twasrqepsqgtttfavtsilrvaaedwkkgdtfscmvghdalplaftqktidrla | |
| 220 | evqlvesggglvqpggslrlscaasgfnikdtyihwvrqapgkglewvariyptngytryadsv | 3278 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvlpgcaqpwnhgetftctaahpelkt | |
| | pltanitksgntfrpevhllpppseelalnelvtltclargfspkdvlvrwlqgsqelprekyl | |
| | twasrqepsqgtttfavtsilrvaaedwkkgdtfscmvghealplaftqktidrfa | |
| 221 | evqlvesggglvqpggslrlscaasgfnikdtyihwvrqapgkglewvariyptngytryadsv | 3279 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvipvcaepwyhwetftctaahpelkt | |
| | pltanitksgntfrpevhllpppseelalnelvtltclargfspkdvlvrwlqgsqelprekyl | |
| | twasrqepsqgtttfavtsilrvaaedwkkgdtfscmvghealplaftqktidrla | |
| 222 | evqlvesggglvqpggslrlscaasgfnikdtyihwvrqapgkglewvariyptngytryadsv | 3280 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvlpgcaqpwhhgetftctaahpelkt | |
| | pltanitksgntfrpevhllpppseelalnelvtltclargfspkdvlvrwlqgsqelprekyl | |
| | twasrqepsqgtttfavtsilrvaaedwkkgdtfscmvghealplaftqktidrla | |
| 223 | evqlvesggglvqpggslrlscaasgfnikdtyihwvrqapgkglewvariyptngytryadsv | 3281 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvitvlhedwynwkeykck | |
| | vsnkalpapiektiskagqprepqvyllppsrdeltknqvsltclakgfypkdvlvrwlqgsqe | |
| | lprenykttasvldsdgsffvyskltvaaedwkkgdtfscmvghealplaftqksisrsp | |
| 224 | Evqlvesggglvqpggslrlscaasgfnikdtyihwvrqapgkglewvariyptngytryadsv | 3282 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvitvlhedwynwke | |
| | ykckvsnkalpapiektiskagqprepqvyllppsrdeltknqvsltclakgfypkdvlvrwlq | |
| | gsqelprenykttasvldsdgsffvyskitvaaedwkkgdtfscmvghealplaftqksisrsp | |
| 225 | evqlvesggglvqpggslrlscaasgfnikdtyihwvrqapgkglewvariyptngytryadsv | 3309 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvipgcaepwnhgetftctaahpelkt | |
| | pltanitksgntfrpevhllpppseelalnelvtltclargfspkdvlvrwlqgdqelprekyl | |
| | twasrqepsqgtttfavtsilrvaaedwkkgdtfscmvghealplaftqktidrla | |
| 226 | evqlvesggglvqpggslrlscaasgfnikdtyihwvrqapgkglewvariyptngytryadsv | 3310 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvipgcaepwnhgetftctaahpelkt | |
| | pltanitksgntfrpevhllpppseelalnelvtltclargfspkdvlvrwlqgsqelprekyl | |
| | twasrqepsqgtttfavtsilrvaaedwkkgdtfscyvghealplaftqktidrla | |
| 227 | evqlvesggglvqpggslrlscaasgfnikdtyihwvrqapgkglewvariyptngytryadsv | 3311 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvipgcaqpwyhgetftctaahpelkt | |
| | pltanitksgntfrpevhllpppseelalnelvtltclargfspkdvlvrwlqgdqelprekyl | |
| | twasrqepsqgtttfavtsilrvaaedwkkgdtfscmvghealplaftqktidrla | |
| 228 | evqlvesggglvqpggslrlscaasgfnikdtyihwvrqapgkglewvariyptngytryadsv | 3312 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvlpgcaqpwyhgetftctaahpelkt | |
| | pltanitksgntfrpevhllpppseelalnelvtltclargfspkdvlvrwlqgsqelprekyl | |
| | twasrqepsqgtttfavtsilrvaaedwkkgdtfscyvghealplaftqktidrla | |
| 229 | evqlvesggglvqpggslrlscaasgfnikdtyihwvrqapgkglewvariyptngytryadsv | 3313 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvipgcaqpwnhgetftctaahpelkt | |
| | pltanitksgntfrpevhllpppseelalnelvtltclargfspkdvlvrwlqgdqelprekyl | |
| | twasrqepsqgtttfavtsilrvaaedwkkgdtfscyvghealplaftqktidrla | |
| 230 | evqlvesggglvqpggslrlscaasgfnikdtyihwvrqapgkglewvariyptngytryadsv | 3314 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvipgcaepwyhgetftctaahpelkt | |
| | pltanitksgntfrpevhllpppseelalnelvtltclargfspkdvlvrwlqgsqelprekyl | |
| | twasrqepsqgtttfavtsilrvaaedwkkgdtfscyvghealplaftqktidrla | |
| 231 | evqlvesggglvqpggslrlscaasgfnikdtyihwvrqapgkglewvariyptngytryadsv | 3315 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvipgcaqpwyhgetftctaahpelkt | |
| | pltanitksgntfrpevhllpppseelalnelvtltclargfspkdvlvrwlqgdqelprekyl | |
| | twasrqepsqgtttfavtsilrvaaedwkkgdtfscyvghealplaftqktidrla | |
| 232 | evqlvesggglvqpggslrlscaasgfnikdtyihwvrqapgkglewvariyptngytryadsv | 3316 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvipgcaepwnhgetftctaahpelkt | |
| | pltanitksgntfrpevhllpppseelalnelvtltclargfspkdvlvrwlqgdqelprekyl | |
| | twasrqepsqgtttfavtsilrvaaedwkkgdtfscyvghealplaftqktidrla | |
| 233 | evqlvesggglvqpggslrlscaasgfnikdtyihwvrqapgkglewvariyptngytryadsv | 3317 |
| | kgrftisadtskntaylqmnslraedtavyycsrwggdgfyamdywgqgtlvtvssasptspkv | |
| | fplsldstpqdgnvvvaclvqgffpqeplsvtwsesgqnvtarnfppsqdasgdlyttssqltl | |
| | patqcpdgksvtchvkhytnpsqdvtvpcrvpppppcchprlslhrpaledlllgseanltctl | |
| | tglrdasgatftwtpssgksavqgpperdlcgcysvssvipgcaepwyhgetftctaahpelkt | |
| | pltanitksgntfrpevhllpppseelalnelvtltclargfspkdvlvrwlqgdqelprekyl | |
| | twasrqepsqgtttfavtsilrvaaedwkkgdtfscyvghealplaftqktidrla | |
| 234 | evqlvesggglvqpggslrlscaasgfnikdtyihwvrqapgkglewvariyptngytryadsv | 3318 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhedwlngkeykck | |
| | vsnkalpapiektiskagqprepqvyllppsrdeltknqvsltclakgfypkdvlvrwlqgdqe | |
| | lprenykttasvldsdgsffvyskltvaaedwkkgdtfscmvghealplaftqksisrsp | |
| 235 | evqlvesggglvqpggslrlscaasgfnikdtyihwvrqapgkglewvariyptngytryadsv | 3319 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhedwlngkeykck | |
| | vsnkalpapiektiskagqprepqvyllppsrdeltknqvsltclakgfypkdvlvrwlqgsqe | |
| | lprenykttasvldsdgsffvyskltvaaedwkkgdtfscyvghealplaftqksisrsp | |
| 236 | evqlvesggglvqpggslrlscaasgfnikdtyihwvrqapgkglewvariyptngytryadsv | 3320 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwyngkeykck | |
| | vsnkalpapiektiskagqprepqvyllppsrdeltknqvsltclakgfypkdvlvrwlqgdqe | |
| | lprenykttasvldsdgsffvyskltvaaedwkkgdtfscmvghealplaftqksisrsp | |
| 237 | evqlvesggglvqpggslrlscaasgfnikdtyihwvrqapgkglewvariyptngytryadsv | 3321 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwyngkeykck | |
| | vsnkalpapiektiskagqprepqvyllppsrdeltknqvsltclakgfypkdvlvrwlqgsqe | |
| | lprenykttasvldsdgsffvyskltvaaedwkkgdtfscyvghealplaftqksisrsp | |
| 238 | evqlvesggglvqpggslrlscaasgfnikdtyihwvrqapgkglewvariyptngytryadsv | 3322 |
| | kgrftisadtskntaylqmnslraedtavyycsrwggdgfyamdywgqgtlvtvssastkgpsv | |
| | fplapsskstsggtaalgclvkdvfpepvtvswnsgaltsgvhtfpavlqssglvslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwlngkeykck | |
| | vsnkalpapiektiskagqprepqvyllppsrdeltknqvsltclakgfypkdvlvrwlqgdqe | |
| | lprenykttasvldsdgsffvyskltvaaedwkkgdtfscyvghealplaftqksisrsp | |
| 239 | evqlvesggglvqpggslrlscaasgfnikdtyihwvrqapgkglewvariyptngytryadsv | 3323 |
| | kgrftisadtskntaylqmnslraedtavyycsrwggdgfyamdywgqgtlvtvssastkgpsv | |
| | fplapsskstsggtaalgclvkdvfpepvtvswnsgaltsgvhtfpavlqssglvslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhedwyngkeykck | |
| | vsnkalpapiektiskagqprepqvyllppsrdeltknqvsltclakgfypkdvlvrwlqgsqe | |
| | lprenykttasvldsdgsffvyskltvaaedwkkgdtfscyvghealplaftqksisrsp | |
| 240 | evqlvesggglvqpggslrlscaasgfnikdtyihwvrqapgkglewvariyptngytryadsv | 3324 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhqdwyngkeykck | |
| | vsnkalpapiektiskagqprepqvyllppsrdeltknqvsltclakgfypkdvlvrwlqgdqe | |
| | lprenykttasvldsdgsffvyskltvaaedwkkgdtfscyvghealplaftqksisrsp | |
| 241 | evqlvesggglvqpggslrlscaasgfnikdtyihwvrqapgkglewvariyptngytryadsv | 3325 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhedwlngkeykck | |
| | vsnkalpapiektiskagqprepqvyllppsrdeltknqvsltclakgfypkdvlvrwlqgdqe | |
| | lprenykttasvldsdgsffvyskltvaaedwkkgdtfscyvghealplaftqksisrsp | |
| 242 | evqlvesggglvqpggslrlscaasgfnikdtyihwvrqapgkglewvariyptngytryadsv | 3326 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpcchprvflfrpaledlllgsean | |
| | vtcvvtglrdedpevkfnwyvdgvevhnaktkpreeqycgcysvvsvltvlhedwyngkeykck | |
| | vsnkalpapiektiskagqprepqvyllppsrdeltknqvsltclakgfypkdvlvrwlqgdqe | |
| | lprenykttasvldsdgsffvyskltvaaedwkkgdtfscyvghealplaftqksisrsp | |
| 243 | evqlvesggglvqpggslrlscaasgfnikdtyihwvrqapgkglewvariyptngytryadsv | 3327 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhedwlngke | |
| | ykckvsnkalpapiektiskagqprepqvyllppsrdeltknqvsltclakgfypkdvlvrwlq | |
| | gdqelprenykttasvldsdgsffvyskltvaaedwkkgdtfscmvghealplaftqksisrsp | |
| 244 | evqlvesggglvqpggslrlscaasgfnikdtyihwvrqapgkglewvariyptngytryadsv | 3328 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhedwlngke | |
| | ykckvsnkalpapiektiskagqprepqvyllppsrdeltknqvsltclakgfypkdvlvrwlq | |
| | gsqelprenykttasvidsdgsffvyskltvaaedwkkgdtfscyvghealplaftqksisrsp | |
| 245 | evqlvesggglvqpggslrlscaasgfnikdtyihwvrqapgkglewvariyptngytryadsv | 3329 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhqdwyngke | |
| | ykckvsnkalpapiektiskagqprepqvyllppsrdeltknqvsltclakgfypkdvlvrwlq | |
| | gdqelprenykttasvidsdgsffvyskltvaaedwkkgdtfscmvghealplaftqksisrsp | |
| 246 | evqlvesggglvqpggslrlscaasgfnikdtyihwvrqapgkglewvariyptngytryadsv | 3330 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhqdwyngke | |
| | ykckvsnkalpapiektiskagqprepqvyllppsrdeltknqvsltclakgfypkdvlvrwlq | |
| | gsqelprenykttasvidsdgsffvyskltvaaedwkkgdtfscyvghealplaftqksisrsp | |
| 247 | evqlvesggglvqpggslrlscaasgfnikdtyihwvrqapgkglewvariyptngytryadsv | 3331 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhqdwlngke | |
| | ykckvsnkalpapiektiskagqprepqvyllppsrdeltknqvsltclakgfypkdvlvrwlq | |
| | gdqelprenykttasvldsdgsffvyskltvaaedwkkgdtfscyvghealplaftqksisrsp | |
| 248 | evqlvesggglvqpggslrlscaasgfnikdtyihwvrqapgkglewvariyptngytryadsv | 3332 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhedwyngke | |
| | ykckvsnkalpapiektiskagqprepqvyllppsrdeltknqvsltclakgfypkdvlvrwlq | |
| | gsqelprenykttasvldsdgsffvyskltvaaedwkkgdtfscyvghealplaftqksisrsp | |
| 249 | evqlvesggglvqpggslrlscaasgfnikdtyihwvrqapgkglewvariyptngytryadsv | 3333 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhqdwyngke | |
| | ykckvsnkalpapiektiskagqprepqvyllppsrdeltknqvsltclakgfypkdvlvrwlq | |
| | gdqelprenykttasvldsdgsffvyskltvaaedwkkgdtfscyvghealplaftqksisrsp | |
| 250 | evqlvesggglvqpggslrlscaasgfnikdtyihwvrqapgkglewvariyptngytryadsv | 3334 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhedwlngke | |
| | ykckvsnkalpapiektiskagqprepqvyllppsrdeltknqvsltclakgfypkdvlvrwlq | |
| | gdqelprenykttasvldsdgsffvyskltvaaedwkkgdtfscyvghealplaftqksisrsp | |
| 251 | evqlvesggglvqpggslrlscaasgfnikdtyihwvrqapgkglewvariyptngytryadsv | 3335 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhedwyngke | |
| | ykckvsnkalpapiektiskagqprepqvyllppsrdeltknqvsltclakgfypkdvlvrwlq | |
| | gdqelprenykttasvldsdgsffvyskltvaaedwkkgdtfscyvghealplaftqksisrsp | |
| 252 | Evqlvesggglvqpggslrlscaasgfnikdtyihwvrqapgkglewvariyptngytryadsv | 3367 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqynstyrvvsvltvlhedwyngke | |
| | ykckvsnkalpapiektiskagqprepqvcllppsrdeltknqvslscaakgfypkdvlvrwlq | |
| | gdqelprenykttasvldsdgsffvvskitvaaedwkkgdtfscmvghealplaftqksisrsp | |
| 253 | qegdfpmpfisaksspvipidgsvkiqcqaireayltqlmiiknstyreigrrlkfwnetdpef | 2922 |
| | vidhmdankagryqcqyrighyrfrysdtlelvvtglygkpfisadrglvlmpgenisitessa | |
| | hipfdrfslakegelslpqhqsgehpanfslgpvdlnvsgiyrcygwynrspylwsfpsnalel | |
| | vvtdsihqdyttqnhhhhhhglndifeaqkiewhe | |
| 254 | cchprlslhrpaledlllgseanltctltglrdasgvtftwtpssgksavqgpperdlcgcysv | |
| | ssvlpgcaepwnhgktftctaaypesktpltatlsksgntfrpevhllpppseelalnelvtlt | |
| | clargfspkdvlvrwlqgsqelprekyltwasrqepsqgtttfavtsilrvaaedwkkgdtfsc | |
| | mvghealplaftqktidrla | |
| 255 | hhhhhh | |
| 256 | evqlvesggglvqpggslrlscaasgfnikdtyihwvrqapgkglewvariyptngytryadsv | 3400 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seaavtcvvvdvshedpevkfnwyvdgvevhnaktkpreeqynstyrvvsvltvlhedwyngke | |
| | ykckvsnkalpapiektiskagqprepqvyllppsrdeltknqvsltclakgfypkdvlvrwlq | |
| | gdqelprenykttasvldsdgsffvyskltvaaedwkkgdtfscmvghealplaftqksisrsp | |
| 257 | evqlvesggglvqpggslrlscaasgfnikdtyihwvrqapgkglewvariyptngytryadsv | 3401 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seanvtcvvvdvshedpevkfnwyvdgvevhnaktkpreeqyastyrvvsvltvlhedwyngke | |
| | ykckvsnkalpapiektiskagqprepqvyllppsrdeltknqvsltclakgfypkdvlvrwlq | |
| | gdqelprenykttasvldsdgsffvyskltvaaedwkkgdtfscmvghealplaftqksisrsp | |
| 258 | evqlvesggglvqpggslrlscaasgfnikdtyihwvrqapgkglewvariyptngytryadsv | 3402 |
| | kgrftisadtskntaylqmnslraedtavyycsrwggdgfyamdywgqgtlvtvssastkgpsv | |
| | fplapsskstsggtaalgclvkdyfpepvtvswnsgaltsgvhtfpavlqssglyslssvvtvp | |
| | ssslgtqtyicnvnhkpsntkvdkkvepkscdkthtcppcpapellggpsvflfrpaledlllg | |
| | seaavtcvvvdvshedpevkfnwyvdgvevhnaktkpreeqyastyrvvsvltvlhedwyngke | |
| | ykckvsnkalpapiektiskagqprepqvyllppsrdeltknqvsltclakgfypkdvlvrwlq | |
| | gdqelprenykttasvldsdgsffvyskltvaaedwkkgdtfscmvghealplaftqksisrsp | |

1. An engineered, human IgG1 immunoglobulin or fragment thereof comprising an Fc region comprising a first and second Fc domain, wherein an Fc domain comprises a CH2 and CH3 domain, and wherein the first Fc domain comprises at least one amino acid modification, and wherein the first Fc domain has an amino acid sequence at least 65% identical to an Fc domain of wild-type IgG1 (amino acids CH2-1.6 to CH3-125 (IMGT numbering for C-domain) of SEQ ID NO: 1), and wherein the engineered IgG1 immunoglobulin or fragment thereof binds to and activates human FcαRI.

2. The engineered immunoglobulin or fragment thereof of claim 1, wherein the at least one amino acid modification in the first Fc domain is a substitution that corresponds to an amino acid in an Fc domain of IgA1 (SEQ ID NO: 254), an Fc domain of wild-type IgA2 (amino acids CH2-1.2 to CH3-125 of SEQ ID NO: 2 (IMGT numbering for C-domain)), an Fc domain of parental IgA2 (amino acids CH2-1.2 to CH3-125 of SEQ ID NO: 3 (IMGT numbering for C-domain)) or an affinity matured variant Fc domain of IgA1 or IgA2.

3. The engineered immunoglobulin or fragment thereof of claim 1, wherein, as a result of the at least one amino acid modification in the first Fc domain, the engineered immunoglobulin or fragment thereof is capable of binding to human FcRn.

4. The engineered immunoglobulin or fragment thereof of claim 3, that is capable of binding to human FcRn with an affinity comparable to wild-type IgG1.

5. The engineered immunoglobulin or fragment thereof of claim 1, wherein the first Fc domain further comprises at least one additional amino acid modification, such that the engineered immunoglobulin or fragment thereof is capable of binding to human FcγRs.

6. The engineered immunoglobulin or fragment thereof of claim 1, wherein the engineered immunoglobulin or fragment thereof is a homodimer and wherein the second Fc domain has an amino acid sequence at least 65% identical to an Fc domain from wild-type IgG1 (SEQ ID NO: 1).

7. The engineered immunoglobulin or fragment thereof of claim 1, wherein the engineered immunoglobulin or fragment thereof is a heterodimer and wherein the second Fc domain has an amino acid sequence at least 70% identical to an Fc domain from wild-type IgG1 (SEQ ID NO: 1), and wherein the engineered immunoglobulin or fragment thereof binds to and activates human FcγRs.

8. The engineered immunoglobulin or fragment thereof of claim 7, wherein the second Fc domain binds to FcRn.

9. The engineered immunoglobulin or fragment thereof of claim 7, wherein the FcγRs are human FcγRIa and human FcγRIIIa.

10. The engineered immunoglobulin or fragment thereof of claim 1, wherein the first Fc domain has an amino acid sequence comprised within SEQ ID NO: 122, SEQ ID NO: 148 or SEQ ID NO: 214, and wherein the second Fc domain has an amino acid sequence comprised within SEQ ID NO: 122, SEQ ID NO: 148 or SEQ ID NO: 214, respectively.

11. The engineered immunoglobulin or fragment thereof of claim 3, wherein the first Fc domain has an amino acid sequence comprised within SEQ ID NO: 188 to 193, 234 to 242, 205 to 209 or 223 or has an amino acid sequence comprised within SEQ ID NO: 194 to 199, 244 to 251, 210 to 214 or 224.

12. The engineered immunoglobulin or fragment thereof of claim 5, wherein the first Fc domain has an amino acid sequence comprised within SEQ ID NO: 150, 151, 152 or 153.

13. The engineered immunoglobulin or fragment thereof of claim 7, wherein the first Fc domain has an amino acid sequence comprised within SEQ ID NO: 133, 135, 137, 139, 141, 143, 145, 155, 156, 157, 158 or 252 and wherein the second Fc domain has an amino acid sequence comprised within SEQ ID NO: 132, 134, 136, 138, 140, 142, 144, 154, 159, 160, 161, 162, 163 or 164.

14. The engineered immunoglobulin or fragment thereof of claim 13, wherein the first Fc domain has an amino acid sequence comprised within SEQ ID NO: 157 and the second Fc domain has an amino acid sequence comprised within SEQ ID NO: 159 or 161.

15. The engineered immunoglobulin or fragment thereof of claim 13, wherein the first Fc domain has an amino acid sequence comprised within SEQ ID NO: 252 and the second Fc domain has an amino acid sequence comprised within SEQ ID NO: 159 or 161.

16. The engineered immunoglobulin or fragment thereof of claim 1, further comprising a variable heavy chain domain and a light chain and wherein the engineered immunoglobulin or fragment thereof recognizes and binds to a target antigen.

17. The engineered immunoglobulin or fragment thereof of 16, wherein the target antigen is selected from the group consisting of: fibronectin EDA, HER2, EGFR, CD20, CD30, EpCAM, GD2 and solid tumor antigens.

18. A pharmaceutical composition comprising an engineered immunoglobulin or fragment thereof of claim 1 in combination with one or more pharmaceutically acceptable excipient, diluent or carrier.

19. The pharmaceutical composition according to claim 18, further comprising one or more additional therapeutic agents.

20.-21. (canceled)

22. A method of treating a cell proliferative disorder, the method comprising administering a therapeutically effective amount of an engineered immunoglobulin or fragment thereof of claim 1, to a subject in need thereof.

23. The method according to claim 22, wherein the proliferative disorder is selected from the group consisting of: fibrosis, breast cancer, neuroblastoma, lymphoma, colon carcinoma, epithelial cancer, colorectal cancer, renal cancer and mucosal tumors.

24. An isolated nucleic acid molecule or set of nucleic acid molecules encoding the engineered immunoglobulin or fragment thereof according to claim 1.

25. The isolated nucleic acid molecule or set of nucleic acid molecules according to claim 24, which is complementary DNA (cDNA) or messenger RNA (mRNA).

26. A cloning or expression vector comprising one or more nucleic acid molecules or set of nucleic acid molecules according to claim 24, wherein the vector is suitable for the recombinant production of the engineered immunoglobulin or fragment thereof.

27. A host cell comprising one or more cloning or expression vectors according to claim 26.

28. A process for the production of the engineered immunoglobulin or fragment thereof according to claim 1, comprising culturing a host cell according to claim 27 under conditions sufficient to express the engineered immunoglobulin or fragment thereof, and thereafter purifying and recovering the engineered immunoglobulin or fragment thereof from the host cell culture.

29. A kit comprising the engineered immunoglobulin or fragment thereof according to claim 1, wherein the kit additionally comprises instructions for use and means for administering the engineered immunoglobulin or fragment thereof or the pharmaceutical composition to a subject in need thereof.
